(19) United States
(12) Patent Application Publication
(10) Pub. No.: US 2025/0283071 A1
(43) Pub. Date: Sep. 11, 2025

(54) MODIFIED DOUBLE STRANDED OLIGONUCLEOTIDES

(71) Applicant: ALNYLAM PHARMACEUTICALS, INC., Cambridge, MA (US)

(72) Inventors: Mark K. SCHLEGEL, Cambridge, MA (US); Adam CASTORENO, Cambridge, MA (US); Christopher BROWN, Cambridge, MA (US); Lucas D. BONDURANT, Cambridge, MA (US); Anna BORODOVSKY, Cambridge, MA (US); William CANTLEY, Cambridge, MA (US); Elena CASTELLANOS-RIZALDOS, Cambridge, MA (US); Aimee M. DEATON, Cambridge, MA (US); Jonathan Edward FARLEY, Cambridge, MA (US); Elane FISHILEVICH, Cambridge, MA (US); John Michael GANSNER, Cambridge, MA (US); Jason GILBERT, Cambridge, MA (US); Vasant R. JADHAV, Cambridge, MA (US); Charalambos KAITTANIS, Cambridge, MA (US); Mark KEATING, Cambridge, MA (US); Jingxuan LIU, Cambridge, MA (US); James MCININCH, Cambridge, MA (US); Stuart MILSTEIN, Cambridge, MA (US); Bhaumik A. PANDYA, Cambridge, MA (US); Mangala Meenakshi SOUNDARAPANDIAN, Cambridge, MA (US); Jeffrey ZUBER, Cambridge, MA (US)

(73) Assignee: ALNYLAM PHARMACEUTICALS, INC., Cambridge, MA (US)

(21) Appl. No.: 18/262,302

(22) PCT Filed: Oct. 28, 2021
(86) PCT No.: PCT/US21/57016

§ 371 Date: Jul. 20, 2023

Related U.S. Application Data
(60) Provisional application No. 63/140,714, filed on Jan. 22, 2021.
(60) Provisional application No. 63/146,115, filed on Feb. 5, 2021.
(60) Provisional application No. 63/148,991, filed on Feb. 12, 2021.
(60) Provisional application No. 63/153,983, filed on Feb. 26, 2021.
(60) Provisional application No. 63/156,476, filed on Mar. 4, 2021.
(60) Provisional application No. 63/161,313, filed on Mar. 15, 2021.
(60) Provisional application No. 63/164,467, filed on Mar. 22, 2021.
(60) Provisional application No. 63/179,607, filed on Apr. 26, 2021.
(60) Provisional application No. 63/181,748, filed on Apr. 29, 2021.

(51) Int. Cl. C12N 15/113

(52) U.S. Cl. CPC C12N 15/113; C12N 2310/11; C12N 2310/312; C12N 2310/315; C12N 2310/321; C12N 2310/322; C12N 2310/3515

(57) ABSTRACT

One aspect of the present invention relates to double-stranded RNA (dsRNA) agent capable of inhibiting the expression of a target gene. Other aspects of the invention relate to pharmaceutical compositions comprising these dsRNA molecules suitable for therapeutic use, and methods of inhibiting the expression of a target gene by administering these dsRNA molecules, e.g., for the treatment of various disease conditions.

### CROSS-REFERENCE TO RELATED APPLICATIONS

This application is a 35 U.S.C. § 371 National Phase Entry Application of International Patent Application No. PCT/US2021/057016 filed on Oct. 28, 2021, which designated the U.S., and which claims benefit under 35 U.S.C. § 119(e) of U.S. Provisional Application No. 63/140,714 filed, Jan. 22, 2021, U.S. Provisional Application No. 63/146,115 filed Feb. 5, 2021, U.S. Provisional Application No. 63/148,991 filed, Feb. 12, 2021, U.S. Provisional Application No. 63/153,983 filed Feb. 26, 2021, U.S. Provisional Application No. 63/156,476 filed Mar. 4, 2021, U.S. Provisional Application No. 63/161,313 filed Mar. 15, 2021, U.S. Provisional Application No. 63/164,467 filed Mar. 22, 2021, U.S. Provisional Application No. 63/179,607 filed Apr. 26, 2021, and U.S. Provisional Application No. 63/181,748 filed Apr. 29, 2021, the contents of each of which are incorporated herein by reference in their entireties.

### SEQUENCE LISTING

The instant application contains a Sequence Listing which has been submitted electronically in ASCII format and is hereby incorporated by reference in its entirety. Said ASCII copy, created on Dec. 10, 2021, is named O51058-O99300WOPT_SL.txt and is 837,545 bytes in size.

### FIELD OF THE INVENTION

The invention relates to dsRNA molecules having particular motifs that are advantageous for inhibition of target gene expression, as well dsRNA agent compositions, suitable for therapeutic use. Additionally, the invention provides methods of inhibiting the expression of a target gene by administering these dsRNA agents, e.g., for the treatment of various diseases.

### BACKGROUND

RNA interference or “RNAi” is a term initially coined by Fire and co-workers to describe the observation that double-stranded RNAi (dsRNA) can block gene expression (Fire et al. (1998) Nature 391, 806-811; Elbashir et al. (2001) Genes Dev. 15, 188-200). Short dsRNA directs gene-specific, post-transcriptional silencing in many organisms, including vertebrates, and has provided a new tool for studying gene function. RNAi is mediated by RNA-induced silencing complex (RISC), a sequence-specific, multi-component nuclease that destroys messenger RNAs homologous to the silencing trigger. RISC is known to contain short RNAs (approximately 22 nucleotides) derived from the double-stranded RNA trigger, but the protein components of this activity remained unknown.

There remains a need in the art for effective nucleotide or chemical motifs for dsRNA molecules, which are advantageous for inhibition of target gene expression. This invention is directed to that effort.

### SUMMARY

This invention provides effective nucleotide or chemical motifs for dsRNA molecules, which are advantageous for inhibition of target gene expression, as well as RNAi compositions suitable for therapeutic use.

Inventors have discovered inter alia that double stranded RNA (dsRNA) molecules having a 2′-fluoro nucleotide at least at position 10 of the sense strand unexpectedly and surprisingly have imporved in vitro potentcy, i.e., increased RNA interference (RNAi) activity. Accordingly, in one aspect provided herein is a double stranded RNA (dsRNA) molecule comprising a sense strand and an antisense strand, each strand independently having a length of 15 to 35 nucleotides, and wherein the sense strand comprises a 2′-fluoro nucleotide at position 10, counting from 5′-end of the sense strand.

It is noted that the sense strand can further comprises one or more, e.g., 1, 2, 3, 4 or 5 additional 2′-fluoro nucleotides. Accordingly, in some embodiments, the sense strand comprises 1, 2, 3, 4, or 5 additional 2′-fluoro nucleotides. The additional 2′-fluoro nucleotides can be located anywhere in the sense strand. Thus, in some embodiments, the sense strand further comprises a 2′-fluoro nucleotide at position 8, 9, 11 or 12, counting from 5′-end of the sense strand. For example, the sense strand further comprises a 2′-fluoro nucleotide at position 9, counting from 5′-end of the sense strand. In other words, the sense strand comprises a 2′-fluoro nucleotide at positions 9 and 10, counting from 5′-end of the sense strand. In another example, the sense strand further comprises a 2′-fluoro nucleotide at position 11, counting from 5′-end of the sense strand. For example, the sense strand comprises a 2′-fluoro nucleotide at positions 10 and 11, counting from 5′-end of the sense strand.

In some embodiments, the sense strand comprises a 2′-fluoro nucleotide at positions 9, 10 and 11, counting from 5′-end of the sense strand. In some other embodiments, the sense strand comprises a 2′-fluoro nucleotide at positions 8, 9 and 10, counting from 5′-end of the sense strand. In yet some other embodiments, the sense strand comprises a 2′-fluoro nucleotide at positions 10, 11 and 12, counting from 5′-end of the sense strand.

In some embodiments of any one of the aspects, the sense strand does not comprise a 2′-fluoro nucleotide at position 7, counting from 5′-end of the sense strand. For example, the sense strand comprises a 2′-OMe nucleotide at position 7, counting from the 5′-end of the sense strand.

The antisense strand of the dsRNA molecules described herein can comprise one or more 2′-deoxy, e.g., 2′-H nucleotides. For example, the antisense strand comprises 1, 2, 3, 4, 5, 6 or more 2′-deoxy nucleotides. In some embodiments, the antisense strand comprises 2, 3, 4, 5 or 6 2′-deoxy nucleotides. The 2′-deoxy nucleotides can be located anywhere in the antisense strand. For example, the antisense strand comprises a 2′-deoxy nucleotide at 1, 2, 3, 4, 5 or 6 of positions 2, 5, 7, 12, 14 and 16, counting from 5′-end of the antisense strand. In some embodiments, the antisense comprises a 2′-deoxy nucleotide at positions 2 and 12, counting from 5′-end of the antisense strand. In some embodiments, the antisense comprises a 2′-deoxy nucleotide at positions 5 and 7, counting from 5′-end of the antisense strand. In some embodiments, the antisense comprises a 2′-deoxy nucleotide at positions 2, 5, 7 and 12, counting from 5′-end of the antisense strand.

In some embodiments, the antisense can comprise one or more, e.g., 1, 2, 3, 4, 5 or more of 2′-fluoro nucleotides. For example, the antisense strand can comprise a 2′-fluoro nucleotide at position 14, counting from 5′-end of the antisense strand.

In some embodiments, the antisense strand comprises a 2′-fluoro nucleotide at position 14 and a nucleotide other than a 2′-deoxy or 2′-fluoro at position 16, ounting from 5′-end of the antisense strand. For example, the antisense strand comprises a 2′-fluoro nucleotide at position 14 and a nucleotide other than a 2′-deoxy or 2′-fluoro at position 16, ounting from 5′-end of the antisense strand

In some embodiments, the antisense strand comprises a 2′-deoxy nucleotide at positions 2 and 12 and 2′-fluoro nucleotide at position 14, counting from 5′-end of the antisense strand. In some embodiments, the antisense strand comprises a 2′-deoxy nucleotide at positions 2 and 12, a 2′-fluoro nucleotide at position 14, and a nucleotide other than a 2′-deoxy or 2′-fluoro at position 16, counting from 5′-end of the antisense strand. For example, the antisense strand comprises a 2′-deoxy nucleotide at positions 2 and 12, a 2′-fluoro nucleotide at position 14, and a 2′-OMe nucleotide at position 16, counting from 5′-end of the antisense strand.

In some embodiments, the antisense strand comprises a 2′-deoxy nucleotide at position 14, counting from the 5′-end of the antisense stand, and the sense strand comprises a nucleotide other than a 2′-fluoro at position 7, counting from 5′-end of the sense strand. For example, the antisense strand comprises a 2′-deoxy nucleotide at positions 2, 12 and 14, counting from the 5′-end of the antisense stand, and the sense strand comprises a 2′-fluoro nucleotide at position 10 and anucleotide other than a 2′-fluoro at position 7, counting from 5′-end of the sense strand.

In various embodiments, the dsRNA molecule has a double stranded (duplex) region of between 19 to 25 base pairs. For example, the dsRNA molecule has a duplex region of 20, 21, 22, 23 or 24 basepairs. In some particular embodiments, the dsRNA molecule has a double duplex) region of 20, 21 or 22 base pairs.

In some embodiments, the dsRNA molecule comprises a ligand. For example, the sense strand of the dsRNA molecule comprises a ligand. Exemplary ligands include, but are not limited to, ASGPR ligands and ligands comprising a lipophilic group.

The dsRNA molecule can comprise one or more, e.g., 1, 2, 3, 4, 5, 6, 7, 8 or more phosphorothioate linkages. The phosphorothioate linkages can be present only in one of the strands or in both strands of the dsRNA. For example, the sense strand can comprise 1, 2, 3 or 4 phosphorothioate linkages. In another non-limiting example, the antisense strand can comprise 1, 2, 3, 4, 5 or 6 phosphorothioate linkages. In some embodiments, the sense strand comprises 1, 2, 3 or 4 phosphorothioate linkages and the antisense independently comprises 1, 2, 3, 4, 5, or 6 phosphorothioate linkages. For example, the sense strand comprises 1 or 2 phosphorothioate linkages and the antisense strand comprises 1, 2, 3 or 4 phosphorothioate linkages.

In some embodiments, the sense strand comprises at least two phosphorothioate internucleotide linkages between the first five nucleotides counting from the 5′ end of the sense strand, the antisense strand comprises at least two phosphorothioate internucleotide linkages between the first five nucleotides counting from the 5′-end of the antisense strand and the antisense further comprises at least two phosphorothioate internucleotide linkages between the first five nucleotides counting from the 3′-end of the antisense strand. For example, the sense strand comprises phosphorothioate linkages between nucleotides 1 and 2, and between nucleotides 2 and 3, counting from 5′-end of the sense strand, and the antisense strand comprises phosphorothioate linkages and between nucleotides 1 and 2, and between nucleotides 2 and 3, counting from 5′-end of the antisense strand, and between nucleotides 1 and 2, and between nucleotides 2 and 3, counting from 3′-end of the antisense strand.

In some embodiments, the remaning nucleotides in the dsRNA are 2′-OMe nucleotides. For example, all of the remaining nucleotides in the sense strand are 2′-OMe nucleotides. In other words, the sense strand solely comprises 2′-fluoro and 2′-OMe nucleotides.

It is understood that the antisense strand has sufficient complementarity to a target sequence to mediate RNA interference. In other words, the dsRNA molecules of the invention are capable of inhibiting the expression of a target gene.

In another aspect, the invention further provides a method for delivering the dsRNA molecule of the invention to a specific target in a subject by subcutaneous or intravenous administration. The invention further provides the dsRNA molecules of the invention for use in a method for delivering said agents to a specific target in a subject by subcutaneous or intravenous administration.

### BRIEF DESCRIPTION OF THE DRAWINGS

This patent or application file contains at least one drawing executed in color. Copies of this patent or patent application publication with color drawing(s) will be provided by the Office upon request and payment of the necessary fee.

FIGS. 1A and 1B are graphs showing dsRNAs according to exemplary embodiments of the invention have improved in vitro potency relative to the parent dsRNA molecules when dosed at 10 nM (FIG. 1A) or at 1 nM (FIG. 1).

FIGS. 2A-2D are graphs showing that dsRNA molecules according to embodiments of the invention have improved in vivo efficacy compared to the parent molecules. Parent duplexes are AD-1181401(Sequence 1, FIG. 2A); AD-1181410(Sequence 2, FIG. 2B); AD-1181426 (Sequence 3, FIG. 2C); and AD-1181451(Sequence 4, FIG. 2D)

FIGS. 3A-3D are graphs showing dsRNAs according to exemplary embodiments of the invention and targeting different targets have improved in vitro potency relative to the parent dsRNA molecules when dosed at 1 nM (FIGS. 3A and 3B) or at 0.1 nM (FIG. 3C).

FIGS. 4A-4C are graphs showing that presence of a 2′-fluoro nucleotide at position 10 of the sense strand, counting from the 5′-end of the sense strand, enhances the RNAi efficacy of the dsRNA molecule comprared to the parent.

FIGS. 5A and 5B are schematic representation of some exemplary designs of dsRNA molecules accorsing to embodiments of the invention.

FIGS. 6A and 6B are graphs showing improved in vitro target knockdown (FIG. 6A) and log 2 activity (FIG. 6B) of exemplary dsRNA according to some embodiments of the disclosure relative to exemplary parent dsRNA molecules.

FIGS. 7A and 7B are graphs showing improved in vitro target knockdown of AGT (FIG. 6A) and log 2 activity (FIG. 6B) of exemplary dsRNA according to some embodiments of the disclosure relative to exemplary parent dsRNA molecules targeting AGT.

FIGS. 8A-8H are showing similar or improved metabolic stability of sense strand (FIGS. 8A-8D) and antisense strand (FIGS. 8E-8H) of exemplary dsRNA molecules in mouse liver homogenate (FIGS. 8A and 8E), rat liver homogenate (FIGS. 8B and 8F), rat brain homogenate (FIGS. 8C and 8G), and cynomologus liver homogenate (FIGS. 8D and 8H). Parent duplexes are AD-1181401(TTR Seq 1); AD-1181410(TTR Seq 2); and AD-74210(F12).

FIGS. 9A and 9B are showing similar or improved metabolic stability of exemplary dsRNA molecules in mouse. Parent duplexes are AD-1181401(TTR Seq 1); AD-1181410(TTR Seq 2); and AD-74210(F12).

FIGS. 10A-10D are graphs showing that dsRNA molecules according to embodiments of the invention have improved in vivo efficacy and/or duration in non-human primates, mice (FIGS. 10A and 10B) and in cynomologus monkeys (FIGS. 10C and 10D) compared to the parent molecules AD-74210(FIGS. 10A and 10C) and AD-75885(FIGS. 10B and 10D).

### DETAILED DESCRIPTION

In one aspect, the invention provides a double-stranded RNA (dsRNA) agent capable of inhibiting expression of a target gene. Without limitations, the dsRNA agents of the invention can be substituted for the dsRNA molecules and can be used in RNA interference based gene silencing techniques, including, but not limited to, in vitro or in vivo applications.

Generally, the dsRNA molecule comprises a sense strand (also referred to as passenger strand) and an antisense strand (also referred to as guide strand). Each strand of the dsRNA molecule can range from 15-35 nucleotides in length. For example, each strand can be between, 17-35 nucleotides in length, 17-30 nucleotides in length, 25-35 nucleotides in length, 27-30 nucleotides in length, 17-23 nucleotides in length, 17-21 nucleotides in length, 17-19 nucleotides in length, 19-25 nucleotides in length, 19-23 nucleotides in length, 19-21 nucleotides in length, 21-25 nucleotides in length, or 21-23 nucleotides in length. Without limitations, the sense and antisense strands can be equal length or unequal length. For example, the sense strand and the antisense strand independently have a length of 18, 19, 20, 21, 22, 23, 24 or 25 nucleotides.

In some embodiments, the antisense strand is of length 15-35 nucleotides. In some embodiments, the antisense strand is 15-35, 17-35, 17-30, 25-35, 27-30, 17-23, 17-21, 17-19, 19-25, 19-23, 19-21, 21-25, 21-25, or 21-23 nucleotides in length. For example, the antisense strand can be 15, 16, 17, 18, 19, 20, 21, 22, 23, 24, 25, 26, 27, 28, 29, 30, 31, 32, 33, 34 or 35 nucleotides in length. In some embodiments, the antisense strand is 19, 20, 21, 22, 23, 24 or 25 nucleotides in length. For example, the antisense strand is 21, 22, 23, 24 or 25 nucleotides in length. In some particular embodiments, the antisense strand is 22, 23 or 24 nucleotides in length. For example, the antisense strand is 23 nucleotides in length.

Similar to the antisense strand, the sense strand can be, in some embodiments, 15-35 nucleotides in length. In some embodiments, the sense strand is 15-35, 17-35, 17-30, 25-35, 27-30, 17-23, 17-21, 17-19, 19-25, 19-23, 19-21, 21-25, 21-25, or 21-23 nucleotides in length. For example, the sense strand can be 15, 16, 17, 18, 19, 20, 21, 22, 23, 24, 25, 26, 27, 28, 29, 30, 31, 32, 33, 34 or 35 nucleotides in length. In some embodiments, the sense strand is 17, 18, 19, 20, 21, 22, 23, 24 or 25 nucleotides in length. For example, the sense strand is 19, 20, 21, 22 or 23 nucleotides in length. In some particular embodiments, the sense strand is 20, 21 or 22 nucleotides in length. For example, the sense strand is 21nucleotides in length

In some embodiments, the sense strand can be 15-35 nucleotides in length, and the antisense strand can be independent from the sense strand, 15-35 nucleotides in length. In some embodiments, the sense strand is 15-35, 17-35, 17-30, 25-35, 27-30, 17-23, 17-21, 17-19, 19-25, 19-23, 19-21, 21-25, 21-25, or 21-23 nucleotides in length, and the antisense strand is independently 15-35, 17-35, 17-30, 25-35, 27-30, 17-23, 17-21, 17-19, 19-25, 19-23, 19-21, 21-25, 21-25, or 21-23 nucleotides in length. For example, the sense and the antisense strand can be independently 15, 16, 17, 18, 19, 20, 21, 22, 23, 24, 25, 26, 27, 28, 29, 30, 31, 32, 33, 34 or 35 nucleotides in length. In some embodiments, the sense strand and the antisense strand are independently 17, 18, 19, 20, 21, 22, 23, 24 or 25 nucleotides in length. For example, the sense strand is 19, 20, 21, 22 or 23 nucleotides in length and the antisense strand is 21, 22, 23, 24 or 25 nucleotides in length. In some particular embodiments, the sense strand is 20, 21 or 22 nucleotides in length and the antisense strand is 22, 23 or 24 nucleotides in length. For example, the sense strand is 21 nucleotides in length and the antisense strand is 23 nucleotides in length.

The sense strand and antisense strand typically form a double-stranded or duplex region. Without limitations, the duplex region of a dsRNA agent described herein can be 12-35 nucleotide (or base) pairs in length. For example, the duplex region can be between 14-35 nucleotide pairs in length, 17-30 nucleotide pairs in length, 25-35 nucleotides in length, 27-35 nucleotide pairs in length, 17-23 nucleotide pairs in length, 17-21 nucleotide pairs in length, 17-19 nucleotide pairs in length, 19-25 nucleotide pairs in length, 19-23 nucleotide pairs in length, 19-21 nucleotide pairs in length, 21-25 nucleotide pairs in length, or 21-23 nucleotide pairs in length. In another example, the duplex region is selected from 15, 16, 17, 18, 19, 20, 21, 22, 23, 24, 25, 26, and 27 nucleotide pairs in length. In some embodiments, the duplex region is 18, 19, 20, 21, 22, 23, 24 or 25 nucleotide pairs in length. For example, the duplex region is 19, 20, 21, 22 or 23 nucleotide pairs in length. In some embodiments, the duplex region is 20, 21 or 22 nucleotide pairs in length. For example, the dsRNA molecule has a duplex region of 21 base pairs.

As described herein, the dsRNA molecule of the invention can further comprise at least one, e.g., 1, 2, 3, 4, 5, 6, 7, 8, 9, 10 or more 2′-deoxy, e.g., 2′-H nucleotides. For example, the dsRNA can comprise 1, 2, 3, 4, 5, 6, 7, 8, 9 or 10 2′-deoxy, e.g., 2′-H nucleotides. The 2′-deoxy nucleotide may occur on any nucleotide of the sense strand or antisense strand or both in any position of the strand. In one non-limiting example, the sense strand does not comprise a 2′-deoxy nucleotide at position 11, counting from 5′-end of the sense strand.

In some embodiments, the antisense strand the antisense strand comprises 1, 2, 3, 4, 5 or 6 of 2′-deoxy nucleotides. For example, antisense strand can comprise 2, 3, 4, 5 or 6 of 2′-deoxy nucleotides. The 2′-deoxy nucleotides can be located anywhere in the antisense strand. For example, the antisense strand comprises a 2′-deoxy nucleotide at 1, 2, 3, 4, 5 or 6 of positions 2, 5, 7, 12, 14 and 16, counting from 5′-end of the antisense strand. In one non-limiting example, the antisense strand comprises a 2′-deoxy nucleotide at 1, 2, 3 or 4 of positions 2, 5, 7, and 12, counting from 5′-end of the antisense strand.

In some embodiments, the antisense comprises a 2′-deoxy nucleotide at positions 5 and 7, counting from 5′-end of the antisense strand. For example, the antisense strand comprises a 2′-deoxy nucleotide at positions 5, 7 and 12, counting from 5′-end of the antisense strand. In some embodiments, the antisense strand comprises a 2′-deoxy nucleotide at positions 2, 5 and 7, counting from 5′-end of the antisense strand. For example, the antisense strand comprises a 2′-deoxy nucleotide at positions 2, 5, 7 and 12, counting from 5′-end of the antisense strand. In some embodiments, the antisense strand comprises a 2′-deoxy nucleotide at positions 2, 5, 7, 12 and 14, counting, from 5′-end of the antisense strand. For example, the antisense strand comprises a 2′-deoxy nucleotide at positions 2, 5, 7, 12, 14 and 16, counting from 5′-end of the antisense strand

In some embodiments, the antisense comprises a 2′-deoxy nucleotide at position 2 or 12, counting from 5′-end of the antisense strand. For example, the antisense comprises a 2′-deoxy nucleotide at position 12, counting from 5′-end of the antisense strand.

In some embodiments, the sense strand comprises a 2′-fluoro nucleotide at position 10, counting from 5′-end of the sense strand, and the antisense comprises a 2′-deoxy nucleotide at positions 5 and 7, counting from 5′-end of the antisense strand. For example, the sense strand comprises a 2′-fluoro nucleotide at positions 9 and 10, counting from 5′-end of the sense strand, and the antisense comprises a 2′-deoxy nucleotide at positions 5 and 7, counting from 5′-end of the antisense strand. In another example, the sense strand comprises a 2′-fluoro nucleotide at positions 8, 9 and 10, counting from 5′-end of the sense strand, and the antisense comprises a 2′-deoxy nucleotide at positions 5 and 7, counting from 5′-end of the antisense strand.

In some embodiments, the sense strand comprises a 2′-fluoro nucleotide at positions 10 and 11, counting from 5′-end of the sense strand, and the antisense comprises a 2′-deoxy nucleotide at positions 5 and 7, counting from 5′-end of the antisense strand. For example, the sense strand comprises a 2′-fluoro nucleotide at positions 10, 11 and 12, counting from 5′-end of the sense strand, and the antisense comprises a 2′-deoxy nucleotide at positions 5 and 7, counting from 5′-end of the antisense strand.

In some embodiments of any one of the aspects, the sense strand does not comprise a 2′-fluoro nucleotide at position 7, counting from 5′-end of the sense strand. For example, the sense strand comprises a 2′-fluoro nucleotide at at least one e.g., 1, 2 or 3 of positions 9, 10 and 11 but does not comprise a 2′-fluoro nucleotide at position 7, counting from 5′-end of the sense strand. In some embodiments, the sense strand comprises a 2′-fluoro nucleotide at position 10 and a nucleotide other than a 2′-fluoro nucleotide at position 7, counting from 5′-end of the sense strand.

For example, the sense strand comprises a 2′-fluoro nucleotide position 10 and a 2′-OMe nucleotide at position 7, counting from the 5′-end of the sense strand.

In some embodiments, the sense strand comprises a 2′-fluoro nucleotide at positions 9 and 10, and a nucleotide other than a 2′-fluoro nucleotide at position 7, counting from 5′-end of the sense strand. For example, the sense strand comprises a 2′-fluoro nucleotide at positions 9 and 10, and a 2′-OMe nucleotide at position 7, counting from 5′-end of the sense strand.

In some embodiments, the sense strand comprises a 2′-fluoro nucleotide at positions 10 and 11, and a nucleotide other than a 2′-fluoro nucleotide at position 7, counting from 5′-end of the sense strand. For example, the sense strand comprises a 2′-fluoro nucleotide at positions 10 and 11, and a 2′-OMe nucleotide at position 7, counting from 5′-end of the sense strand.

In some embodiments, the sense strand comprises a 2′-fluoro nucleotide at positions 9, 10 and 11, and a nucleotide other than a 2′-fluoro nucleotide at position 7, counting from 5′-end of the sense strand. For example, the sense strand comprises a 2′-fluoro nucleotide at positions 9, 10 and 11, and a 2′-OMe nucleotide at position 7, counting from 5′-end of the sense strand.

In some embodiments, the sense strand comprises a 2′-fluoro nucleotide at positions 9, 10 and 11, counting from 5′-end of the sense strand, and the remaining nucleotides in the sense strand are 2′-OMe nucleotides.

2′-flouro modifications (antisense strand)

The dsRNA molecules of the invention comprise one or more (e.g., one, two, three, four, five, six, seven, eight, nine, ten or more)₂′-fluoro nucleotides. Without limitations, the 2′-fluoro nucleotides all can be present in one strand. In some embodiments, both the sense and the antisense strands comprise at least one, 2′-fluoro nucleotide. The 2′-fluoro modification can occur on any nucleotide of the sense strand or antisense strand. For instance, the 2′-fluoro modification can occur on every nucleotide on the sense strand and/or antisense strand; each 2′-fluoro modification can occur in an alternating pattern on the sense strand or antisense strand; or the sense strand or antisense strand comprises both 2′-fluoro modifications in an alternating pattern. The alternating pattern of the 2′-fluoro modifications on the sense strand may be the same or different from the antisense strand, and the alternating pattern of the 2′-fluoro modifications on the sense strand can have a shift relative to the alternating pattern of the 2′-fluoro modifications on the antisense strand.

In some embodiments, the antisense strand comprises at least two (e.g., two, three, four, five, six, seven, eight, nine, ten or more)₂′-fluoro nucleotides. Without limitations, a 2′-fluoro modification in the antisense strand can be present at any positions.

In some embodiments, the antisense comprises one or more, e.g., 1, 2, 3, 4, 5 or more 2′-fluoro nucleotides. For example, the antisense strand comprises 1, 2, 3, 4, or 5 more 2′-fluoro nucleotides. In some embodiments, the antisense strand comprises 1, 2 or 3 2′-fluoro nucleotides. For example, the antisense strand comprises a single 2′-fluoro nucleotide. It is noted that a 2′-fluoro nucleotide can be located anywhere in the antisense strand. For example, a 2′-fluoro nucleotide can be at position 2 or 14, counting from 5′-end, of the antisense strand. In some embodiments, the antisense comprises a 2′-fluoro nucleotide at position 14, counting from 5′-end, of the antisense strand.

In some embodiments, the antisense comprises a 2′-fluoro nucleotide at position 14 and 2′-deoxy nucleotides at positions 5 and 7, counting from 5′-end of the antisense strand. For example, the antisense comprises a 2′-fluoro nucleotide at position 14 and 2′-deoxy nucleotides at positions 5, 7 and 12, counting from 5′-end of the antisense strand. In a further example, the antisense comprises a 2′-fluoro nucleotide at position 14 and 2′-deoxy nucleotides at positions 2, 5, 7 and 12, counting from 5′-end of the antisense strand.

In some embodiments, the antisense strand comprises a 2′-deoxy nucleotide at positions 2 and 12, counting from the 5′-end of the antisense strand. In some embodiments, the antisense strand further comprises a 2′-fluoro nucleotide at position 14, counting from the 5′-end of the antisense strand. For example, the antisense strand comprises a 2′-deoxy nucleotide at positions 2 and 12 and a 2′-fluoro nucleotide at position 14, counting from the 5′-end of the antisense strand.

In some embodiments, the antisense strand comprises a nucleotide other than a 2′-deoxy nucleotide at position 16, counting from the 5′-end of the antisense strand. In some embodiments, antisense strand comprises a nucleotide other than a 2′-fluoro nucleotide at position 16, counting from the 5′-end of the antisense strand. For example, the antisense strand comprises a 2′-deoxy nucleotide at positions 2 and 12 and a nucleotide other than a 2′-deoxy or 2′-fluoro nucleotide at position 16, counting from the 5′-end of the antisense strand. In some embodiments, the antisense strand comprises a 2′-deoxy nucleotide at positions 2 and 12 and a 2′-OMe nucleotide at position 16, counting from the 5′-end of the antisense strand.

In some embodiments, the antisense strand comprises a 2′-deoxy nucleotide at positions 2 and 12, a 2′-fluoro nucleotide at position 14 and a nucleotide other than a 2′-deoxy at position 16, counting from the 5′-end of the antisense strand. In some embodiments, the antisense strand comprises a 2′-deoxy nucleotide at positions 2 and 12, a 2′-fluoro nucleotide at position 14 and a nucleotide other than a 2′-fluoro at position 16, counting from the 5′-end of the antisense strand. For example, the antisense strand comprises a 2′-deoxy nucleotide at positions 2 and 12, a 2′-fluoro nucleotide at position 14 and a nucleotide other than a 2′-deoxy or 2′-fluoro nucleotide at position 16, counting from the 5′-end of the antisense strand. In some embodiments, the antisense strand comprises a 2′-deoxy nucleotide at positions 2 and 12, a 2′-fluoro nucleotide at position 14, and a 2′-OMe nucleotide at position 16, counting from the 5′-end of the antisense strand.

In some embodiments, the antisense strand comprises a 2′-deoxy nucleotide at positions 2, 5, 7 and 12 and a 2′-fluoro nucleotide at position 14, counting from the 5′-end of the antisense strand, and the remaining nucleotides in the antisense strand are 2′-OMe nucleotides.

In some embodiments, the antisense strand comprises a 2′-deoxy nucleotide at positions 2, 5, 7 and 12 and a 2′-fluoro nucleotide at position 14, counting from the 5′-end of the antisense strand, the sense strand comprises a 2′-fluoro nucleotide at positions 9, 10 and 11, counting from 5′-end of the sense strand, and the remaining nucleotides in the antisense strand and the sense strand are 2′-OMe nucleotides.

In some embodiments, the antisense strand comprises a 2′-deoxy nucleotide at positions 2, 5, 7, 12 and 14, counting from the 5′-end of the antisense strand, and the remaining nucleotides in the antisense strand are 2′-OMe nucleotides.

In some embodiments, the antisense strand comprises a 2′-deoxy nucleotide at positions 2, 5, 7, 12 and 14, counting from the 5′-end of the antisense strand, the sense strand comprises a 2′-fluoro nucleotide at positions 9, 10 and 11, counting from 5′-end of the sense strand, and the remaining nucleotides in the antisense strand and the sense strand are 2′-OMe nucleotides.

In some embodiments, the antisense strand comprises a 2′-deoxy nucleotide at positions 2, 5, 7, 12 and 16, counting from the 5′-end of the antisense strand, and the remaining nucleotides in the antisense strand are 2′-OMe nucleotides.

In some embodiments, the antisense strand comprises a 2′-deoxy nucleotide at positions 2, 5, 7, 12 and 16, counting from the 5′-end of the antisense strand, the sense strand comprises a 2′-fluoro nucleotide at positions 9, 10 and 11, counting from 5′-end of the sense strand, and the remaining nucleotides in the antisense strand and the sense strand are 2′-OMe nucleotides.

In some embodiments, the antisense strand comprises a 2′-deoxy nucleotide at positions 2, 5, 7, 12, 14 and 16, counting from the 5′-end of the antisense strand, and the remaining nucleotides in the antisense strand are 2′-OMe nucleotides.

In some embodiments, the antisense strand comprises a 2′-deoxy nucleotide at positions 2, 5, 7, 12, 14 and 16, counting from the 5′-end of the antisense strand, the sense strand comprises a 2′-fluoro nucleotide at positions 9, 10 and 11, counting from 5′-end of the sense strand, and the remaining nucleotides in the antisense strand and the sense strand are 2′-OMe nucleotides.

It is noted the remaining nucleotides, i.e., at positions not explicitly defined in the sense strand and/or the antisense strand can be unmodified or modified nucleotides. Accordingly, in some embodiments, the remaining nucleotides, i.e., at positions not explicitly defined in the sense strand are unmodified or modified nucleotides. For example, the remaining nucleotides, i.e., at positions not explicitly defined in the sense strand can be modified nucleotides selected from the group consisting of 2′-OMe, 2′-F, 2′-H, and an 2′-O—C10-30aliphatic group, optionally provided no more than one modified nucleotide is an 2′-O—C10-30aliphatic group.

In some embodiments, the remaining nucleotides, i.e., at positions not explicitly defined in the antisense strand are unmodified or modified nucleotides. For example, the remaining nucleotides, i.e., at positions not explicitly defined in the antisense strand can be modified nucleotides. In some embodiments, the remaining nucleotides, i.e., at positions not explicitly defined in the antisense strand can be selected from the group consisting of 2′-OMe, 2′-F, 2′-H, GNA and 3′-RNA, the 3′-RNA being optionally 3′-OH, provided no more than one modified nucleotide is GNA or 3′-RNA.

In some embodiments, the remaining nucleotides in the sense strand and/or the antisense strand are 2′-OMe nucleotides.

As described herein, the dsRNA agent can comprise one or more, e.g., 1, 2, 3, 4, 5, 6, 7, 8, 9, 10 or more nucleotides comprising a modified sugar. By a “modified sugar” is meant a sugar other than 2′-deoxy (i.e, 2′-H), 2′-OH, 2′-F or 2′-OMe ribose sugar. Some exemplary nucleotides comprising a modified sugar are locked nucleic acid (LNA), HNA, CeNA, 2′-methoxyethyl, 2′-O-allyl, 2′-C-allyl, 2′-O—N-methylacetamido (2′-O-NMA), a 2′-O-dimethylaminoethoxyethyl(2′-O-DMAEOE), 2′-O-aminopropyl(2′-O-AP), and 2′-ara-F. Accordingly, in some embodiments, the dsRNA agent can comprise one or more, e.g., 1, 2, 3, 4, 5, 6, 7, 8, 9, 10 or more nucleotides independently selected from the group consisting of acyclic nucleotides, locked nucleic acid (LNA), HNA, CeNA, 2′-methoxyethyl, 2′-O-allyl, 2′-C-allyl, 2′-O—N-methylacetamido (2′-O-NMA), a 2′-O-dimethylaminoethoxyethyl(2′-O-DMAEOE), 2′-O-aminopropyl(2′-O-AP), and 2′-ara-F. A nucleotide comprising modified sugar can be present anywherein the dsRNA molecule. For example, a nucleotide comprising a modified sugar can be present in the sense strand or a nucleotide comprising a modified sugar can be present in the antisense strand. When two or more nuelcotides comprising a modified sugar are present in the dsRNA molecule, they can all be in the sense strand, antisense strand or both in the sense and antisense strands.

In some embodiments, an unmodified nucleotide is a 2′-OH nucleotide comprising an unmodified nucleobase, i.e., adenine, guanine, cytosine, or uracil.

In some embodiments, the dsRNA can comprise one or more, e.g., 1, 2, 3, 4, 5, 6, 7, 8, 9, 10 or more nucleotides comprising a non-natural nucleobase. By a “non-natural nucleobase” is meant a nucleobase other than adenine, guanine, cytosine, uracil, or thymine. Exemplary non-natural nucleobases include, but are not limited to, inosine, xanthine, hypoxanthine, nubularine, isoguanisine, tubercidine, and substituted or modified analogs of adenine, guanine, cytosine and uracil, such as 2-aminoadenine, 6-methyl and other alkyl derivatives of adenine and guanine, 2-propyl and other alkyl derivatives of adenine and guanine, 5-halouracil and cytosine, 5-propynyl uracil and cytosine, 6-azo uracil, cytosine and thymine, 5-uracil (pseudouracil), 4-thiouracil, 5-halouracil, 5-(2-aminopropyl) uracil, 5-amino allyl uracil, 8-halo, amino, thiol, thioalkyl, hydroxyl and other 8-substituted adenines and guanines, 5-trifluoromethyl and other 5-substituted uracils and cytosines, 7-methylguanine, 5-substituted pyrimidines, 6-azapyrimidines and N-2, N-6 and O-6 substituted purines, including 2-aminopropyladenine, 5-propynyluracil and 5-propynylcytosine, dihydrouracil, 3-deaza-5-azacytosine, 2-aminopurine, 5-alkyluracil, 7-alkylguanine, 5-alkyl cytosine, 7-deazaadenine, N6, N6-dimethyladenine, 2,6-diaminopurine, 5-amino-allyl-uracil, N3-methyluracil, substituted 1,2,4-triazoles, 2-pyridinone, 5-nitroindole, 3-nitropyrrole, 5-methoxyuracil, uracil-5-oxyacetic acid, 5-methoxycarbonylmethyluracil, 5-methyl-2-thiouracil, 5-methoxycarbonylmethyl-2-thiouracil, 5-methylaminomethyl-2-thiouracil, 3-(3-amino-3carboxypropyl) uracil, 3-methylcytosine, 5-methylcytosine, N4-acetyl cytosine, 2-thiocytosine, N6-methyladenine, N6-isopentyladenine, 2-methylthio-N6-isopentenyladenine, N-methylguanines, or O-alkylated bases. Further purines and pyrimidines include those disclosed in U.S. Pat. No. 3,687,808, those disclosed in the Concise Encyclopedia of Polymer Science and Engineering, pages 858-859, Kroschwitz, J. I., ed. John Wiley & Sons, 1990, and those disclosed by Englisch et al., Angewandte Chemie, International Edition, 1991, 30, 613.

In some embodiments, the non-natural nucleobase can be selected from the group consisting of inosine, xanthine, hypoxanthine, nubularine, isoguanisine, tubercidine, 2-(halo) adenine, 2-(alkyl) adenine, 2-(propyl) adenine, 2-(amino) adenine, 2-(aminoalkyll) adenine, 2-(aminopropyl) adenine, 2-(methylthio)-N⁶-(isopentenyl) adenine, 6-(alkyl) adenine, 6-(methyl) adenine, 7-(deaza) adenine, 8-(alkenyl) adenine, 8-(alkyl) adenine, 8-(alkynyl) adenine, 8-(amino) adenine, 8-(halo) adenine, 8-(hydroxyl) adenine, 8-(thioalkyl) adenine, 8-(thiol) adenine, N⁶-(isopentyl) adenine, N⁶-(methyl) adenine, N⁶, N⁶-(dimethyl) adenine, 2-(alkyl) guanine, 2-(propyl) guanine, 6-(alkyl) guanine, 6-(methyl) guanine, 7-(alkyl) guanine, 7-(methyl) guanine, 7-(deaza) guanine, 8-(alkyl) guanine, 8-(alkenyl) guanine, 8-(alkynyl) guanine, 8-(amino) guanine, 8-(halo) guanine, 8-(hydroxyl) guanine, 8-(thioalkyl) guanine, 8-(thiol) guanine, N-(methyl) guanine, 2-(thio) cytosine, 3-(deaza)-5-(aza) cytosine, 3-(alkyl) cytosine, 3-(methyl) cytosine, 5-(alkyl) cytosine, 5-(alkynyl) cytosine, 5-(halo) cytosine, 5-(methyl) cytosine, 5-(propynyl) cytosine, 5-(propynyl) cytosine, 5-(trifluoromethyl) cytosine, 6-(azo) cytosine, N⁴-(acetyl) cytosine, 3-(3-amino-3-carboxypropyl) uracil, 2-(thio) uracil, 5-(methyl)-2-(thio) uracil, 5-(methylaminomethyl)-2-(thio) uracil, 4-(thio) uracil, 5-(methyl)-4-(thio) uracil, 5-(methylaminomethyl)-4-(thio) uracil, 5-(methyl)-2,4-(dithio) uracil, 5-(methylaminomethyl)-2,4-(dithio) uracil, 5-(2-aminopropyl) uracil, 5-(alkyl) uracil, 5-(alkynyl) uracil, 5-(allylamino) uracil, 5-(aminoallyl) uracil, 5-(aminoalkyl) uracil, 5-(guanidiniumalkyl) uracil, 5-(1,3-diazole-1-alkyl) uracil, 5-(cyanoalkyl) uracil, 5-(dialkylaminoalkyl) uracil, 5-(dimethylaminoalkyl) uracil, 5-(halo) uracil, 5-(methoxy) uracil, uracil-5-oxyacetic acid, 5-(methoxycarbonylmethyl)-2-(thio) uracil, 5-(methoxycarbonyl-methyl) uracil, 5-(propynyl) uracil, 5-(propynyl) uracil, 5-(trifluoromethyl) uracil, 6-(azo) uracil, dihydrouracil, N³-(methyl) uracil, 5-uracil (i.e., pseudouracil), 2-(thio) pseudouracil, 4-(thio) pseudouracil, 2,4-(dithio) psuedouracil, 5-(alkyl) pseudouracil, 5-(methyl) pseudouracil, 5-(alkyl)-2-(thio) pseudouracil, 5-(methyl)-2-(thio) pseudouracil, 5-(alkyl)-4-(thio) pseudouracil, 5-(methyl)-4-(thio) pseudouracil, 5-(alkyl)-2,4-(dithio) pseudouracil, 5-(methyl)-2,4-(dithio) pseudouracil, 1-substituted pseudouracil, 1-substituted 2(thio)-pseudouracil, 1-substituted 4-(thio) pseudouracil, 1-substituted 2,4-(dithio) pseudouracil, 1-(aminocarbonylethylenyl)-pseudouracil, 1-(aminocarbonylethylenyl)-2(thio)-pseudouracil, 1-(aminocarbonylethylenyl)-4-(thio) pseudouracil, 1-(aminocarbonylethylenyl)-2,4-(dithio) pseudouracil, 1-(aminoalkylaminocarbonylethylenyl)-pseudouracil, 1-(aminoalkylamino-carbonylethylenyl)-2(thio)-pseudouracil, 1-(aminoalkylaminocarbonylethylenyl)-4-(thio) pseudouracil, 1-(aminoalkylaminocarbonylethylenyl)-2,4-(dithio) pseudouracil, 1,3-(diaza)-2-(oxo)-phenoxazin-1-yl, 1-(aza)-2-(thio)-3-(aza)-phenoxazin-1-yl, 1,3-(diaza)-2-(oxo)-phenthiazin-1-yl, 1-(aza)-2-(thio)-3-(aza)-phenthiazin-1-yl, 7-substituted 1,3-(diaza)-2-(oxo)-phenoxazin-1-yl, 7-substituted 1-(aza)-2-(thio)-3-(aza)-phenoxazin-1-yl, 7-substituted 1,3-(diaza)-2-(oxo)-phenthiazin-1-yl, 7-substituted 1-(aza)-2-(thio)-3-(aza)-phenthiazin-1-yl, 7-(aminoalkylhydroxy)-1,3-(diaza)-2-(oxo)-phenoxazin-1-yl, 7-(aminoalkylhydroxy)-1-(aza)-2-(thio)-3-(aza)-phenoxazin-1-yl, 7-(aminoalkylhydroxy)-1,3-(diaza)-2-(oxo)-phenthiazin-1-yl, 7-(aminoalkylhydroxy)-1-(aza)-2-(thio)-3-(aza)-phenthiazin-1-yl, 7-(guanidiniumalkylhydroxy)-1,3-(diaza)-2-(oxo)-phenoxazin-1-yl, 7-(guanidiniumalkylhydroxy)-1-(aza)-2-(thio)-3-(aza)-phenoxazin-1-yl, 7-(guanidiniumalkyl-hydroxy)-1,3-(diaza)-2-(oxo)-phenthiazin-1-yl, 7-(guanidiniumalkylhydroxy)-1-(aza)-2-(thio)-3-(aza)-phenthiazin-1-yl, 1,3,5-(triaza)-2,6-(dioxa)-naphthalene, inosine, xanthine, hypoxanthine, nubularine, tubercidine, isoguanisine, inosinyl, 2-aza-inosinyl, 7-deaza-inosinyl, nitroimidazolyl, nitropyrazolyl, nitrobenzimidazolyl, nitroindazolyl, aminoindolyl, pyrrolopyrimidinyl, 3-(methyl) isocarbostyrilyl, 5-(methyl) isocarbostyrilyl, 3-(methyl)-7-(propynyl) isocarbostyrilyl, 7-(aza) indolyl, 6-(methyl)-7-(aza) indolyl, imidizopyridinyl, 9-(methyl)-imidizopyridinyl, pyrrolopyrizinyl, isocarbostyrilyl, 7-(propynyl) isocarbostyrilyl, propynyl-7-(aza) indolyl, 2,4,5-(trimethyl)phenyl, 4-(methyl) indolyl, 4,6-(dimethyl) indolyl, phenyl, napthalenyl, anthracenyl, phenanthracenyl, pyrenyl, stilbenyl, tetracenyl, pentacenyl, difluorotolyl, 4-(fluoro)-6-(methyl)benzimidazole, 4-(methyl)benzimidazole, 6-(azo) thymine, 2-pyridinone, 5-nitroindole, 3-nitropyrrole, 6-(aza)pyrimidine, 2-(amino) purine, 2,6-(diamino) purine, 5-substituted pyrimidines, N²-substituted purines, N⁶-substituted purines, O⁶-substituted purines, substituted 1,2,4-triazoles, and any O-alkylated or N-alkylated derivatives thereof.

A nucleotide comprising a non-natural nucleobase can be present anywherein the dsRNA molecule. For example, a nucleotide comprising a non-natural nucleobase can be present in the sense strand or a nucleotide comprising a non-natural nucleobase can be present in the antisense strand. When two or more nuelcotides comprising a non-natural nucleobase are present in the dsRNA molecule, they can all be in the sense strand, antisense strand or both in the sense and antisense strands.

The dsRNA molecule of the invention can further comprise at least one phosphorothioate or methylphosphonate internucleotide linkage. The phosphorothioate or methylphosphonate internucleotide linkage modification may occur on any nucleotide of the sense strand or antisense strand or both in any position of the strand. For instance, the internucleotide linkage modification may occur on every nucleotide on the sense strand and/or antisense strand; each internucleotide linkage modification may occur in an alternating pattern on the sense strand or antisense strand; or the sense strand or antisense strand comprises both internucleotide linkage modifications in an alternating pattern. The alternating pattern of the internucleotide linkage modification on the sense strand may be the same or different from the antisense strand, and the alternating pattern of the internucleotide linkage modification on the sense strand may have a shift relative to the alternating pattern of the internucleotide linkage modification on the antisense strand.

In some embodiments, the dsRNA molecule comprises the phosphorothioate or methylphosphonate internucleotide linkage modification in the overhang region. For example, the overhang region comprises two nucleotides having a phosphorothioate or methylphosphonate internucleotide linkage between the two nucleotides. Internucleotide linkage modifications also may be made to link the overhang nucleotides with the terminal paired nucleotides within duplex region. For example, at least 2, 3, 4, or all the overhang nucleotides may be linked through phosphorothioate or methylphosphonate internucleotide linkage, and optionally, there may be additional phosphorothioate or methylphosphonate internucleotide linkages linking the overhang nucleotide with a paired nucleotide that is next to the overhang nucleotide. For instance, there may be at least two phosphorothioate internucleotide linkages between the terminal three nucleotides, in which two of the three nucleotides are overhang nucleotides, and the third is a paired nucleotide next to the overhang nucleotide. Preferably, these terminal three nucleotides may be at the 3′-end of the antisense strand.

In some embodiments, the sense strand of the dsRNA molecule comprises 1-10 blocks of two to ten phosphorothioate or methylphosphonate internucleotide linkages separated by 1, 2, 3, 4, 5, 6, 7, 8, 9, 10, 11, 12, 13, 14, 15 or 16 phosphate internucleotide linkages, wherein one of the phosphorothioate or methylphosphonate internucleotide linkages is placed at any position in the oligonucleotide sequence and the said sense strand is paired with an antisense strand comprising any combination of phosphorothioate, methylphosphonate and phosphate internucleotide linkages or an antisense strand comprising either phosphorothioate or methylphosphonate or phosphate linkage.

In some embodiments, the antisense strand of the dsRNA molecule comprises two blocks of two phosphorothioate or methylphosphonate internucleotide linkages separated by 1, 2, 3, 4, 5, 6, 7, 8, 9, 10, 11, 12, 13, 14, 15, 16, 17, or 18 phosphate internucleotide linkages, wherein one of the phosphorothioate or methylphosphonate internucleotide linkages is placed at any position in the oligonucleotide sequence and the said antisense strand is paired with a sense strand comprising any combination of phosphorothioate, methylphosphonate and phosphate internucleotide linkages or an antisense strand comprising either phosphorothioate or methylphosphonate or phosphate linkage.

In some embodiments, the antisense strand of the dsRNA molecule comprises two blocks of three phosphorothioate or methylphosphonate internucleotide linkages separated by 1, 2, 3, 4, 5, 6, 7, 8, 9, 10, 11, 12, 13, 14, 15 or 16 phosphate internucleotide linkages, wherein one of the phosphorothioate or methylphosphonate internucleotide linkages is placed at any position in the oligonucleotide sequence and the said antisense strand is paired with a sense strand comprising any combination of phosphorothioate, methylphosphonate and phosphate internucleotide linkages or an antisense strand comprising either phosphorothioate or methylphosphonate or phosphate linkage.

In some embodiments, the antisense strand of the dsRNA molecule comprises two blocks of four phosphorothioate or methylphosphonate internucleotide linkages separated by 1, 2, 3, 4, 5, 6, 7, 8, 9, 10, 11, 12, 13 or 14 phosphate internucleotide linkages, wherein one of the phosphorothioate or methylphosphonate internucleotide linkages is placed at any position in the oligonucleotide sequence and the said antisense strand is paired with a sense strand comprising any combination of phosphorothioate, methylphosphonate and phosphate internucleotide linkages or an antisense strand comprising either phosphorothioate or methylphosphonate or phosphate linkage.

In some embodiments, the antisense strand of the dsRNA molecule comprises two blocks of five phosphorothioate or methylphosphonate internucleotide linkages separated by 1, 2, 3, 4, 5, 6, 7, 8, 9, 10, 11 or 12 phosphate internucleotide linkages, wherein one of the phosphorothioate or methylphosphonate internucleotide linkages is placed at any position in the oligonucleotide sequence and the said antisense strand is paired with a sense strand comprising any combination of phosphorothioate, methylphosphonate and phosphate internucleotide linkages or an antisense strand comprising either phosphorothioate or methylphosphonate or phosphate linkage.

In some embodiments, the antisense strand of the dsRNA molecule comprises two blocks of six phosphorothioate or methylphosphonate internucleotide linkages separated by 1, 2, 3, 4, 5, 6, 7, 8, 9 or 10 phosphate internucleotide linkages, wherein one of the phosphorothioate or methylphosphonate internucleotide linkages is placed at any position in the oligonucleotide sequence and the said antisense strand is paired with a sense strand comprising any combination of phosphorothioate, methylphosphonate and phosphate internucleotide linkages or an antisense strand comprising either phosphorothioate or methylphosphonate or phosphate linkage.

In some embodiments, the antisense strand of the dsRNA molecule comprises two blocks of seven phosphorothioate or methylphosphonate internucleotide linkages separated by 1, 2, 3, 4, 5, 6, 7 or 8 phosphate internucleotide linkages, wherein one of the phosphorothioate or methylphosphonate internucleotide linkages is placed at any position in the oligonucleotide sequence and the said antisense strand is paired with a sense strand comprising any combination of phosphorothioate, methylphosphonate and phosphate internucleotide linkages or an antisense strand comprising either phosphorothioate or methylphosphonate or phosphate linkage.

In some embodiments, the antisense strand of the dsRNA molecule comprises two blocks of eight phosphorothioate or methylphosphonate internucleotide linkages separated by 1, 2, 3, 4, 5 or 6 phosphate internucleotide linkages, wherein one of the phosphorothioate or methylphosphonate internucleotide linkages is placed at any position in the oligonucleotide sequence and the said antisense strand is paired with a sense strand comprising any combination of phosphorothioate, methylphosphonate and phosphate internucleotide linkages or an antisense strand comprising either phosphorothioate or methylphosphonate or phosphate linkage.

In some embodiments, the antisense strand of the dsRNA molecule comprises two blocks of nine phosphorothioate or methylphosphonate internucleotide linkages separated by 1, 2, 3 or 4 phosphate internucleotide linkages, wherein one of the phosphorothioate or methylphosphonate internucleotide linkages is placed at any position in the oligonucleotide sequence and the said antisense strand is paired with a sense strand comprising any combination of phosphorothioate, methylphosphonate and phosphate internucleotide linkages or an antisense strand comprising either phosphorothioate or methylphosphonate or phosphate linkage.

In some embodiments, the dsRNA molecule of the invention further comprises one or more phosphorothioate or methylphosphonate internucleotide linkage modification within 1-10 of the termini position(s) of the sense and/or antisense strand. For example, at least 2, 3, 4, 5, 6, 7, 8, 9 or 10 nucleotides may be linked through phosphorothioate or methylphosphonate internucleotide linkage at one end or both ends of the sense and/or antisense strand.

In some embodiments, the dsRNA molecule of the invention comprises one or more phosphorothioate or methylphosphonate internucleotide linkage modification within 1-10 of the internal region of the duplex of each of the sense and/or antisense strand. For example, at least 2, 3, 4, 5, 6, 7, 8, 9 or 10 nucleotides may be linked through phosphorothioate methylphosphonate internucleotide linkage at position 8-16 of the duplex region counting from the 5′-end of the sense strand; the dsRNA molecule can optionally further comprise one or more phosphorothioate or methylphosphonate internucleotide linkage modification within 1-10 of the termini position(s).

In some embodiments, the dsRNA molecule of the invention further comprises one to five phosphorothioate or methylphosphonate internucleotide linkage modification(s) within position 1-5 and one to five phosphorothioate or methylphosphonate internucleotide linkage modification(s) within the last 3 positions of the sense strand (counting from the 5′-end), and one to five phosphorothioate or methylphosphonate internucleotide linkage modification at positions 1 and 2 and one to five phosphorothioate or methylphosphonate internucleotide linkage modification within the last six positions of the antisense strand (counting from the 5′-end).

In some embodiments, the dsRNA molecule of the invention further comprises one phosphorothioate internucleotide linkage modification within position 1-5 and one phosphorothioate or methylphosphonate internucleotide linkage modification within the last six positions of the sense strand (counting from the 5′-end), and one phosphorothioate internucleotide linkage modification at positions 1 and 2 and two phosphorothioate or methylphosphonate internucleotide linkage modifications within the last six the last six positions of the antisense strand (counting from the 5′-end).

In some embodiments, the dsRNA molecule of the invention further comprises two phosphorothioate internucleotide linkage modifications within position 1-5 and one phosphorothioate internucleotide linkage modification within the last six positions of the sense strand (counting from the 5′-end), and one phosphorothioate internucleotide linkage modification at positions 1 and 2 and two phosphorothioate internucleotide linkage modifications within the last six positions of the antisense strand (counting from the 5′-end).

In some embodiments, the dsRNA molecule of the invention further comprises two phosphorothioate internucleotide linkage modifications within position 1-5 and two phosphorothioate internucleotide linkage modifications within the last four positions of the sense strand (counting from the 5′-end), and one phosphorothioate internucleotide linkage modification at positions 1 and 2 and two phosphorothioate internucleotide linkage modifications within the last six positions of the antisense strand (counting from the 5′-end).

In some embodiments, the dsRNA molecule of the invention further comprises two phosphorothioate internucleotide linkage modifications within position 1-5 and two phosphorothioate internucleotide linkage modifications within the last four positions of the sense strand (counting from the 5′-end), and one phosphorothioate internucleotide linkage modification at positions 1 and 2 and one phosphorothioate internucleotide linkage modification within the last six positions of the antisense strand (counting from the 5′-end).

In some embodiments, the dsRNA molecule of the invention further comprises one phosphorothioate internucleotide linkage modification within position 1-5 and one phosphorothioate internucleotide linkage modification within the last four positions of the sense strand (counting from the 5′-end), and two phosphorothioate internucleotide linkage modifications at positions 1 and 2 and two phosphorothioate internucleotide linkage modifications within the last six positions of the antisense strand (counting from the 5′-end).

In some embodiments, the dsRNA molecule of the invention further comprises one phosphorothioate internucleotide linkage modification within position 1-5 and one within the last six positions of the sense strand (counting from the 5′-end), and two phosphorothioate internucleotide linkage modification at positions 1 and 2 and one phosphorothioate internucleotide linkage modification within the last six positions of the antisense strand (counting from the 5′-end).

In some embodiments, the dsRNA molecule of the invention further comprises one phosphorothioate internucleotide linkage modification within position 1-5(counting from the 5′-end) of the sense strand, and two phosphorothioate internucleotide linkage modifications at positions 1 and 2 and one phosphorothioate internucleotide linkage modification within the last six positions of the antisense strand (counting from the 5′-end).

In some embodiments, the dsRNA molecule of the invention further comprises two phosphorothioate internucleotide linkage modifications within position 1-5(counting from the 5′-end) of the sense strand, and one phosphorothioate internucleotide linkage modification at positions 1 and 2 and two phosphorothioate internucleotide linkage modifications within the last six positions of the antisense strand (counting from the 5′-end).

In some embodiments, the dsRNA molecule of the invention further comprises two phosphorothioate internucleotide linkage modifications within position 1-5 and one within the last six positions of the sense strand (counting from the 5′-end), and two phosphorothioate internucleotide linkage modifications at positions 1 and 2 and one phosphorothioate internucleotide linkage modification within the last six positions of the antisense strand (counting from the 5′-end).

In some embodiments, the dsRNA molecule of the invention further comprises two phosphorothioate internucleotide linkage modifications within position 1-5 and one phosphorothioate internucleotide linkage modification within the last six positions of the sense strand (counting from the 5′-end), and two phosphorothioate internucleotide linkage modifications at positions 1 and 2 and two phosphorothioate internucleotide linkage modifications within the last six positions of the antisense strand (counting from the 5′-end).

In some embodiments, the dsRNA molecule of the invention further comprises two phosphorothioate internucleotide linkage modifications within position 1-5 and one phosphorothioate internucleotide linkage modification within the last six positions of the sense strand (counting from the 5′-end), and one phosphorothioate internucleotide linkage modification at positions 1 and 2 and two phosphorothioate internucleotide linkage modifications within the last six positions of the antisense strand (counting from the 5′-end).

In some embodiments, the dsRNA molecule of the invention further comprises two phosphorothioate internucleotide linkage modifications at position 1 and 2, and two phosphorothioate internucleotide linkage modifications at position 20 and 21 of the sense strand (counting from the 5′-end), and one phosphorothioate internucleotide linkage modification at positions 1 and one at position 21 of the antisense strand (counting from the 5′-end).

In some embodiments, the dsRNA molecule of the invention further comprises one phosphorothioate internucleotide linkage modification at position 1, and one phosphorothioate internucleotide linkage modification at position 21 of the sense strand (counting from the 5′-end), and two phosphorothioate internucleotide linkage modifications at positions 1 and 2 and two phosphorothioate internucleotide linkage modifications at positions 20 and 21 the antisense strand (counting from the 5′-end).

In some embodiments, the dsRNA molecule of the invention further comprises two phosphorothioate internucleotide linkage modifications at position 1 and 2, and two phosphorothioate internucleotide linkage modifications at position 21 and 22 of the sense strand (counting from the 5′-end), and one phosphorothioate internucleotide linkage modification at positions 1 and one phosphorothioate internucleotide linkage modification at position 21 of the antisense strand (counting from the 5′-end).

In some embodiments, the dsRNA molecule of the invention further comprises one phosphorothioate internucleotide linkage modification at position 1, and one phosphorothioate internucleotide linkage modification at position 21 of the sense strand (counting from the 5′-end), and two phosphorothioate internucleotide linkage modifications at positions 1 and 2 and two phosphorothioate internucleotide linkage modifications at positions 21 and 22 the antisense strand (counting from the 5′-end).

In some embodiments, the dsRNA molecule of the invention further comprises two phosphorothioate internucleotide linkage modifications at position 1 and 2, and two phosphorothioate internucleotide linkage modifications at position 22 and 23 of the sense strand (counting from the 5′-end), and one phosphorothioate internucleotide linkage modification at positions 1 and one phosphorothioate internucleotide linkage modification at position 21 of the antisense strand (counting from the 5′-end).

In some embodiments, the dsRNA molecule of the invention further comprises one phosphorothioate internucleotide linkage modification at position 1, and one phosphorothioate internucleotide linkage modification at position 21 of the sense strand (counting from the 5′-end), and two phosphorothioate internucleotide linkage modifications at positions 1 and 2 and two phosphorothioate internucleotide linkage modifications at positions 22 and 23 the antisense strand (counting from the 5′-end).

In some embodiments, the sense strand comprises at least two phosphorothioate internucleotide linkages between the first five nucleotides counting from the 5′ end of the sense strand. For example, the sense strand comprises phosphorothioate linkages between nucleotides 1 and 2, and between nucleotides 2 and 3, counting from 5′-end of the sense strand.

In some embodiments, the antisense strand comprises at least two phosphorothioate internucleotide linkages between the first five nucleotides counting from the 5′-end of the antisense strand. For example, the antisense strand comprises phosphorothioate linkages between nucleotides 1 and 2, and between nucleotides 2 and 3, counting from 5′-end of the antisense strand.

In some embodiments, the antisense strand comprises at least two phosphorothioate internucleotide linkages between the first five nucleotides counting from the 3′ end of the antisense strand. For example, the antisense strand comprises phosphorothioate linkages between nucleotides n and n-1, and between nucleotides n-1 and n-2, where n is length of the antisense strand, i.e, number of nucleotides in the antisense strand. In other words, the antisense strand comprises phosphorothioate linkages between nucleotides 1 and 2, and between nucleotides 2 and 3, counting from 3′-end of the antisense strand.

In some embodiments, the antisense strand comprises at least two phosphorothioate internucleotide linkages between the first five nucleotides counting from the 5′-end of the antisense strand and at least two phosphorothioate internucleotide linkages between the first five nucleotides counting from the 5′-end of the antisense strand. For example, the antisense strand comprises phosphorothioate linkages between nucleotides 1 and 2, and between nucleotides 2 and 3, counting from 5′-end of the antisense strand and between nucleotides 1 and 2, and between nucleotides 2 and 3, counting from 3′-end of the antisense strand.

In some embodiments, the sense strand comprises at least two phosphorothioate internucleotide linkages between the first five nucleotides counting from the 5′ end of the sense strand and the antisense strand comprises at least two phosphorothioate internucleotide linkages between the first five nucleotides counting from the 5′-end of the antisense strand. For example, the sense strand comprises phosphorothioate linkages between nucleotides 1 and 2, and between nucleotides 2 and 3, counting from 5′-end of the sense strand, and the antisense strand comprises phosphorothioate linkages between nucleotides 1 and 2, and between nucleotides 2 and 3, counting from 5′-end of the antisense strand.

In some embodiments, the sense strand comprises at least two phosphorothioate internucleotide linkages between the first five nucleotides counting from the 5′ end of the sense strand and the antisense strand comprises at least two phosphorothioate internucleotide linkages between the first five nucleotides counting from the 3′-end of the antisense strand. For example, the sense strand comprises phosphorothioate linkages between nucleotides 1 and 2, and between nucleotides 2 and 3, counting from 5′-end of the sense strand, and the antisense strand comprises phosphorothioate linkages between nucleotides 1 and 2, and between nucleotides 2 and 3, counting from 3′-end of the antisense strand.

In some embodiments, compound of the invention comprises a pattern of backbone chiral centers. In some embodiments, a common pattern of backbone chiral centers comprises at least 5 internucleotidic linkages in the Sp configuration. In some embodiments, a common pattern of backbone chiral centers comprises at least 6 internucleotidic linkages in the Sp configuration. In some embodiments, a common pattern of backbone chiral centers comprises at least 7 internucleotidic linkages in the Sp configuration. In some embodiments, a common pattern of backbone chiral centers comprises at least 8 internucleotidic linkages in the Sp configuration. In some embodiments, a common pattern of backbone chiral centers comprises at least 9 internucleotidic linkages in the Sp configuration. In some embodiments, a common pattern of backbone chiral centers comprises at least 10 internucleotidic linkages in the Sp configuration. In some embodiments, a common pattern of backbone chiral centers comprises at least 11 internucleotidic linkages in the Sp configuration. In some embodiments, a common pattern of backbone chiral centers comprises at least 12 internucleotidic linkages in the Sp configuration. In some embodiments, a common pattern of backbone chiral centers comprises at least 13 internucleotidic linkages in the Sp configuration. In some embodiments, a common pattern of backbone chiral centers comprises at least 14 internucleotidic linkages in the Sp configuration. In some embodiments, a common pattern of backbone chiral centers comprises at least 15 internucleotidic linkages in the Sp configuration. In some embodiments, a common pattern of backbone chiral centers comprises at least 16 internucleotidic linkages in the Sp configuration. In some embodiments, a common pattern of backbone chiral centers comprises at least 17 internucleotidic linkages in the Sp configuration. In some embodiments, a common pattern of backbone chiral centers comprises at least 18 internucleotidic linkages in the Sp configuration. In some embodiments, a common pattern of backbone chiral centers comprises at least 19 internucleotidic linkages in the Sp configuration. In some embodiments, a common pattern of backbone chiral centers comprises no more than 8 internucleotidic linkages in the Rp configuration. In some embodiments, a common pattern of backbone chiral centers comprises no more than 7 internucleotidic linkages in the Rp configuration. In some embodiments, a common pattern of backbone chiral centers comprises no more than 6 internucleotidic linkages in the Rp configuration. In some embodiments, a common pattern of backbone chiral centers comprises no more than 5 internucleotidic linkages in the Rp configuration. In some embodiments, a common pattern of backbone chiral centers comprises no more than 4 internucleotidic linkages in the Rp configuration. In some embodiments, a common pattern of backbone chiral centers comprises no more than 3 internucleotidic linkages in the Rp configuration. In some embodiments, a common pattern of backbone chiral centers comprises no more than 2 internucleotidic linkages in the Rp configuration. In some embodiments, a common pattern of backbone chiral centers comprises no more than 1 internucleotidic linkages in the Rp configuration. In some embodiments, a common pattern of backbone chiral centers comprises no more than 8 internucleotidic linkages which are not chiral (as a non-limiting example, a phosphodiester). In some embodiments, a common pattern of backbone chiral centers comprises no more than 7 internucleotidic linkages which are not chiral. In some embodiments, a common pattern of backbone chiral centers comprises no more than 6 internucleotidic linkages which are not chiral. In some embodiments, a common pattern of backbone chiral centers comprises no more than 5 internucleotidic linkages which are not chiral. In some embodiments, a common pattern of backbone chiral centers comprises no more than 4 internucleotidic linkages which are not chiral. In some embodiments, a common pattern of backbone chiral centers comprises no more than 3 internucleotidic linkages which are not chiral. In some embodiments, a common pattern of backbone chiral centers comprises no more than 2 internucleotidic linkages which are not chiral. In some embodiments, a common pattern of backbone chiral centers comprises no more than 1 internucleotidic linkages which are not chiral. In some embodiments, a common pattern of backbone chiral centers comprises at least 10 internucleotidic linkages in the Sp configuration, and no more than 8 internucleotidic linkages which are not chiral. In some embodiments, a common pattern of backbone chiral centers comprises at least 11 internucleotidic linkages in the Sp configuration, and no more than 7 internucleotidic linkages which are not chiral. In some embodiments, a common pattern of backbone chiral centers comprises at least 12 internucleotidic linkages in the Sp configuration, and no more than 6 internucleotidic linkages which are not chiral. In some embodiments, a common pattern of backbone chiral centers comprises at least 13 internucleotidic linkages in the Sp configuration, and no more than 6 internucleotidic linkages which are not chiral. In some embodiments, a common pattern of backbone chiral centers comprises at least 14 internucleotidic linkages in the Sp configuration, and no more than 5 internucleotidic linkages which are not chiral. In some embodiments, a common pattern of backbone chiral centers comprises at least 15 internucleotidic linkages in the Sp configuration, and no more than 4 internucleotidic linkages which are not chiral. In some embodiments, the internucleotidic linkages in the Sp configuration are optionally contiguous or not contiguous. In some embodiments, the internucleotidic linkages in the Rp configuration are optionally contiguous or not contiguous. In some embodiments, the internucleotidic linkages which are not chiral are optionally contiguous or not contiguous.

In some embodiments, compound of the invention comprises a block is a stereochemistry block. In some embodiments, a block is an Rp block in that each internucleotidic linkage of the block is Rp. In some embodiments, a 5′-block is an Rp block. In some embodiments, a 3′-block is an Rp block. In some embodiments, a block is an Sp block in that each internucleotidic linkage of the block is Sp. In some embodiments, a 5′-block is an Sp block. In some embodiments, a 3′-block is an Sp block. In some embodiments, provided oligonucleotides comprise both Rp and Sp blocks. In some embodiments, provided oligonucleotides comprise one or more Rp but no Sp blocks. In some embodiments, provided oligonucleotides comprise one or more Sp but no Rp blocks. In some embodiments, provided oligonucleotides comprise one or more PO blocks wherein each internucleotidic linkage in a natural phosphate linkage.

In some embodiments, compound of the invention comprises a 5′-block is an Sp block wherein each sugar moiety comprises a 2′-fluoro modification. In some embodiments, a 5′-block is an Sp block wherein each of internucleotidic linkage is a modified internucleotidic linkage and each sugar moiety comprises a 2′-fluoro modification. In some embodiments, a 5′-block is an Sp block wherein each of internucleotidic linkage is a phosphorothioate linkage and each sugar moiety comprises a 2′-fluoro modification. In some embodiments, a 5′-block comprises 4 or more nucleoside units. In some embodiments, a 5′-block comprises 5 or more nucleoside units. In some embodiments, a 5′-block comprises 6 or more nucleoside units. In some embodiments, a 5′-block comprises 7 or more nucleoside units. In some embodiments, a 3′-block is an Sp block wherein each sugar moiety comprises a 2′-fluoro modification. In some embodiments, a 3′-block is an Sp block wherein each of internucleotidic linkage is a modified internucleotidic linkage and each sugar moiety comprises a 2′-fluoro modification. In some embodiments, a 3′-block is an Sp block wherein each of internucleotidic linkage is a phosphorothioate linkage and each sugar moiety comprises a 2′-fluoro modification. In some embodiments, a 3′-block comprises 4 or more nucleoside units. In some embodiments, a 3′-block comprises 5 or more nucleoside units. In some embodiments, a 3′-block comprises 6 or more nucleoside units. In some embodiments, a 3′-block comprises 7 or more nucleoside units.

In some embodiments, compound of the invention comprises a type of nucleoside in a region or an oligonucleotide is followed by a specific type of internucleotidic linkage, e.g., natural phosphate linkage, modified internucleotidic linkage, Rp chiral internucleotidic linkage, Sp chiral internucleotidic linkage, etc. In some embodiments, A is followed by Sp. In some embodiments, A is followed by Rp. In some embodiments, A is followed by natural phosphate linkage (PO). In some embodiments, U is followed by Sp. In some embodiments, U is followed by Rp. In some embodiments, U is followed by natural phosphate linkage (PO). In some embodiments, C is followed by Sp. In some embodiments, C is followed by Rp. In some embodiments, C is followed by natural phosphate linkage (PO). In some embodiments, G is followed by Sp. In some embodiments, G is followed by Rp. In some embodiments, G is followed by natural phosphate linkage (PO). In some embodiments, C and U are followed by Sp. In some embodiments, C and U are followed by Rp. In some embodiments, C and U are followed by natural phosphate linkage (PO). In some embodiments, A and G are followed by Sp. In some embodiments, A and G are followed by Rp.

Various publications describe multimeric siRNAs which can all be used with the dsRNA of the invention. Such publications include WO2007/091269, U.S. Pat. No. 7,858,769, WO2010/141511, WO2007/117686, WO2009/014887 and WO2011/031520 which are hereby incorporated by their entirely.

### Ligands

A wide variety of entities can be coupled to the dsRNA agents described herein. Preferred moieties are ligands, which are coupled, preferably covalently, either directly or indirectly via an intervening tether. Generally, a ligand alters the distribution, targeting or lifetime of the molecule, e.g., a dsRNA described herein, into which it is incorporated. In some embodiments a ligand provides an enhanced affinity for a selected target, e.g., molecule, cell or cell type, compartment, receptor e.g., a cellular or organ compartment, tissue, organ or region of the body, as, e.g., compared to a species absent such a ligand. Ligands providing enhanced affinity for a selected target are also termed targeting ligands herein.

Some ligands can have endosomolytic properties. The endosomolytic ligands promote the lysis of the endosome and/or transport of the composition of the invention, or its components, from the endosome to the cytoplasm of the cell. The endosomolytic ligand may be a polyanionic peptide or peptidomimetic which shows pH-dependent membrane activity and fusogenicity. In some embodiments, the endosomolytic ligand assumes its active conformation at endosomal pH. The “active” conformation is that conformation in which the endosomolytic ligand promotes lysis of the endosome and/or transport of the composition of the invention, or its components, from the endosome to the cytoplasm of the cell. Exemplary endosomolytic ligands include the GALA peptide (Subbarao et al., Biochemistry, 1987, 26:2964-2972, which is incorporated by reference in its entirety), the EALA peptide (Vogel et al., J. Am. Chem. Soc., 1996, 118:1581-1586, which is incorporated by reference in its entirety), and their derivatives (Turk et al., Biochem. Biophys. Acta, 2002, 1559:56-68, which is incorporated by reference in its entirety). In some embodiments, the endosomolytic component may contain a chemical group (e.g., an amino acid) which will undergo a change in charge or protonation in response to a change in pH. The endosomolytic component may be linear or branched.

Ligands can improve transport, hybridization, and specificity properties and can also improve nuclease resistance of the resultant natural or modified oligoribonucleotide, or a polymeric molecule comprising any combination of monomers described herein and/or natural or modified ribonucleotides.

Ligands in general can include therapeutic modifiers, e.g., for enhancing uptake; diagnostic compounds or reporter groups e.g., for monitoring distribution; cross-linking agents; and nuclease-resistance conferring moieties. General examples include lipids, steroids, vitamins, sugars, proteins, peptides, polyamines, and peptide mimics.

Ligands can include a naturally occurring substance, such as a protein (e.g., human serum albumin (HSA), low-density lipoprotein (LDL), high-density lipoprotein (HDL), or globulin); a carbohydrate (e.g., a dextran, pullulan, chitin, chitosan, inulin, cyclodextrin or hyaluronic acid); or a lipid. The ligand may also be a recombinant or synthetic molecule, such as a synthetic polymer, e.g., a synthetic polyamino acid, an oligonucleotide (e.g. an aptamer). Examples of polyamino acids include polyamino acid is a polylysine (PLL), poly L-aspartic acid, poly L-glutamic acid, styrene-maleic acid anhydride copolymer, poly(L-lactide-co-glycolide) copolymer, divinyl ether-maleic anhydride copolymer, N-(2-hydroxypropyl) methacrylamide copolymer (HMPA), polyethylene glycol (PEG), polyvinyl alcohol (PVA), polyurethane, poly(2-ethylacryllic acid), N-isopropylacrylamide polymers, or polyphosphazine. Example of polyamines include: polyethylenimine, polylysine (PLL), spermine, spermidine, polyamine, pseudopeptide-polyamine, peptidomimetic polyamine, dendrimer polyamine, arginine, amidine, protamine, cationic lipid, cationic porphyrin, quaternary salt of a polyamine, or an alpha helical peptide.

Ligands can also include targeting groups, e.g., a cell or tissue targeting agent, e.g., a lectin, glycoprotein, lipid or protein, e.g., an antibody, nanobody, or portion of an antibody of nanobody that binds to a specified cell type such as a kidney cell or a cell of the blood-brain barrier.

A targeting group can be a thyrotropin, melanotropin, lectin, glycoprotein, surfactant protein A, Mucin carbohydrate, multivalent lactose, multivalent galactose, N-acetyl-galactosamine, N-acetyl-glucosamine multivalent mannose, multivalent fucose, glycosylated polyamino acids, multivalent galactose, transferrin-targeting group, bisphosphonate, polyglutamate, polyaspartate, a lipid, cholesterol, a steroid, bile acid, folate, vitamin B12, biotin, an RGD peptide, an RGD peptide mimetic or an aptamer.

Other examples of ligands include dyes, intercalating agents (e.g. acridines), cross-linkers (e.g. psoralen, mitomycin C), porphyrins (TPPC4, texaphyrin, Sapphyrin), polycyclic aromatic hydrocarbons (e.g., phenazine, dihydrophenazine), artificial endonucleases or a chelating agent (e.g. EDTA), lipophilic molecules, e.g., cholesterol, cholic acid, adamantane acetic acid, 1-pyrene butyric acid, dihydrotestosterone, 1,3-Bis —O (hexadecyl)glycerol, geranyloxyhexyl group, hexadecylglycerol, borneol, menthol, 1,3-propanediol, heptadecyl group, palmitic acid, myristic acid, O3-(oleoyl) lithocholic acid, O3-(oleoyl) cholenic acid, dimethoxytrityl, or phenoxazine) and peptide conjugates (e.g., antennapedia peptide, Tat peptide), alkylating agents, phosphate, amino, mercapto, PEG (e.g., PEG-40K), MPEG, [MPEG]₂, polyamino, alkyl, substituted alkyl, radiolabeled markers, enzymes, haptens (e.g. biotin), transport/absorption facilitators (e.g., aspirin, vitamin E, folic acid), synthetic ribonucleases (e.g., imidazole, bisimidazole, histamine, imidazole clusters, acridine-imidazole conjugates, Eu3+ complexes of tetraazamacrocycles), dinitrophenyl, HRP, or AP.

Ligands can be proteins, e.g., glycoproteins, or peptides, e.g., molecules having a specific affinity for a co-ligand, or antibodies e.g., an antibody, that binds to a specified cell type such as a cancer cell, endothelial cell, or bone cell. Ligands may also include hormones and hormone receptors. They can also include non-peptide species, such as lipids, lectins, carbohydrates, vitamins, cofactors, multivalent lactose, multivalent galactose, N-acetyl-galactosamine, N-acetyl-glucosamine multivalent mannose, multivalent fucose, or aptamers. The ligand can be, for example, a lipopolysaccharide, an activator of p38 MAP kinase, or an activator of NF-κB.

The ligand can be a substance, e.g., a drug, which can increase the uptake of the iRNA agent into the cell, for example, by disrupting the cell's cytoskeleton, e.g., by disrupting the cell's microtubules, microfilaments, and/or intermediate filaments. The drug can be, for example, taxon, vincristine, vinblastine, cytochalasin, nocodazole, japlakinolide, latrunculin A, phalloidin, swinholide A, indanocine, or myoservin.

The ligand can increase the uptake of the dsRNA into the cell by activating an inflammatory response, for example. Exemplary ligands that would have such an effect include tumor necrosis factor alpha (TNF-alpha), interleukin-1 beta, or gamma interferon.

In some embodiments, the ligand is a lipid or lipid-based molecule. Such a lipid or lipid-based molecule preferably binds a serum protein, e.g., human serum albumin (HSA). An HSA binding ligand allows for distribution of the conjugate to a target tissue, e.g., a non-kidney target tissue of the body. For example, the target tissue can be the liver, including parenchymal cells of the liver. Other molecules that can bind HSA can also be used as ligands. For example, naproxen or aspirin can be used. A lipid or lipid-based ligand can (a) increase resistance to degradation of the conjugate, (b) increase targeting or transport into a target cell or cell membrane, and/or (c) can be used to adjust binding to a serum protein, e.g., HSA. A lipid based ligand can be used to modulate, e.g., control the binding of the conjugate to a target tissue. For example, a lipid or lipid-based ligand that binds to HSA more strongly will be less likely to be targeted to the kidney and therefore less likely to be cleared from the body. A lipid or lipid-based ligand that binds to HSA less strongly can be used to target the conjugate to the kidney.

In a preferred embodiment, the lipid based ligand binds HSA. Preferably, it binds HSA with a sufficient affinity such that the conjugate will be preferably distributed to a non-kidney tissue. However, it is preferred that the affinity not be so strong that the HSA-ligand binding cannot be reversed.

In another preferred embodiment, the lipid based ligand binds HSA weakly or not at all, such that the conjugate will be preferably distributed to the kidney. Other moieties that target to kidney cells can also be used in place of or in addition to the lipid based ligand.

In some embodiments, the ligand is a moiety, e.g., a vitamin, which is taken up by a target cell, e.g., a proliferating cell. These are particularly useful for treating disorders characterized by unwanted cell proliferation, e.g., of the malignant or non-malignant type, e.g., cancer cells. Exemplary vitamins include vitamin A, E, and K. Other exemplary vitamins include B vitamins, e.g., folic acid, B12, riboflavin, biotin, pyridoxal or other vitamins or nutrients taken up by cancer cells. Also included are HAS, low density lipoprotein (LDL) and high-density lipoprotein (HDL).

In another aspect, the ligand is a cell-permeation agent, preferably a helical cell-permeation agent. Preferably, the agent is amphipathic. An exemplary agent is a peptide such as tat or antennapedia. If the agent is a peptide, it can be modified, including a peptidylmimetic, invertomers, non-peptide or pseudo-peptide linkages, and use of D-amino acids. The helical agent is preferably an alpha-helical agent, which preferably has a lipophilic and a lipophobic phase.

The ligand can be a peptide or peptidomimetic. A peptidomimetic (also referred to herein as an oligopeptidomimetic) is a molecule capable of folding into a defined three-dimensional structure similar to a natural peptide. The peptide or peptidomimetic moiety can be about 5-50 amino acids long, e.g., about 5, 10, 15, 20, 25, 30, 35, 40, 45, or 50 amino acids long. A peptide or peptidomimetic can be, for example, a cell permeation peptide, cationic peptide, amphipathic peptide, or hydrophobic peptide (e.g., consisting primarily of Tyr, Trp or Phe). The peptide moiety can be a dendrimer peptide, constrained peptide or cross-linked peptide. In another alternative, the peptide moiety can include a hydrophobic membrane translocation sequence (MTS). An exemplary hydrophobic MTS-containing peptide is RFGF having the amino acid sequence AAVALLPAVLLALLAP(SEQ ID NO: 1). An RFGF analogue (e.g., amino acid sequence AALLPVLLAAP(SEQ ID NO: 2)) containing a hydrophobic MTS can also be a targeting moiety. The peptide moiety can be a “delivery” peptide, which can carry large polar molecules including peptides, oligonucleotides, and protein across cell membranes. For example, sequences from the HIV Tat protein (GRKKRRQRRRPPQ (SEQ ID NO: 3)) and the Drosophila Antennapedia protein (RQIKIWFQNRRMKWKK (SEQ ID NO: 4) have been found to be capable of functioning as delivery peptides. A peptide or peptidomimetic can be encoded by a random sequence of DNA, such as a peptide identified from a phage-display library, or one-bead-one-compound (OBOC) combinatorial library (Lam et al., Nature, 354:82-94, 1991, which is incorporated by reference in its entirety). Preferably the peptide or peptidomimetic tethered to an iRNA agent via an incorporated monomer unit is a cell targeting peptide such as an arginine-glycine-aspartic acid (RGD)-peptide, or RGD mimic. A peptide moiety can range in length from about 5 amino acids to about 40 amino acids. The peptide moieties can have a structural modification, such as to increase stability or direct conformational properties. Any of the structural modifications described below can be utilized. An RGD peptide moiety can be used to target a tumor cell, such as an endothelial tumor cell or a breast cancer tumor cell (Zitzmann et al., Cancer Res., 62:5139-43, 2002, which is incorporated by reference in its entirety). An RGD peptide can facilitate targeting of an iRNA agent to tumors of a variety of other tissues, including the lung, kidney, spleen, or liver (Aoki et al., Cancer Gene Therapy 8:783-787, 2001, which is incorporated by reference in its entirety). Preferably, the RGD peptide will facilitate targeting of an iRNA agent to the kidney. The RGD peptide can be linear or cyclic, and can be modified, e.g., glycosylated or methylated to facilitate targeting to specific tissues. For example, a glycosylated RGD peptide can deliver an iRNA agent to a tumor cell expressing ανβ₃(Haubner et al., Jour. Nucl. Med., 42:326-336, 2001, which is incorporated by reference in its entirety). Peptides that target markers enriched in proliferating cells can be used. For example, RGD containing peptides and peptidomimetics can target cancer cells, in particular cells that exhibit an integrin. Thus, one could use RGD peptides, cyclic peptides containing RGD, RGD peptides that include D-amino acids, as well as synthetic RGD mimics. In addition to RGD, one can use other moieties that target the integrin ligand. Generally, such ligands can be used to control proliferating cells and angiogenesis. Preferred conjugates of this type ligands that targets PECAM-1, VEGF, or other cancer gene, e.g., a cancer gene described herein.

A “cell permeation peptide” is capable of permeating a cell, e.g., a microbial cell, such as a bacterial or fungal cell, or a mammalian cell, such as a human cell. A microbial cell-permeating peptide can be, for example, an α-helical linear peptide (e.g., LL-37 or Ceropin P1), a disulfide bond-containing peptide (e.g., α-defensin, β-defensin or bactenecin), or a peptide containing only one or two dominating amino acids (e.g., PR-39 or indolicidin). A cell permeation peptide can also include a nuclear localization signal (NLS). For example, a cell permeation peptide can be a bipartite amphipathic peptide, such as MPG, which is derived from the fusion peptide domain of HIV-1 gp41 and the NLS of SV40 large T antigen (Simeoni et al., Nucl. Acids Res. 31:2717-2724, 2003, which is incorporated by reference in its entirety).

In some embodiments, a targeting peptide can be an amphipathic α-helical peptide. Exemplary amphipathic α-helical peptides include, but are not limited to, cecropins, lycotoxins, paradaxins, buforin, CPF, bombinin-like peptide (BLP), cathelicidins, ceratotoxins, S. clava peptides, hagfish intestinal antimicrobial peptides (HFIAPs), magainines, brevinins-2, dermaseptins, melittins, pleurocidin, H₂A peptides, Xenopus peptides, esculentinis-1, and caerins. A number of factors will preferably be considered to maintain the integrity of helix stability. For example, a maximum number of helix stabilization residues will be utilized (e.g., leu, ala, or lys), and a minimum number of helix destabilization residues will be utilized (e.g., proline, or cyclic monomeric units. The capping residue will be considered (for example Gly is an exemplary N-capping residue and/or C-terminal amidation can be used to provide an extra H-bond to stabilize the helix. Formation of salt bridges between residues with opposite charges, separated by i±3, or i±4 positions can provide stability. For example, cationic residues such as lysine, arginine, homo-arginine, ornithine or histidine can form salt bridges with the anionic residues glutamate or aspartate.

Peptide and peptidomimetic ligands include those having naturally occurring or modified peptides, e.g., D or L peptides; a, B, or y peptides; N-methyl peptides; azapeptides; peptides having one or more amide, i.e., peptide, linkages replaced with one or more urea, thiourea, carbamate, or sulfonyl urea linkages; or cyclic peptides.

The targeting ligand can be any ligand that is capable of targeting a specific receptor. Examples are: folate, GalNAc, galactose, mannose, mannose-6P, clusters of sugars such as GalNAc cluster, mannose cluster, galactose cluster, or an aptamer. A cluster is a combination of two or more sugar units. The targeting ligands also include integrin receptor ligands, Chemokine receptor ligands, transferrin, biotin, serotonin receptor ligands, PSMA, endothelin, GCPII, somatostatin, LDL and HDL ligands. The ligands can also be based on nucleic acid, e.g., an aptamer. The aptamer can be unmodified or have any combination of modifications disclosed herein.

Endosomal release agents include imidazoles, poly or oligoimidazoles, PEIs, peptides, fusogenic peptides, polycarboxylates, polycations, masked oligo or poly cations or anions, acetals, polyacetals, ketals/polyketals, orthoesters, polymers with masked or unmasked cationic or anionic charges, dendrimers with masked or unmasked cationic or anionic charges.

PK modulator stands for pharmacokinetic modulator. PK modulator include lipophiles, bile acids, steroids, phospholipid analogues, peptides, protein binding agents, PEG, vitamins etc. Exemplary PK modulator include, but are not limited to, cholesterol, fatty acids, cholic acid, lithocholic acid, dialkylglycerides, diacylglyceride, phospholipids, sphingolipids, naproxen, ibuprofen, vitamin E, biotin etc. Oligonucleotides that comprise a number of phosphorothioate linkages are also known to bind to serum protein, thus short oligonucleotides, e.g. oligonucleotides of about 5 bases, 10 bases, 15 bases or 20 bases, comprising multiple of phosphorothioate linkages in the backbone are also amenable to the present invention as ligands (e.g. as PK modulating ligands).

In addition, aptamers that bind serum components (e.g. serum proteins) are also amenable to the present invention as PK modulating ligands.

Other ligand conjugates amenable to the invention are described in U.S. patent application Ser. No. 10/916,185, filed Aug. 10, 2004; U.S. Ser. No. 10/946,873, filed Sep. 21, 2004; USSN: U.S. Ser. No. 10/833,934, filed Aug. 3, 2007; USSN: U.S. Ser. No. 11/115,989 filed Apr. 27, 2005 and USSN: U.S. Ser. No. 11/944,227 filed Nov. 21, 2007, which are incorporated by reference in their entireties for all purposes.

In some embodiments, the dsRNA molecule can comprise two or more, e.g., 2, 3, 4 or 5 ligands. When two or more ligands are present, the ligands can all have same properties, all have different properties or some ligands have the same properties while others have different properties. For example, a ligand can have targeting properties, have endosomolytic activity or have PK modulating properties. In a preferred embodiment, all the ligands have different properties.

In some embodiments the dsRNA molecule comprises two ligands. For example, the sense strand of the dsRNA molecule comprises a first ligand attached at the 3′-end of the sense strand and a second ligand attached at the 5′-end of the sense strand. In some embodiments, the dsRNA molecule comprises two ligands linked to the sense strand, where the first ligand comprises an inverted abasic nucleotide (i.e., an abasic nucleotide linked via 3′->3′ linkage) and the second ligand comprises an ASGPR ligand.

Ligands can be coupled to the dsRNA at various places, for example, 3′-end, 5′-end, and/or at an internal position of the sense and/or antisense strand. In preferred embodiments, the ligand is attached to the sense and/or antisense strand of the dsRNA via a linker or tether. The ligand or tethered ligand can be present on a monomer when said monomer is incorporated into the growing strand. In some embodiments, the ligand may be incorporated via coupling to a “precursor” monomer after said “precursor” monomer has been incorporated into the growing strand. For example, a monomer having, e.g., an amino-terminated tether (i.e., having no associated ligand), e.g., TAP-(CH2),NH2 may be incorporated into a growing oligonucleotide strand. In a subsequent operation, i.e., after incorporation of the precursor monomer into the strand, a ligand having an electrophilic group, e.g., a pentafluorophenyl ester or aldehyde group, can subsequently be attached to the precursor monomer by coupling the electrophilic group of the ligand with the terminal nucleophilic group of the precursor monomer's tether.

In another example, a monomer having a chemical group suitable for taking part in Click Chemistry reaction may be incorporated e.g., an azide or alkyne terminated tether/linker. In a subsequent operation, i.e., after incorporation of the precursor monomer into the strand, a ligand having complementary chemical group, e.g. an alkyne or azide can be attached to the precursor monomer by coupling the alkyne and the azide together.

The ligands can be attached to one or both strands. In some embodiments, a dsRNA described herein comprises a ligand conjugated to the sense strand. In some embodiments, a dsRNA described herein comprises a ligand conjugated to the antisense strand.

In some embodiments, the ligand is conjugated to the sense strand. As described herein, the ligand can be conjugated at the 3′-end, 5′-end or at an internal position of the sense strand. In some embodiments, the ligand is conjugated to the 3′-end of the sense strand. In some embodiments, the ligand is conjugated to the 5′-end of the sense strand. In some embodiments, the ligand is conjugated at an internal position of the sense strand. In other words, the ligand is conjugated to a non-terminal nucleotide of the sense strand. It is noted that the ligand can be conjugated to a nucleobase, sugar moiety or internucleotide linkage of the sense strand.

In some embodiments, the ligand is conjugated at the 2′-position of a nucleotide in the sense strand. For example, the ligand is conjugated at the 2′-position of a nucleotide at an internal, i.e., non-terminal position of the sense strand.

In some embodiments, ligand can be conjugated to nucleobases, sugar moieties, or internucleosidic linkages of nucleic acid molecules. Conjugation to purine nucleobases or derivatives thereof can occur at any position including, endocyclic and exocyclic atoms. In some embodiments, the 2-, 6-, 7-, or 8-positions of a purine nucleobase are attached to a conjugate moiety. Conjugation to pyrimidine nucleobases or derivatives thereof can also occur at any position. In some embodiments, the 2-, 5-, and 6-positions of a pyrimidine nucleobase can be substituted with a conjugate moiety. Conjugation to sugar moieties of nucleosides can occur at any carbon atom. Example carbon atoms of a sugar moiety that can be attached to a conjugate moiety include the 2′, 3′, and 5′ carbon atoms. The 1′ position can also be attached to a conjugate moiety, such as in an abasic residue. Internucleosidic linkages can also bear conjugate moieties. For phosphorus-containing linkages (e.g., phosphodiester, phosphorothioate, phosphorodithioate, phosphoroamidate, and the like), the conjugate moiety can be attached directly to the phosphorus atom or to an O, N, or S atom bound to the phosphorus atom. For amine-or amide-containing internucleosidic linkages (e.g., PNA), the conjugate moiety can be attached to the nitrogen atom of the amine or amide or to an adjacent carbon atom.

In some embodiments, the ligand is conjugated to the sense strand. As described herein, the ligand can be conjugated at the 3′-end, 5′-end or at an internal position of the sense strand. In some embodiments, the ligand is conjugated to the 3′-end of the sense strand. Further, the ligand can be conjugated to a nucleobase, sugar moiety or internucleotide linkage of the sense strand.

Any suitable ligand in the field of RNA interference may be used, although the ligand is typically a carbohydrate e.g. monosaccharide (such as GalNAc), disaccharide, trisaccharide, tetrasaccharide, polysaccharide.

Linkers that conjugate the ligand to the nucleic acid include those discussed above. For example, the ligand can be one or more carbohydrates, e.g., GalNAc (N-acetylgalactosamine) derivatives attached through a monovalent, bivalent or trivalent branched linker.

In some embodiments, the dsRNA of the invention is conjugated to a bivalent and trivalent branched linkers include the structures shown in any of Formula (IV)-(VII):

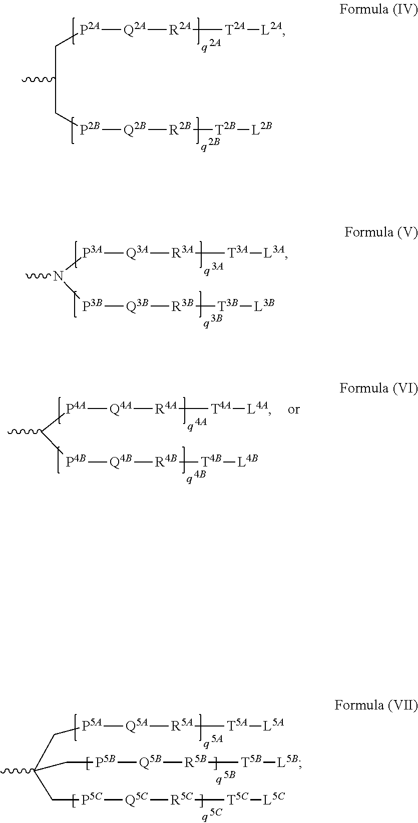

wherein:
q^{2A}, q^{2B}, q^{3A}, q^{3B}, q^{4A}, q^{4B}, q^{5A}, q^{5B} and q^{5C} represent independently for each occurrence 0-20 and wherein the repeating unit can be the same or different;
P^{2A}, P^{2B}, P^{3A}, P^{3B}, P^{4A}, P^{4B}, P^{5A}, P^{5B}, P^{5C}, T^{2A}, T^{2B}, T^{3A}, T^{3B}, T^{4A}, T^{4B}, T^{5A}, T^{5B}, T^{5C} are each independently for each occurrence absent, CO, NH, O, S, OC(O), NHC(O), CH₂, CH₂NH or CH₂O;
Q^{2A}, Q^{2B}, Q^{3A}, Q^{3B}, Q^{4A}, Q^{4B}, Q^{5A}, Q^{5B}, Q^{5C} are independently for each occurrence absent, alkylene, substituted alkylene wherein one or more methylenes can be interrupted or terminated by one or more of O, S, S(O), SO₂, N(RN), C(R′)═C(R″), C═C or C(O);
R^{2A}, R^{2B}, R^{3A}, R^{3B}, R^{4A}, R^{4B}, R^{5A}, R^{5B}, R^{5C} are each independently for each occurrence absent, NH, O, S, CH₂, C(O) O, C(O) NH, NHCH(R^{a}) C(O), —C(O)—CH(R^{a})—NH—, CO, CH═N—O,

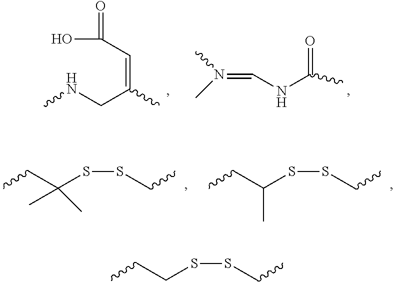

or heterocyclyl;
L^{2A}, L^{2B}, L^{3A}, L^{3B}, L^{4A}, L^{4B}, L^{5A}, L^{5B} and L^{5C} represent the ligand; i.e. each independently for each occurrence a monosaccharide (such as GalNAc), disaccharide, trisaccharide, tetrasaccharide, oligosaccharide, or polysaccharide; and
R^{a} is H or amino acid side chain.

Trivalent conjugating GalNAc derivatives are particularly useful for use with dsRNA agents described herein for inhibiting the expression of a target gene, such as those of Formula (VII):

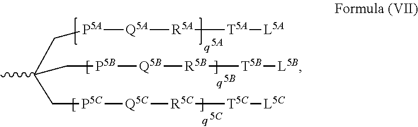

wherein L^{5A}, L^{5B} and L^{5C} represent a monosaccharide, such as GalNAc derivative.

Examples of suitable bivalent and trivalent branched linker groups conjugating GalNAc derivatives include, but are not limited to, the following compounds:

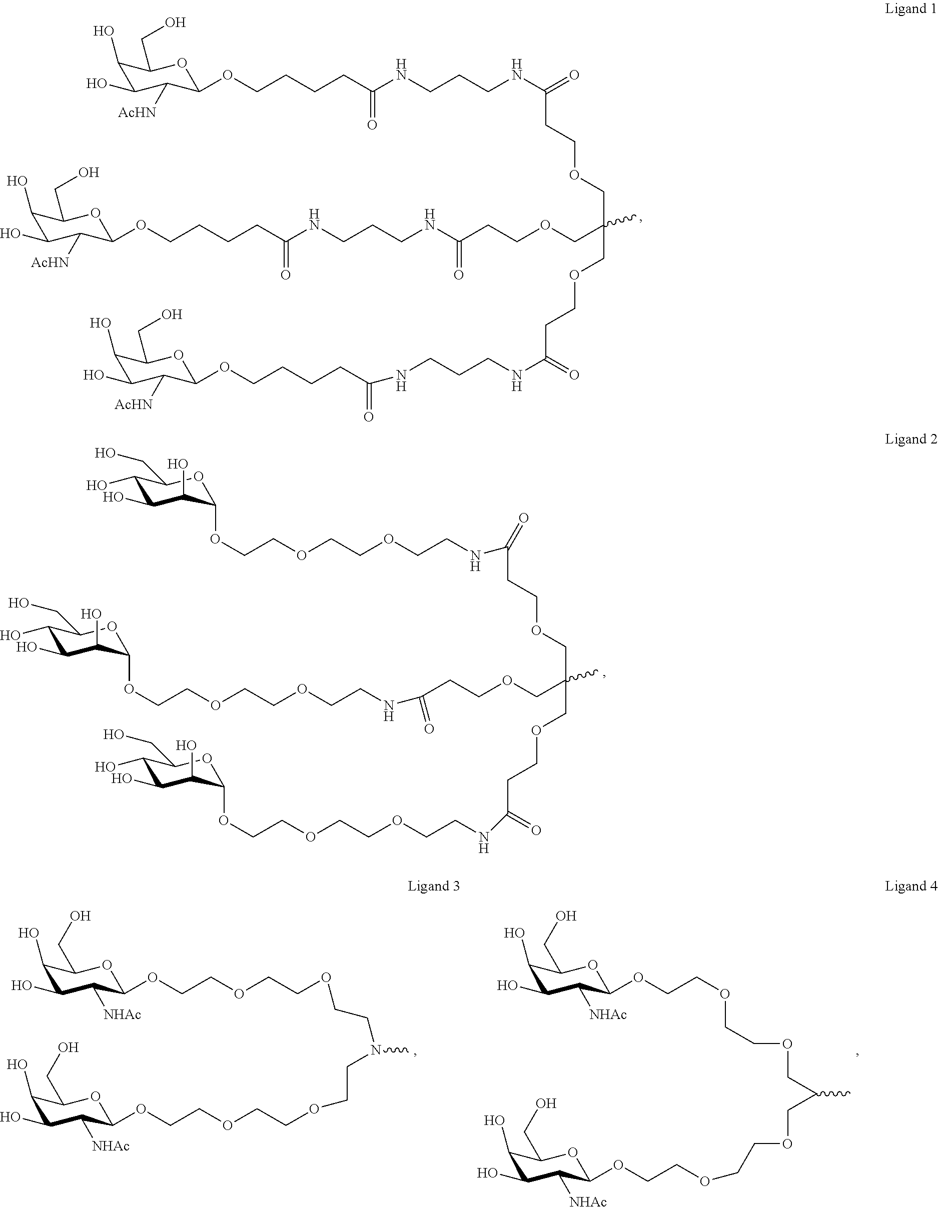

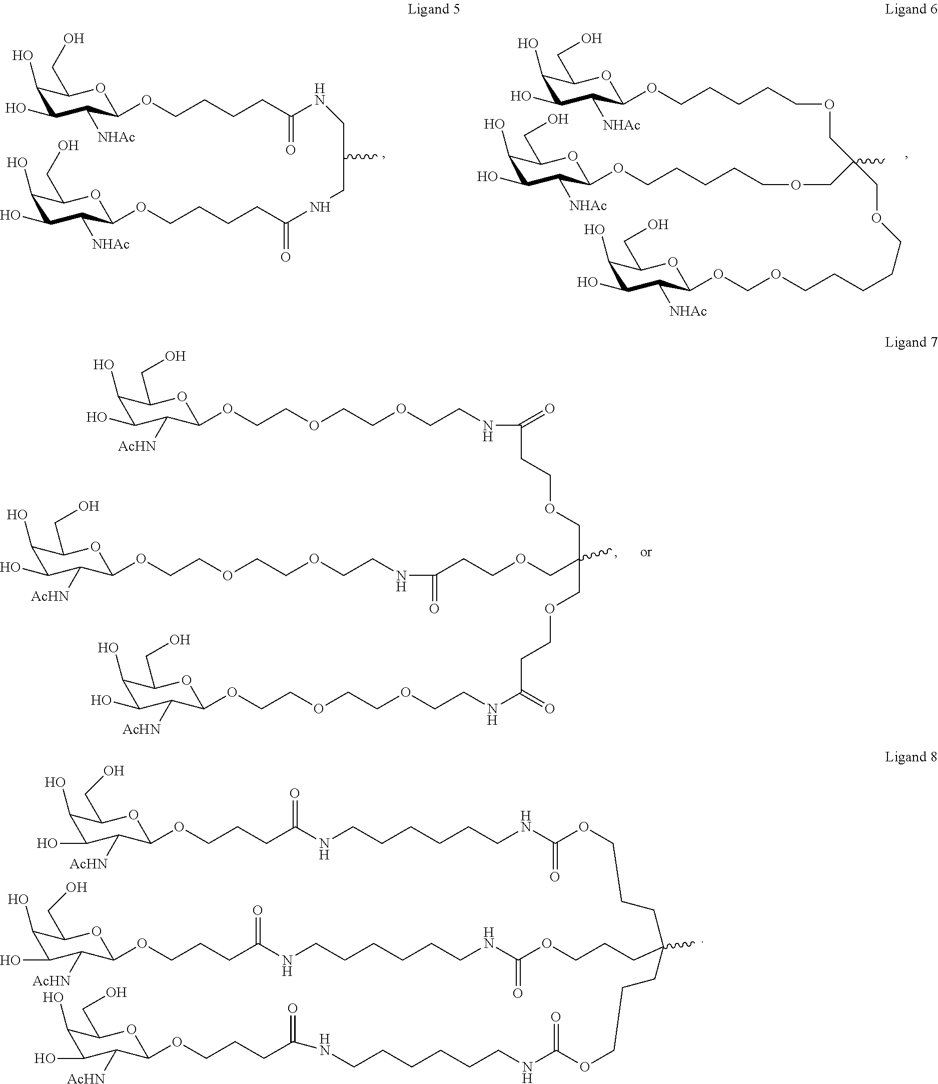

In some embodiments, a dsRNA described herein comprises Ligand 1, i.e., a ligand having the following structure:

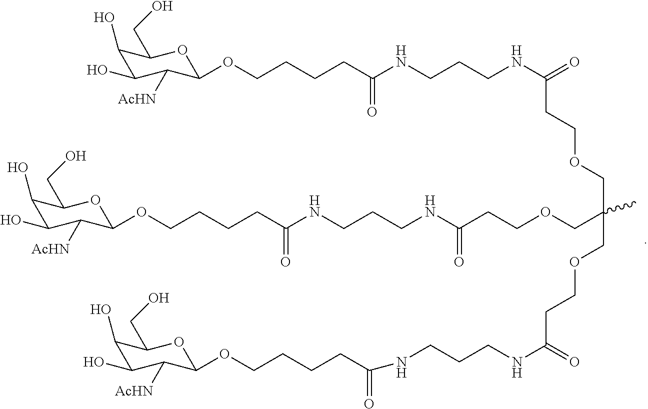 [SMILES: CC(=O)NC1C(OCCCCC(=O)NCCCNC(=O)CCOCC(C)(COCCC(=O)NCCCNC(=O)CCCCOC2OC(CO)C(O)C(O)C2NC(C)=O)COCCC(=O)NCCCNC(=O)CCCCOC2OC(CO)C(O)C(O)C2NC(C)=O)OC(CO)C(O)C1O]

In some embodiments, a dsRNA described herein comprises a ligand described in U.S. Pat. No. 5,994,517 or U.S. Pat. No. 6,906,182, content of each of which is incorporated herein by reference in its entirety.

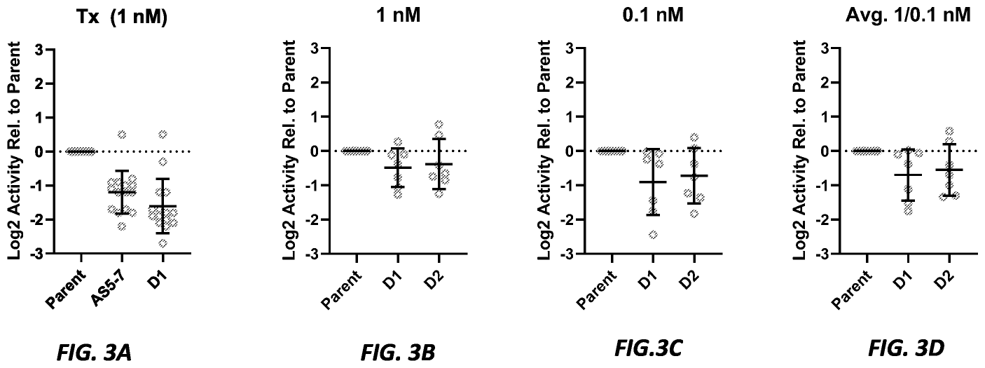

In some embodiments, the ligand can be a tri-antennary ligand described in FIG. 3 of U.S. Pat. No. 6,906,182. For example, a dsRNA described herein can comprise a ligand selected from the following tri-antennary ligands:

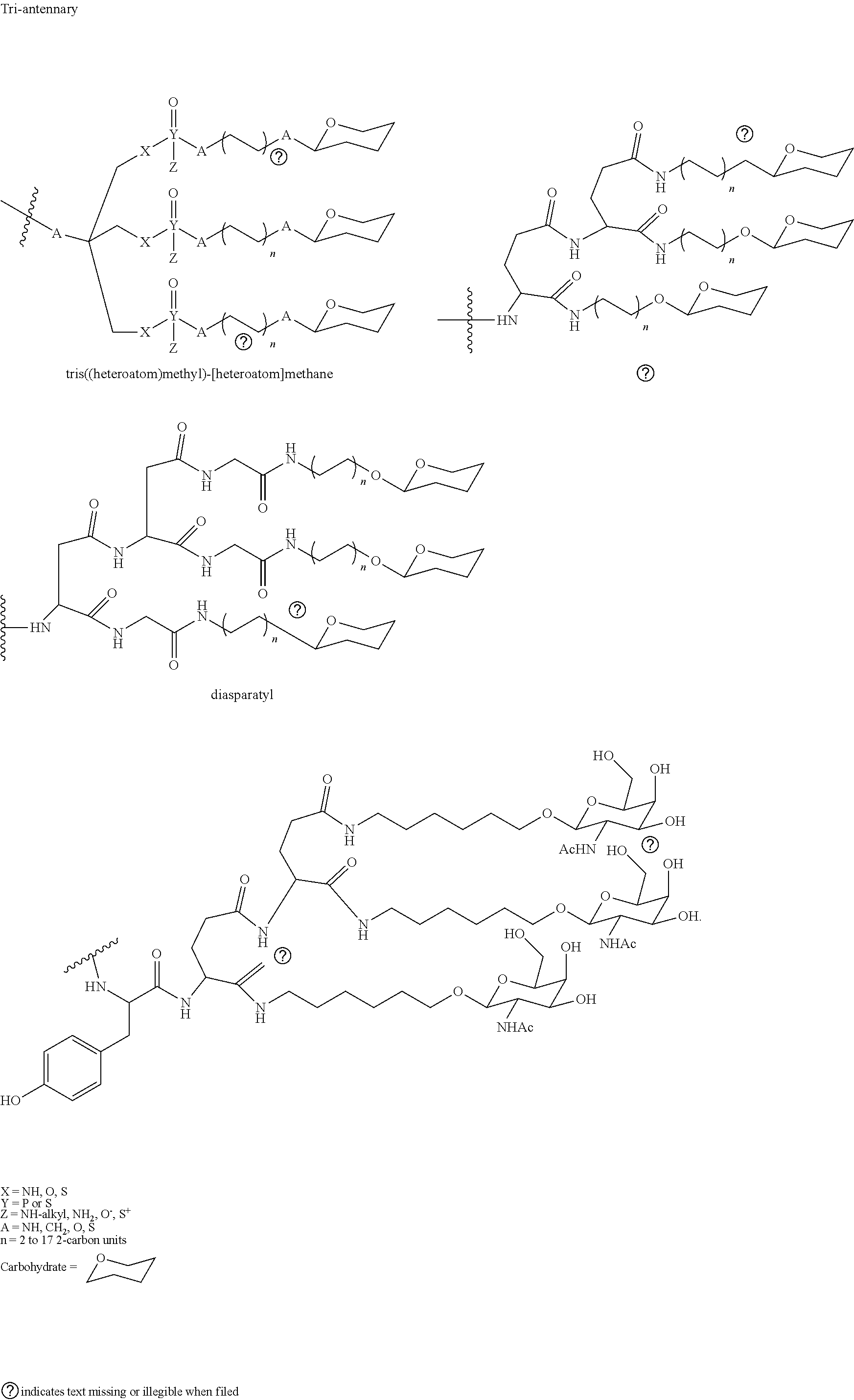

In some embodiments of any one of the aspects, the antisense strand comprises a phosphoryl analog or phosphate mimic at the 5′-terminus. In some embodiments, the antisense strand comprises an alkenylphosphonates, i.e., a vinyl phosphonate at the 5′-terminus. For example, the antisense strand comprises a 5′-E-vinyl phosphonate.

In some embodiments, the antisense strand comprises a cyclopropylphosphonate at the

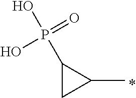

5′-terminus. For example, the antisense comprises at the 5′-terminus, where * is a bond to C5 position of the nucleotide at the 5′-terminus.

In some embodiments of any one of the aspects, at least one of the strands, e.g., the sense and/or the antisense strand of the double-stranded RNA comprises a monomer or ligand selected from the following:

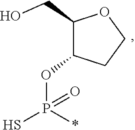 [SMILES: *P(=O)(S)O[C@H]1CCO[C@@H]1CO]

where * is a bond to a 5′, 3′-terminal hydroxyl group of the strand;

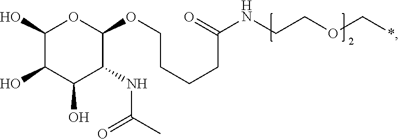

where * is a bond to 2′-hydroxyl of a nucleotide in the strand;

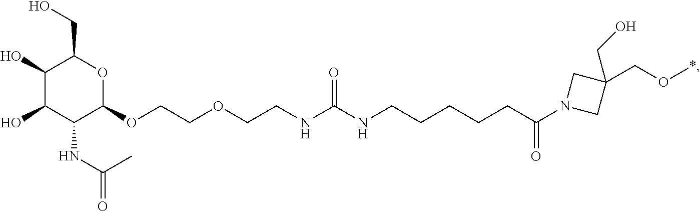

where * is a bond to a 5′ or 3′-terminus of the strand;

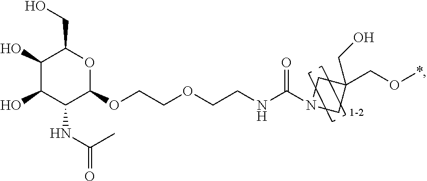

where * is a bond to a 5′ or 3′-terminus of the strand;

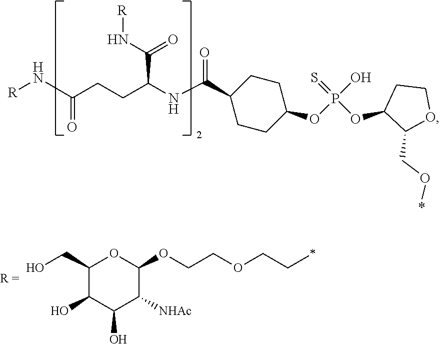

where —O—* is a connection to a 5′ or 3′-terminus of the strand; and/or

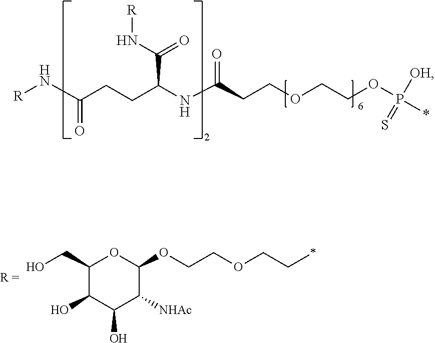

where —P—* is a connection to a 5′ or 3′-hydroxyl group of the strand.

In some embodiments, the ligand comprises a lipophilic group. For example, the ligand can be a C₆₋₃₀aliphatic group or a C₁₀₋₃₀ aliphatic group. “Aliphatic” as used herein means a saturated or unsaturated and straight, branched, and/or cyclic hydrocarbon having the defined number of carbon atoms;l examples include alkyl, alkenyl, alkynyl, cycloalkyl, cycloalkylalkyl, cycloalkylalkenyl, and cycloalkylalkynyl, having the defined number of carbon atoms. In some embodiments, the ligand is a C₁₀₋₃₀alkyl group. For example, the ligand is a straight-chain or branched hexyl, octyl, decyl, dodecyl, tetradecyl, hexadecyl, octadecyl, icosyl, docosyl, or tetracosyl group. For example, the ligand is a straight-chain hexyl, octyl, decyl, dodecyl, tetradecyl, hexadecyl, octadecyl, icosyl, docosyl, or tetracosyl group. For example, the ligand is a straight-chain hexyl, octyl, decyl, dodecyl, hexadecyl, octadecyl, icosyl, or docosyl group. For example, the ligand is a straight-chain hexadecyl group. For example, the ligand is a straight-chain docosyl group.

In certain embodiments, the ligand is conjugated at the 2′-position of a nucleotide at an internal, i.e., non-terminal position of the sense strand and is a straight-chain or branched tetradecyl, hexadecyl, octadecyl, icosyl, docosyl, or tetracosyl group. For example, the ligand is conjugated at the 2′-position of a nucleotide at an internal, i.e., non-terminal position of the sense strand and is a straight-chain or branched hexyl, octyl, decyl, dodecyl, tetradecyl, hexadecyl, octadecyl, icosyl, docosyl, or tetracosyl group. For example, the ligand is conjugated at the 2′-position of a nucleotide at an internal, i.e., non-terminal position of the sense strand and is a straight-chain hexyl, octyl, decyl, dodecyl, tetradecyl, hexadecyl, octadecyl, icosyl, docosyl, or tetracosyl group. For example, the ligand is conjugated at the 2′-position of a nucleotide at an internal, i.e., non-terminal position of the sense strand and is a straight-chain hexadecyl, octadecyl, icosyl, or docosyl group.

The internal sense strand nucleoide position can be all positions except the three terminal positions from each end of the at sense strand. In some embodiments, the internal positions exclude a cleavage site region of the sense strand. In some embodiments, the internal positions exclude positions 9-12 or positions 11-13, counting from the 5′-end of the sense strand. For example, the internal nucleotide position can be one or more of positions 4-8 and 13-18 on the sense strand, such as one or more of positions 5, 6, 7, 15, and 17 on the sense strand counting from the 5′-end of the sense strand. In one embodiment, the internal nucleotide position can be one of positions 5, 6, 7, or 8 of the sense strand, counting from the 5′-end. For example, each of these embodiments, the internal nucleoide position is position 6 or 7 of the sense strand, counting from the 5′-end. For example, each of these embodiments, the internal nucleoide position is position 6 of the sense strand, counting from the 5′-end. For example, each of these embodiments, the internal nucleoide position is position 7 of the sense strand, counting from the 5′-end. In certain embodiments, the internal nucleoide comprising the ligand has the formula,

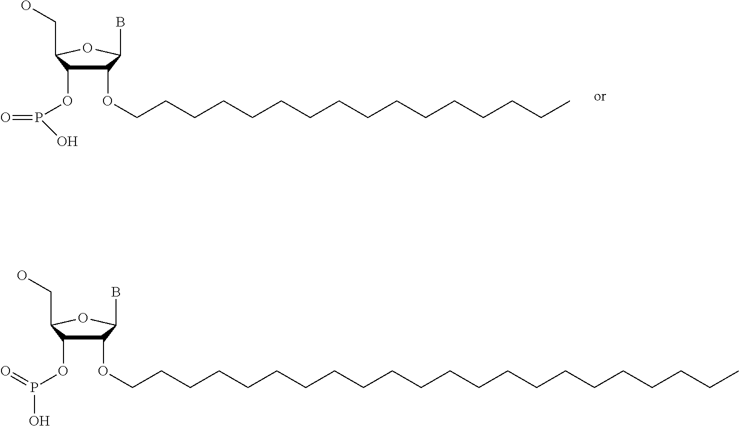

where B is a nucleotide base or a nucleotide base analog, optionally where B is adenine, guanine, cytosine, thymine or uracil.

In some embodiments, the ligand comprises an inverted nucleotide or an inverted abasic nucleotide. For example, the ligand comprises an abasic nucleotide linked via a 5′->5′ or 3′->3′ linkage to a strand of the dsRNA molecule. In some embodiments, the ligand comprises an abasic nucleotide linked via a 3′->3′ linkage to the 3′-end of the sense strand.

In other embodiments, the ligand comprises a lipophilic group that is comprises a steroidal fused ring system. For example, the ligand can comprise cholesterol or corticosterone. Examples of such ligands, include, for example,

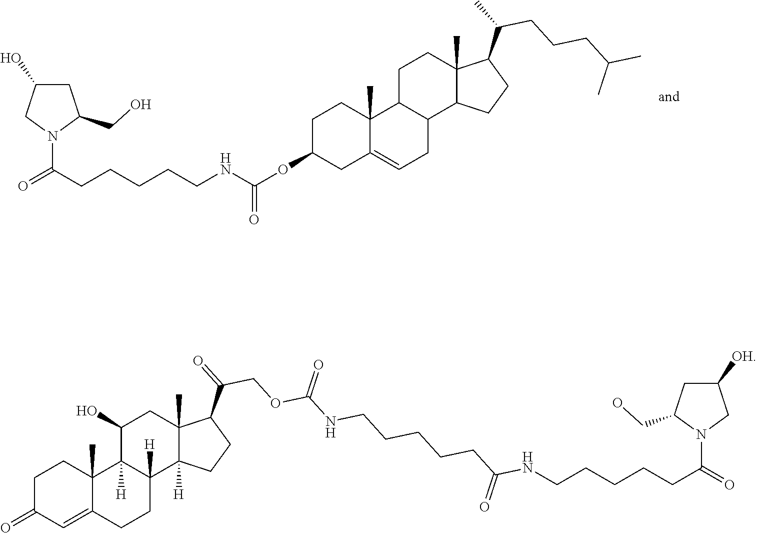

For example, such ligands may be attached to the 5′ and/or 3′ ends of a strand of the dsRNA molecule. In some embodiments, the ligand may be attached to the 5′ or 3′-end of the sense strand. In some embodiments, the ligand may be attached to the 5′-end of the sense strand. In some embodiments, the ligand may be attached to the 3′-end of the sense strand. Attachment to the dsRNA molecule may be via a bond to the oxygen atom illustrated above having an open valence, or a bond formed with the 4-hydoxyl group of the pyrollidine ring.

The ligand may be attached to the polynucleotide via a carrier. The carriers include (i) at least one “backbone attachment point,” preferably two “backbone attachment points” and (ii) at least one “tethering attachment point.” A “backbone attachment point” as used herein refers to a functional group, e.g. a hydroxyl group, or generally, a bond available for, and that is suitable for incorporation of the carrier into the backbone, e.g., the phosphate, or modified phosphate, e.g., sulfur containing, backbone, of a ribonucleic acid. A “tethering attachment point” (TAP) in some embodiments refers to a constituent ring atom of the cyclic carrier, e.g., a carbon atom or a heteroatom (distinct from an atom which provides a backbone attachment point), that connects a selected moiety. The moiety can be, e.g., a carbohydrate, e.g. monosaccharide, disaccharide, trisaccharide, tetrasaccharide, oligosaccharide and polysaccharide. Optionally, the selected moiety is connected by an intervening tether to the cyclic carrier. Thus, the cyclic carrier will often include a functional group, e.g., an amino group, or generally, provide a bond, that is suitable for incorporation or tethering of another chemical entity, e.g., a ligand to the constituent ring.

In one embodiment the dsRNA molecule of the invention is conjugated to a ligand via a carrier, wherein the carrier can be cyclic group or acyclic group; preferably, the cyclic group is selected from pyrrolidinyl, pyrazolinyl, pyrazolidinyl, imidazolinyl, imidazolidinyl, piperidinyl, piperazinyl, [1,3]dioxolane, oxazolidinyl, isoxazolidinyl, morpholinyl, thiazolidinyl, isothiazolidinyl, quinoxalinyl, pyridazinonyl, tetrahydrofuryl and decalin; preferably, the acyclic group is selected from serinol backbone or diethanolamine backbone.

The ligand can be attached to the sense strand, antisense strand or both strands, at the 3′-end, 5′-end or both ends. For instance, the ligand can be conjugated to the sense strand, in particular, the 3′-end of the sense strand.

The ligand can be conjugated to the sense strand or the antisense strand via a linker comprising a cleavable group. A cleavable linking group is one which is sufficiently stable outside the cell, but which upon entry into a target cell is cleaved to release the two parts the linker is holding together. In a preferred embodiment of the dsRNA molecule according to the present invention, the cleavable linking group is cleaved at least 10 times or more, preferably at least 100 times faster in the target cell or under a first reference condition (which can, e.g., be selected to mimic or represent intracellular conditions) than in the blood of a subject, or under a second reference condition (which can, e.g., be selected to mimic or represent conditions found in the blood or serum).

Cleavable linking groups are susceptible to cleavage agents, e.g., pH, redox potential or the presence of degradative molecules. Generally, cleavage agents are more prevalent or found at higher levels or activities inside cells than in serum or blood. Examples of such degradative agents include: redox agents which are selected for particular substrates or which have no substrate specificity, including, e.g., oxidative or reductive enzymes or reductive agents such as mercaptans, present in cells, that can degrade a redox cleavable linking group by reduction; esterases; endosomes or agents that can create an acidic environment, e.g., those that result in a pH of five or lower; enzymes that can hydrolyze or degrade an acid cleavable linking group by acting as a general acid, peptidases (which can be substrate specific), and phosphatases.

A cleavable linkage group, such as a disulfide bond can be susceptible to pH. The pH of human serum is 7.4, while the average intracellular pH is slightly lower, ranging from about 7.1-7.3. Endosomes have a more acidic pH, in the range of 5.5-6.0, and lysosomes have an even more acidic pH at around 5.0. Some linkers will have a cleavable linking group that is cleaved at a preferred pH, thereby releasing the cationic lipid from the ligand inside the cell, or into the desired compartment of the cell.

A linker can include a cleavable linking group that is cleavable by a particular enzyme. The type of cleavable linking group incorporated into a linker can depend on the cell to be targeted.

For example, liver targeting ligands can be linked to the cationic lipids through a linker that includes an ester group. Liver cells are rich in esterases, and therefore the linker will be cleaved more efficiently in liver cells than in cell types that are not esterase-rich. Other cell-types rich in esterases include cells of the lung, renal cortex, and testis.

Linkers that contain peptide bonds can be used when targeting cell types rich in peptidases, such as liver cells and synoviocytes.

In general, the suitability of a candidate cleavable linking group can be evaluated by testing the ability of a degradative agent (or condition) to cleave the candidate linking group. It will also be desirable to also test the candidate cleavable linking group for the ability to resist cleavage in the blood or when in contact with other non-target tissue. Thus one can determine the relative susceptibility to cleavage between a first and a second condition, where the first is selected to be indicative of cleavage in a target cell and the second is selected to be indicative of cleavage in other tissues or biological fluids, e.g., blood or serum. The evaluations can be carried out in cell free systems, in cells, in cell culture, in organ or tissue culture, or in whole animals. It may be useful to make initial evaluations in cell-free or culture conditions and to confirm by further evaluations in whole animals. In preferred embodiments, useful candidate compounds are cleaved at least 2, 4, 10 or 100 times faster in the cell (or under in vitro conditions selected to mimic intracellular conditions) as compared to blood or serum (or under in vitro conditions selected to mimic extracellular conditions).

One class of cleavable linking groups is redox cleavable linking groups, which may be used in the dsRNA molecule according to the present invention that are cleaved upon reduction or oxidation. An example of reductively cleavable linking group is a disulfide linking group (—S—S—). To determine if a candidate cleavable linking group is a suitable “reductively cleavable linking group,” or for example is suitable for use with a particular iRNA moiety and particular targeting agent one can look to methods described herein. For example, a candidate can be evaluated by incubation with dithiothreitol (DTT), or other reducing agent using reagents know in the art, which mimic the rate of cleavage which would be observed in a cell, e.g., a target cell. The candidates can also be evaluated under conditions which are selected to mimic blood or serum conditions. In a preferred embodiment, candidate compounds are cleaved by at most 10% in the blood. In preferred embodiments, useful candidate compounds are degraded at least 2, 4, 10 or 100 times faster in the cell (or under in vitro conditions selected to mimic intracellular conditions) as compared to blood (or under in vitro conditions selected to mimic extracellular conditions). The rate of cleavage of candidate compounds can be determined using standard enzyme kinetics assays under conditions chosen to mimic intracellular media and compared to conditions chosen to mimic extracellular media.

Phosphate-based cleavable linking groups, which may be used in the dsRNA molecule according to the present invention, are cleaved by agents that degrade or hydrolyze the phosphate group. An example of an agent that cleaves phosphate groups in cells are enzymes such as phosphatases in cells. Examples of phosphate-based linking groups are —O—P(O)(ORk)—O—, —O—P(S)(ORk)—O—, —O—P(S)(SRk)—O—, —S—P(O)(ORk)—O—, —O—P(O)(ORk)—S—, —S—P(O)(ORk)—S—, —O—P(S)(ORk)—S—, —S—P(S)(ORk)—O—, —O—P(O)(Rk)—O—, —O—P(S)(Rk)—O—, —S—P(O)(Rk)—O—, —S—P(S)(Rk)—O—, —S—P(O)(Rk)—S—, —O—P(S)(Rk)—S—, wherein Rk at each occurrence can be, independently, hydrogen, C1-C20 alkyl, C1-C20 haloalkyl, C6-C10 aryl, C7-C12 aralkyl. Preferred embodiments are —O—P(O)(OH)—O—, —O—P(S)(OH)—O—, —O—P(S)(SH)—O—, —S—P(O)(OH)—O—, —O—P(O)(OH)—S—, —S—P(O)(OH)—S—, —O—P(S)(OH)—S—, —S—P(S)(OH)—O—, —O—P(O)(H)—O—, —O—P(S)(H)—O—, —S—P(O)(H)—O—, —S—P(S)(H)—O—, —S—P(O)(H)—S—, —O—P(S)(H)—S—. A preferred embodiment is —O—P(O)(OH)—O—. These candidates can be evaluated using methods analogous to those described above.

Acid cleavable linking groups, which may be used in the dsRNA molecule according to the present invention, are linking groups that are cleaved under acidic conditions. In preferred embodiments acid cleavable linking groups are cleaved in an acidic environment with a pH of about 6.5 or lower (e.g., about 6.0, 5.5, 5.0, or lower), or by agents such as enzymes that can act as a general acid. In a cell, specific low pH organelles, such as endosomes and lysosomes can provide a cleaving environment for acid cleavable linking groups. Examples of acid cleavable linking groups include but are not limited to hydrazones, esters, and esters of amino acids. Acid cleavable groups can have the general formula —C═NN—, C(O) O, or —OC(O). A preferred embodiment is when the carbon attached to the oxygen of the ester (the alkoxy group) is an aryl group, substituted alkyl group, or tertiary alkyl group such as dimethyl pentyl or t-butyl. These candidates can be evaluated using methods analogous to those described above.

Ester-based cleavable linking groups, which may be used in the dsRNA molecule according to the present invention, are cleaved by enzymes such as esterases and amidases in cells. Examples of ester-based cleavable linking groups include but are not limited to esters of alkylene, alkenylene and alkynylene groups. Ester cleavable linking groups have the general formula —C(O)O—, or —OC(O)—. These candidates can be evaluated using methods analogous to those described above.

Peptide-based cleavable linking groups, which may be used in the dsRNA molecule according to the present invention, are cleaved by enzymes such as peptidases and proteases in cells. Peptide-based cleavable linking groups are peptide bonds formed between amino acids to yield oligopeptides (e.g., dipeptides, tripeptides etc.) and polypeptides. Peptide-based cleavable groups do not include the amide group (—C(O) NH—). The amide group can be formed between any alkylene, alkenylene or alkynylene. A peptide bond is a special type of amide bond formed between amino acids to yield peptides and proteins. The peptide based cleavage group is generally limited to the peptide bond (i.e., the amide bond) formed between amino acids yielding peptides and proteins and does not include the entire amide functional group. Peptide-based cleavable linking groups have the general formula —NHCHR^{A}C(O) NHCHR^{B}C(O)—, where R^{A} and R^{B} are the R groups of the two adjacent amino acids. These candidates can be evaluated using methods analogous to those described above. As used herein, “carbohydrate” refers to a compound which is either a carbohydrate per se made up of one or more monosaccharide units having at least 6 carbon atoms (which may be linear, branched or cyclic) with an oxygen, nitrogen or sulfur atom bonded to each carbon atom; or a compound having as a part thereof a carbohydrate moiety made up of one or more monosaccharide units each having at least six carbon atoms (which may be linear, branched or cyclic), with an oxygen, nitrogen or sulfur atom bonded to each carbon atom. Representative carbohydrates include the sugars (mono-, di-, tri- and oligosaccharides containing from about 4-9 monosaccharide units), and polysaccharides such as starches, glycogen, cellulose and polysaccharide gums. Specific monosaccharides include C₅ and above (preferably C₅-C₈) sugars; di- and trisaccharides include sugars having two or three monosaccharide units (preferably C₅-C₈).

In some embodiments, the dsRNA molecule of the invention comprises one or more overhang regions and/or capping groups of dsRNA molecule at the 3′-end, or 5′-end or both ends of a strand. The overhang can be 1-10 nucleotides in length. For example, the overhang can be 1, 2, 3, 4, 5, 6, 7, 8, 9 or 10 nucleotides in length. In some embodiments, the overhang is 1-6 nucleotides in length, for instance 2-6 nucleotides in length, 1-5 nucleotides in length, 2-5 nucleotides in length, 1-4 nucleotides in length, 2-4 nucleotides in length, 1-3 nucleotides in length, 2-3 nucleotides in length, or 1-2 nucleotides in length. The overhangs can be the result of one strand being longer than the other, or the result of two strands of the same length being staggered. The overhang can form a mismatch with the target sequence or it can be complementary to the gene sequences being targeted or it can be the other sequence. The first and second strands can also be joined, e.g., by additional bases to form a hairpin, or by other non-base linkers.

In some embodiments, the nucleotides in the overhang region of the dsRNA molecule of the invention can each independently be a modified or unmodified nucleotide including, but not limited to 2′-sugar modified, such as, 2′-Fluoro 2′-O-methyl, thymidine (T), 2′-O-methoxyethyl-5-methyluridine, 2′-O-methoxyethyladenosine, 2′-O-methoxyethyl-5-methylcytidine, GNA, SNA, hGNA, hhGNA, mGNA, TNA, h′GNA, and any combinations thereof. For example, dTdT can be an overhang sequence for either end on either strand. The overhang can form a mismatch with the target mRNA or it can be complementary to the gene sequences being targeted or can be other sequence.

The 5′- or 3′-overhangs at the sense strand, antisense strand or both strands of the dsRNA molecule of the invention may be phosphorylated. In some embodiments, the overhang region contains two nucleotides having a phosphorothioate between the two nucleotides, where the two nucleotides can be the same or different. In some embodiments, the overhang is present at the 3′-end of the sense strand, antisense strand or both strands. In some embodiments, this 3′-overhang is present in the antisense strand. In some embodiments, this 3′-overhang is present in the sense strand.

The dsRNA molecule of the invention may comprise only a single overhang, which can strengthen the interference activity of the dsRNA, without affecting its overall stability. For example, the single-stranded overhang is located at the 3′-terminal end of the sense strand or, alternatively, at the 3′-terminal end of the antisense strand. The dsRNA can also have a blunt end, located at the 5′-end of the antisense strand (or the 3′-end of the sense strand) or vice versa.

Generally, the antisense strand of the dsRNA has a nucleotide overhang at the 3′-end, and the 5′-end is blunt. While not bound by theory, the asymmetric blunt end at the 5′-end of the antisense strand and 3′-end overhang of the antisense strand favor the guide strand loading into RISC process. For example, the single overhang is at least one, two, three, four, five, six, seven, eight, nine, or ten nucleotides in length. In some embodiments, the dsRNA has a 2 nucleotide overhang on the 3′-end of the antisense strand and a blunt end at the 5′-end of the antisense strand.

The dsRNA of the invention can comprise one or more modified nucleotides. For example, every nucleotide in the sense strand and antisense strand of the dsRNA molecule can be modified. Each nucleotide can be modified with the same or different modification which can include one or more alteration of one or both of the non-linking phosphate oxygens and/or of one or more of the linking phosphate oxygens; alteration of a constituent of the ribose sugar; replacement of the ribose sugar; wholesale replacement of the phosphate moiety with “dephospho” linkers; modification or replacement of a naturally occurring base; and replacement or modification of the ribose-phosphate backbone.

As nucleic acids are polymers of subunits, many of the modifications occur at a position which is repeated within a nucleic acid, e.g., a modification of a base, or a phosphate moiety, or a non-linking O of a phosphate moiety. In some cases, the modification will occur at all of the subject positions in the nucleic acid but in many cases it will not. By way of example, a modification may only occur at a 3′ or 5′ terminal position, may only occur in a central region, may only occur at a non-terminal region, or may only occur in a terminal region, e.g., at a position on a terminal nucleotide or in the last 2, 3, 4, 5, or 10 nucleotides of a strand. A modification may occur in a double strand region, a single strand region, or in both. A modification may occur only in the double strand region of a RNA or may only occur in a single strand region of a RNA. For example, a phosphorothioate modification at a non-linking O position may only occur at one or both termini, may only occur in a terminal region, e.g., at a position on a terminal nucleotide or in the last 2, 3, 4, 5, or 10 nucleotides of a strand, or may occur in double strand and single strand regions, particularly at termini. The 5′ end or ends can be phosphorylated.

It may be possible, e.g., to enhance stability, to include particular bases in overhangs, or to include modified nucleotides or nucleotide surrogates, in single strand overhangs, e.g., in a 5′ or 3′ overhang, or in both. For example, it can be desirable to include purine nucleotides in overhangs. In some embodiments all or some of the bases in a 3′ or 5′ overhang may be modified, e.g., with a modification described herein. Modifications can include, e.g., the use of modifications at the 2′ position of the ribose sugar with modifications that are known in the art, e.g., the use of deoxyribonucleotides, 2′-deoxy-2′-fluoro (2′-F) or 2′-O-methyl modified instead of the ribosugar of the nucleobase, and modifications in the phosphate group, e.g., phosphorothioate modifications. Overhangs need not be homologous with the target sequence.

In some embodiments, the dsRNA molecule of the invention comprises modifications of an alternating pattern, particular in the B1, B2, B3, B1′, B2′, B3′, B4′ regions. The term “alternating motif” or “alternative pattern” as used herein refers to a motif having one or more modifications, each modification occurring on alternating nucleotides of one strand. The alternating nucleotide may refer to one per every other nucleotide or one per every three nucleotides, or a similar pattern. For example, if A, B and C each represent one type of modification to the nucleotide, the alternating motif can be “ABABABABABAB . . . ” “AABBAABBAABB . . . ” “AABAABAABAAB . . . ” “AAABAAABAAAB . . . “AAABBBAAABBB . . . ,” or “ABCABCABCABC . . . ,” etc.

The type of modifications contained in the alternating motif may be the same or different. For example, if A, B, C, D each represent one type of modification on the nucleotide, the alternating pattern, i.e., modifications on every other nucleotide, may be the same, but each of the sense strand or antisense strand can be selected from several possibilities of modifications within the alternating motif such as “ABABAB . . . “, “ACACAC . . . ” “BDBDBD . . . ” or “CDCDCD . . . ,” etc.

In some embodiments, the dsRNA molecule of the invention comprises the modification pattern for the alternating motif on the sense strand relative to the modification pattern for the alternating motif on the antisense strand is shifted. The shift may be such that the modified group of nucleotides of the sense strand corresponds to a differently modified group of nucleotides of the antisense strand and vice versa. For example, the sense strand when paired with the antisense strand in the dsRNA duplex, the alternating motif in the sense strand may start with “ABABAB” from 5′-3′ of the strand and the alternating motif in the antisense strand may start with “BABABA” from 3′-5′ of the strand within the duplex region. As another example, the alternating motif in the sense strand may start with “AABBAABB” from 5′-3′ of the strand and the alternating motif in the antisense strand may start with “BBAABBAA” from 3′-5′ of the strand within the duplex region, so that there is a complete or partial shift of the modification patterns between the sense strand and the antisense strand.

### 5′-Modifications

In some embodiments dsRNA molecules of the invention are 5′ phosphorylated or include a phosphoryl analog or phosphate mimic at the 5′ terminus. 5′-phosphate modifications include those which are compatible with RISC mediated gene silencing. Suitable modifications include: 5′-monophosphate ((HO)₂(O)P—O-5′); 5′-diphosphate ((HO)₂(O)P—O—P(HO)(O)—O-5′); 5′-triphosphate ((HO)₂(O)P—O—(HO)(O)P—O—P(HO)(O)—O—5′); 5′-guanosine cap (7-methylated or non-methylated)(7m-G-O—5′-(HO)(O)P—O—(HO)(O)P—O—P(HO)(O)—O—5′); 5′-adenosine cap (Appp), and any modified or unmodified nucleotide cap structure (N—O—5′-(HO)(O)P—O—(HO)(O)P—O-P(HO)(O)—O—5′); 5′-monothiophosphate (phosphorothioate; (HO)₂(S)P—O—5′); 5′-monodithiophosphate (phosphorodithioate; (HO)(HS)(S)P—O—5′), 5′-phosphorothiolate ((HO)₂(O)P—S-5′); any additional combination of oxygen/sulfur replaced monophosphate, diphosphate and triphosphates (e.g. 5′-alpha-thiotriphosphate, 5′-gamma-thiotriphosphate, etc.), 5′-phosphoramidates ((HO)₂(O)P—NH-5′, (HO)(NH2)(O)P—O—5′), 5′-alkylphosphonates (e.g. RP(OH)(O)—O—5′-, R=alkyl, such as methyl, ethyl, isopropyl, propyl, etc.), 5′-alkenylphosphonates (i.e. vinyl, substituted vinyl), (OH)₂(O)P—5′—CH₂-), cyclopropylphosphonates, 5′-alkyletherphosphonates (R=alkylether-methoxymethyl(MeOCH2—), ethoxymethyl, etc., e.g. RP(OH)(O)—O—5′-). In one example, the modification can in placed in the antisense strand of a dsRNA molecule.

In some embodiments of any one of the aspects, the antisense strand comprises a phosphoryl analog or phosphate mimic at the 5′-terminus. In some embodiments, the antisense strand comprises an alkenylphosphonates, i.e., a vinyl phosphonate at the 5′-terminus. For example, the antisense strand comprises a 5′-E-vinyl phosphonate. In exemplary embodiments, a 5′ vinyl phosphonate modified nucleotide at the 5′-terminus may have the structure:

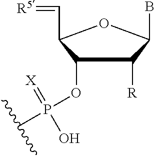

wherein X is O or S; R is hydrogen, hydroxy, fluoro, or C₁₋₂₀alkoxy (e.g., methoxy); R⁵′ is ═C(H)—P(O)(OH)₂ and the double bond between the C₅′ carbon and R⁵′ is in the E or Z orientation (e.g., E orientation); and B is a nucleobase or a modified nucleobase, optionally where B is adenine, guanine, cytosine, thymine, or uracil.

In some embodiments, the antisense strand comprises a cyclopropylphosphonate at the 5′-terminus. For example, the antisense comprises

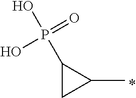

at the 5′-terminus (which, for example, may replace the 4′-group in immediately preceding structure.

The dsRNA agents of the invention can comprise thermally destabilizing modifications in the seed region of the antisense strand (i.e., at positions 2-9 of the 5′-end of the antisense strand) to reduce or inhibit off-target gene silencing. Without wishing to be bound by a theory, dsRNAs with an antisense strand comprising at least one thermally destabilizing modification of the duplex within the first 9 nucleotide positions, counting from the 5′ end, of the antisense strand have reduced off-target gene silencing activity. Accordingly, in some embodiments, the antisense strand comprises at least one (e.g., one, two, three, four, five or more) thermally destabilizing modification of the duplex within the first 9 nucleotide positions of the 5′ region of the antisense strand. In some embodiments, thermally destabilizing modification of the duplex is located in positions 2-9, or preferably positions 4-8, from the 5′-end of the antisense strand. In some further embodiments, the thermally destabilizing modification of the duplex is located at position 5, 6, 7 or 8 from the 5′-end of the antisense strand.

In still some further embodiments, the thermally destabilizing modification of the duplex is located at position 7 from the 5′-end of the antisense strand.

The term “thermally destabilizing modification(s)” includes modification(s) that would result with a dsRNA with a lower overall melting temperature (Tm)(preferably a Tm with one, two, three or four degrees lower than the Tm of the dsRNA without having such modification(s). In some embodiments, the thermally destabilizing modification of the duplex is located at position 2, 3, 4, 5, 6, 7, 8 or 9 from the 5′-end of the antisense strand.

The thermally destabilizing modifications can include, but are not limited to, abasic modification; mismatch with the opposing nucleotide in the opposing strand; and sugar modification such as 2′-deoxy modification or acyclic nucleotide, e.g., unlocked nucleic acids (UNA) or glycol nucleic acid (GNA), or a 5′-2′-linked nucleotide (e.g., having 3′-OMe, 3′-F, 3′-H or 3′-OH, herein a “3′-RNA”). For example, the thermally destabilizing modifications can include, but are not limited to, mUNA and GNA building blocks as follows:

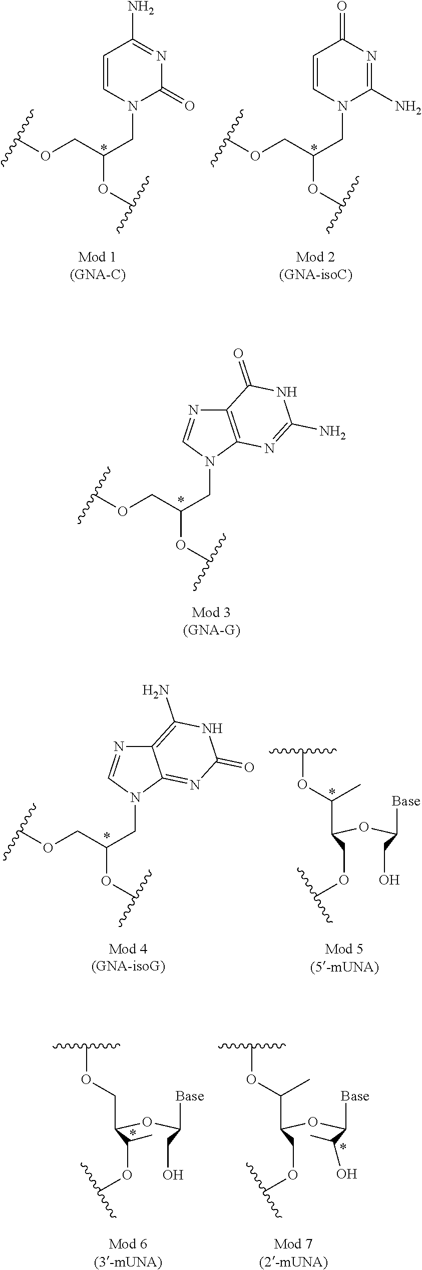

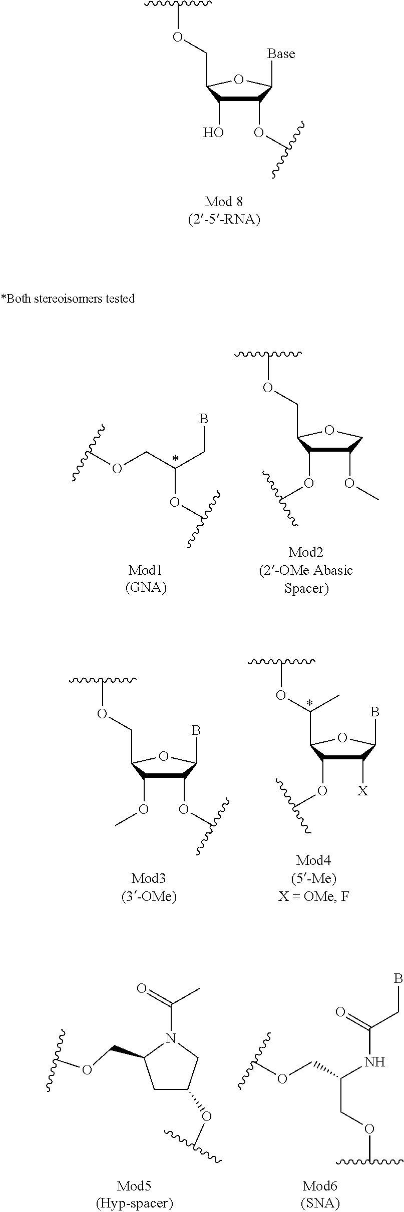

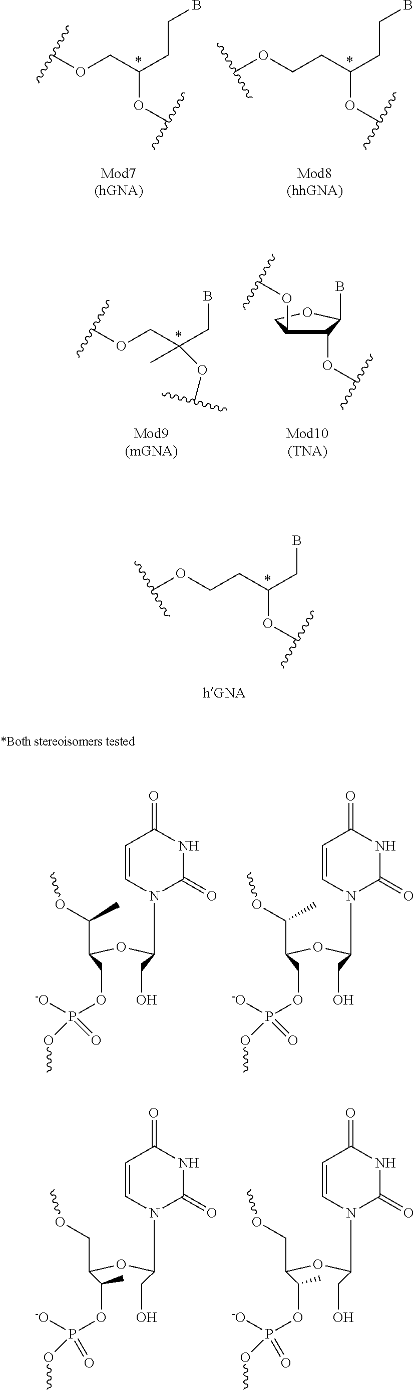

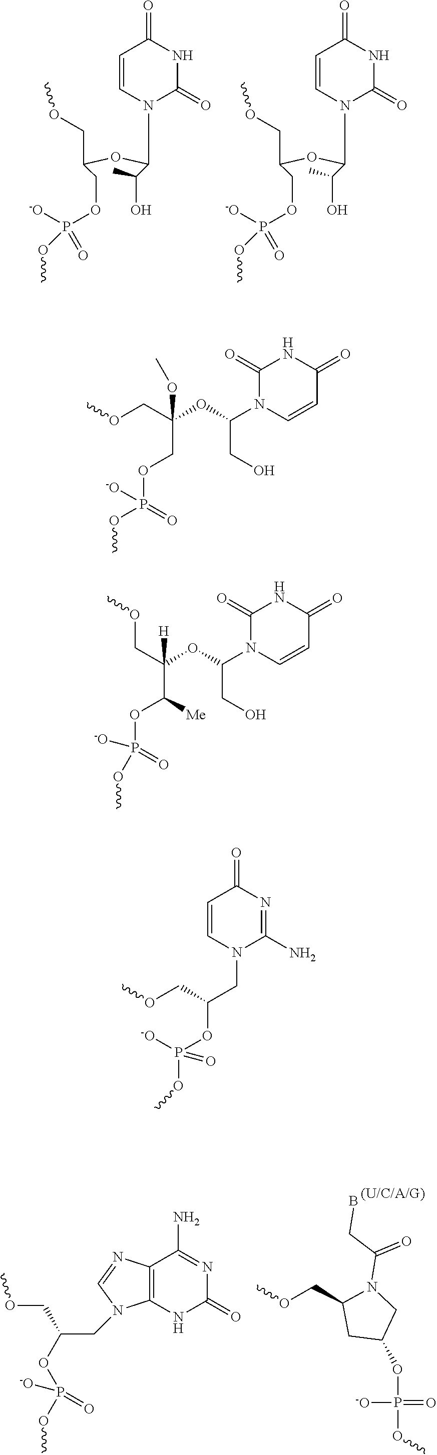

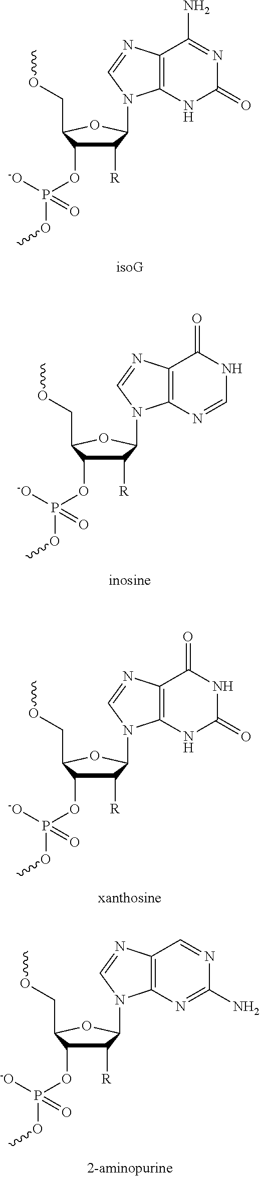

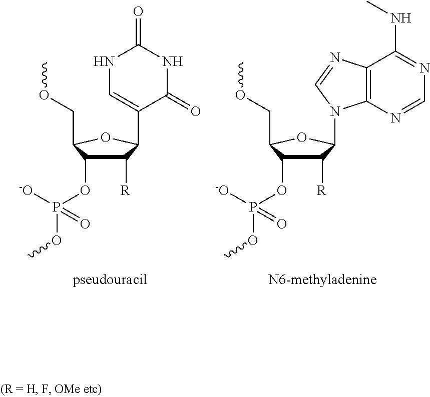

In some embodiments, the destabilizing modification is a 5′-2′-linked nucleotide (e.g., having 3′-OMe, 3′-F, 3′-H or 3′-OH. In some embodiments, the destabilizing modification is a 5′-2′-linked nucleotide having 3′-OMe, 3′-F, 3′-H or 3′-OH. In some embodiments, the destabilizing modification is a 5′-2′-linked nucleotide having 3′-OMe. In some embodiments, the destabilizing modification is a 5′-2′-linked nucleotide having 3′-F. In some embodiments, the destabilizing modification is a 5′-2′-linked nucleotide having 3′-H. In some embodiments, the destabilizing modification is a 5′-2′-linked nucleotide having 3′-OH, e.g., having the formula

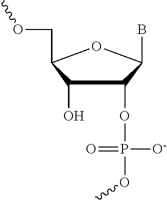

wherein B is a nucleotide base or a nucleotide base analog, optionally where B is adenine, guanine, cytosine, thymine or uracil.

In some embodiments, the destabilizing modification is selected from the group consisting of GNA-isoC, GNA-isoG, 5′-mUNA, 4′-mUNA, 3′-mUNA, and 2′-mUNA.

In some embodiments, the destabilizing modification mUNA is selected from the group consisting of

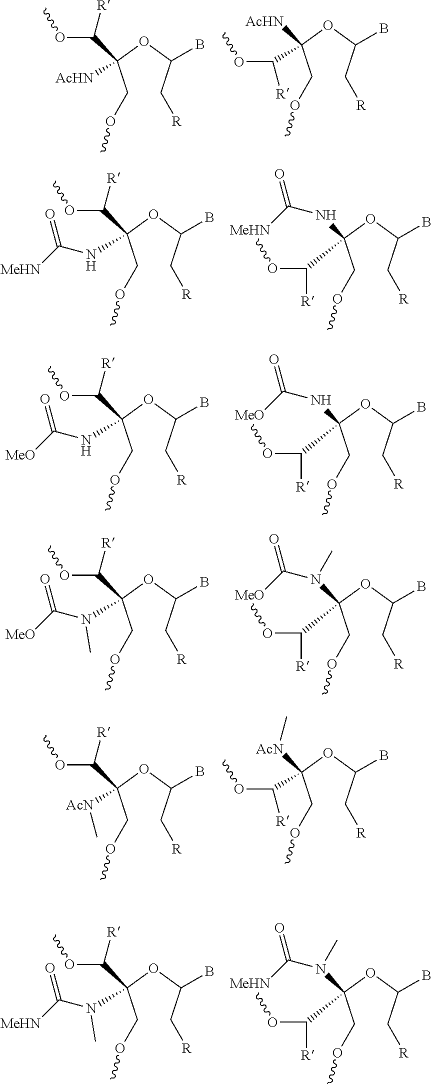

R═H, OH; OMe; Cl, F; OH; O—(CH₂)₂OMe; SMe, NMe₂; NH₂; Me; CCH(alkyne), O-nPr; O-alkyl; O-alkylamino;
R′═H, Me;
B=A; C; 5-Me-C; G; I; U; T; Y; 2-thiouridine; 4-thiouridine; C5-modified pyrimidines; C2-modified purines; N8-modiifed purines; phenoxazine; G-clamp; non-canonical mono, bi and tricyclic heterocycles; pseudouracil; isoC; isoG; 2,6-diamninopurine; pseudocytosine; 2-aminopurine; xanthosine; N⁶-alkyl-A; O⁶-alkyl-G; 2-thiouridine; 4-thiouridine; C5-modified pyrimidines; C2-modified purines; N8-modiifed purines; 7-deazapurines, phenoxazine; G-clamp; non-canonical mono, bi and tricyclic heterocycles; and
Stereochemistry is R or S and combination of R and S for the unspecified chiral centers.

In some embodiments, the destabilizing modification mUNA is selected from the group consisting of

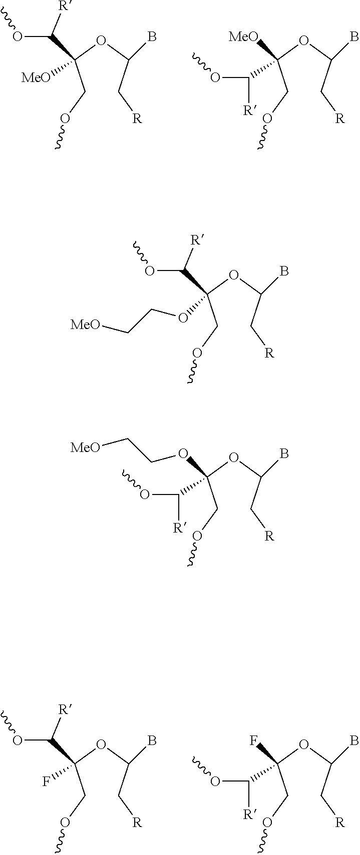

R═H, OH; OMe; CI, F; OH; O—(CH₂)₂OMe; SMe, NMe₂; NH₂; Me; CCH(alkyne), O-nPr; O-alkyl; O-alkylamino;
R′═H, Me;
B=A; C; 5-Me-C; G; I; U; T; Y; 2-thiouridine; 4-thiouridine; C₅-modified pyrimidines; C2-modified purines; N8-modiifed purines; phenoxazine; G-clamp; non-canonical mono, bi and tricyclic heterocycles; pseudouracil; isoC; isoG; 2,6-diamninopurine; pseudocytosine; 2-aminopurine; xanthosine; N⁶-alkyl-A; O⁶-alkyl-G; 2-thiouridine; 4-thiouridine; C₅-modified pyrimidines; C2-modified purines; N8-modiifed purines; 7-deazapurines, phenoxazine; G-clamp; non-canonical mono, bi and tricyclic heterocycles; and
Stereochemistry is R or S and combination of R and S for the unspecified chiral centers.

In some embodiments, the destabilizing modification mUNA is selected from the group consisting of

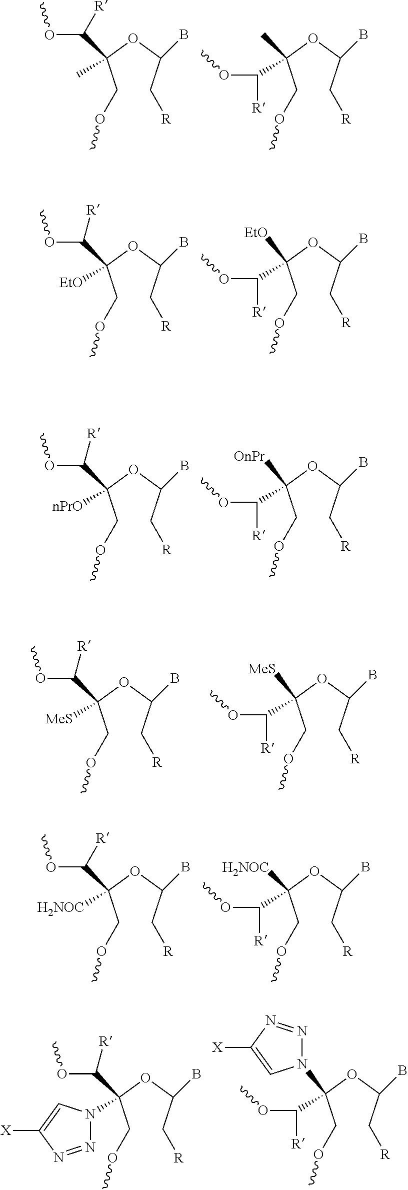

R═H, OMe; F; OH; O—(CH₂)₂OMe; SMe, NMe₂; NH₂; Me; O-nPr; O-alkyl; O-alkylamino; R′═H, Me;
B=A; C; 5-Me-C; G; I; U; T; Y; 2-thiouridine; 4-thiouridine; C₅-modified pyrimidines; C2-modified purines; N8-modiifed purines; phenoxazine; G-clamp; non-canonical mono, bi and tricyclic heterocycles; pseudouracil; isoC; isoG; 2,6-diamninopurine; pseudocytosine; 2-aminopurine; xanthosine; N⁶-alkyl-A; O⁶-alkyl-G; 7-deazapurines; and Stereochemistry is R or S and combination of R and S for the unspecified chiral centers.

In some embodiments, the destabilizing modification mUNA is selected from the group consisting of

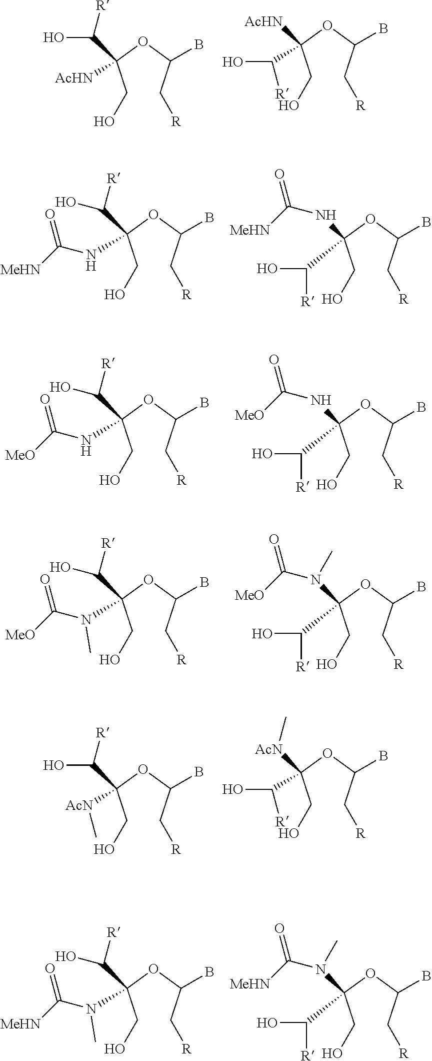

R═H, OH; OMe; Cl, F; OH; O—(CH₂)₂OMe; SMe, NMe₂; NH₂; Me; CCH(alkyne), O-nPr; O-alkyl; O-alkylamino;
R′═H, Me;
B=A; C; 5-Me-C; G; I; U; T; Y; 2-thiouridine; 4-thiouridine; C₅-modified pyrimidines; C2-modified purines; N8-modiifed purines; phenoxazine; G-clamp; non-canonical mono, bi and tricyclic heterocycles; pseudouracil; isoC; isoG; 2,6-diamninopurine; pseudocytosine; 2-aminopurine; xanthosine; N⁶-alkyl-A; O⁶-alkyl-G; 2-thiouridine; 4-thiouridine; C₅-modified pyrimidines; C2-modified purines; N8-modiifed purines; 7-deazapurines, phenoxazine; G-clamp; non-canonical mono, bi and tricyclic heterocycles; and
Stereochemistry is R or S and combination of R and S for the unspecified chiral centers

In some embodiments, the destabilizing modification mUNA is selected from the group consisting of

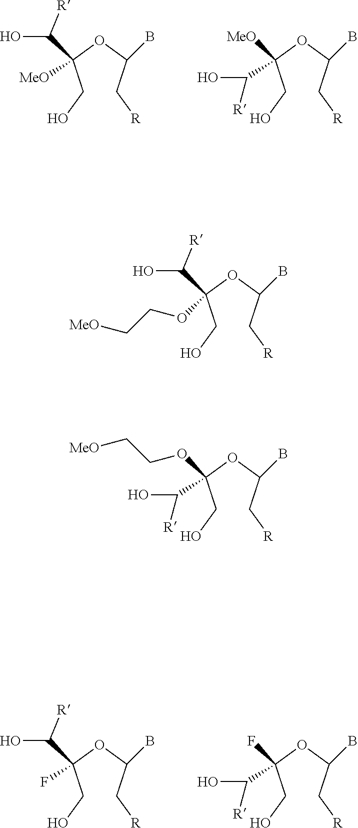

R═H, OH; OMe; Cl, F; OH; O—(CH₂)₂OMe; SMe, NMe₂; NH₂; Me; CCH(alkyne), O-nPr; O-alkyl; O-alkylamino;
R′═H, Me;
B=A; C; 5-Me-C; G; I; U; T; Y; 2-thiouridine; 4-thiouridine; C₅-modified pyrimidines; C2-modified purines; N8-modiifed purines; phenoxazine; G-clamp; non-canonical mono, bi and tricyclic heterocycles; pseudouracil; isoC; isoG; 2,6-diamninopurine; pseudocytosine; 2-aminopurine; xanthosine; N⁶-alkyl-A; O⁶-alkyl-G; 2-thiouridine; 4-thiouridine; C₅-modified pyrimidines; C2-modified purines; N8-modiifed purines; 7-deazapurines, phenoxazine; G-clamp; non-canonical mono, bi and tricyclic heterocycles; and
Stereochemistry is R or S and combination of R and S for the unspecified chiral centers

In some embodiments, the modification mUNA is selected from the group consisting of

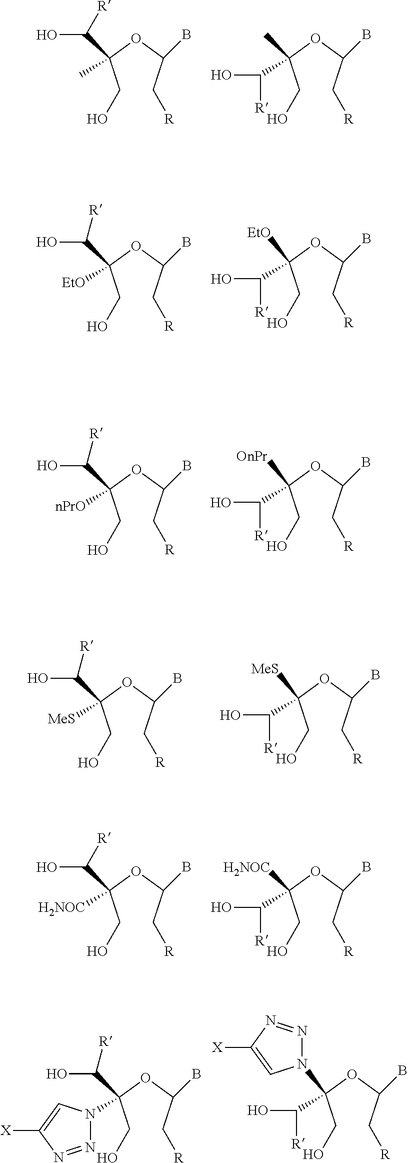

R═H, OMe; F; OH; O—(CH₂)₂OMe; SMe, NMe₂; NH₂; Me; O-nPr; O-alkyl; O-alkylamino;
R′═H, Me;
B=A; C; 5-Me-C; G; I; U; T; Y; 2-thiouridine; 4-thiouridine; C₅-modified pyrimidines; C2-modified purines; N8-modiifed purines; phenoxazine; G-clamp; non-canonical mono, bi and tricyclic heterocycles; pseudouracil; isoC; isoG; 2,6-diamninopurine; pseudocytosine; 2-aminopurine; xanthosine; N⁶-alkyl-A; O⁶-alkyl-G; 7-deazapurines; and Stereochemistry is R or S and combination of R and S for the unspecified chiral centers

Exemplary abasic modifications include, but are not limited to the following:

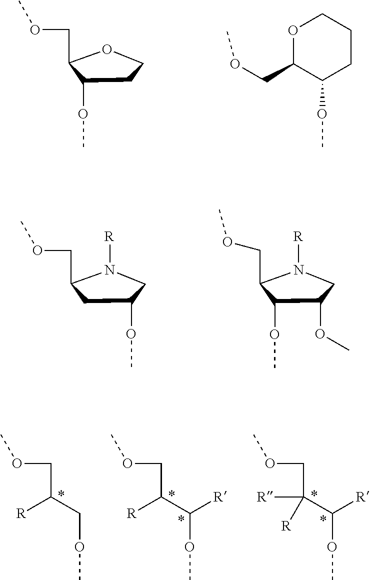

Wherein R═H, Me, Et or OMe; R′═H, Me, Et or OMe; R″═H, Me, Et or OMe

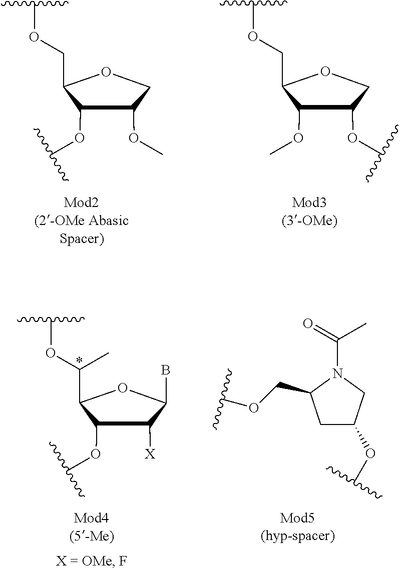

wherein B is a modified or unmodified nucleobase and the asterisk on each structure represents either R, S or racemic.

Exemplified sugar modifications include, but are not limited to the following:

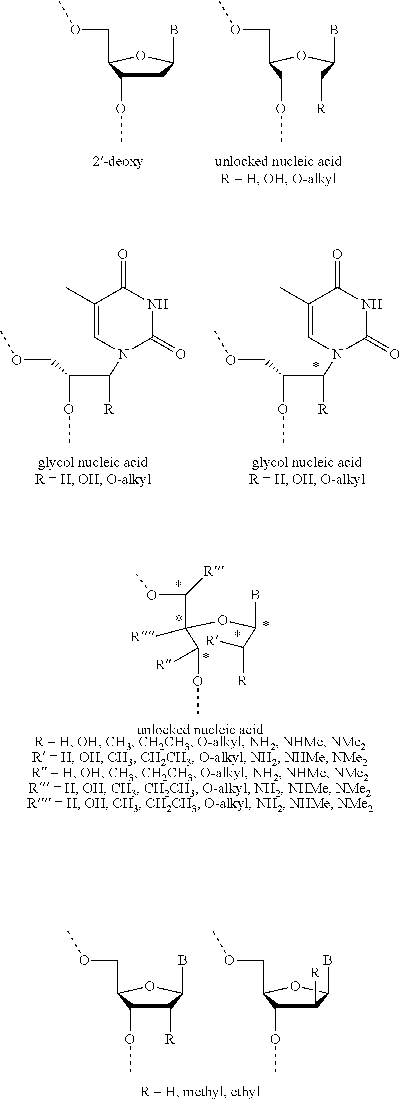

wherein B is a modified or unmodified nucleobase and the asterisk on each structure represents either R, S or racemic.

In some embodiments the thermally destabilizing modification of the duplex is selected from the mUNA and GNA building blocks described in Examples 1-3 herein. In some embodiments, the destabilizing modification is selected from the group consisting of GNA-isoC, GNA-isoG, 5′-mUNA, 4′-mUNA, 3′-mUNA, and 2′-mUNA. In some further embodiments of this, the dsRNA molecule further comprises at least one thermally destabilizing modification selected from the group consisting of GNA, 2′-OMe, 3′-OMe, 5′-Me, Hy p-spacer, SNA, hGNA, hhGNA, mGNA, TNA and h′GNA (Mod A-Mod K).

The term “acyclic nucleotide” refers to any nucleotide having an acyclic ribose sugar, for example, where any of bonds between the ribose carbons (e.g., C1′-C2′, C2′-C3′, C3′-C4′, C4′-O4′, or C1′-O4′) is absent and/or at least one of ribose carbons or oxygen (e.g., C1′, C2′, C3′, C4′ or O4′) are independently or in combination absent from the nucleotide. In some embodiments, acyclic nucleotide is

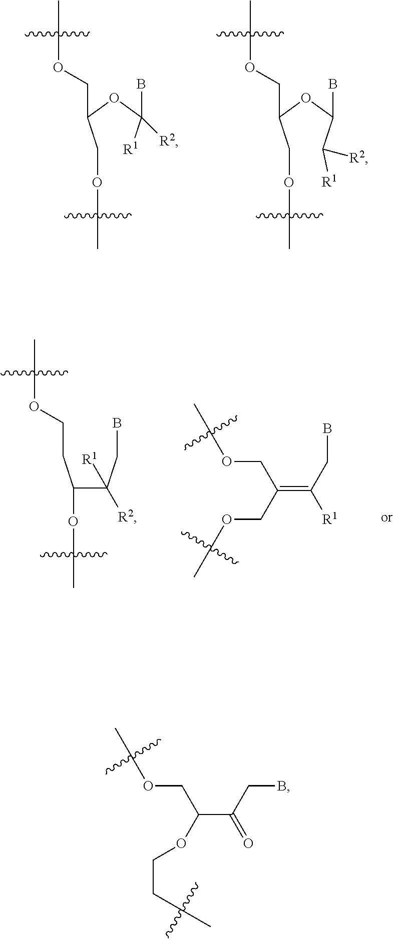

wherein B is a modified or unmodified nucleobase, R1 and R2 independently are H, halogen, OR3, or alkyl; and R3 is H, alkyl, cycloalkyl, aryl, aralkyl, heteroaryl or sugar). The term “UNA” refers to unlocked acyclic nucleic acid, wherein any of the bonds of the sugar has been removed, forming an unlocked “sugar” residue. In one example, UNA also encompasses monomers with bonds between C1′-C4′ being removed (i.e. the covalent carbon-oxygen-carbon bond between the C1′ and C4′ carbons). In another example, the C2′-C3′ bond (i.e. the covalent carbon-carbon bond between the C2′ and C3′ carbons) of the sugar is removed (see Mikhailov et. al., Tetrahedron Letters, 26(17): 2059(1985); and Fluiter et al., Mol. Biosyst., 10:1039 (2009), which are hereby incorporated by reference in their entirety). The acyclic derivative provides greater backbone flexibility without affecting the Watson-Crick pairings. The acyclic nucleotide can be linked via 2′-5′ or 3′-5′ linkage.

The term ‘GNA’ refers to glycol nucleic acid which is a polymer similar to DNA or RNA but differing in the composition of its “backbone” in that is composed of repeating glycerol units linked by phosphodiester bonds:

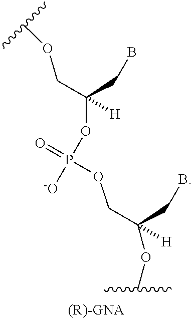

The thermally destabilizing modification of the duplex can be mismatches (i.e., noncomplementary base pairs) between the thermally destabilizing nucleotide and the opposing nucleotide in the opposite strand within the dsRNA duplex. Exemplary mismatch base pairs include G: G, G: A, G: U, G: T, A: A, A: C, C: C, C: U, C: T, U: U, T: T, U: T, or a combination thereof. Other mismatch base pairings known in the art are also amenable to the present invention. A mismatch can occur between nucleotides that are either naturally occurring nucleotides or modified nucleotides, i.e., the mismatch base pairing can occur between the nucleobases from respective nucleotides independent of the modifications on the ribose sugars of the nucleotides. In certain embodiments, the dsRNA molecule contains at least one nucleobase in the mismatch pairing that is a 2′-deoxy nucleobase; e.g., the 2′-deoxy nucleobase is in the sense strand.

In some embodiments, the thermally destabilizing modification of the duplex in the seed region of the antisense strand includes nucleotides with impaired W-C H-bonding to complementary base on the target mRNA, such as:

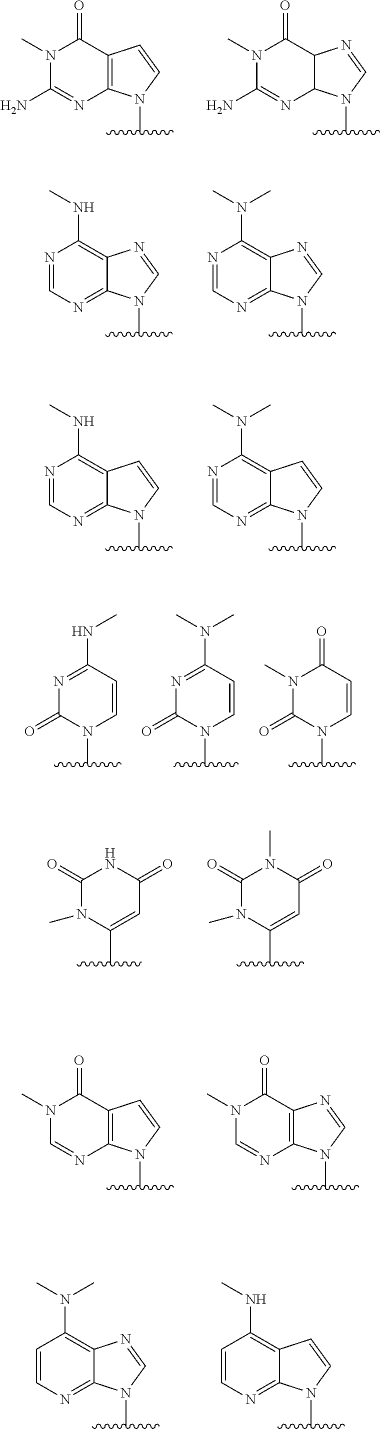

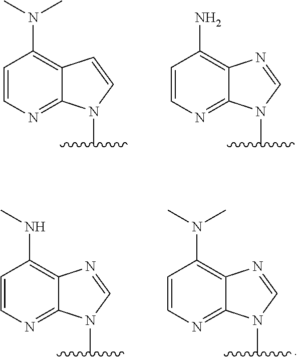 [SMILES: *n1ccc2c(N(C)C)ccnc21 ; *n1cnc2c(N)ccnc21 ; *n1cnc2c(NC)ccnc21 ; *n1cnc2c(N(C)C)ccnc21]

More examples of abasic nucleotide, acyclic nucleotide modifications (including UNA and GNA), and mismatch modifications have been described in detail in WO 2011/133876, which is herein incorporated by reference in its entirety.

The thermally destabilizing modifications may also include universal base with reduced or abolished capability to form hydrogen bonds with the opposing bases, and phosphate modifications.

In some embodiments, the thermally destabilizing modification of the duplex includes nucleotides with non-canonical bases such as, but not limited to, nucleobase modifications with impaired or completely abolished capability to form hydrogen bonds with bases in the opposite strand. These nucleobase modifications have been evaluated for destabilization of the central region of the dsRNA duplex as described in WO 2010/0011895, which is herein incorporated by reference in its entirety. Exemplary nucleobase modifications are:

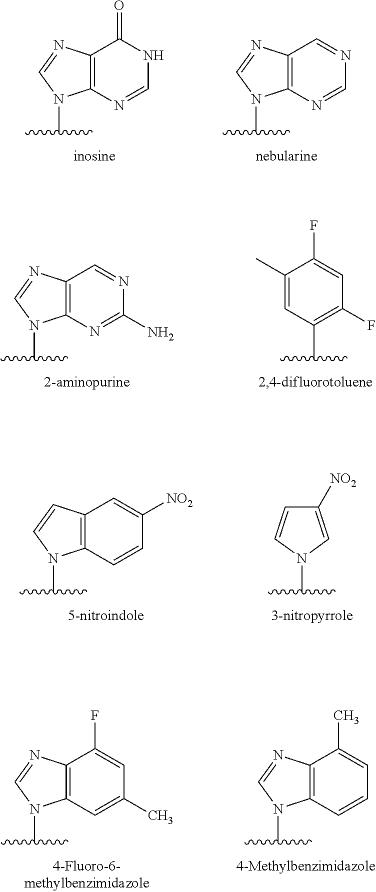

In some embodiments, the thermally destabilizing modification of the duplex in the seed region of the antisense strand includes one or more α-nucleotide complementary to the base on the target mRNA, such as:

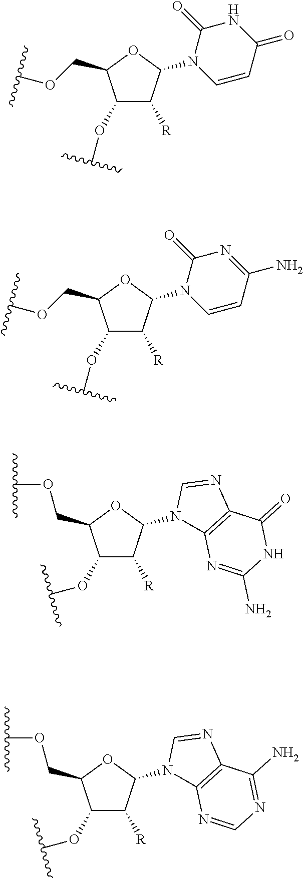

Wherein R is H, OH, OCH₃, F, NH₂, NHMe, NMe₂ or O-alkyl

Exemplary phosphate modifications known to decrease the thermal stability of dsRNA duplexes compared to natural phosphodiester linkages are:

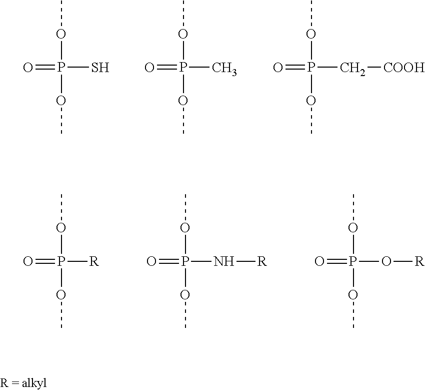

The alkyl for the R group can be a C₁-C₆alkyl. Specific alkyls for the R group include, but are not limited to methyl, ethyl, propyl, isopropyl, butyl, pentyl and hexyl.

It is noted a thermally destabilizing modification can replace a 2′-doexy nucleotide in the antisense strand. For example, a 2′-deoxy nucleotide at positions 2, 5, 7, 12, 14 and/or 16, counting from 5′-end, of the antisense strand can be replaced with a thermally destabilizing modification described herein. Thus, in some embodiments, the antisense strand comprises a thermally destabilizing modification at 1, 2, 3, 4, 5 and/or 6 of positions 2, 5, 7, 12, 14 and/or 16, counting from 5′-end of the antisense strand. For example, the antisense strand comprises a thermally destabilizing modification at positions 5 and 7, counting from 5′-end of the antisense strand.

In addition to the antisense strand comprising a thermally destabilizing modification, the dsRNA can also comprise one or more stabilizing modifications. For example, the dsRNA can comprise at least two (e.g., two, three, four, five, six, seven, eight, nine, ten or more) stabilizing modifications. Without limitations, the stabilizing modifications all can be present in one strand. In some embodiments, both the sense and the antisense strands comprise at least two stabilizing modifications. The stabilizing modification can occur on any nucleotide of the sense strand or antisense strand. For instance, the stabilizing modification can occur on every nucleotide on the sense strand and/or antisense strand; each stabilizing modification can occur in an alternating pattern on the sense strand or antisense strand; or the sense strand or antisense strand comprises both stabilizing modification in an alternating pattern. The alternating pattern of the stabilizing modifications on the sense strand may be the same or different from the antisense strand, and the alternating pattern of the stabilizing modifications on the sense strand can have a shift relative to the alternating pattern of the stabilizing modifications on the antisense strand.

In some embodiments, the antisense strand comprises at least two (e.g., two, three, four, five, six, seven, eight, nine, ten or more) stabilizing modifications. Without limitations, a stabilizing modification in the antisense strand can be present at any positions. In some embodiments, the antisense comprises stabilizing modifications at positions 2, 6, 8, 9, 14 and 16 from the 5′-end. In some other embodiments, the antisense comprises stabilizing modifications at positions 2, 6, 14 and 16 from the 5′-end. In still some other embodiments, the antisense comprises stabilizing modifications at positions 2, 14 and 16 from the 5′-end.

In some embodiments, the antisense strand comprises at least one stabilizing modification adjacent to the destabilizing modification. For example, the stabilizing modification can be the nucleotide at the 5′-end or the 3′-end of the destabilizing modification, i.e., at position-1 or +1 from the position of the destabilizing modification. In some embodiments, the antisense strand comprises a stabilizing modification at each of the 5′-end and the 3′-end of the destabilizing modification, i.e., positions-1 and +1 from the position of the destabilizing modification.

In some embodiments, the antisense strand comprises at least two stabilizing modifications at the 3′-end of the destabilizing modification, i.e., at positions +1 and +2 from the position of the destabilizing modification. In some embodiments, the sense strand comprises at least two (e.g., two, three, four, five, six, seven, eight, nine, ten or more) stabilizing modifications. Without limitations, a stabilizing modification in the sense strand can be present at any positions. In some embodiments, the sense strand comprises stabilizing modifications at positions 7, 10 and 11 from the 5′-end. In some other embodiments, the sense strand comprises stabilizing modifications at positions 7, 9, 10 and 11 from the 5′-end. In some embodiments, the sense strand comprises stabilizing modifications at positions opposite or complimentary to positions 11, 12 and 15 of the antisense strand, counting from the 5′-end of the antisense strand. In some other embodiments, the sense strand comprises stabilizing modifications at positions opposite or complimentary to positions 11, 12, 13 and 15 of the antisense strand, counting from the 5′-end of the antisense strand. In some embodiments, the sense strand comprises a block of two, three or four stabilizing modifications.

In some embodiments, the sense strand does not comprise a stabilizing modification in position opposite or complimentary to the thermally destabilizing modification of the duplex in the antisense strand.

Exemplary thermally stabilizing modifications include, but are not limited to 2′-fluoro modifications. Other thermally stabilizing modifications include, but are not limited to LNA.

It is noted a thermally stabilizing modification can replace a 2′-fluoro nucleotide in the sense and/or antisense strand. For example, a 2′-fluoro nucleotide at positions 8, 9, 10, 11 and/or 12, counting from 5′-end, of the sense strand, can be replaced with a thermally stabilizing modification. Similarly, a 2′-fluoro nucleotide at position 14, counting from 5′-end, of the antisense strand, can be replaced with a thermally stabilizing modification.

For the dsRNA molecules to be more effective in vivo, the antisense strand must have some metabolic stability. In other words, for the dsRNA molecules to be more effective in vivo, some amount of the antisense stand may need to be present in vivo after a period time after administration. Accordingly, in some embodiments, at least 40%, for example at least 45%, at least 50%, at least 55%, at least 60%., at least 65%, at least 70%, at least 75%, or at least 80% of the antisense strand of the dsRNA is present in vivo, for example in mouse liver, at day 5 after in vivo administration. In some embodiments, at least 40%, for example at least 45%, at least 50%, at least 55%, at least 60%., at least 65%, at least 70%, at least 75%, or at least 80% of the antisense strand of the dsRNA is present in vivo, for example in mouse liver, at day 6 after in vivo administration. In some embodiments, at least 40%, for example at least 45%, at least 50%, at least 55%, at least 60%., at least 65%, at least 70%, at least 75%, or at least 80% of the antisense strand of the dsRNA is present in vivo, for example in mouse liver, at day 7 after in vivo administration. In some embodiments, at least 40%, for example at least 45%, at least 50%, at least 55%, at least 60%., at least 65%, at least 70%, at least 75%, or at least 80% of the antisense strand of the dsRNA is present in vivo, for example in mouse liver, at day 8 after in vivo administration. In some embodiments, at least 40%, for example at least 45%, at least 50%, at least 55%, at least 60%., at least 65%, at least 70%, at least 75%, or at least 80% of the antisense strand of the dsRNA is present in vivo, for example in mouse liver, at day 9 after in vivo administration. In some embodiments, at least 40%, for example at least 45%, at least 50%, at least 55%, at least 60%., at least 65%, at least 70%, at least 75%, or at least 80% of the antisense strand of the dsRNA is present in vivo, for example in mouse liver, at day 10 after in vivo administration. In some embodiments, at least 40%, for example at least 45%, at least 50%, at least 55%, at least 60%., at least 65%, at least 70%, at least 75%, or at least 80% of the antisense strand of the dsRNA is present in vivo, for example in mouse liver, at day 11 after in vivo administration. In some embodiments, at least 40%, for example at least 45%, at least 50%, at least 55%, at least 60%., at least 65%, at least 70%, at least 75%, or at least 80% of the antisense strand of the dsRNA is present in vivo, for example in mouse liver, at day 12 after in vivo administration. In some embodiments, at least 40%, for example at least 45%, at least 50%, at least 55%, at least 60%., at least 65%, at least 70%, at least 75%, or at least 80% of the antisense strand of the dsRNA is present in vivo, for example in mouse liver, at day 13 after in vivo administration. In some embodiments, at least 40%, for example at least 45%, at least 50%, at least 55%, at least 60%., at least 65%, at least 70%, at least 75%, or at least 80% of the antisense strand of the dsRNA is present in vivo, for example in mouse liver, at day 14 after in vivo administration. In some embodiments, at least 40%, for example at least 45%, at least 50%, at least 55%, at least 60%., at least 65%, at least 70%, at least 75%, or at least 80% of the antisense strand of the dsRNA is present in vivo, for example in mouse liver, at day 15 after in vivo administration.

Uses of dsRNA

The present invention further relates to a use of a dsRNA molecule as defined herein for inhibiting expression of a target gene. In some embodiments, the present invention further relates to a use of a dsRNA molecule for inhibiting expression of a target gene in vitro.

The present invention further relates to a dsRNA molecule as defined herein for use in inhibiting expression of a target gene in a subject. The subject may be any animal, such as a mammal, e.g., a mouse, a rat, a sheep, a cattle, a dog, a cat, or a human

In some embodiments, the dsRNA molecule of the invention is administered in buffer. In some embodiments, siRNA compounds described herein can be formulated for administration to a subject. A formulated siRNA composition can assume a variety of states. In some examples, the composition is at least partially crystalline, uniformly crystalline, and/or anhydrous (e.g., less than 80, 50, 30, 20, or 10% water). In another example, the siRNA is in an aqueous phase, e.g., in a solution that includes water.

The aqueous phase or the crystalline compositions can, e.g., be incorporated into a delivery vehicle, e.g., a liposome (particularly for the aqueous phase) or a particle (e.g., a microparticle as can be appropriate for a crystalline composition). Generally, the siRNA composition is formulated in a manner that is compatible with the intended method of administration, as described herein. For example, in particular embodiments the composition is prepared by at least one of the following methods: spray drying, lyophilization, vacuum drying, evaporation, fluid bed drying, or a combination of these techniques; or sonication with a lipid, freeze-drying, condensation and other self-assembly.

A dsRNA preparation can be formulated in combination with another agent, e.g., another therapeutic agent or an agent that stabilizes a dsRNA, e.g., a protein that complexes with dsRNA to form an iRNP. Still other agents include chelating agents, e.g., EDTA (e.g., to remove divalent cations such as Mg²⁺), salts, RNAse inhibitors (e.g., a broad specificity RNAse inhibitor such as RNAsin) and so forth.

In some embodiments, the dsRNA preparation includes another dsRNA compound, e.g., a second dsRNA that can mediate RNAi with respect to a second gene, or with respect to the same gene. Still other preparation can include at least 3, 5, ten, twenty, fifty, or a hundred or more different siRNA species. Such dsRNAs can mediate RNAi with respect to a similar number of different genes.

In some embodiments, the dsRNA preparation includes at least a second therapeutic agent (e.g., an agent other than a RNA or a DNA). For example, a dsRNA composition for the treatment of a viral disease, e.g., HIV, might include a known antiviral agent (e.g., a protease inhibitor or reverse transcriptase inhibitor). In another example, a dsRNA composition for the treatment of a cancer might further comprise a chemotherapeutic agent.

Exemplary formulations which can be used for administering the dsRNA molecule according to the present invention are discussed below.

Liposomes. A dsRNA preparation can be formulated for delivery in a membranous molecular assembly, e.g., a liposome or a micelle. As used herein, the term “liposome” refers to a vesicle composed of amphiphilic lipids arranged in at least one bilayer, e.g., one bilayer or a plurality of bilayers. Liposomes include unilamellar and multilamellar vesicles that have a membrane formed from a lipophilic material and an aqueous interior. The aqueous portion contains the siRNA composition. The lipophilic material isolates the aqueous interior from an aqueous exterior, which typically does not include the siRNA composition, although in some examples, it may. Liposomes are useful for the transfer and delivery of active ingredients to the site of action. Because the liposomal membrane is structurally similar to biological membranes, when liposomes are applied to a tissue, the liposomal bilayer fuses with bilayer of the cellular membranes. As the merging of the liposome and cell progresses, the internal aqueous contents that include the dsRNA are delivered into the cell where the dsRNA can specifically bind to a target RNA and can mediate RNAi. In some cases, the liposomes are also specifically targeted, e.g., to direct the dsRNA to particular cell types.

A liposome containing a dsRNA can be prepared by a variety of methods. In one example, the lipid component of a liposome is dissolved in a detergent so that micelles are formed with the lipid component. For example, the lipid component can be an amphipathic cationic lipid or lipid conjugate. The detergent can have a high critical micelle concentration and may be nonionic. Exemplary detergents include cholate, CHAPS, octylglucoside, deoxycholate, and lauroyl sarcosine. The dsRNA preparation is then added to the micelles that include the lipid component. The cationic groups on the lipid interact with the siRNA and condense around the dsRNA to form a liposome. After condensation, the detergent is removed, e.g., by dialysis, to yield a liposomal preparation of dsRNA.

If necessary a carrier compound that assists in condensation can be added during the condensation reaction, e.g., by controlled addition. For example, the carrier compound can be a polymer other than a nucleic acid (e.g., spermine or spermidine). pH can also be adjusted to favor condensation.

Further description of methods for producing stable polynucleotide delivery vehicles, which incorporate a polynucleotide/cationic lipid complex as structural components of the delivery vehicle, are described in, e.g., WO 96/37194. Liposome formation can also include one or more aspects of exemplary methods described in Felgner, P. L. et al., Proc. Natl. Acad. Sci., USA 8:7413-7417, 1987; U.S. Pat. Nos. 4,897,355; 5,171,678; Bangham, et al. M. Mol. Biol. 23:238, 1965; Olson, et al. Biochim. Biophys. Acta 557:9, 1979; Szoka, et al. Proc. Natl. Acad. Sci. 75:4194, 1978; Mayhew, et al. Biochim. Biophys. Acta 775:169, 1984; Kim, et al. Biochim. Biophys. Acta 728:339, 1983; and Fukunaga, et al. Endocrinol. 115:757, 1984, which are incorporated by reference in their entirety. Commonly used techniques for preparing lipid aggregates of appropriate size for use as delivery vehicles include sonication and freeze-thaw plus extrusion (see, e.g., Mayer, et al. Biochim. Biophys. Acta 858:161, 1986, which is incorporated by reference in its entirety). Microfluidization can be used when consistently small (50 to 200 nm) and relatively uniform aggregates are desired (Mayhew, et al. Biochim. Biophys. Acta 775:169, 1984, which is incorporated by reference in its entirety). These methods are readily adapted to packaging siRNA preparations into liposomes.

Liposomes that are pH-sensitive or negatively-charged entrap nucleic acid molecules rather than complex with them. Since both the nucleic acid molecules and the lipid are similarly charged, repulsion rather than complex formation occurs. Nevertheless, some nucleic acid molecules are entrapped within the aqueous interior of these liposomes. pH-sensitive liposomes have been used to deliver DNA encoding the thymidine kinase gene to cell monolayers in culture. Expression of the exogenous gene was detected in the target cells (Zhou et al., Journal of Controlled Release, 19, (1992) 269-274, which is incorporated by reference in its entirety).

One major type of liposomal composition includes phospholipids other than naturally-derived phosphatidylcholine. Neutral liposome compositions, for example, can be formed from dimyristoyl phosphatidylcholine (DMPC) or dipalmitoyl phosphatidylcholine (DPPC). Anionic liposome compositions generally are formed from dimyristoyl phosphatidylglycerol, while anionic fusogenic liposomes are formed primarily from dioleoyl phosphatidylethanolamine (DOPE). Another type of liposomal composition is formed from phosphatidylcholine (PC) such as, for example, soybean PC, and egg PC. Another type is formed from mixtures of phospholipid and/or phosphatidylcholine and/or cholesterol.

Examples of other methods to introduce liposomes into cells in vitro and include U.S. Pat. Nos. 5,283,185; 5,171,678; WO 94/00569; WO 93/24640; WO 91/16024; Felgner, J. Biol. Chem. 269:2550, 1994; Nabel, Proc. Natl. Acad. Sci. 90:11307, 1993; Nabel, Human Gene Ther. 3:649, 1992; Gershon, Biochem. 32:7143, 1993; and Strauss EMBO J. 11:417, 1992.

In some embodiments, cationic liposomes are used. Cationic liposomes possess the advantage of being able to fuse to the cell membrane. Non-cationic liposomes, although not able to fuse as efficiently with the plasma membrane, are taken up by macrophages in vivo and can be used to deliver siRNAs to macrophages.

Further advantages of liposomes include: liposomes obtained from natural phospholipids are biocompatible and biodegradable; liposomes can incorporate a wide range of water and lipid soluble drugs; liposomes can protect encapsulated siRNAs in their internal compartments from metabolism and degradation (Rosoff, in “Pharmaceutical Dosage Forms,” Lieberman, Rieger and Banker (Eds.), 1988, volume 1, p. 245). Important considerations in the preparation of liposome formulations are the lipid surface charge, vesicle size and the aqueous volume of the liposomes.

A positively charged synthetic cationic lipid, N—[1-(2,3-dioleyloxy) propyl]-N,N,N-trimethylammonium chloride (DOTMA) can be used to form small liposomes that interact spontaneously with nucleic acid to form lipid-nucleic acid complexes which are capable of fusing with the negatively charged lipids of the cell membranes of tissue culture cells, resulting in delivery of siRNA (see, e.g., Felgner, P. L. et al., Proc. Natl. Acad. Sci., USA 8:7413-7417, 1987 and U.S. Pat. No. 4,897,355 for a description of DOTMA and its use with DNA, which are incorporated by reference in their entirety).

A DOTMA analogue, 1,2-bis(oleoyloxy)-3-(trimethylammonia) propane (DOTAP) can be used in combination with a phospholipid t to form DNA-complexing vesicles. Lipofectin™ Bethesda Research Laboratories, Gaithersburg, Md.) is an effective agent for the delivery of highly anionic nucleic acids into living tissue culture cells that comprise positively charged DOTMA liposomes which interact spontaneously with negatively charged polynucleotides to form complexes. When enough positively charged liposomes are used, the net charge on the resulting complexes is also positive. Positively charged complexes prepared in this way spontaneously attach to negatively charged cell surfaces, fuse with the plasma membrane, and efficiently deliver functional nucleic acids into, for example, tissue culture cells. Another commercially available cationic lipid, 1,2-bis(oleoyloxy)-3,3-(trimethylammonia) propane (“DOTAP”)(Boehringer Mannheim, Indianapolis, Indiana) differs from DOTMA in that the oleoyl moieties are linked by ester, rather than ether linkages.

Other reported cationic lipid compounds include those that have been conjugated to a variety of moieties including, for example, carboxyspermine which has been conjugated to one of two types of lipids and includes compounds such as 5-carboxyspermylglycine dioctaoleoylamide (“DOGS”)(Transfectam™, Promega, Madison, Wisconsin) and dipalmitoylphosphatidylethanolamine 5-carboxyspermyl-amide (“DPPES”)(see, e.g., U.S. Pat. No. 5,171,678).

Another cationic lipid conjugate includes derivatization of the lipid with cholesterol (“DC-Chol”) which has been formulated into liposomes in combination with DOPE (See, Gao, X. and Huang, L., Biochim. Biophys. Res. Commun. 179:280, 1991). Lipopolylysine, made by conjugating polylysine to DOPE, has been reported to be effective for transfection in the presence of serum (Zhou, X. et al., Biochim. Biophys. Acta 1065:8, 1991, which is incorporated by reference in its entirety). For certain cell lines, these liposomes containing conjugated cationic lipids, are said to exhibit lower toxicity and provide more efficient transfection than the DOTMA-containing compositions. Other commercially available cationic lipid products include DMRIE and DMRIE-HP(Vical, La Jolla, California) and Lipofectamine (DOSPA)(Life Technology, Inc., Gaithersburg, Maryland). Other cationic lipids suitable for the delivery of oligonucleotides are described in WO 98/39359 and WO 96/37194.

Liposomal formulations are particularly suited for topical administration. Liposomes present several advantages over other formulations. Such advantages include reduced side effects related to high systemic absorption of the administered drug, increased accumulation of the administered drug at the desired target, and the ability to administer siRNA, into the skin. In some implementations, liposomes are used for delivering siRNA to epidermal cells and also to enhance the penetration of siRNA into dermal tissues, e.g., into skin. For example, the liposomes can be applied topically. Topical delivery of drugs formulated as liposomes to the skin has been documented (see, e.g., Weiner et al., Journal of Drug Targeting, 1992, vol. 2,405-410 and du Plessis et al., Antiviral Research, 18, 1992, 259-265; Mannino, R. J. and Fould-Fogerite, S., Biotechniques 6:682-690, 1988; Itani, T. et al. Gene 56:267-276. 1987; Nicolau, C. et al. Meth. Enz. 149:157-176, 1987; Straubinger, R. M. and Papahadjopoulos, D. Meth. Enz. 101:512-527, 1983; Wang, C. Y. and Huang, L., Proc. Natl. Acad. Sci. USA 84:7851-7855, 1987, which are incorporated by reference in their entirety).

Non-ionic liposomal systems have also been examined to determine their utility in the delivery of drugs to the skin, in particular systems comprising non-ionic surfactant and cholesterol. Non-ionic liposomal formulations comprising Novasome I (glyceryl dilaurate/cholesterol/polyoxyethylene-10-stearyl ether) and Novasome II (glyceryl distearate/cholesterol/polyoxyethylene-10-stearyl ether) were used to deliver a drug into the dermis of mouse skin. Such formulations with dsRNA described herein are useful for treating a dermatological disorder.

Liposomes that include dsRNA described herein can be made highly deformable. Such deformability can enable the liposomes to penetrate through pore that are smaller than the average radius of the liposome. For example, transfersomes are a type of deformable liposomes. Transfersomes can be made by adding surface edge activators, usually surfactants, to a standard liposomal composition. Transfersomes that include dsRNA described herein can be delivered, for example, subcutaneously by infection in order to deliver dsRNA to keratinocytes in the skin. In order to cross intact mammalian skin, lipid vesicles must pass through a series of fine pores, each with a diameter less than 50 nm, under the influence of a suitable transdermal gradient. In addition, due to the lipid properties, these transfersomes can be self-optimizing (adaptive to the shape of pores, e.g., in the skin), self-repairing, and can frequently reach their targets without fragmenting, and often self-loading.

Other formulations amenable to the present invention are described in U.S. provisional application Ser. No. 61/018,616, filed Jan. 2, 2008; 61/018,611, filed Jan. 2, 2008; 61/039,748, filed Mar. 26, 2008; 61/047,087, filed Apr. 22, 2008 and 61/051,528, filed May 8, 2008. PCT application no PCT/US2007/080331, filed Oct. 3, 2007 also describes formulations that are amenable to the present invention.

Surfactants. The dsRNA compositions can include a surfactant. In some embodiments, the dsRNA is formulated as an emulsion that includes a surfactant. The most common way of classifying and ranking the properties of the many different types of surfactants, both natural and synthetic, is by the use of the hydrophile/lipophile balance (HLB). The nature of the hydrophilic group provides the most useful means for categorizing the different surfactants used in formulations (Rieger, in “Pharmaceutical Dosage Forms,” Marcel Dekker, Inc., New York, NY, 1988, p. 285).

If the surfactant molecule is not ionized, it is classified as a nonionic surfactant. Nonionic surfactants find wide application in pharmaceutical products and are usable over a wide range of pH values. In general, their HLB values range from 2 to about 18 depending on their structure. Nonionic surfactants include nonionic esters such as ethylene glycol esters, propylene glycol esters, glyceryl esters, polyglyceryl esters, sorbitan esters, sucrose esters, and ethoxylated esters. Nonionic alkanolamides and ethers such as fatty alcohol ethoxylates, propoxylated alcohols, and ethoxylated/propoxylated block polymers are also included in this class. The polyoxyethylene surfactants are the most popular members of the nonionic surfactant class.

If the surfactant molecule carries a negative charge when it is dissolved or dispersed in water, the surfactant is classified as anionic. Anionic surfactants include carboxylates such as soaps, acyl lactylates, acyl amides of amino acids, esters of sulfuric acid such as alkyl sulfates and ethoxylated alkyl sulfates, sulfonates such as alkyl benzene sulfonates, acyl isethionates, acyl taurates and sulfosuccinates, and phosphates. The most important members of the anionic surfactant class are the alkyl sulfates and the soaps.

If the surfactant molecule carries a positive charge when it is dissolved or dispersed in water, the surfactant is classified as cationic. Cationic surfactants include quaternary ammonium salts and ethoxylated amines. The quaternary ammonium salts are the most used members of this class.

If the surfactant molecule has the ability to carry either a positive or negative charge, the surfactant is classified as amphoteric. Amphoteric surfactants include acrylic acid derivatives, substituted alkylamides, N-alkylbetaines and phosphatides.

The use of surfactants in drug products, formulations and in emulsions has been reviewed (Rieger, in “Pharmaceutical Dosage Forms,” Marcel Dekker, Inc., New York, NY, 1988, p. 285).

Micelles and other Membranous Formulations. For ease of exposition the micelles and other formulations, compositions and methods in this section are discussed largely with regard to unmodified siRNA compounds. It may be understood, however, that these micelles and other formulations, compositions and methods can be practiced with other siRNA compounds, e.g., modified siRNA compounds, and such practice is within the invention. The siRNA compound, e.g., a double-stranded siRNA compound, or ssiRNA compound, (e.g., a precursor, e.g., a larger siRNA compound which can be processed into a ssiRNA compound, or a DNA which encodes an siRNA compound, e.g., a double-stranded siRNA compound, or ssiRNA compound, or precursor thereof)) composition can be provided as a micellar formulation. “Micelles” are defined herein as a particular type of molecular assembly in which amphipathic molecules are arranged in a spherical structure such that all the hydrophobic portions of the molecules are directed inward, leaving the hydrophilic portions in contact with the surrounding aqueous phase. The converse arrangement exists if the environment is hydrophobic.

A mixed micellar formulation suitable for delivery through transdermal membranes may be prepared by mixing an aqueous solution of the dsRNA composition, an alkali metal C₈ to C₂₂ alkyl sulphate, and a micelle forming compounds. Exemplary micelle forming compounds include lecithin, hyaluronic acid, pharmaceutically acceptable salts of hyaluronic acid, glycolic acid, lactic acid, chamomile extract, cucumber extract, oleic acid, linoleic acid, linolenic acid, monoolein, monooleates, monolaurates, borage oil, evening of primrose oil, menthol, trihydroxy oxo cholanyl glycine and pharmaceutically acceptable salts thereof, glycerin, polyglycerin, lysine, polylysine, triolein, polyoxyethylene ethers and analogues thereof, polidocanol alkyl ethers and analogues thereof, chenodeoxycholate, deoxycholate, and mixtures thereof. The micelle forming compounds may be added at the same time or after addition of the alkali metal alkyl sulphate. Mixed micelles will form with substantially any kind of mixing of the ingredients but vigorous mixing in order to provide smaller size micelles.

In one method, a first micellar composition is prepared which contains the dsRNA composition and at least the alkali metal alkyl sulphate. The first micellar composition is then mixed with at least three micelle forming compounds to form a mixed micellar composition. In another method, the micellar composition is prepared by mixing the dsRNA composition, the alkali metal alkyl sulphate and at least one of the micelle forming compounds, followed by addition of the remaining micelle forming compounds, with vigorous mixing.

Phenol and/or m-cresol may be added to the mixed micellar composition to stabilize the formulation and protect against bacterial growth. Alternatively, phenol and/or m-cresol may be added with the micelle forming ingredients. An isotonic agent such as glycerin may also be added after formation of the mixed micellar composition.

For delivery of the micellar formulation as a spray, the formulation can be put into an aerosol dispenser and the dispenser is charged with a propellant. The propellant, which is under pressure, is in liquid form in the dispenser. The ratios of the ingredients are adjusted so that the aqueous and propellant phases become one, i.e., there is one phase. If there are two phases, it is necessary to shake the dispenser prior to dispensing a portion of the contents, e.g., through a metered valve. The dispensed dose of pharmaceutical agent is propelled from the metered valve in a fine spray.

Propellants may include hydrogen-containing chlorofluorocarbons, hydrogen-containing fluorocarbons, dimethyl ether and diethyl ether. In certain embodiments, HFA 134a (1,1,1,2 tetrafluoroethane) may be used.

The specific concentrations of the essential ingredients can be determined by relatively straightforward experimentation. For absorption through the oral cavities, it is often desirable to increase, e.g., at least double or triple, the dosage for through injection or administration through the gastrointestinal tract.

Particles. In some embodiments, dsRNA preparations can be incorporated into a particle, e.g., a microparticle. Microparticles can be produced by spray-drying, but may also be produced by other methods including lyophilization, evaporation, fluid bed drying, vacuum drying, or a combination of these techniques.

### Pharmaceutical Compositions

The dsRNA agents of the invention can be formulated for pharmaceutical use. The present invention further relates to a pharmaceutical composition comprising the dsRNA molecule as defined herein. Pharmaceutically acceptable compositions comprise a therapeutically-effective amount of one or more of the dsRNA molecules in any of the preceding embodiments, taken alone or formulated together with one or more pharmaceutically acceptable carriers (additives), excipient and/or diluents.

The pharmaceutical compositions may be specially formulated for administration in solid or liquid form, including those adapted for the following: (1) oral administration, for example, drenches (aqueous or non-aqueous solutions or suspensions), tablets, e.g., those targeted for buccal, sublingual, and systemic absorption, boluses, powders, granules, pastes for application to the tongue; (2) parenteral administration, for example, by subcutaneous, intramuscular, intravenous or epidural injection as, for example, a sterile solution or suspension, or sustained-release formulation; (3) topical application, for example, as a cream, ointment, or a controlled-release patch or spray applied to the skin; (4) intravaginally or intrarectally, for example, as a pessary, cream or foam; (5) sublingually; (6) ocularly; (7) transdermally; or (8) nasally. Delivery using subcutaneous or intravenous methods can be particularly advantageous.

The phrase “therapeutically-effective amount” as used herein means that amount of a compound, material, or composition comprising a compound of the invention which is effective for producing some desired therapeutic effect in at least a sub-population of cells in an animal at a reasonable benefit/risk ratio applicable to any medical treatment.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically-acceptable carrier” as used herein means a pharmaceutically-acceptable material, composition or vehicle, such as a liquid or solid filler, diluent, excipient, manufacturing aid (e.g., lubricant, talc magnesium, calcium or zinc stearate, or steric acid), or solvent encapsulating material, involved in carrying or transporting the subject compound from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) lubricating agents, such as magnesium state, sodium lauryl sulfate and talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; (22) bulking agents, such as polypeptides and amino acids (23) serum component, such as serum albumin, HDL and LDL; and (22) other non-toxic compatible substances employed in pharmaceutical formulations.

The formulations may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient which can be combined with a carrier material to produce a single dosage form will generally be that amount of the compound which produces a therapeutic effect. Generally, out of one hundred percent, this amount will range from about 0.1 percent to about ninety-nine percent of active ingredient, preferably from about 5 percent to about 70 percent, most preferably from about 10 percent to about 30 percent.

In certain embodiments, a formulation of the present invention comprises an excipient selected from the group consisting of cyclodextrins, celluloses, liposomes, micelle forming agents, e.g., bile acids, and polymeric carriers, e.g., polyesters and polyanhydrides; and a compound of the present invention. In certain embodiments, an aforementioned formulation renders orally bioavailable a compound of the present invention.

The dsRNA agent preparation can be formulated in combination with another agent, e.g., another therapeutic agent or an agent that stabilizes a dsRNA, e.g., a protein that complexes with the dsRNA to form an iRNP. Still other agents include chelating agents, e.g., EDTA (e.g., to remove divalent cations such as Mg²⁺), salts, RNAse inhibitors (e.g., a broad specificity RNAse inhibitor such as RNAsin) and so forth.

Methods of preparing these formulations or compositions include the step of bringing into association a compound of the present invention with the carrier and, optionally, one or more accessory ingredients. In general, the formulations are prepared by uniformly and intimately bringing into association a compound of the present invention with liquid carriers, or finely divided solid carriers, or both, and then, if necessary, shaping the product.

In some cases, in order to prolong the effect of a drug, it is desirable to slow the absorption of the drug from subcutaneous or intramuscular injection. This may be accomplished by the use of a liquid suspension of crystalline or amorphous material having poor water solubility.

The rate of absorption of the drug then depends upon its rate of dissolution which, in turn, may depend upon crystal size and crystalline form. Alternatively, delayed absorption of a parenterally-administered drug form is accomplished by dissolving or suspending the drug in an oil vehicle.

The compounds according to the invention may be formulated for administration in any convenient way for use in human or veterinary medicine, by analogy with other pharmaceuticals.

The term “treatment” is intended to encompass therapy and cure. The patient receiving this treatment is any animal in need, including primates, in particular humans, and other mammals such as equines, cattle, swine and sheep; and poultry and pets in general.

Double-stranded RNA agents are produced in a cell in vivo, e.g., from exogenous DNA templates that are delivered into the cell. For example, the DNA templates can be inserted into vectors and used as gene therapy vectors. Gene therapy vectors can be delivered to a subject by, for example, intravenous injection, local administration (U.S. Pat. No. 5,328,470, which is incorporated by reference in its entirety), or by stereotactic injection (see, e.g., Chen et al. (1994) Proc. Natl. Acad. Sci. USA 91:3054-3057, which is incorporated by reference in its entirety). The pharmaceutical preparation of the gene therapy vector can include the gene therapy vector in an acceptable diluent, or can comprise a slow release matrix in which the gene delivery vehicle is imbedded. The DNA templates, for example, can include two transcription units, one that produces a transcript that includes the top strand of a dsRNA molecule and one that produces a transcript that includes the bottom strand of a dsRNA molecule. When the templates are transcribed, the dsRNA molecule is produced, and processed into siRNA agent fragments that mediate gene silencing.

The dsRNA molecule as defined herein or a pharmaceutical composition comprising a dsRNA molecule as defined herein can be administered to a subject using different routes of delivery. A composition that includes a dsRNA described herein can be delivered to a subject by a variety of routes. Exemplary routes include: intravenous, subcutaneous, topical, rectal, anal, vaginal, nasal, pulmonary, ocular.

The dsRNA molecule of the invention can be incorporated into pharmaceutical compositions suitable for administration. Such compositions typically include one or more species of dsRNAs and a pharmaceutically acceptable carrier. As used herein the language “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

The compositions of the present invention may be administered in a number of ways depending upon whether local or systemic treatment is desired and upon the area to be treated. Administration may be topical (including ophthalmic, vaginal, rectal, intranasal, transdermal), oral or parenteral. Parenteral administration includes intravenous drip, subcutaneous, intraperitoneal or intramuscular injection, or intrathecal or intraventricular administration.

The route and site of administration may be chosen to enhance targeting. For example, to target muscle cells, intramuscular injection into the muscles of interest would be a logical choice. Lung cells might be targeted by administering the dsRNA in aerosol form. The vascular endothelial cells could be targeted by coating a balloon catheter with the dsRNA and mechanically introducing the dsRNA.

In one aspect, the invention features a method of administering a dsRNA molecule, e.g., a dsRNA agent described herein, to a subject (e.g., a human subject). In another aspect, the present invention relates to a dsRNA molecule as defined herein for use in inhibiting expression of a target gene in a subject. The method or the medical use includes administering a unit dose of the dsRNA molecule, e.g., a dsRNA agent described herein. In some embodiments, the unit dose is less than 10 mg per kg of bodyweight, or less than 10, 5, 2, 1, 0.5, 0.1, 0.05, 0.01, 0.005, 0.001, 0.0005, 0.0001, 0.00005 or 0.00001 mg per kg of bodyweight, and less than 200 nmole of RNA agent (e.g., about 4.4×10¹⁶ copies) per kg of bodyweight, or less than 1500, 750, 300, 150, 75, 15, 7.5, 1.5, 0.75, 0.15, 0.075, 0.015, 0.0075, 0.0015, 0.00075, 0.00015 nmole of RNA agent per kg of bodyweight.

The defined amount can be an amount effective to treat or prevent a disease or disorder, e.g., a disease or disorder associated with the target gene. The unit dose, for example, can be administered by injection (e.g., intravenous, subcutaneous or intramuscular), an inhaled dose, or a topical application. In some embodiments dosages may be less than 10, 5, 2, 1, or 0.1 mg/kg of body weight.

In some embodiments, the unit dose is administered less frequently than once a day, e.g., less than every 2, 4, 8 or 30 days. In another embodiment, the unit dose is not administered with a frequency (e.g., not a regular frequency). For example, the unit dose may be administered a single time.

In some embodiments, the effective dose is administered with other traditional therapeutic modalities. In some embodiments, the subject has a viral infection and the modality is an antiviral agent other than a dsRNA molecule, e.g., other than a siRNA agent. In another embodiment, the subject has atherosclerosis and the effective dose of a dsRNA molecule, e.g., a siRNA agent, is administered in combination with, e.g., after surgical intervention, e.g., angioplasty.

In some embodiments, a subject is administered an initial dose and one or more maintenance doses of a dsRNA molecule, e.g., a siRNA agent, (e.g., a precursor, e.g., a larger dsRNA molecule which can be processed into a siRNA agent, or a DNA which encodes a dsRNA molecule, e.g., a siRNA agent, or precursor thereof). The maintenance dose or doses can be the same or lower than the initial dose, e.g., one-half less of the initial dose. A maintenance regimen can include treating the subject with a dose or doses ranging from 0.01 μg to 15 mg/kg of body weight per day, e.g., 10, 1, 0.1, 0.01, 0.001, or 0.00001 mg per kg of bodyweight per day. The maintenance doses are, for example, administered no more than once every 2, 5, 10, or 30 days. Further, the treatment regimen may last for a period of time which will vary depending upon the nature of the particular disease, its severity and the overall condition of the patient. In certain embodiments the dosage may be delivered no more than once per day, e.g., no more than once per 24, 36, 48, or more hours, e.g., no more than once for every 5 or 8 days. Following treatment, the patient can be monitored for changes in his condition and for alleviation of the symptoms of the disease state. The dosage of the compound may either be increased in the event the patient does not respond significantly to current dosage levels, or the dose may be decreased if an alleviation of the symptoms of the disease state is observed, if the disease state has been ablated, or if undesired side-effects are observed.

The effective dose can be administered in a single dose or in two or more doses, as desired or considered appropriate under the specific circumstances. If desired to facilitate repeated or frequent infusions, implantation of a delivery device, e.g., a pump, semi-permanent stent (e.g., intravenous, intraperitoneal, intracisternal or intracapsular), or reservoir may be advisable.

In some embodiments, the composition includes a plurality of dsRNA molecule species. In another embodiment, the dsRNA molecule species has sequences that are non-overlapping and non-adjacent to another species with respect to a naturally occurring target sequence. In another embodiment, the plurality of dsRNA molecule species is specific for different naturally occurring target genes. In another embodiment, the dsRNA molecule is allele specific.

The dsRNA molecules of the invention described herein can be administered to mammals, particularly large mammals such as nonhuman primates or humans in a number of ways.

In some embodiments, the administration of the dsRNA molecule, e.g., a siRNA agent, composition is parenteral, e.g., intravenous (e.g., as a bolus or as a diffusible infusion), intradermal, intraperitoneal, intramuscular, intrathecal, intraventricular, intracranial, subcutaneous, transmucosal, buccal, sublingual, endoscopic, rectal, oral, vaginal, topical, pulmonary, intranasal, urethral or ocular. Administration can be provided by the subject or by another person, e.g., a health care provider. The medication can be provided in measured doses or in a dispenser which delivers a metered dose. Selected modes of delivery are discussed in more detail below.

The invention provides methods, compositions, and kits, for rectal administration or delivery of dsRNA molecules described herein

In particular embodiments, the present invention relates to the dsRNA molecules of the present invention for use in the methods described above.

Methods of inhibiting expression of the target gene

Embodiments of the invention also relate to methods for inhibiting the expression of a target gene. The method comprises the step of administering the dsRNA molecules in any of the preceding embodiments, in an amount sufficient to inhibit expression of the target gene. The present invention further relates to a use of a dsRNA molecule as defined herein for inhibiting expression of a target gene in a target cell. In a preferred embodiment, the present invention further relates to a use of a dsRNA molecule for inhibiting expression of a target gene in a target cell in vitro.

Another aspect the invention relates to a method of modulating the expression of a target gene in a cell, comprising providing to said cell a dsRNA molecule of this invention. In some embodiments, the target gene is selected from the group consisting of Factor VII, Eg5, PCSK9, TPX2, apoB, SAA, TTR, RSV, PDGF beta gene, Erb-B gene, Src gene, CRK gene, GRB2 gene, RAS gene, MEKK gene, JNK gene, RAF gene, Erk1/2 gene, PCNA (p21) gene, MYB gene, JUN gene, FOS gene, BCL-2 gene, hepcidin, Activated Protein C, Cyclin D gene, VEGF gene, EGFR gene, Cyclin A gene, Cyclin E gene, WNT-1 gene, beta-catenin gene, c-MET gene, PKC gene, NFKB gene, STAT3 gene, survivin gene, Her2/Neu gene, topoisomerase I gene, topoisomerase II alpha gene, mutations in the p73 gene, mutations in the p21 (WAF1/CIP1) gene, mutations in the p27 (KIP1) gene, mutations in the PPMID gene, mutations in the RAS gene, mutations in the caveolin I gene, mutations in the MIB I gene, mutations in the MTAI gene, mutations in the M68 gene, mutations in tumor suppressor genes, and mutations in the p53 tumor suppressor gene.

In particular embodiments, the present invention relates to the dsRNA molecules of the present invention for use in the methods described above.

Exemplary embodiments of the various aspects can be described by the following lettered embodiments:
Embodiment A: A dsRNA agent comprising a sense strand and antisense, each strand independently having a length of 15-35 nucleotides, wherein the sense strand comprises a 2′-fluoro nucleotide at position 10, counting from 5′-end of the sense strand, and wherein the antisense strand comprises a 2′-deoxy nucleotide at positions 5 and 7, counting from 5′-end of the antisense strand.
Embodiment B: The dsRNA agent of Embodiment A, wherein the sense strand further comprises a 2′-fluoro nucleotide at position 11, counting from 5′-end of the sense strand.
Embodiment C: The dsRNA agent of Embodiment A or B, wherein the sense strand further comprises a 2′-fluoro nucleotide at position 9, counting from 5′-end of the sense strand.
Embodiment D: The dsRNA of any one of Embodiments A to C, wherein the sense strand further comprises a 2′-fluoro nucleotide at positions 9 and 11, counting from 5-end of the sense strand.
Embodiment E: The dsRNA agent of any one of Embodiments A to D, wherein the sense strand comprises a 2′-fluoro nucleotide at positions 8 and 9, counting from 5-end of the sense strand.
Embodiment F: The dsRNA agent of any one of Embodiments A to E, wherein the sense strand comprises a 2′-fluoro nucleotide at positions 11 and 12, counting from 5-end of the sense strand.
Embodiment G: The dsRNA agent of any one of Embodiments A to F, wherein the sense strand comprises at least one 2′-OMe nucleotide.
Embodiment H: The dsRNA agent of any one of Embodiments A to G, wherein the antisense strand comprises a 2′-deoxy nucleotide at position 2, counting from 5′-end of the antisense strand.
Embodiment I: The dsRNA agent of any one of Embodiments A to H, wherein the antisense strand comprises a 2′-deoxy nucleotide at positions 2, 5 and 7, counting from 5′-end of the antisense strand
Embodiment J: The dsRNA agent of any one of Embodiments A to I, wherein the antisense strand comprises at least one 2′-fluoro nucleotide.
Embodiment K: The dsRNA agent of any one of Embodiments A to J, wherein the antisense strand comprises a 2′-fluoro nucleotide at position 14 of the antisense strand, counting from 5′-end of the antisense strand.
Embodiment L: The dsRNA agent of any one of Embodiments A to K, wherein the dsRNA agent comprises a ligand.
Embodiment M: The dsRNA agent of any one of Embodiments A to L, wherein the sense strand comprises a ligand.
Embodiment N: The dsRNA agent of Embodiment L or M, wherein the ligand is an ASGPR ligand.
Embodiment O: The dsRNA agent of any one of Embodiments A to N, wherein the dsRNA agent comprises at least two phosphorothioate internucleotide linkages.
Embodiment P: The dsRNA agent of any one of Embodiments A to O, wherein the sense strand comprises at least two phosphorothioate internucleotide linkages between the first five nucleotides counting from the 5′ end of the sense strand.
Embodiment Q: The dsRNA agent of any one of Embodiments A to P, wherein the antisense strand comprises at least two phosphorothioate internucleotide linkages between the first five nucleotides counting from the 5′ end of the antisense strand and at least two phosphorothioate internucleotide linkages between the first five nucleotides counting from the 3′ end of the antisense strand.
Embodiment R: The dsRNA agent of any one of Embodiments A to Q, wherein the dsRNA has a duplex region of from 18 to about 25 basepairs.
Embodiment S: The dsRNA agent of any one of Embodiments A to R, wherein the sense strand is 18-23 nucleotides in length.
Embodiment T: The dsRNA agent of any one of Embodiments A to S, wherein the antisense strand is 18-25 nucleotides in length.
Embodiment U: A dsRNA agent comprising a sense strand and an antisense strand, wherein the sense strand is 18-23 nucleotides in length and comprises a 2′-fluoro nucleotide at position 10, counting from 5′-end of the sense strand and a 2′-fluoro nucleotide at position 9 or 11, counting from 5′-end of the sense strand, and wherein the antisense strand is 18-25 nucleotide in length and comprises a 2′-deoxy nucleotide at positions 5 and 7, counting from 5′-end of the antisense strand.
Embodiment V: A dsRNA agent comprising a sense strand and an antisense strand, wherein the sense strand is 18-23 nucleotides in length and comprises a 2′-fluoro nucleotide at positions 9, 10 and 11, counting from 5′-end of the sense strand, and wherein the antisense is 18-25 nucleotide in length and comprises a 2′-deoxy nucleotide at position 5 and 7, counting from 5′-end of the antisense strand.

Additional exemplary embodiments can be described by one or more of the following numbered embodiments:
Embodiment 1: dsRNA agent comprising a sense strand and antisense, each strand independently having a length of 15-35 nucleotides wherein each nucleotide is independently modified or unmodified, wherein: the sense strand comprises a 2′-fluoro nucleotide at position 10, counting from 5′-end of the sense strand, and the antisense strand comprises a 2′-deoxy nucleotide at positions 2, 5, 7 and 12, counting from 5′-end of the antisense strand, and wherein: (i) the antisense strand comprises a 2′-fluoro nucleotide at position 14 and a nucleotide other than a 2′-deoxy or 2′-fluoro nucleotide at position 16, counting from the 5′-end of the antisense strand; or (ii) the antisense strand comprises a 2′-deoxy nucleotide at position 14 or 16, counting from the 5′-end of the antisense strand, and the sense strand comprises a nucleotide other than a 2′-fluoro nucleotide at position 7, counting from the 5′-end of the sense strand.
Embodiment 2: The dsRNA agent of Embodiment 1, wherein the sense strand further comprises a 2′-fluoro nucleotide at position 11, counting from 5′-end of the sense strand.
Embodiment 3: The dsRNA agent of Embodiment 1 or 2, wherein the sense strand further comprises a 2′-fluoro nucleotide at position 9, counting from 5′-end of the sense strand.
Embodiment 4: The dsRNA of any one of Embodiments 1-3, wherein the sense strand further comprises a 2′-fluoro nucleotide at positions 9 and 11, counting from 5-end of the sense strand.
Embodiment 5: The dsRNA agent of any one of Embodiments 1-4, wherein the sense strand comprises a 2′-fluoro nucleotide at positions 8 and 9, counting from 5-end of the sense strand.
Embodiment 6: The dsRNA agent of any one of Embodiments 1-5, wherein the sense strand comprises a 2′-fluoro nucleotide at positions 11 and 12, counting from 5-end of the sense strand.
Embodiment 7: The dsRNA agent of any one of Embodiments 1-6, wherein the sense strand comprises a nucleotide other than a 2′-fluoro at position 7, counting from the 5′-end of the sense strand.
Embodiment 8: The dsRNA of any one of Embodiments 1-7, wherein the sense strand comprises a 2′-fluoro nucleotide at positions 9, 10 and 11, and a nucleotide other than a 2′-fluoro at position 7, counting from the 5′-end of the sense strand
Embodiment 9: The dsRNA agent of any one of Embodiments 1-8, wherein the sense strand comprises at least one 2′-OMe nucleotide.
Embodiment 10: The dsRNA agent of any one of Embodiments 1-9, wherein the sense strand comprises a 2′-OMe nucleotide at position 7, counting from the 5′-end of the sense strand.
Embodiment 11: The dsRNA agent of any one of Embodiments 1-10, wherein the sense strand comprises a 2′-fluoro nucleotide at positions 9, 10 and 11, and a 2′-OMe nucleotide at position 7, counting from the 5′-end of the sense strand.
Embodiment 12: The dsRNA agent of any one of Embodiments 1-11, wherein the antisense strand comprises a 2′-fluoro nucleotide at position 14 of the antisense strand, and a nucleotide other than a 2′-deoxy or 2′-fluoro nucleotide at position 16, counting from 5′-end of the antisense strand.
Embodiment 13: The dsRNA agent of any one of Embodiments 1-12, wherein the antisense strand comprises a 2′-deoxy nucleotide at position 2, 5, 7, and 12, a 2′-fluoro nucleotide at position 14 and a nucleotide other than a 2′-deoxy or 2′-fluoro nucleotide at position 16, counting from the 5′-end of the antisense strand.
Embodiment 14: The dsRNA agent of any one of Embodiments 1-13, wherein the antisense strand comprises a 2′-OMe nucleotide at position 16, counting from the 5′-end of the antisense strand.
Embodiment 15: The dsRNA agent of any one of Embodiments 1-14, wherein the antisense strand comprises a 2′-deoxy nucleotide at position 2, 5, 7, and 12, a 2′-fluoro nucleotide at position 14, and a 2′-OMe nucleotide at position 16, counting from the 5′-end of the antisense strand.
Embodiment 16: The dsRNA agent of any one of Embodiments 1-11, wherein the antisense strand comprises a 2′-deoxy nucleotide at position 14 of the antisense strand, counting from 5′-end of the antisense strand, and the sense strand comprises a nucleotide other than a 2′-fluoro nucleotide at position 7, counting from 5′-end of the sense strand.
Embodiment 17: The dsRNA agent of any one Embodiments 1-11 or 16, wherein the antisense strand comprises a 2′-deoxy nucleotide at position 14 of the antisense strand, counting from 5′-end of the antisense strand, and the sense strand comprises a 2′-OMe nucleotide at position 7, counting from 5′-end of the sense strand.
Embodiment 18: The dsRNA agent of any one Embodiments 1-11 or 16-17, wherein the antisense strand comprises a 2′-deoxy nucleotide at position 2, 5, 7, 12 and 14 of the antisense strand, counting from 5′-end of the antisense strand, and the sense strand comprises a nucleotide other than 2′-fluoro nucleotide at position 7, counting from 5′-end of the sense strand.
Embodiment 19: The dsRNA agent of any one of Embodiments 1-18, wherein the antisense strand comprises a 2′-deoxy nucleotide at position 16 of the antisense strand, counting from 5′-end of the antisense strand, and the sense strand comprises a nucleotide other than a 2′-fluoro nucleotide at position 7, counting from 5′-end of the sense strand.
Embodiment 20: The dsRNA agent of any one of Embodiments 1-19, wherein the antisense strand comprises a 2′-deoxy nucleotide at position 16 of the antisense strand, counting from 5′-end of the antisense strand, and the sense strand comprises 2′-OMe nucleotide at position 7, counting from 5′-end of the sense strand.
Embodiment 21: The dsRNA agent of any one of Embodiments 1-20, wherein the antisense strand comprises a 2′-deoxy nucleotide at position 2, 5, 7, 12 and 16 of the antisense strand, counting from 5′-end of the antisense strand, and the sense strand comprises a nucleotide other than a 2′-fluoro at position 7, counting from 5′-end of the sense strand.
Embodiment 22: The dsRNA agent of any one of Embodiments 1-11 or 16-21, wherein the antisense strand comprises a 2′-deoxy nucleotide at position 2, 5, 7, 12, 14 and 16 of the antisense strand, counting from 5′-end of the antisense strand, and the sense strand comprises a nucleotide other than a 2′-fluoro at position 7, counting from 5′-end of the sense strand.
Embodiment 23: The dsRNA agent of any one of Embodiments 1-11 or 16-22, wherein the antisense strand comprises a 2′-deoxy nucleotide at position 2, 5, 7, 12, 14 and 16, and the sense strand comprises a nucleotide other than a 2′-fluoro at position 7, counting from 5′-end of the sense strand.
Embodiment 24: The dsRNA agent of any one of Embodiments 1-15 or 19-20, wherein the antisense strand comprises a 2′-deoxy nucleotide at position 2, 5, 7, 12, and 16 and a 2′-fluoro at postion 14 of the antisense strand, counting from 5′-end of the antisense strand, and the sense strand comprises a nucleotide other than a 2′-fluoro at position 7, counting from 5′-end of the sense strand.
Embodiment 25: The dsRNA agent of any one of Embodiments 1-15, 19-20 or 24, wherein the antisense strand comprises a 2′-deoxy nucleotide at position 2, 5, 7, 12, and 16 and a 2′-fluoro at postion 14 of the antisense strand, counting from 5′-end of the antisense strand, and the sense strand comprises a 2′-OMe nucleotide at position 7, counting from 5′-end of the sense strand.
Embodiment 26: The dsRNA agent of any one of Embodiments 1-25, wherein the dsRNA agent comprises a ligand.
Embodiment 27: The dsRNA agent of any one of Embodiments 1-26, wherein the sense strand comprises a ligand.
Embodiment 28: The dsRNA agent of Embodiment 27, wherein the ligand is at 3′-end of the sense strand.
Embodiment 29: The dsRNA agent of Embodiment 27, wherein the ligand is at 5′-end of the sense strand.
Embodiment 30: The dsRNA agent of any one of Embodiments 26-29, wherein the ligand comprises an ASGPR ligand.
Embodiment 31: The dsRNA agent of any one of Embodiments 26-29, wherein the ligand is lipophilic group.
Embodiment 32: The dsRNA agent of Embodiment 31, wherein the ligand is a C10-30aliphatic group.
Embodiment 33: The dsRNA agent of Embodiment 32, wherein the C10-30aliphatic

group is a C₁₀₋₃₀alkyl group.
Embodiment 34: The dsRNA agent of Embodiment 33, wherein the C₁₀₋₃₀alkyl group is a straight-chain or branched tetradecyl, hexadecyl, octadecyl, icosyl, docosyl, or tetracosyl group.
Embodiment 35: The dsRNA agent of any one of Embodiments 27, wherein the ligand is conjugated to a non-terminal nucleotide of the sense strand.
Embodiment 36: The dsRNA agent of Embodiment 35, wherein the ligand is conjugated to the 2′-position of a non-terminal nucleotide of the sense strand, optionally conjugated to one of positions 5, 6, 7, or 8 of the sense strand, counting from the 5′ end).
Embodiment 37: The dsRNA agent of Embodiment 26-36, wherein the ligand comprises an abasic nucleotide, optionally the abasic nucleotide is an inverted nucleotide and linked via a 5′->5′ or a 3′->3′ linkage to a strand of the dsRNA agent.
Embodiment 38: The dsRNA agent of any one of Embodiments 26-37, wherein the ligand is attached at the 3′-end of the sense strand.
Embodiment 39: The dsRNA agent of Embodiment 38, wherein the ligand is attached at the 3′-end of the sense strand via a 3′->3′ linkage.
Embodiment 40: The dsRNA agent of any one of Embodiments 1-39, wherein the dsRNA comprises two ligands.
Embodiment 41: The dsRNA of Embodiment 40, wherein the sense strand comprises a first ligand attached at the 3′-end of the sense strand and a second ligand attached at the 5′-end of the sense strand.
Embodiment 42: The dsRNA of Embodiment 41, wherein the first ligand comprises an abasic nucleotide and the second ligand comprises an ASGPR ligand, optionally the abasic nucleotide is an inverted nucleotide and linked via a 3′->3′ linkage to the sense strand.
Embodiment 43: The dsRNA agent of any one of Embodiments 1-42, wherein the dsRNA agent comprises at least two phosphorothioate internucleotide linkages.
Embodiment 44: The dsRNA agent of any one of Embodiments 1-43, wherein the sense strand comprises at least two phosphorothioate internucleotide linkages between the first five nucleotides counting from the 5′ end of the sense strand.
Embodiment 45: The dsRNA agent of any one of Embodiments 1-44, wherein the antisense strand comprises at least two phosphorothioate internucleotide linkages between the first five nucleotides counting from the 5′ end of the antisense strand and at least two phosphorothioate internucleotide linkages between the first five nucleotides counting from the 3′ end of the antisense strand.
Embodiment 46: The dsRNA agent of any one of Embodiments 1-45, wherein the dsRNA has a duplex region of from 18 to about 25 basepairs.
Embodiment 47: The dsRNA agent of any one of Embodiments 1-46, wherein the sense strand is 18-23 nucleotides in length.
Embodiment 48: The dsRNA agent of any one of Embodiments 1-47, wherein the antisense strand is 18-25 nucleotides in length.
Embodiment 49: A dsRNA agent comprising a sense strand and an antisense strand, wherein the sense strand is 18-23 nucleotides in length and comprises a 2′-fluoro nucleotide at position 10, counting from 5′-end of the sense strand and a 2′-fluoro nucleotide at position 9 or 11, counting from 5′-end of the sense strand, and the antisense strand is 18-25 nucleotide in length and comprises a 2′-deoxy nucleotide at positions 2, 5, 7, and 12, counting from 5′-end of the antisense strand, wherein: (i) the antisense strand comprises a 2′-fluoro nucleotide at position 14 and a nucleotide other than a 2′-deoxy or 2′-fluoro nucleotide at position 16, counting from the 5′-end of the antisense strand; or (ii) the antisense strand comprises a 2′-deoxy nucleotide at position 14 or 16, counting from the 5′-end of the antisense strand, and the sense strand comprises a nucleotide other than a 2′-fluoro nucleotide at position 7, counting from the 5′-end of the sense strand.
Embodiment 50: A dsRNA agent comprising a sense strand and an antisense strand, wherein the sense strand is 18-23 nucleotides in length and comprises a 2′-fluoro nucleotide at positions 9, 10 and 11, counting from 5′-end of the sense strand, and the antisense is 18-25 nucleotide in length and comprises a 2′-deoxy nucleotide at position 2, 5, 7, and 12, counting from 5′-end of the antisense strand, wherein: (i) the antisense strand comprises a 2′-fluoro nucleotide at position 14 and a nucleotide other than a 2′-deoxy or 2′-fluoro nucleotide at position 16, counting from the 5′-end of the antisense strand; or (ii) the antisense strand comprises a 2′-deoxy nucleotide at position 14 or 16, counting from the 5′-end of the antisense strand, and the sense strand comprises a nucleotide other than a 2′-fluoro nucleotide at position 7, counting from the 5′-end of the sense strand.
Embodiment 51: The dsRNA agent of any one of Embodiments 1-50, comprising a phosphate mimic at the 5′-end of the antisense strand.
Embodiment 52: The dsRNA agent of Embodiment 51, wherein the phosphate mimic is a 5′-E-vinyl phosphonate.
Embodiment 53: The dsRNA agent of Embodiment 52, wherein the phosphate mimic is a 5′-cyclopropylphosphonate having the structure:

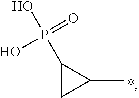

where * is a bond to C₅ position of the nucleotide at the 5′-terminus.
Embodiment 54: The dsRNA agent of any one of Embodiments 1-53, wherein remaining nucleotides (i.e., nucleotides at positions not otherwise defined) in the sense strand are unmodified nucleotides or modified nucleotides, optioally selected from the groups consisting of of 2′-OMe, 2′-F, 2′-H, and an 2′-O—C10-30aliphatic group, provided no more than one modified nucleotide is an 2′-O—C10-30aliphatic group.
Embodiment 55: The dsRNA agent of any one of Embodiments 1-54, wherein remaining nucleotides (i.e., nucleotides at positions not otherwise defined) in the sense strand are modified nucleotides selected from the group consisting of 2′-OMe, 2′-F, 2′-H, and an 2′-O—C10-30aliphatic group, provided no more than one modified nucleotide is an 2′-O—C10-30aliphatic group.
Embodiment 56: The dsRNA agent of any one of Embodiments 1-55, wherein remaining nucleotides (i.e., nucleotides at positions not otherwise defined) in the antisense strand are unmodified nucleotides or modified nucleotides, optioally selected from the group consisting of 2′-OMe, 2′-F, 2′-H, GNA and 3′-RNA, the 3′-RNA being optionally 3′-OH, provided no more than one modified nucleotide is GNA or 3′-RNA.
Embodiment 57: The dsRNA agent of any one of Embodiments 1-56, wherein remaining nucleotides (i.e., nucleotides at positions not otherwise defined) in the antisense strand are modified nucleotides selected from the group consisting of 2′-OMe, 2′-F, 2′-H, GNA, and 3′-RNA, the 3′-RNA being optionally 3′-OH, provided no more than one modified nucleotide is GNA or 3′-RNA.

### Some Selected Definitions

For convenience, certain terms employed herein, in the specification, examples and appended claims are collected herein. Unless stated otherwise, or implicit from context, the following terms and phrases include the meanings provided below. Unless explicitly stated otherwise, or apparent from context, the terms and phrases below do not exclude the meaning that the term or phrase has acquired in the art to which it pertains. The definitions are provided to aid in describing particular embodiments, and are not intended to limit the claimed invention, because the scope of the invention is limited only by the claims. Further, unless otherwise required by context, singular terms shall include pluralities and plural terms shall include the singular.

Unless defined otherwise, all technical and scientific terms used herein have the same meaning as those commonly understood to one of ordinary skill in the art to which this invention pertains. Although any known methods, devices, and materials may be used in the practice or testing of the invention, the methods, devices, and materials in this regard are described herein.

Further, the practice of the present invention can employ, unless otherwise indicated, conventional techniques of molecular biology (including recombinant techniques), microbiology, cell biology, biochemistry, and immunology, which are within the skill of the art. Such techniques are explained fully in the literature, such as, “Molecular Cloning: A Laboratory Manual”, second edition (Sambrook et al., 1989); “Oligonucleotide Synthesis” (M. J. Gait, ed., 1984); “Animal Cell Culture” (R. I. Freshney, ed., 1987); “Methods in Enzymology” (Academic Press, Inc.); “Current Protocols in Molecular Biology” (F. M. Ausubel et al., eds., 1987, and periodic updates); “PCR: The Polymerase Chain Reaction”, (Mullis et al., ed., 1994); “A Practical Guide to Molecular Cloning” (Perbal Bernard V., 1988); “Phage Display: A Laboratory Manual” (Barbas et al., 2001).

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

As used herein the term “comprising” or “comprises” is used in reference to compositions, methods, and respective component(s) thereof, that are essential to the invention, yet open to the inclusion of unspecified elements, whether essential or not.

The singular terms “a,” “an,” and “the” include plural referents unless context clearly indicates otherwise. Similarly, the word “or” is intended to include “and” unless the context clearly indicates otherwise. It is further noted that the claims can be drafted to exclude any optional element. As such, this statement is intended to serve as antecedent basis for use of such exclusive terminology as “solely,” “only” and the like in connection with the recitation of claim elements, or use of a “negative” limitation.

As used herein, the terms “dsRNA”, “siRNA”, and “iRNA agent” are used interchangeably to refer to agents that can mediate silencing of a target RNA, e.g., mRNA, e.g., a transcript of a gene that encodes a protein. For convenience, such mRNA is also referred to herein as mRNA to be silenced. Such a gene is also referred to as a target gene. In general, the RNA to be silenced is an endogenous gene, exogenous gene or a pathogen gene. In addition, RNAs other than mRNA, e.g., tRNAs, and viral RNAs, can also be targeted.

As used herein, the phrase “mediates RNAi” refers to the ability to silence, in a sequence specific manner, a target gene, e.g., mRNA. While not wishing to be bound by theory, it is believed that silencing uses the RNAi machinery or process and a guide RNA, e.g., antisense strand of a dsRNA, where the antisense strand is 21 to 23 nucleotides in length.

As used herein, “specifically hybridizable” and “complementary” are terms which are used to indicate a sufficient degree of complementarity such that stable and specific binding occurs between a compound of the invention and a target RNA molecule. Specific binding requires a sufficient degree of complementarity to avoid non-specific binding of the oligomeric compound to non-target sequences under conditions in which specific binding is desired, i.e., under physiological conditions in the case of assays or therapeutic treatment, or in the case of in vitro assays, under conditions in which the assays are performed. The non-target sequences typically differ by at least 5 nucleotides.

In some embodiments, a dsRNA molecule of the invention is “sufficiently complementary” to a target RNA, e.g., a target mRNA, such that the dsRNA molecule silences production of protein encoded by the target mRNA. In another embodiment, the dsRNA molecule of the invention is “exactly complementary” to a target RNA, e.g., the target RNA and the dsRNA duplex agent anneal, for example to form a hybrid made exclusively of Watson-Crick base pairs in the region of exact complementarity. A “sufficiently complementary” target RNA can include an internal region (e.g., of at least 10 nucleotides) that is exactly complementary to a target RNA. Moreover, in some embodiments, the dsRNA molecule of the invention specifically discriminates a single-nucleotide difference. In this case, the dsRNA molecule only mediates RNAi if exact complementary is found in the region (e.g., within 7 nucleotides of) the single-nucleotide difference.

The term ‘BNA’ refers to bridged nucleic acid, and is often referred as constrained or inaccessible RNA. BNA can contain a 5-, 6-membered, or even a 7-membered bridged structure with a “fixed” C3′-endo sugar puckering. The bridge is typically incorporated at the 2′-, 4′-position of the ribose to afford a 2′, 4′-BNA nucleotide (e.g., LNA, or ENA). Examples of BNA nucleotides include the following nucleosides:

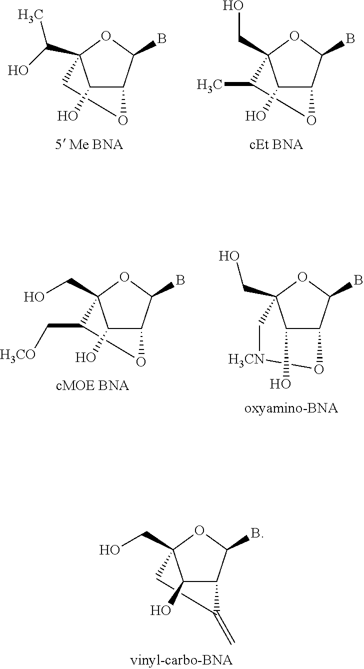

The term ‘LNA’ refers to locked nucleic acid, and is often referred as constrained or inaccessible RNA. LNA is a modified RNA nucleotide. The ribose moiety of an LNA nucleotide is modified with an extra bridge (e.g., a methylene bridge or an ethylene bridge) connecting the 2′ hydroxyl to the 4′ carbon of the same ribose sugar. For instance, the bridge can “lock” the ribose in the 3′-endo North) conformation:

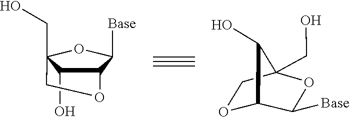

The term ‘ENA’ refers to ethylene-bridged nucleic acid, and is often referred as constrained or inaccessible RNA.

The “cleavage site” herein means the backbone linkage in the target gene or the sense strand that is cleaved by the RISC mechanism by utilizing the iRNA agent. And the target cleavage site region comprises at least one or at least two nucleotides on both side of the cleavage site. For the sense strand, the cleavage site is the backbone linkage in the sense strand that would get cleaved if the sense strand itself was the target to be cleaved by the RNAi mechanism. The cleavage site can be determined using methods known in the art, for example the 5′-RACE assay as detailed in Soutschek et al., Nature (2004) 432, 173-178, which is incorporated by reference in its entirety. As is well understood in the art, the cleavage site region for a conical double stranded RNAi agent comprising two 21-nucleotides long strands (wherein the strands form a double stranded region of 19 consecutive base pairs having 2-nucleotide single stranded overhangs at the 3′-ends), the cleavage site region corresponds to positions 9-12 from the 5′-end of the sense strand.

The terms “decrease”, “reduced”, “reduction”, or “inhibit” are all used herein to mean a decrease by a statistically significant amount. In some embodiments, “reduce,” “reduction” or “decrease” or “inhibit” typically means a decrease by at least 10% as compared to a reference level (e.g. the absence of a given treatment) and can include, for example, a decrease by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, or more. As used herein, “reduction” or “inhibition” does not encompass a complete inhibition or reduction as compared to a reference level. “Complete inhibition” is a 100% inhibition as compared to a reference level. A decrease can be preferably down to a level accepted as within the range of normal for an individual without a given disorder.

As used herein, a “central region” of a strand refers to positions 5-17, e.g., positions 6-16, positions 6-15, positions 6-14, positions 6-13, positions 6-12, positions 7-15, positions 7-14, positions 7-13, positions, 7-12, positions 8-16, positions 8-15, positions 8-14, positions 8-13, positions 8-12, positions 9-16, positions 9-15, positions 9-14, positions 9-13, positions 9-12, positions 10-16, positions 10-15, positions 10-14, positions 10-13 or positions 10-12, counting from the 5′-end of the strand. For example, the central region of a strand means positions 5, 6, 7, 8, 9, 10, 11, 12, 13, 14, 15, 16 or 17 of the strand. A preferred central region for the sense strand is positions 6, 7, 8, 9, 10, 11, 12, 13, and 14, counting from the 5′-end of the sense strand. A more preferred central region for the sense strand is positions 7, 8, 9, 10, 11, 12 and 13, counting from the 5′-end of the sense strand. A preferred central region for the antisense strand is positions 9, 10, 11, 12, 13, 14, 15 16 and 17, counting from 5′-end of the antisense strand. A more preferred central region for the antisense strand is positions 10, 11, 12, 13, 14, 15 and 16, counting from 5′-end of the antisense strand.

As will be apparent to those of skill in the art upon reading this disclosure, each of the individual aspects described and illustrated herein has discrete components and features which can be readily separated from or combined with the features of any of the other several aspects without departing from the scope or spirit of the present invention. Any recited method can be carried out in the order of events recited or in any other order which is logically possible.

The invention is further illustrated by the following examples, which should not be construed as further limiting. The contents of all references, pending patent applications and published patents, cited throughout this application are hereby expressly incorporated by reference.

### Examples

### Oligonucleotide Synthesis and Purification

All oligonucleotides were prepared on a MerMade 192 synthesizer on a 1 μmole scale using universal or custom supports. All phosphoramidites were used at a concentration 100 mM in 100% Acetonitrile or 9:1 Acetonitrile: DMF with a standard protocol for 2-cyanoethyl phosphoramidites, except that the coupling time was extended to 400 seconds. Oxidation of the newly formed linkages was achieved using a solution of 50 mM 12 in 9:1 Acetonitrile: Water to create phosphate linkages and 100 mM DDTT in 9:1 Pyridine: Acetonitrile to create phosphorothioate linkages. After the trityl-off synthesis, columns were incubated with 150 μL of 40% aqueous Methylamine for 45 minutes and the solution drained via vacuum into a 96-well plate. After repeating the incubation and draining with a fresh portion of aqueous Methylamine, the plate containing crude oligonucleotide solution was sealed and shaken at room temperature for an additional 60 minutes to completely remove all protecting groups. Precipitation of the crude oligonucleotides was accomplished via the addition of 1.2 mL of 9:1 Acetonitrile: EtOH to each well followed by incubation at −20° C. overnight. The plate was then centrifuged at 3000 RPM for 45 minutes, the supernatant removed from each well, and the pellets resuspended in 950 μL of 20 mM aqueous NaOAc. Each crude solution was finally desalted over a GE Hi-Trap Desalting Column (Sephadex G25 Superfine) using water to elute the final oligonucleotide products. All identities and purities were confirmed using ESI-MS and IEX HPLC, respectively.

### Cell Culture and Transfections

Primary Mouse or Cyno Hepatocytes (Thermo Fisher Scientific/Gibco) were transfected by adding 4.9 μL of Opti-MEM plus 0.1 μL of Lipofectamine RNAiMax per well (Invitrogen, cat #13778-150) to 5 μL of siRNA duplexes per well into a 384-well plate and incubated at room temperature for 15 minutes. 40 μL of Dulbecco's Modified Eagle Medium (PCH) or William's Medium (PMH) containing ˜5 ×10³ cells were then added to the siRNA mixture. Cells were incubated for 24 hours at 37° C. and then processed for RNA purification. Experiments were performed at 10 nM, 1 nM, and 0.1 nM doses of siRNA.

Sequences of parent dsRNA molecules are shown in Tables 1-3 and abbrevations used in the sequences are summarized in Table 4.

TABLE 1
Sequences of parent dsRNA molecules

| | | | SEQ | | SEQ |
|---|---|---|---|---|---|
| | Duplex | | ID | | ID |
| target | ID | Sense Sequence (5′→3′) | NO: | Antisense Sequence (5′→3′) | NO: |
| AGT | AD-67327 | uscsucccAfcCfUfUfuucuucuaauL96 | 5 | asUfsuagAfagaaaagGfuGfggagascsu | 37 |
| AGT | AD-85435 | csascaauGfaGfAfGfuaccugugaaL96 | 6 | usUfscacagguacucUfcAfuugugsgsa | 38 |
| AGT | AD-85438 | cscsucaaCfuGfGfAfugaagaaacuL96 | 7 | asGfsuuucuucauccAfgUfugaggsgsa | 39 |
| AGT | AD-85446 | gscsugagAfaGfAfUfugacagguuaL96 | 8 | usAfsaccugucaaucUfuCfucagcsasg | 40 |
| F9 | AD-674282 | gsasagcaCfcUfGfAfacaccgucauL96 | 9 | asUfsgacGfgUfGfuucaGfgUfgcuucsasu | 41 |
| F9 | AD-674283 | asasugugCfaGfUfUfuuccuugauuL96 | 10 | asAfsucaAfgGfAfaaacUfgCfacauuscsg | 42 |
| F9 | AD-674287 | uscscuugUfuUfAfAfaugguggaauL96 | 11 | asUfsuccAfcCfAfuuuaAfaCfaaggasusu | 43 |
| F9 | AD-674289 | asasaaugGfcAfGfGfugcaagcaguL96 | 12 | asCfsugcUfuGfCfaccuGfcCfauuuususa | 44 |
| F9 | AD-674293 | uscsauucAfaGfAfUfgacaucacuuL96 | 13 | asAfsgugAfuGfUfcaucUfuGfaaugasasu | 45 |
| F9 | AD-674299 | gsasacugGfaUfAfAfaccuuuaauaL96 | 14 | usAfsuuaAfaGfGfuuuaUfcCfaguucscsa | 46 |
| F9 | AD-674303 | usgsaaggAfgGfCfAfaagauucguuL96 | 15 | asAfscgaAfuCfUfuugcCfuCfcuucascsg | 47 |
| F9 | AD-674305 | asasuauaUfaCfUfAfagguuucccuL96 | 16 | asGfsggaAfaCfCfuuagUfaUfauauuscsc | 48 |
| F9 | AD-674307 | ascsagacUfaGfUfCfuuucuaccuuL96 | 17 | asAfsgguAfgAfAfagacUfaGfucugusasa | 49 |
| F9 | AD-674310 | csasuaagGfcGfUfGfaugguucuuuL96 | 18 | asAfsagaAfcCfAfucacGfcCfuuaugsgsg | 50 |
| F9 | AD-674312 | ususuaugGfcCfAfGfaagaauacaaL96 | 19 | usUfsguaUfuCfUfucugGfcCfauaaasusg | 51 |
| F9 | AD-674314 | csuscuccGfuCfAfUfucucaccuuuL96 | 20 | asAfsaggUfgAfGfaaugAfcGfgagagsgsu | 52 |
| F9 | AD-674323 | uscsucauCfuGfAfGfacuuggugaaL96 | 21 | usUfscacCfaAfGfucucAfgAfugagasasa | 53 |
| F9 | AD-674325 | gsascucuGfcUfAfAfcguuccacuaL96 | 22 | usAfsgugGfaAfCfguuaGfcAfgagucsasg | 54 |
| F9 | AD-68435 | usgsugcaAfuGfAfAfaggcaaauauL96 | 23 | asUfsauuUfgCfCfuuucAfuUfgcacascsu | 55 |
| mTTR | AD- | csasguguUfcUfUfGfcucuauaaaaL96 | 24 | usUfsuuaUfagagcaaGfaAfcacugsusu | 56 |
| | 1181392 | | | | |
| mTTR | AD- | uscsuugcUfcUfAfUfaaaccguguuL96 | 25 | asAfscacGfguuuauaGfaGfcaagasasc | 57 |
| | 1181401 | | | | |
| | (Sequence | | | | |
| | 1) | | | | |
| mTTR | AD- | asgsuguuCfuUfGfCfucuauaaacaL96 | 26 | usGfsuuuAfuagagcaAfgAfacacusgsu | 58 |
| | 1181410 | | | | |
| | (Sequence | | | | |
| | 2) | | | | |
| mTTR | AD- | asgsaacuGfgAfCfAfccaaaucguaL96 | 27 | usAfscgaUfuugguguCfcAfguucusasc | 59 |
| | 1181417 | | | | |
| mTTR | AD- | ascsagugUfuCfUfUfgcucuauaaaL96 | 28 | usUfsuauAfgagcaagAfaCfacugususu | 60 |
| | 1181426 | | | | |
| | (Sequence | | | | |
| | 3) | | | | |
| mTTR | AD- | ususcuugCfuCfUfAfuaaaccguguL96 | 29 | asCfsacgGfuuuauagAfgCfaagaascsa | 61 |
| | 1181443 | | | | |
| mTTR | AD- | gsusguucUfuGfCfUfcuauaaaccaL96 | 30 | usGfsguuUfauagagcAfaGfaacacsusg | 62 |
| | 1181451 | | | | |
| | (Sequence | | | | |
| | 4) | | | | |
| mTTR | AD- | asascuggAfcAfCfCfaaaucguacuL96 | 31 | asGfsuacGfauuugguGfuCfcaguuscsu | 63 |
| | 1181460 | | | | |
| mTTR | AD | csasggagGfaCfCfAfggaucuugcaL96 | 32 | usGfscaaGfauccuggUfcCfuccugsgsg | 64 |
| | 1181469 | | | | |
| TTR | AD-157448 | csasauaaAfaCfAfUfuccugugaaaL96 | 33 | usUfsucacaggaaugUfuUfuauugsusc | 65 |
| TTR | AD-157464 | csusaaagCfaGfUfGfuuuucaccuaL96 | 34 | usAfsggugaaaacacUfgCfuuuagsusa | 66 |
| TTR | AD-157468 | csasgagaCfaAfUfAfaaacauuccuL96 | 35 | asGfsgaauguuuuauUfgUfcucugscsc | 67 |
| none | AD-64972 | uscscucuGfaUfGfGfucaaaguccuL96 | 36 | asGfsgacUfuugaccaUfcAfgaggascsa | 68 |

TABLE 2
Additional exemplary sequences of parent dsRNA molecules.

| | | | SEQ | | SEQ |
|---|---|---|---|---|---|
| Duplex | | | ID | | ID |
| ID | Target | Sense sequence (5′→3′) | NO: | Antisense sequence (5′→3′) | NO: |
| AD- | Marc1 | asasaaauGfuUfCfUfcaaaaaugaaL96 | 69 | usUfscauUfuUfUfgagaAfcAfuuuuusasa | 237 |
| 1531684 | | | | | |
| AD- | Marc1 | asasaaucAfcCfAfCfucuuugggcaL96 | 70 | usGfscccAfaAfGfagugGfuGfauuuuscsc | 238 |
| 1531719 | | | | | |
| AD- | Marc1 | asasagugGfgAfGfAfcccuguguaaL96 | 71 | usUfsacaCfaGfGfgucuCfcCfacuuusgsa | 239 |
| 1531682 | | | | | |
| AD-75247 | PNPLA3 | asasaugaAfaGfAfCfaaagguggauL96 | 72 | asUfsccaCfcUfUfugucUfuUfcauuuscsu | 240 |
| AD- | C3 | asasaugaGfgGfUfUfucacagucaaL96 | 73 | usUfsgacUfgUfGfaaacCfcUfcauuususc | 241 |
| 1531655 | | | | | |
| AD-67589 | PNPLA3 | asascuugCfuAfCfCfcauuaggauaL96 | 74 | usAfsuccUfaAfUfggguAfgCfaaguusgsc | 242 |
| AD- | Marc1 | asasucacCfaCfUfCfuuugggcaguL96 | 75 | asCfsugcCfcAfAfagagUfgGfugauususu | 243 |
| 1531703 | | | | | |
| AD- | C3 | ascsauggGfcCfAfGfuggaagaucaL96 | 76 | usGfsaucUfuCfCfacugGfcCfcaugususg | 244 |
| 1531665 | | | | | |
| AD- | C3 | ascscaggAfaCfUfGfaaccuugauaL96 | 77 | usAfsucaAfgGfUfucagUfuCfcuggusgsg | 245 |
| 1531660 | | | | | |
| AD- | C3 | ascscaggAfuGfCfCfacuaugucuaL96 | 78 | usAfsgacAfuAfGfuggcAfuCfcugguscsu | 246 |
| 1531672 | | | | | |
| AD- | PNPLA3 | ascscuaaCfuAfAfAfauaauguuuaL96 | 79 | usAfsaacAfuUfAfuuuuAfgUfuaggusgsa | 247 |
| 1010735 | | | | | |
| AD- | Marc1 | ascscucgCfcUfGfGfuccugauuuaL96 | 80 | usAfsaauCfaGfGfaccaGfgCfgaggususc | 248 |
| 1531721 | | | | | |
| AD-67605 | PNPLA3 | ascscuguUfgAfAfUfuuuguauuauL96 | 81 | asUfsaauAfcAfAfaauuCfaAfcaggusasa | 249 |
| AD- | C3 | ascsuacaUfgAfAfCfcuacagagauL96 | 82 | asUfscucUfgUfAfgguuCfaUfguagususg | 250 |
| 571552 | | | | | |
| AD- | Marc1 | ascsugauUfaUfGfGfaauaguucuuL96 | 83 | asAfsgaaCfuAfUfuccaUfaAfucagususa | 251 |
| 1531716 | | | | | |
| AD- | C3 | asgsaaauUfcUfAfCfuacaucuauuL96 | 84 | asAfsuagAfuGfUfaguaGfaAfuuucuscsu | 252 |
| 569269 | | | | | |
| AD- | Marc1 | asgsacagGfaUfUfCfugaaaacucaL96 | 85 | usGfsaguUfuUfCfagaaUfcCfugucususg | 253 |
| 1531718 | | | | | |
| AD- | C3 | asgsagcgGfgUfAfCfcucuucaucaL96 | 86 | usGfsaugAfaGfAfgguaCfcCfgcucusgsc | 254 |
| 1531663 | | | | | |
| AD- | SCN9A | asgscaaaGfgUfCfAfcaauuuccuaL96 | 87 | usAfsggaAfaUfUfgugaCfcUfuugcuscsa | 255 |
| 1531765 | | | | | |
| AD- | SCN9A | asgscauaAfaUfGfUfuuucgaaauaL96 | 88 | usAfsuuuCfgAfAfaacaUfuUfaugcususc | 256 |
| 1531735 | | | | | |
| AD- | PNPLA3 | asgscaugAfgGfUfUfcuuagaauguL96 | 89 | asCfsauuCfuAfAfgaacCfuCfaugcusgsg | 257 |
| 519933 | | | | | |
| AD- | PNPLA3 | asgsgaagCfaAfCfCfuuucgccuguL96 | 90 | asCfsaggCfgAfAfagguUfgCfuuccusasg | 258 |
| 519780 | | | | | |
| AD- | Marc1 | asgsgaccAfgAfUfUfgcuuacucaaL96 | 91 | usUfsgagUfaAfGfcaauCfuGfguccususg | 259 |
| 1531679 | | | | | |
| AD- | Marc1 | asgsgagaAfgAfAfAfagugauucaaL96 | 92 | usUfsgaaUfcAfCfuuuuCfuUfcuccuscsc | 260 |
| 1531692 | | | | | |
| AD- | SCN9A | asgsucuuCfaAfGfUfuggcaaaauaL96 | 93 | usAfsuuuUfgCfCfaacuUfgAfagacuscsg | 261 |
| 1531741 | | | | | |
| AD- | C3 | asgsuggaCfuAfUfGfuguacaagauL96 | 94 | asUfscuuGfuAfCfacauAfgUfccacuscsc | 262 |
| 572575 | | | | | |
| AD- | SCN9A | asgsuuccUfaUfCfUfccuuucagaaL96 | 95 | usUfscugAfaAfGfgagaUfaGfgaacusasc | 263 |
| 1531744 | | | | | |
| AD- | PNPLA3 | asusaaugUfcUfUfAfuguaaugcuuL96 | 96 | asAfsgcaUfuAfCfauaaGfaCfauuauscsc | 264 |
| 519354 | | | | | |
| AD-67551 | PNPLA3 | asusacauGfaGfCfAfagauuugcaaL96 | 97 | usUfsgcaAfaUfCfuugcUfcAfuguauscsc | 265 |
| AD- | Marc1 | asuscaacCfaGfGfAfgggaaacauaL96 | 98 | usAfsuguUfuCfCfcuccUfgGfuugauscsa | 266 |
| 1531687 | | | | | |
| AD- | SCN9A | asuscaucUfuUfGfGfgucauucuuuL96 | 99 | asAfsagaAfuGfAfcccaAfaGfaugausasa | 267 |
| 1531736 | | | | | |
| AD- | Marc1 | asuscugaUfgAfAfGfuauauuuuuuL96 | 100 | asAfsaaaAfuAfUfacuuCfaUfcagauscsu | 268 |
| 1531688 | | | | | |
| AD- | SCN9A | asuscuucUfuUfGfUfcguagugauuL96 | 101 | asAfsucaCfuAfCfgacaAfaGfaagauscsa | 269 |
| 1531747 | | | | | |
| AD- | SCN9A | asusgcugAfgAfAfAfuugucgaaaaL96 | 102 | usUfsuucGfaCfAfauuuCfuCfagcauscsu | 270 |
| 1531756 | | | | | |
| AD- | Marc1 | asusggcuUfgUfUfCfcagaugcauuL96 | 103 | asAfsugcAfuCfUfggaaCfaAfgccauscsa | 271 |
| 1531696 | | | | | |
| AD- | C3 | asusguacCfaUfGfCfuaaggccaaaL96 | 104 | usUfsuggCfcUfUfagcaUfgGfuacaususg | 272 |
| 1531658 | | | | | |
| AD- | SCN9A | asusgucgAfgUfAfCfacuuuuacuuL96 | 105 | asAfsguaAfaAfGfuguaCfuCfgacaususu | 273 |
| 1531757 | | | | | |
| AD-67565 | PNPLA3 | asusguuaGfuAfGfAfauaagccuuaL96 | 106 | usAfsaggCfuUfAfuucuAfcUfaacauscsu | 274 |
| AD- | PNPLA3 | asusuaggAfuAfAfUfgucuuauguaL96 | 107 | usAfscauAfaGfAfcauuAfuCfcuaausgsg | 275 |
| 1010714 | | | | | |
| AD- | SCN9A | asusucucUfuCfGfUfucacagaugaL96 | 108 | usCfsaucUfgUfGfaacgAfaGfagaauscsc | 276 |
| 1531728 | | | | | |
| AD- | Marc1 | csasacacUfuGfAfAfgcaugguguuL96 | 109 | asAfscacCfaUfGfcuucAfaGfuguugsusc | 277 |
| 1531686 | | | | | |
| AD-67583 | PNPLA3 | csasagauUfuGfCfAfacuugcuacaL96 | 110 | usGfsuagCfaAfGfuugcAfaAfucuugscsu | 278 |
| AD- | C3 | csasagguCfuAfCfGfccuauuacauL96 | 111 | asUfsguaAfuAfGfgcguAfgAfccuugsasc | 279 |
| 572388 | | | | | |
| AD- | C3 | csascccuCfaUfCfAfucuaccuggaL96 | 112 | usCfscagGfuAfGfaugaUfgAfgggugsusu | 280 |
| 1531662 | | | | | |
| AD- | C3 | csasccguAfuCfCfAfcugggaaucuL96 | 113 | asGfsauuCfcCfAfguggAfuAfcggugsgsg | 281 |
| 571901 | | | | | |
| AD- | PNPLA3 | csasccuuUfuUfCfAfccuaacuaaaL96 | 114 | usUfsuagUfuAfGfgugaAfaAfaggugsusu | 282 |
| 1010732 | | | | | |
| AD- | PNPLA3 | csasccuuUfuUfCfAfccuaacuaauL96 | 115 | asUfsuagUfuAfGfgugaAfaAfaggugsusu | 283 |
| 520061 | | | | | |
| AD- | SCN9A | csascuccUfuCfCfUfgauuguguuuL96 | 116 | asAfsacaCfaAfUfcaggAfaGfgagugsgsa | 284 |
| 1531770 | | | | | |
| AD- | Marc1 | csasgaacGfaAfAfGfuuauauggaaL96 | 117 | usUfsccaUfaUfAfacuuUfcGfuucugsasa | 285 |
| 1531705 | | | | | |
| AD- | C3 | csasgagaAfaUfUfCfuacuacaucuL96 | 118 | asGfsaugUfaGfUfagaaUfuUfcucugsusa | 286 |
| 569266 | | | | | |
| AD- | SCN9A | csasugaaCfgAfCfUfucuuccacuuL96 | 119 | asAfsgugGfaAfGfaaguCfgUfucaugsusg | 287 |
| 1531740 | | | | | |
| AD-75265 | PNPLA3 | csasugagCfaAfGfAfuuugcaacuuL96 | 120 | asAfsguuGfcAfAfaucuUfgCfucaugsusa | 288 |
| AD- | Marc1 | csasugguGfuUfUfCfagaacugagaL96 | 121 | usCfsucaGfuUfCfugaaAfcAfccaugscsu | 289 |
| 1531709 | | | | | |
| AD- | C3 | csasuugaGfaAfCfCfcggaaggcauL96 | 122 | asUfsgccUfuCfCfggguUfcUfcaaugsusu | 290 |
| 569048 | | | | | |
| AD- | C3 | cscsacagCfcAfAfAfgauaagaacuL96 | 123 | asGfsuucUfuAfUfcuuuGfgCfuguggsusc | 291 |
| 571633 | | | | | |
| AD- | C3 | cscsagauCfcAfCfUfucaccaagauL96 | 124 | asUfscuuGfgUfGfaaguGfgAfucuggsusa | 292 |
| 569516 | | | | | |
| AD- | PNPLA3 | cscsauuaGfgAfUfAfaugucuuauuL96 | 125 | asAfsuaaGfaCfAfuuauCfcUfaauggsgsu | 293 |
| 519346 | | | | | |
| AD- | C3 | cscscgucGfuGfCfGfuuggcucaauL96 | 126 | asUfsugaGfcCfAfacgcAfcGfacgggsasg | 294 |
| 571748 | | | | | |
| AD- | Marc1 | cscsgaccCfaAfGfGfaccagauuguL96 | 127 | asCfsaauCfuGfGfuccuUfgGfgucggsasa | 295 |
| 1531708 | | | | | |
| AD- | C3 | cscsgagcCfgUfUfCfucuacaauuuL96 | 128 | asAfsauuGfuAfGfagaaCfgGfcucggsasu | 296 |
| 570712 | | | | | |
| AD- | Marc1 | cscsguauGfuCfCfUfggaauauuauL96 | 129 | asUfsaauAfuUfCfcaggAfcAfuacggsusu | 297 |
| 1531706 | | | | | |
| AD-67584 | PNPLA3 | cscsuaacUfaAfAfAfuaauguuuaaL96 | 130 | usUfsaaaCfaUfUfauuuUfaGfuuaggsusg | 298 |
| AD- | C3 | cscsuuguCfuUfCfUfcagaaccagaL96 | 131 | usCfsuggUfuCfUfgagaAfgAfcaaggsasg | 299 |
| 1531668 | | | | | |
| AD-67577 | PNPLA3 | csgsacauCfuGfCfCfcuaaagucaaL96 | 132 | usUfsgacUfuUfAfgggcAfgAfugucgsusa | 300 |
| AD- | C3 | csgsugcgUfuGfGfCfucaaugaacuL96 | 133 | asGfsuucAfuUfGfagccAfaCfgcacgsasc | 301 |
| 571753 | | | | | |
| AD- | C3 | csusacccUfaCfUfCfuguuguucgaL96 | 134 | usCfsgaaCfaAfCfagagUfaGfgguagscsc | 302 |
| 1531657 | | | | | |
| AD- | C3 | csusacugCfaGfCfUfaaaagacuuuL96 | 135 | asAfsaguCfuUfUfuagcUfgCfaguagsgsg | 303 |
| 571715 | | | | | |
| AD- | C3 | csusagugCfuGfUfCfcagugagaaaL96 | 136 | usUfsucuCfaCfUfggacAfgCfacuagsusu | 304 |
| 1531659 | | | | | |
| AD-67560 | PNPLA3 | csusauuaAfuGfGfUfcagacuguuaL96 | 137 | usAfsacaGfuCfUfgaccAfuUfaauagsgsg | 305 |
| AD- | PNPLA3 | csusccauGfgCfGfGfggguaacaaaL96 | 138 | usUfsuguUfaCfCfcccgCfcAfuggagsasc | 306 |
| 1531674 | | | | | |
| AD- | PNPLA3 | csusgaguUfgGfUfUfuuaugaaaauL96 | 139 | asUfsuuuCfaUfAfaaacCfaAfcucagscsu | 307 |
| 519757 | | | | | |
| AD- | SCN9A | csusgcccAfaAfAfUfacugauaauaL96 | 140 | usAfsuuaUfcAfGfuauuUfuGfggcagscsa | 308 |
| 1531732 | | | | | |
| AD- | C3 | csusgggaGfgAfCfCfcugguaagcaL96 | 141 | usGfscuuAfcCfAfggguCfcUfcccagscsg | 309 |
| 1531656 | | | | | |
| AD- | Marc1 | csusguggAfgGfAfGfaagaaaaguaL96 | 142 | usAfscuuUfuCfUfucucCfuCfcacagsasa | 310 |
| 1531678 | | | | | |
| AD- | Marc1 | csusucuuAfuUfGfGfugacguggaaL96 | 143 | usUfsccaCfgUfCfaccaAfuAfagaagscsu | 311 |
| 1531701 | | | | | |
| AD- | Marc1 | csusuguuCfcAfGfAfugcauuuuaaL96 | 144 | usUfsaaaAfuGfCfaucuGfgAfacaagscsc | 312 |
| 1531680 | | | | | |
| AD- | SCN9A | csusuuguCfgUfAfGfugauuuuccuL96 | 145 | asGfsgaaAfaUfCfacuaCfgAfcaaagsasa | 313 |
| 1531734 | | | | | |
| AD- | C3 | gsascagaCfaAfGfAfccaucuacauL96 | 146 | asUfsguaGfaUfGfgucuUfgUfcugucsusg | 314 |
| 568977 | | | | | |
| AD- | C3 | gsasgaaaUfuCfUfAfcuacaucuauL96 | 147 | asUfsagaUfgUfAfguagAfaUfuucucsusg | 315 |
| 569268 | | | | | |
| AD- | C3 | gsasgaacCfaGfAfAfacaaugccauL96 | 148 | asUfsggcAfuUfGfuuucUfgGfuucucsusu | 316 |
| 572818 | | | | | |
| AD- | SCN9A | gsasggucAfaGfAfCfaucuuuaugaL96 | 149 | usCfsauaAfaGfAfugucUfuGfaccucscsa | 317 |
| 1531729 | | | | | |
| AD- | Marc1 | gsasgugcUfcCfUfUfcuccaguucuL96 | 150 | asGfsaacUfgGfAfgaagGfaGfcacucscsg | 318 |
| 1531689 | | | | | |
| AD-75275 | PNPLA3 | gsasuuugCfaAfCfUfugcuacccauL96 | 151 | asUfsgggUfaGfCfaaguUfgCfaaaucsusu | 319 |
| AD-67568 | PNPLA3 | gscsacagGfgAfAfCfcucuaccuuaL96 | 152 | usAfsaggUfaGfAfgguuCfcCfugugcsasg | 320 |
| AD- | C3 | gscsaggcAfgAfGfCfgcagcgggauL96 | 153 | asUfscccGfcUfGfcgcuCfuGfccugcsasc | 321 |
| 569494 | | | | | |
| AD- | Marc1 | gscscauuCfcCfCfUfcagcuaaugaL96 | 154 | usCfsauuAfgCfUfgaggGfgAfauggcsasa | 322 |
| 1531722 | | | | | |
| AD- | C3 | gscscucuUfcUfUfAfacaaauuucuL96 | 155 | asGfsaaaUfuUfGfuuaaGfaAfgaggcscsc | 323 |
| 571610 | | | | | |
| AD- | SCN9A | gscsguugUfaGfUfUfccuaucuccuL96 | 156 | asGfsgagAfuAfGfgaacUfaCfaacgcscsu | 324 |
| 1531753 | | | | | |
| AD- | SCN9A | gscsucauCfaUfGfUfgcacuauucuL96 | 157 | asGfsaauAfgUfGfcacaUfgAfugagcsasu | 325 |
| 1531759 | | | | | |
| AD- | C3 | gscsugagGfaGfAfAfuugcuucauuL96 | 158 | asAfsugaAfgCfAfauucUfcCfucagcsasc | 326 |
| 572495 | | | | | |
| AD- | PNPLA3 | gscsugagUfuGfGfUfuuuaugaaaaL96 | 159 | usUfsuucAfuAfAfaaccAfaCfucagcsusc | 327 |
| 1010719 | | | | | |
| AD- | Marc1 | gscsuucuCfaGfAfCfagcauuggauL96 | 160 | asUfsccaAfuGfCfugucUfgAfgaagcsasg | 328 |
| 1531683 | | | | | |
| AD- | Marc1 | gsgsaccaGfaUfUfGfcuuacucagaL96 | 161 | usCfsugaGfuAfAfgcaaUfcUfgguccsusu | 329 |
| 1531711 | | | | | |
| AD- | C3 | gsgsagguUfgUfGfCfugagccggaaL96 | 162 | usUfsccgGfcUfCfagcaCfaAfccuccscsc | 330 |
| 1531664 | | | | | |
| AD- | SCN9A | gsgscacaUfgAfAfCfgacuucuucaL96 | 163 | usGfsaagAfaGfUfcguuCfaUfgugccsasc | 331 |
| 1531762 | | | | | |
| AD-67526 | PNPLA3 | gsgsccuuAfuCfCfCfuccuuccuuaL96 | 164 | usAfsaggAfaGfGfagggAfuAfaggccsasc | 332 |
| AD- | SCN9A | gsgscuggAfuUfUfCfcuaauuguuuL96 | 165 | asAfsacaAfuUfAfggaaAfuCfcagccsasa | 333 |
| 1531725 | | | | | |
| AD- | SCN9A | gsgsgaaaAfcAfAfUfcuuccguuuaL96 | 166 | usAfsaacGfgAfAfgauuGfuUfuucccsusu | 334 |
| 1531737 | | | | | |
| AD- | PNPLA3 | gsgsgguaAfcAfAfGfaugauaaucuL96 | 167 | asGfsauuAfuCfAfucuuGfuUfaccccscsg | 335 |
| 1531673 | | | | | |
| AD- | Marc1 | gsgsugucUfcAfAfUfgcuucaauguL96 | 168 | asCfsauuGfaAfGfcauuGfaGfacaccsasg | 336 |
| 1531717 | | | | | |
| AD- | C3 | gsusaaugCfaGfGfAfcuucuucauuL96 | 169 | asAfsugaAfgAfAfguccUfgCfauuacsusg | 337 |
| 570644 | | | | | |
| AD- | C3 | gsusaccuCfuUfCfAfuccagacaguL96 | 170 | asCfsuguCfuGfGfaugaAfgAfgguacscsc | 338 |
| 568962 | | | | | |
| AD- | Marc1 | gsusauaaCfuCfUfAfagaucugauuL96 | 171 | asAfsucaGfaUfCfuuagAfgUfuauacsasa | 339 |
| 1531690 | | | | | |
| AD- | SCN9A | gsusccucUfaAfGfAfagaauaucuaL96 | 172 | usAfsgauAfuUfCfuucuUfaGfaggacsusg | 340 |
| 1531750 | | | | | |
| AD- | Marc1 | gsusgaccCfuUfCfAfgaacgaaaguL96 | 173 | asCfsuuuCfgUfUfcugaAfgGfgucacsasc | 341 |
| 1531710 | | | | | |
| AD-67578 | PNPLA3 | gsusgaguGfaCfAfAfcguacccuuaL96 | 174 | usAfsaggGfuAfCfguugUfcAfcucacsusc | 342 |
| AD-67582 | PNPLA3 | gsusgcuaAfaGfUfUfucccaucuuuL96 | 175 | asAfsagaUfgGfGfaaacUfuUfagcacscsu | 343 |
| AD- | C3 | gsusgggaGfaAfGfUfucggccuagaL96 | 176 | usCfsuagGfcCfGfaacuUfcUfcccacsusg | 344 |
| 1531666 | | | | | |
| AD- | Marc1 | usasacucUfaAfGfAfucugaugaauL96 | 177 | asUfsucaUfcAfGfaucuUfaGfaguuasusa | 345 |
| 1531695 | | | | | |
| AD- | SCN9A | usascaugAfuCfUfUfcuuugucguaL96 | 178 | usAfscgaCfaAfAfgaagAfuCfauguasgsg | 346 |
| 1531723 | | | | | |
| AD-75270 | PNPLA3 | usasccugUfuGfAfAfuuuuguauuaL96 | 179 | usAfsauaCfaAfAfauucAfaCfagguasasc | 347 |
| AD- | C3 | usascgugCfuGfCfCfcaguuucgaaL96 | 180 | usUfscgaAfaCfUfgggcAfgCfacguascsu | 348 |
| 1531671 | | | | | |
| AD- | PNPLA3 | usasggauAfaUfGfUfcuuauguaauL96 | 181 | asUfsuacAfuAfAfgacaUfuAfuccuasasu | 349 |
| 519350 | | | | | |
| AD- | SCN9A | usasugccAfaAfAfUfccuuuuuauaL96 | 182 | usAfsuaaAfaAfGfgauuUfuGfgcauasgsa | 350 |
| 1531764 | | | | | |
| AD- | SCN9A | usasugugAfaAfCfAfaaccuuacgaL96 | 183 | usCfsguaAfgGfUfuuguUfuCfacauasasu | 351 |
| 1531739 | | | | | |
| AD- | Marc1 | usasuuguAfaUfUfUfcaggaugcgaL96 | 184 | usCfsgcaUfcCfUfgaaaUfuAfcaauasusu | 352 |
| 1531681 | | | | | |
| AD- | Marc1 | uscsaaugCfuUfCfAfaugucccaguL96 | 185 | asCfsuggGfaCfAfuugaAfgCfauugasgsa | 353 |
| 1531704 | | | | | |
| AD-67564 | PNPLA3 | uscsacuuGfaGfGfAfggcgagucuaL96 | 186 | usAfsgacUfcGfCfcuccUfcAfagugascsu | 354 |
| AD- | Marc1 | uscsagaaCfgAfAfAfguuauauggaL96 | 187 | usCfscauAfuAfAfcuuuCfgUfucugasasg | 355 |
| 1531676 | | | | | |
| AD- | Marc1 | uscsaggaUfgCfGfAfugucuaugcaL96 | 188 | usGfscauAfgAfCfaucgCfaUfccugasasa | 356 |
| 1531700 | | | | | |
| AD- | Marc1 | uscscauaGfaUfCfUfggaucuggcaL96 | 189 | usGfsccaGfaUfCfcagaUfcUfauggasasa | 357 |
| 1531702 | | | | | |
| AD- | SCN9A | uscscauuGfuCfUfUfgacaucuuauL96 | 190 | asUfsaagAfuGfUfcaagAfcAfauggasusc | 358 |
| 1531730 | | | | | |
| AD- | SCN9A | uscscucuAfaGfAfAfgaauaucuauL96 | 191 | asUfsagaUfaUfUfcuucUfuAfgaggascsu | 359 |
| 1531738 | | | | | |
| AD- | SCN9A | uscscugcAfaGfUfCfaaguuccaaaL96 | 192 | usUfsuggAfaCfUfugacUfuGfcaggasasa | 360 |
| 1531733 | | | | | |
| AD-67554 | PNPLA3 | uscsugagCfuGfAfGfuugguuuuauL96 | 193 | asUfsaaaAfcCfAfacucAfgCfucagasgsg | 361 |
| AD- | Marc1 | usgsacccUfuCfAfGfaacgaaaguuL96 | 194 | asAfscuuUfcGfUfucugAfaGfggucascsa | 362 |
| 1531685 | | | | | |
| AD-75269 | PNPLA3 | usgsagugAfaGfAfAfaugaaagacaL96 | 195 | usGfsucuUfuCfAfuuucUfuCfacucasgsu | 363 |
| AD- | SCN9A | usgsauagUfuAfCfCfuaguuugcaaL96 | 196 | usUfsgcaAfaCfUfagguAfaCfuaucasasa | 364 |
| 1531752 | | | | | |
| AD- | SCN9A | usgscagaCfaAfGfAfucuucacuuaL96 | 197 | usAfsaguGfaAfGfaucuUfgUfcugcasusa | 365 |
| 1531754 | | | | | |
| AD- | PNPLA3 | usgsccaaAfaCfAfAfccaucaccguL96 | 198 | asCfsgguGfaUfGfguugUfuUfuggcasusc | 366 |
| 518942 | | | | | |
| AD- | C3 | usgscgauCfaGfAfAfgagaccaaguL96 | 199 | asCfsuugGfuCfUfcuucUfgAfucgcasgsg | 367 |
| 571932 | | | | | |
| AD- | C3 | usgscuaaGfgCfCfAfaagaucaacuL96 | 200 | asGfsuugAfuCfUfuuggCfcUfuagcasusg | 368 |
| 572022 | | | | | |
| AD- | SCN9A | usgscucuCfcAfUfAfuuggauaaaaL96 | 201 | usUfsuuaUfcCfAfauauGfgAfgagcasasu | 369 |
| 1531760 | | | | | |
| AD- | SCN9A | usgscucuCfcUfUfUfgugguuucauL96 | 202 | asUfsgaaAfcCfAfcaaaGfgAfgagcasusc | 370 |
| 1531766 | | | | | |
| AD- | C3 | usgsgacuAfuGfUfGfuacaagaccuL96 | 203 | asGfsgucUfuGfUfacacAfuAfguccascsu | 371 |
| 572577 | | | | | |
| AD- | Marc1 | usgsgaggAfgAfAfGfaaaagugauuL96 | 204 | asAfsucaCfuUfUfucuuCfuCfcuccascsa | 372 |
| 1531697 | | | | | |
| AD-67561 | PNPLA3 | usgsgauaCfaUfGfAfgcaagauuuaL96 | 205 | usAfsaauCfuUfGfcucaUfgUfauccascsc | 373 |
| AD-67586 | PNPLA3 | usgsggagAfgAfUfAfugccuucgaaL96 | 206 | usUfscgaAfgGfCfauauCfuCfucccasgsc | 374 |
| AD- | C3 | usgsggcaAfcUfCfCfaacaauuacuL96 | 207 | asGfsuaaUfuGfUfuggaGfuUfgcccascsg | 375 |
| 569763 | | | | | |
| AD- | SCN9A | usgsggucAfuUfCfUfucacuuugaaL96 | 208 | usUfscaaAfgUfGfaagaAfuGfacccasasa | 376 |
| 1531726 | | | | | |
| AD- | SCN9A | usgsgucuUfuAfCfUfggaaucuuuuL96 | 209 | asAfsaagAfuUfCfcaguAfaAfgaccasasa | 377 |
| 1531742 | | | | | |
| AD-67573 | PNPLA3 | usgsgugaCfaUfGfGfcuuccagauaL96 | 210 | usAfsucuGfgAfAfgccaUfgUfcaccasgsu | 378 |
| AD- | SCN9A | usgsguguCfaUfCfAfuagauaauuuL96 | 211 | asAfsauuAfuCfUfaugaUfgAfcaccasasu | 379 |
| 1531761 | | | | | |
| AD- | Marc1 | usgsguguCfuCfAfAfugcuucaauaL96 | 212 | usAfsuugAfaGfCfauugAfgAfcaccasgsa | 380 |
| 1531712 | | | | | |
| AD- | C3 | usgsuacaAfgAfCfCfcgacuggucaL96 | 213 | usGfsaccAfgUfCfggguCfuUfguacascsa | 381 |
| 1531669 | | | | | |
| AD- | SCN9A | usgsuaggAfgAfAfUfucacuuuucaL96 | 214 | usGfsaaaAfgUfGfaauuCfuCfcuacascsa | 382 |
| 1531745 | | | | | |
| AD- | SCN9A | usgsuaggAfgAfAfUfucacuuuucuL96 | 215 | asGfsaaaAfgUfGfaauuCfuCfcuacascsa | 383 |
| 1531724 | | | | | |
| AD- | SCN9A | usgsucgaGfuAfCfAfcuuuuacugaL96 | 216 | usCfsaguAfaAfAfguguAfcUfcgacasusu | 384 |
| 1531731 | | | | | |
| AD- | C3 | usgsuucgUfgCfUfGfaauaagaaguL96 | 217 | asCfsuucUfuAfUfucagCfaCfgaacascsg | 385 |
| 570132 | | | | | |
| AD- | SCN9A | usgsuucuGfuCfUfGfaguguguuuaL96 | 218 | usAfsaacAfcAfCfucagAfcAfgaacascsa | 386 |
| 1531769 | | | | | |
| AD- | Marc1 | usgsuuuaAfaAfCfCfcaauaucuauL96 | 219 | asUfsagaUfaUfUfggguUfuUfaaacasasc | 387 |
| 1531694 | | | | | |
| AD- | Marc1 | ususaaaaCfuGfUfGfaauaaauggaL96 | 220 | usCfscauUfuAfUfucacAfgUfuuuaasasa | 388 |
| 1531677 | | | | | |
| AD- | SCN9A | ususaccuAfuCfUfCfugcuucaaguL96 | 221 | asCfsuugAfaGfCfagagAfuAfgguaascsc | 389 |
| 1531727 | | | | | |
| AD-67575 | PNPLA3 | ususaccuGfuUfGfAfauuuuguauuL96 | 222 | asAfsuacAfaAfAfuucaAfcAfgguaascsa | 390 |
| AD-75274 | PNPLA3 | ususauguAfaUfGfCfugcccuguaaL96 | 223 | usUfsacaGfgGfCfagcaUfuAfcauaasgsa | 391 |
| AD- | SCN9A | ususccucAfaGfGfAfaaaagauaaaL96 | 224 | usUfsuauCfuUfUfuuccUfuGfaggaasasu | 392 |
| 1531768 | | | | | |
| AD- | C3 | ususcgugCfuGfAfAfuaagaagaauL96 | 225 | asUfsucuUfcUfUfauucAfgCfacgaascsa | 393 |
| 570134 | | | | | |
| AD- | Marc1 | ususgccaUfuUfUfGfuccuuugauuL96 | 226 | asAfsucaAfaGfGfacaaAfaUfggcaasusa | 394 |
| 1531707 | | | | | |
| AD-75272 | PNPLA3 | ususgcuaCfcCfAfUfuaggauaauaL96 | 227 | usAfsuuaUfcCfUfaaugGfgUfagcaasgsu | 395 |
| AD-67567 | PNPLA3 | ususgguuUfuAfUfGfaaaagcuagaL96 | 228 | usCfsuagCfuUfUfucauAfaAfaccaascsu | 396 |
| AD- | Marc1 | ususguaaUfuUfCfAfggaugcgauaL96 | 229 | usAfsucgCfaUfCfcugaAfaUfuacaasusa | 397 |
| 1531714 | | | | | |
| AD- | Marc1 | ususguucCfaGfAfUfgcauuuuaauL96 | 230 | asUfsuaaAfaUfGfcaucUfgGfaacaasgsc | 398 |
| 1531720 | | | | | |
| AD- | SCN9A | ususuaguAfcAfCfUfccuuauucauL96 | 231 | asUfsgaaUfaAfGfgaguGfuAfcuaaasasu | 399 |
| 1531746 | | | | | |
| AD- | SCN9A | ususuaucAfuCfUfUfugggucauuaL96 | 232 | usAfsaugAfcCfCfaaagAfuGfauaaasgsa | 400 |
| 1531755 | | | | | |
| AD- | Marc1 | ususuccaUfaGfAfUfcuggaucugaL96 | 233 | usCfsagaUfcCfAfgaucUfaUfggaaasasu | 401 |
| 1531675 | | | | | |
| AD- | SCN9A | ususuguaGfaUfCfUfugcaauuacaL96 | 234 | usGfsuaaUfuGfCfaagaUfcUfacaaasasg | 402 |
| 1531767 | | | | | |
| AD- | PNPLA3 | ususuuagAfaCfAfCfcuuuuucacuL96 | 235 | asGfsugaAfaAfAfggugUfuCfuaaaasusu | 403 |
| 520053 | | | | | |
| AD- | PNPLA3 | ususuuucAfcCfUfAfacuaaaauaaL96 | 236 | usUfsauuUfuAfGfuuagGfuGfaaaaasgsg | 404 |
| 1010734 | | | | | |

TABLE 3
More additional exemplary sequences of parent dsRNA molecules.

| | | | SEQ | | SEQ |
|---|---|---|---|---|---|
| Duplex | | | ID | | ID |
| ID | Target | Sense sequence (5′→3′) | NO: | Antisense sequence (5′→3′) | NO: |
| AD- | AGT | asgsccugAfgGfGfCfcaccauccuuL96 | 405 | asAfsggaUfgGfUfggccCfuCfaggcuscsa | 752 |
| 1632799 | | | | | |
| AD- | AGT | cscsugagGfgCfCfAfccauccucuuL96 | 406 | asAfsgagGfaUfGfguggCfcCfucaggscsu | 753 |
| 1632801 | | | | | |
| AD- | AGT | asgsggccAfcCfAfUfccucugccuuL96 | 407 | asAfsggcAfgAfGfgaugGfuGfgcccuscsa | 754 |
| 1632805 | | | | | |
| AD- | AGT | gsusgaccGfgGfUfGfuacauacacuL96 | 408 | asGfsuguAfuGfUfacacCfcGfgucacscsu | 755 |
| 1632838 | | | | | |
| AD- | AGT | cscsggguGfuAfCfAfuacaccccuuL96 | 409 | asAfsgggGfuGfUfauguAfcAfcccggsusc | 756 |
| 1684490 | | | | | |
| AD- | AGT | csgsggugUfaCfAfUfacaccccuuuL96 | 410 | asAfsaggGfgUfGfuaugUfaCfacccgsgsu | 757 |
| 1684492 | | | | | |
| AD- | AGT | gsgsguguAfcAfUfAfcaccccuucuL96 | 411 | asGfsaagGfgGfUfguauGfuAfcacccsgsg | 758 |
| 1684494 | | | | | |
| AD- | AGT | gsgsuguaCfaUfAfCfaccccuuccuL96 | 412 | asGfsgaaGfgGfGfuguaUfgUfacaccscsg | 759 |
| 1684496 | | | | | |
| AD- | AGT | gsusguacAfuAfCfAfccccuuccauL96 | 413 | asUfsggaAfgGfGfguguAfuGfuacacscsc | 760 |
| 1684498 | | | | | |
| AD- | AGT | usgsuacaUfaCfAfCfcccuuccacuL96 | 414 | asGfsuggAfaGfGfggugUfaUfguacascsc | 761 |
| 1684500 | | | | | |
| AD- | AGT | gsusacauAfcAfCfCfccuuccaccuL96 | 415 | asGfsgugGfaAfGfggguGfuAfuguacsasc | 762 |
| 1684502 | | | | | |
| AD- | AGT | usascauaCfaCfCfCfcuuccaccuuL96 | 416 | asAfsgguGfgAfAfggggUfgUfauguascsa | 763 |
| 1684504 | | | | | |
| AD- | AGT | ascsauacAfcCfCfCfuuccaccucuL96 | 417 | asGfsaggUfgGfAfagggGfuGfuaugusasc | 764 |
| 1684506 | | | | | |
| AD- | AGT | csasuacaCfcCfCfUfuccaccucguL96 | 418 | asCfsgagGfuGfGfaaggGfgUfguaugsusa | 765 |
| 1684508 | | | | | |
| AD- | AGT | asusacacCfcCfUfUfccaccucguuL96 | 419 | asAfscgaGfgUfGfgaagGfgGfuguausgsu | 766 |
| 1684510 | | | | | |
| AD- | AGT | cscscuucCfaCfCfUfcgucauccauL96 | 420 | asUfsggaUfgAfCfgaggUfgGfaagggsgsu | 767 |
| 1684512 | | | | | |
| AD- | AGT | cscsuuccAfcCfUfCfgucauccacuL96 | 421 | asGfsuggAfuGfAfcgagGfuGfgaaggsgsg | 768 |
| 1684514 | | | | | |
| AD- | AGT | csusuccaCfcUfCfGfucauccacauL96 | 422 | asUfsgugGfaUfGfacgaGfgUfggaagsgsg | 769 |
| 1632840 | | | | | |
| AD- | AGT | ususccacCfuCfGfUfcauccacaauL96 | 423 | asUfsuguGfgAfUfgacgAfgGfuggaasgsg | 770 |
| 1632841 | | | | | |
| AD- | AGT | uscscaccUfcGfUfCfauccacaauuL96 | 424 | asAfsuugUfgGfAfugacGfaGfguggasasg | 771 |
| 1632842 | | | | | |
| AD- | AGT | cscsaccuCfgUfCfAfuccacaauguL96 | 425 | asCfsauuGfuGfGfaugaCfgAfgguggsasa | 772 |
| 1632843 | | | | | |
| AD- | AGT | csasccucGfuCfAfUfccacaaugauL96 | 426 | asUfscauUfgUfGfgaugAfcGfaggugsgsa | 773 |
| 1632844 | | | | | |
| AD- | AGT | cscsucguCfaUfCfCfacaaugagauL96 | 427 | asUfscucAfuUfGfuggaUfgAfcgaggsusg | 774 |
| 1632846 | | | | | |
| AD- | AGT | csuscgucAfuCfCfAfcaaugagaguL96 | 428 | asCfsucuCfaUfUfguggAfuGfacgagsgsu | 775 |
| 1632847 | | | | | |
| AD- | AGT | uscsgucaUfcCfAfCfaaugagaguuL96 | 429 | asAfscucUfcAfUfugugGfaUfgacgasgsg | 776 |
| 1632848 | | | | | |
| AD- | AGT | csgsucauCfcAfCfAfaugagaguauL96 | 430 | asUfsacuCfuCfAfuuguGfgAfugacgsasg | 777 |
| 1632849 | | | | | |
| AD- | AGT | gsuscaucCfaCfAfAfugagaguacuL96 | 431 | asGfsuacUfcUfCfauugUfgGfaugacsgsa | 778 |
| 1632836 | | | | | |
| AD- | AGT | uscsauccAfcAfAfUfgagaguaccuL96 | 432 | asGfsguaCfuCfUfcauuGfuGfgaugascsg | 779 |
| 1632850 | | | | | |
| AD- | AGT | csasuccaCfaAfUfGfagaguaccuuL96 | 433 | asAfsgguAfcUfCfucauUfgUfggaugsasc | 780 |
| 1632851 | | | | | |
| AD- | AGT | asusccacAfaUfGfAfgaguaccuguL96 | 434 | asCfsaggUfaCfUfcucaUfuGfuggausgsa | 781 |
| 1632852 | | | | | |
| AD- | AGT | uscscacaAfuGfAfGfaguaccuguuL96 | 435 | asAfscagGfuAfCfucucAfuUfguggasusg | 782 |
| 1632853 | | | | | |
| AD- | AGT | cscsacaaUfgAfGfAfguaccuguguL96 | 436 | asCfsacaGfgUfAfcucuCfaUfuguggsasu | 783 |
| 1632854 | | | | | |
| AD- | AGT | csascaauGfaGfAfGfuaccugugauL96 | 437 | asUfscacAfgGfUfacucUfcAfuugugsgsa | 784 |
| 1632855 | | | | | |
| AD- | AGT | ascsaaugAfgAfGfUfaccugugaguL96 | 438 | asCfsucaCfaGfGfuacuCfuCfauugusgsg | 785 |
| 1632856 | | | | | |
| AD- | AGT | csasaugaGfaGfUfAfccugugagcuL96 | 439 | asGfscucAfcAfGfguacUfcUfcauugsusg | 786 |
| 1632857 | | | | | |
| AD- | AGT | asasugagAfgUfAfCfcugugagcauL96 | 440 | asUfsgcuCfaCfAfgguaCfuCfucauusgsu | 787 |
| 1632858 | | | | | |
| AD- | AGT | asusgagaGfuAfCfCfugugagcaguL96 | 441 | asCfsugcUfcAfCfagguAfcUfcucaususg | 788 |
| 1632859 | | | | | |
| AD- | AGT | usgsagagUfaCfCfUfgugagcagcuL96 | 442 | asGfscugCfuCfAfcaggUfaCfucucasusu | 789 |
| 1632860 | | | | | |
| AD- | AGT | gsasgaguAfcCfUfGfugagcagcuuL96 | 443 | asAfsgcuGfcUfCfacagGfuAfcucucsasu | 790 |
| 1632861 | | | | | |
| AD- | AGT | asgsaguaCfcUfGfUfgagcagcuguL96 | 444 | asCfsagcUfgCfUfcacaGfgUfacucuscsa | 791 |
| 1632862 | | | | | |
| AD- | AGT | gsasguacCfuGfUfGfagcagcugguL96 | 445 | asCfscagCfuGfCfucacAfgGfuacucsusc | 792 |
| 1632863 | | | | | |
| AD- | AGT | asgsuaccUfgUfGfAfgcagcuggcuL96 | 446 | asGfsccaGfcUfGfcucaCfaGfguacuscsu | 793 |
| 1632864 | | | | | |
| AD- | AGT | gsusaccuGfuGfAfGfcagcuggcauL96 | 447 | asUfsgccAfgCfUfgcucAfcAfgguacsusc | 794 |
| 1632865 | | | | | |
| AD- | AGT | usasccugUfgAfGfCfagcuggcaauL96 | 448 | asUfsugcCfaGfCfugcuCfaCfagguascsu | 795 |
| 1632866 | | | | | |
| AD- | AGT | asasugguCfgGfGfAfugcuggccauL96 | 449 | asUfsggcCfaGfCfauccCfgAfccauusgsc | 796 |
| 1632991 | | | | | |
| AD- | AGT | asusggucGfgGfAfUfgcuggccaauL96 | 450 | asUfsuggCfcAfGfcaucCfcGfaccaususg | 797 |
| 1632992 | | | | | |
| AD- | AGT | usgsgucgGfgAfUfGfcuggccaacuL96 | 451 | asGfsuugGfcCfAfgcauCfcCfgaccasusu | 798 |
| 1632993 | | | | | |
| AD- | AGT | gsgsucggGfaUfGfCfuggccaacuuL96 | 452 | asAfsguuGfgCfCfagcaUfcCfcgaccsasu | 799 |
| 1632994 | | | | | |
| AD- | AGT | gsuscgggAfuGfCfUfggccaacuuuL96 | 453 | asAfsaguUfgGfCfcagcAfuCfccgacscsa | 800 |
| 1632995 | | | | | |
| AD- | AGT | uscsgggaUfgCfUfGfgccaacuucuL96 | 454 | asGfsaagUfuGfGfccagCfaUfcccgascsc | 801 |
| 1632996 | | | | | |
| AD- | AGT | csgsggauGfcUfGfGfccaacuucuuL96 | 455 | asAfsgaaGfuUfGfgccaGfcAfucccgsasc | 802 |
| 1632997 | | | | | |
| AD- | AGT | gsgsgaugCfuGfGfCfcaacuucuuuL96 | 456 | asAfsagaAfgUfUfggccAfgCfaucccsgsa | 803 |
| 1632998 | | | | | |
| AD- | AGT | gsgsaugcUfgGfCfCfaacuucuuguL96 | 457 | asCfsaagAfaGfUfuggcCfaGfcauccscsg | 804 |
| 1632999 | | | | | |
| AD- | AGT | gsasugcuGfgCfCfAfacuucuugguL96 | 458 | asCfscaaGfaAfGfuuggCfcAfgcaucscsc | 805 |
| 1633000 | | | | | |
| AD- | AGT | gscsuggcCfaAfCfUfucuugggcuuL96 | 459 | asAfsgccCfaAfGfaaguUfgGfccagcsasu | 806 |
| 1633003 | | | | | |
| AD- | AGT | csusggccAfaCfUfUfcuugggcuuuL96 | 460 | asAfsagcCfcAfAfgaagUfuGfgccagscsa | 807 |
| 1633004 | | | | | |
| AD- | AGT | gscscaacUfuCfUfUfgggcuuccguL96 | 461 | asCfsggaAfgCfCfcaagAfaGfuuggcscsa | 808 |
| 1633007 | | | | | |
| AD- | AGT | cscsaacuUfcUfUfGfggcuuccguuL96 | 462 | asAfscggAfaGfCfccaaGfaAfguuggscsc | 809 |
| 1633008 | | | | | |
| AD- | AGT | csasacuuCfuUfGfGfgcuuccguauL96 | 463 | asUfsacgGfaAfGfcccaAfgAfaguugsgsc | 810 |
| 1633009 | | | | | |
| AD- | AGT | asascuucUfuGfGfGfcuuccguauuL96 | 464 | asAfsuacGfgAfAfgcccAfaGfaaguusgsg | 811 |
| 1633010 | | | | | |
| AD- | AGT | ascsuucuUfgGfGfCfuuccguauauL96 | 465 | asUfsauaCfgGfAfagccCfaAfgaagususg | 812 |
| 1633011 | | | | | |
| AD- | AGT | csusucuuGfgGfCfUfuccguauauuL96 | 466 | asAfsuauAfcGfGfaagcCfcAfagaagsusu | 813 |
| 1633012 | | | | | |
| AD- | AGT | ususcuugGfgCfUfUfccguauauauL96 | 467 | asUfsauaUfaCfGfgaagCfcCfaagaasgsu | 814 |
| 1633013 | | | | | |
| AD- | AGT | uscsuuggGfcUfUfCfcguauauauuL96 | 468 | asAfsuauAfuAfCfggaaGfcCfcaagasasg | 815 |
| 1633014 | | | | | |
| AD- | AGT | csusugggCfuUfCfCfguauauauguL96 | 469 | asCfsauaUfaUfAfcggaAfgCfccaagsasa | 816 |
| 1633015 | | | | | |
| AD- | AGT | ususgggcUfuCfCfGfuauauaugguL96 | 470 | asCfscauAfuAfUfacggAfaGfcccaasgsa | 817 |
| 1633016 | | | | | |
| AD- | AGT | gsgsgcuuCfcGfUfAfuauauggcauL96 | 471 | asUfsgccAfuAfUfauacGfgAfagcccsasa | 818 |
| 1633018 | | | | | |
| AD- | AGT | gsgscuucCfgUfAfUfauauggcauuL96 | 472 | asAfsugcCfaUfAfuauaCfgGfaagccscsa | 819 |
| 1633019 | | | | | |
| AD- | AGT | gscsuuccGfuAfUfAfuauggcauguL96 | 473 | asCfsaugCfcAfUfauauAfcGfgaagcscsc | 820 |
| 1633020 | | | | | |
| AD- | AGT | usasuauaUfgGfCfAfugcacaguguL96 | 474 | asCfsacuGfuGfCfaugcCfaUfauauascsg | 821 |
| 1633027 | | | | | |
| AD- | AGT | asusauauGfgCfAfUfgcacagugauL96 | 475 | asUfscacUfgUfGfcaugCfcAfuauausasc | 822 |
| 1633028 | | | | | |
| AD- | AGT | usasuaugGfcAfUfGfcacagugaguL96 | 476 | asCfsucaCfuGfUfgcauGfcCfauauasusa | 823 |
| 1633029 | | | | | |
| AD- | AGT | asusauggCfaUfGfCfacagugagcuL96 | 477 | asGfscucAfcUfGfugcaUfgCfcauausasu | 824 |
| 1633030 | | | | | |
| AD- | AGT | usasuggcAfuGfCfAfcagugagcuuL96 | 478 | asAfsgcuCfaCfUfgugcAfuGfccauasusa | 825 |
| 1633031 | | | | | |
| AD-84731 | AGT | asusggcaUfgCfAfCfagugagcuauL96 | 479 | asUfsagcUfcAfCfugugCfaUfgccausasu | 826 |
| AD- | AGT | usgsgcauGfcAfCfAfgugagcuauuL96 | 480 | asAfsuagCfuCfAfcuguGfcAfugccasusa | 827 |
| 1633032 | | | | | |
| AD- | AGT | gsgscaugCfaCfAfGfugagcuauguL96 | 481 | asCfsauaGfcUfCfacugUfgCfaugccsasu | 828 |
| 1633033 | | | | | |
| AD- | AGT | gscsaugcAfcAfGfUfgagcuaugguL96 | 482 | asCfscauAfgCfUfcacuGfuGfcaugcscsa | 829 |
| 1633034 | | | | | |
| AD- | AGT | csasugcaCfaGfUfGfagcuauggguL96 | 483 | asCfsccaUfaGfCfucacUfgUfgcaugscsc | 830 |
| 1684516 | | | | | |
| AD- | AGT | asusgcacAfgUfGfAfgcuaugggguL96 | 484 | asCfscccAfuAfGfcucaCfuGfugcausgsc | 831 |
| 1684518 | | | | | |
| AD- | AGT | cscsucucCfcCfAfAfcggcugucuuL96 | 485 | asAfsgacAfgCfCfguugGfgGfagaggsasc | 832 |
| 1684520 | | | | | |
| AD- | AGT | usgsgcacCfcUfGfGfccucucucuuL96 | 486 | asAfsgagAfgAfGfgccaGfgGfugccasasa | 833 |
| 1633048 | | | | | |
| AD- | AGT | gsgscaccCfuGfGfCfcucucucuauL96 | 487 | asUfsagaGfaGfAfggccAfgGfgugccsasa | 834 |
| 1633049 | | | | | |
| AD- | AGT | gsascaggCfuAfCfAfggcaauccuuL96 | 488 | asAfsggaUfuGfCfcuguAfgCfcugucsasg | 835 |
| 1633094 | | | | | |
| AD- | AGT | ascsaggcUfaCfAfGfgcaauccuguL96 | 489 | asCfsaggAfuUfGfccugUfaGfccuguscsa | 836 |
| 1633095 | | | | | |
| AD- | AGT | ususccuuGfgAfAfGfgacaagaacuL96 | 490 | asGfsuucUfuGfUfccuuCfcAfaggaascsa | 837 |
| 1633119 | | | | | |
| AD- | AGT | cscsuuggAfaGfGfAfcaagaacuguL96 | 491 | asCfsaguUfcUfUfguccUfuCfcaaggsasa | 838 |
| 1633121 | | | | | |
| AD- | AGT | csusuggaAfgGfAfCfaagaacugcuL96 | 492 | asGfscagUfuCfUfugucCfuUfccaagsgsa | 839 |
| 1633122 | | | | | |
| AD- | AGT | csasccugAfaGfCfAfgccguuuguuL96 | 493 | asAfscaaAfcGfGfcugcUfuCfaggugscsa | 840 |
| 1633254 | | | | | |
| AD- | AGT | csusgaagCfaGfCfCfguuugugcauL96 | 494 | asUfsgcaCfaAfAfcggcUfgCfuucagsgsu | 841 |
| 1633257 | | | | | |
| AD- | AGT | ususugugCfaGfGfGfccuggcucuuL96 | 495 | asAfsgagCfcAfGfgcccUfgCfacaaascsg | 842 |
| 1633269 | | | | | |
| AD- | AGT | ususgugcAfgGfGfCfcuggcucucuL96 | 496 | asGfsagaGfcCfAfggccCfuGfcacaasasc | 843 |
| 1633270 | | | | | |
| AD- | AGT | usgsugcaGfgGfCfCfuggcucucuuL96 | 497 | asAfsgagAfgCfCfaggcCfcUfgcacasasa | 844 |
| 1633271 | | | | | |
| AD- | AGT | gsusgcagGfgCfCfUfggcucucuauL96 | 498 | asUfsagaGfaGfCfcaggCfcCfugcacsasa | 845 |
| 1633272 | | | | | |
| AD- | AGT | usgscaggGfcCfUfGfgcucucuauuL96 | 499 | asAfsuagAfgAfGfccagGfcCfcugcascsa | 846 |
| 1633273 | | | | | |
| AD- | AGT | ascsgcucUfcUfGfGfacuucacaguL96 | 500 | asCfsuguGfaAfGfuccaGfaGfagcgusgsg | 847 |
| 1633290 | | | | | |
| AD- | AGT | csgscucuCfuGfGfAfcuucacagauL96 | 501 | asUfscugUfgAfAfguccAfgAfgagcgsusg | 848 |
| 1633291 | | | | | |
| AD- | AGT | csusgagaAfgAfUfUfgacagguucuL96 | 502 | asGfsaacCfuGfUfcaauCfuUfcucagscsa | 849 |
| 1633324 | | | | | |
| AD-84739 | AGT | usgsagaaGfaUfUfGfacagguucauL96 | 503 | asUfsgaaCfcUfGfucaaUfcUfucucasgsc | 850 |
| AD- | AGT | gsasgaagAfuUfGfAfcagguucauuL96 | 504 | asAfsugaAfcCfUfgucaAfuCfuucucsasg | 851 |
| 1633325 | | | | | |
| AD- | AGT | asgsaagaUfuGfAfCfagguucauguL96 | 505 | asCfsaugAfaCfCfugucAfaUfcuucuscsa | 852 |
| 1633326 | | | | | |
| AD- | AGT | gsasagauUfgAfCfAfgguucaugcuL96 | 506 | asGfscauGfaAfCfcuguCfaAfucuucsusc | 853 |
| 1633327 | | | | | |
| AD- | AGT | asasgauuGfaCfAfGfguucaugcauL96 | 507 | asUfsgcaUfgAfAfccugUfcAfaucuuscsu | 854 |
| 1633328 | | | | | |
| AD- | AGT | asgsauugAfcAfGfGfuucaugcaguL96 | 508 | asCfsugcAfuGfAfaccuGfuCfaaucususc | 855 |
| 1633329 | | | | | |
| AD- | AGT | gsasuugaCfaGfGfUfucaugcagguL96 | 509 | asCfscugCfaUfGfaaccUfgUfcaaucsusu | 856 |
| 1633330 | | | | | |
| AD- | AGT | asusugacAfgGfUfUfcaugcaggcuL96 | 510 | asGfsccuGfcAfUfgaacCfuGfucaauscsu | 857 |
| 1633331 | | | | | |
| AD- | AGT | ususgacaGfgUfUfCfaugcaggcuuL96 | 511 | asAfsgccUfgCfAfugaaCfcUfgucaasusc | 858 |
| 1633332 | | | | | |
| AD- | AGT | usgsacagGfuUfCfAfugcaggcuguL96 | 512 | asCfsagcCfuGfCfaugaAfcCfugucasasu | 859 |
| 1633333 | | | | | |
| AD- | AGT | gsascaggUfuCfAfUfgcaggcuguuL96 | 513 | asAfscagCfcUfGfcaugAfaCfcugucsasa | 860 |
| 1633334 | | | | | |
| AD- | AGT | ascsagguUfcAfUfGfcaggcuguguL96 | 514 | asCfsacaGfcCfUfgcauGfaAfccuguscsa | 861 |
| 1633335 | | | | | |
| AD- | AGT | asusgcagGfcUfGfUfgacaggauguL96 | 515 | asCfsaucCfuGfUfcacaGfcCfugcausgsa | 862 |
| 1633343 | | | | | |
| AD- | AGT | gscsaggcUfgUfGfAfcaggauggauL96 | 516 | asUfsccaUfcCfUfgucaCfaGfccugcsasu | 863 |
| 1633345 | | | | | |
| AD- | AGT | csasggcuGfuGfAfCfaggauggaauL96 | 517 | asUfsuccAfuCfCfugucAfcAfgccugscsa | 864 |
| 1633346 | | | | | |
| AD- | AGT | gscsuuucAfaCfAfCfcuacguccauL96 | 518 | asUfsggaCfgUfAfggugUfuGfaaagcscsa | 865 |
| 1633409 | | | | | |
| AD- | AGT | gsasguucUfgGfGfUfggacaacaguL96 | 519 | asCfsuguUfgUfCfcaccCfaGfaacucscsu | 866 |
| 1633453 | | | | | |
| AD- | AGT | gsgsacaaCfaGfCfAfccucaguguuL96 | 520 | asAfscacUfgAfGfgugcUfgUfuguccsasc | 867 |
| 1633464 | | | | | |
| AD- | AGT | gsascaacAfgCfAfCfcucagugucuL96 | 521 | asGfsacaCfuGfAfggugCfuGfuugucscsa | 868 |
| 1633465 | | | | | |
| AD- | AGT | ascsaacaGfcAfCfCfucagugucuuL96 | 522 | asAfsgacAfcUfGfagguGfcUfguuguscsc | 869 |
| 1633466 | | | | | |
| AD- | AGT | csasacagCfaCfCfUfcagugucuguL96 | 523 | asCfsagaCfaCfUfgaggUfgCfuguugsusc | 870 |
| 1633467 | | | | | |
| AD- | AGT | asascagcAfcCfUfCfagugucuguuL96 | 524 | asAfscagAfcAfCfugagGfuGfcuguusgsu | 871 |
| 1633468 | | | | | |
| AD- | AGT | asusgccuCfuGfAfCfcuggacaaguL96 | 525 | asCfsuugUfcCfAfggucAfgAfggcausasg | 872 |
| 1633604 | | | | | |
| AD- | AGT | asasggugGfaGfGfGfucucacuuuuL96 | 526 | asAfsaagUfgAfGfacccUfcCfaccuusgsu | 873 |
| 1633621 | | | | | |
| AD- | AGT | asgsguggAfgGfGfUfcucacuuucuL96 | 527 | asGfsaaaGfuGfAfgaccCfuCfcaccususg | 874 |
| 1633622 | | | | | |
| AD- | AGT | gsgsuggaGfgGfUfCfucacuuuccuL96 | 528 | asGfsgaaAfgUfGfagacCfcUfccaccsusu | 875 |
| 1633623 | | | | | |
| AD- | AGT | gsasggguCfuCfAfCfuuuccagcauL96 | 529 | asUfsgcuGfgAfAfagugAfgAfcccucscsa | 876 |
| 1633627 | | | | | |
| AD- | AGT | asgsggucUfcAfCfUfuuccagcaauL96 | 530 | asUfsugcUfgGfAfaaguGfaGfacccuscsc | 877 |
| 1633628 | | | | | |
| AD- | AGT | gsgsucucAfcUfUfUfccagcaaaauL96 | 531 | asUfsuuuGfcUfGfgaaaGfuGfagaccscsu | 878 |
| 1633630 | | | | | |
| AD- | AGT | gsuscucaCfuUfUfCfcagcaaaacuL96 | 532 | asGfsuuuUfgCfUfggaaAfgUfgagacscsc | 879 |
| 1633631 | | | | | |
| AD- | AGT | uscsucacUfuUfCfCfagcaaaacuuL96 | 533 | asAfsguuUfuGfCfuggaAfaGfugagascsc | 880 |
| 1633632 | | | | | |
| AD- | AGT | csuscacuUfuCfCfAfgcaaaacucuL96 | 534 | asGfsaguUfuUfGfcuggAfaAfgugagsasc | 881 |
| 1633633 | | | | | |
| AD- | AGT | uscsacuuUfcCfAfGfcaaaacuccuL96 | 535 | asGfsgagUfuUfUfgcugGfaAfagugasgsa | 882 |
| 1633634 | | | | | |
| AD- | AGT | csascuuuCfcAfGfCfaaaacucccuL96 | 536 | asGfsggaGfuUfUfugcuGfgAfaagugsasg | 883 |
| 1633635 | | | | | |
| AD- | AGT | ascsuuucCfaGfCfAfaaacucccuuL96 | 537 | asAfsgggAfgUfUfuugcUfgGfaaagusgsa | 884 |
| 1633636 | | | | | |
| AD- | AGT | csusuuccAfgCfAfAfaacucccucuL96 | 538 | asGfsaggGfaGfUfuuugCfuGfgaaagsusg | 885 |
| 1633637 | | | | | |
| AD- | AGT | ususuccaGfcAfAfAfacucccucauL96 | 539 | asUfsgagGfgAfGfuuuuGfcUfggaaasgsu | 886 |
| 1633638 | | | | | |
| AD- | AGT | ususccagCfaAfAfAfcucccucaauL96 | 540 | asUfsugaGfgGfAfguuuUfgCfuggaasasg | 887 |
| 1633639 | | | | | |
| AD- | AGT | uscscagcAfaAfAfCfucccucaacuL96 | 541 | asGfsuugAfgGfGfaguuUfuGfcuggasasa | 888 |
| 1633640 | | | | | |
| AD- | AGT | cscsagcaAfaAfCfUfcccucaacuuL96 | 542 | asAfsguuGfaGfGfgaguUfuUfgcuggsasa | 889 |
| 1633641 | | | | | |
| AD- | AGT | csasgcaaAfaCfUfCfccucaacuguL96 | 543 | asCfsaguUfgAfGfggagUfuUfugcugsgsa | 890 |
| 1633642 | | | | | |
| AD- | AGT | asgscaaaAfcUfCfCfcucaacugguL96 | 544 | asCfscagUfuGfAfgggaGfuUfuugcusgsg | 891 |
| 1633643 | | | | | |
| AD- | AGT | gscsaaaaCfuCfCfCfucaacuggauL96 | 545 | asUfsccaGfuUfGfagggAfgUfuuugcsusg | 892 |
| 1633644 | | | | | |
| AD- | AGT | csasaaacUfcCfCfUfcaacuggauuL96 | 546 | asAfsuccAfgUfUfgaggGfaGfuuuugscsu | 893 |
| 1633645 | | | | | |
| AD- | AGT | asasaacuCfcCfUfCfaacuggauguL96 | 547 | asCfsaucCfaGfUfugagGfgAfguuuusgsc | 894 |
| 1633646 | | | | | |
| AD- | AGT | asasacucCfcUfCfAfacuggaugauL96 | 548 | asUfscauCfcAfGfuugaGfgGfaguuususg | 895 |
| 1633647 | | | | | |
| AD- | AGT | asascuccCfuCfAfAfcuggaugaauL96 | 549 | asUfsucaUfcCfAfguugAfgGfgaguususu | 896 |
| 1633648 | | | | | |
| AD- | AGT | ascsucccUfcAfAfCfuggaugaaguL96 | 550 | asCfsuucAfuCfCfaguuGfaGfggagususu | 897 |
| 1633649 | | | | | |
| AD- | AGT | csuscccuCfaAfCfUfggaugaagauL96 | 551 | asUfscuuCfaUfCfcaguUfgAfgggagsusu | 898 |
| 1633650 | | | | | |
| AD- | AGT | uscsccucAfaCfUfGfgaugaagaauL96 | 552 | asUfsucuUfcAfUfccagUfuGfagggasgsu | 899 |
| 1633651 | | | | | |
| AD- | AGT | cscscucaAfcUfGfGfaugaagaaauL96 | 553 | asUfsuucUfuCfAfuccaGfuUfgagggsasg | 900 |
| 1633652 | | | | | |
| AD-84707 | AGT | cscsucaaCfuGfGfAfugaagaaacuL96 | 7 | asGfsuuuCfuUfCfauccAfgUfugaggsgsa | 901 |
| AD- | AGT | csuscaacUfgGfAfUfgaagaaacuuL96 | 554 | asAfsguuUfcUfUfcaucCfaGfuugagsgsg | 902 |
| 1633653 | | | | | |
| AD- | AGT | asgsgaucUfuAfUfGfaccugcagguL96 | 555 | asCfscugCfaGfGfucauAfaGfauccususg | 903 |
| 1633678 | | | | | |
| AD- | AGT | csusuaugAfcCfUfGfcaggaccuguL96 | 556 | asCfsaggUfcCfUfgcagGfuCfauaagsasu | 904 |
| 1633683 | | | | | |
| AD- | AGT | csgsagcuGfaAfCfCfugcaaaaauuL96 | 557 | asAfsuuuUfuGfCfagguUfcAfgcucgsgsu | 905 |
| 1633732 | | | | | |
| AD- | AGT | gsasgcugAfaCfCfUfgcaaaaauuuL96 | 558 | asAfsauuUfuUfGfcaggUfuCfagcucsgsg | 906 |
| 1633733 | | | | | |
| AD- | AGT | asgscugaAfcCfUfGfcaaaaauuguL96 | 559 | asCfsaauUfuUfUfgcagGfuUfcagcuscsg | 907 |
| 1633734 | | | | | |
| AD- | AGT | gscsugaaCfcUfGfCfaaaaauugauL96 | 560 | asUfscaaUfuUfUfugcaGfgUfucagcsusc | 908 |
| 1633735 | | | | | |
| AD- | AGT | csusgaacCfuGfCfAfaaaauugaguL96 | 561 | asCfsucaAfuUfUfuugcAfgGfuucagscsu | 909 |
| 1633736 | | | | | |
| AD- | AGT | usgsaaccUfgCfAfAfaaauugagcuL96 | 562 | asGfscucAfaUfUfuuugCfaGfguucasgsc | 910 |
| 1633737 | | | | | |
| AD- | AGT | gsasaccuGfcAfAfAfaauugagcauL96 | 563 | asUfsgcuCfaAfUfuuuuGfcAfgguucsasg | 911 |
| 1633738 | | | | | |
| AD- | AGT | asasccugCfaAfAfAfauugagcaauL96 | 564 | asUfsugcUfcAfAfuuuuUfgCfagguuscsa | 912 |
| 1633739 | | | | | |
| AD- | AGT | ascscugcAfaAfAfAfuugagcaauuL96 | 565 | asAfsuugCfuCfAfauuuUfuGfcaggususc | 913 |
| 1633740 | | | | | |
| AD- | AGT | cscsugcaAfaAfAfUfugagcaauguL96 | 566 | asCfsauuGfcUfCfaauuUfuUfgcaggsusu | 914 |
| 1633741 | | | | | |
| AD- | AGT | csusgcaaAfaAfUfUfgagcaaugauL96 | 567 | asUfscauUfgCfUfcaauUfuUfugcagsgsu | 915 |
| 1633742 | | | | | |
| AD- | AGT | usgscaaaAfaUfUfGfagcaaugacuL96 | 568 | asGfsucaUfuGfCfucaaUfuUfuugcasgsg | 916 |
| 1633743 | | | | | |
| AD- | AGT | gsasggugCfuGfAfAfcagcauuuuuL96 | 569 | asAfsaaaUfgCfUfguucAfgCfaccucscsc | 917 |
| 1633759 | | | | | |
| AD- | AGT | asgsgugcUfgAfAfCfagcauuuuuuL96 | 570 | asAfsaaaAfuGfCfuguuCfaGfcaccuscsc | 918 |
| 1684522 | | | | | |
| AD- | AGT | gsgsugcuGfaAfCfAfgcauuuuuuuL96 | 571 | asAfsaaaAfaUfGfcuguUfcAfgcaccsusc | 919 |
| 1684523 | | | | | |
| AD- | AGT | gsusgcugAfaCfAfGfcauuuuuuuuL96 | 572 | asAfsaaaAfaAfUfgcugUfuCfagcacscsu | 920 |
| 1684524 | | | | | |
| AD- | AGT | usgscugaAfcAfGfCfauuuuuuuuuL96 | 573 | asAfsaaaAfaAfAfugcuGfuUfcagcascsc | 921 |
| 1684525 | | | | | |
| AD- | AGT | gscsugaaCfaGfCfAfuuuuuuuuguL96 | 574 | asCfsaaaAfaAfAfaugcUfgUfucagcsasc | 922 |
| 1684526 | | | | | |
| AD- | AGT | csusgaacAfgCfAfUfuuuuuuugauL96 | 575 | asUfscaaAfaAfAfaaugCfuGfuucagscsa | 923 |
| 1684527 | | | | | |
| AD- | AGT | usgsagagAfgAfGfCfccacagaguuL96 | 576 | asAfscucUfgUfGfggcuCfuCfucucasusc | 924 |
| 1633777 | | | | | |
| AD- | AGT | asgsagagAfgCfCfCfacagagucuuL96 | 577 | asAfsgacUfcUfGfugggCfuCfucucuscsa | 925 |
| 1633779 | | | | | |
| AD- | AGT | gsasgagaGfcCfCfAfcagagucuauL96 | 578 | asUfsagaCfuCfUfguggGfcUfcucucsusc | 926 |
| 1633780 | | | | | |
| AD- | AGT | gsasaccgCfcCfAfUfuccuguuuguL96 | 579 | asCfsaaaCfaGfGfaaugGfgCfgguucsasg | 927 |
| 1633840 | | | | | |
| AD- | AGT | asasccgcCfcAfUfUfccuguuugcuL96 | 580 | asGfscaaAfcAfGfgaauGfgGfcgguuscsa | 928 |
| 1633841 | | | | | |
| AD- | AGT | ascscgccCfaUfUfCfcuguuugcuuL96 | 581 | asAfsgcaAfaCfAfggaaUfgGfgcggususc | 929 |
| 1633842 | | | | | |
| AD- | AGT | cscsgcccAfuUfCfCfuguuugcuguL96 | 582 | asCfsagcAfaAfCfaggaAfuGfggcggsusu | 930 |
| 1633843 | | | | | |
| AD- | AGT | csgscccaUfuCfCfUfguuugcuguuL96 | 583 | asAfscagCfaAfAfcaggAfaUfgggcgsgsu | 931 |
| 1633844 | | | | | |
| AD- | AGT | gscsccauUfcCfUfGfuuugcuguguL96 | 584 | asCfsacaGfcAfAfacagGfaAfugggcsgsg | 932 |
| 1633845 | | | | | |
| AD- | AGT | cscscauuCfcUfGfUfuugcuguguuL96 | 585 | asAfscacAfgCfAfaacaGfgAfaugggscsg | 933 |
| 1633846 | | | | | |
| AD-84712 | AGT | cscsauucCfuGfUfUfugcuguguauL96 | 586 | asUfsacaCfaGfCfaaacAfgGfaauggsgsc | 934 |
| AD- | AGT | csasuuccUfgUfUfUfgcuguguauuL96 | 587 | asAfsuacAfcAfGfcaaaCfaGfgaaugsgsg | 935 |
| 1633847 | | | | | |
| AD- | AGT | asusuccuGfuUfUfGfcuguguauguL96 | 588 | asCfsauaCfaCfAfgcaaAfcAfggaausgsg | 936 |
| 1633848 | | | | | |
| AD- | AGT | ususccugUfuUfGfCfuguguaugauL96 | 589 | asUfscauAfcAfCfagcaAfaCfaggaasusg | 937 |
| 1633849 | | | | | |
| AD- | AGT | uscscuguUfuGfCfUfguguaugauuL96 | 590 | asAfsucaUfaCfAfcagcAfaAfcaggasasu | 938 |
| 1633850 | | | | | |
| AD- | AGT | cscsuguuUfgCfUfGfuguaugaucuL96 | 591 | asGfsaucAfuAfCfacagCfaAfacaggsasa | 939 |
| 1633851 | | | | | |
| AD- | AGT | csusguuuGfcUfGfUfguaugaucauL96 | 592 | asUfsgauCfaUfAfcacaGfcAfaacagsgsa | 940 |
| 1633852 | | | | | |
| AD- | AGT | usgsuuugCfuGfUfGfuaugaucaauL96 | 593 | asUfsugaUfcAfUfacacAfgCfaaacasgsg | 941 |
| 1633853 | | | | | |
| AD- | AGT | gsusuugcUfgUfGfUfaugaucaaauL96 | 594 | asUfsuugAfuCfAfuacaCfaGfcaaacsasg | 942 |
| 1633854 | | | | | |
| AD- | AGT | ususugcuGfuGfUfAfugaucaaaguL96 | 595 | asCfsuuuGfaUfCfauacAfcAfgcaaascsa | 943 |
| 1633855 | | | | | |
| AD- | AGT | cscsccagUfcUfCfCfcaccuuuucuL96 | 596 | asGfsaaaAfgGfUfgggaGfaCfuggggsgsu | 944 |
| 1684528 | | | | | |
| AD- | AGT | cscscaguCfuCfCfCfaccuuuucuuL96 | 597 | asAfsgaaAfaGfGfugggAfgAfcugggsgsg | 945 |
| 1684530 | | | | | |
| AD- | AGT | cscsagucUfcCfCfAfccuuuucuuuL96 | 598 | asAfsagaAfaAfGfguggGfaGfacuggsgsg | 946 |
| 1684532 | | | | | |
| AD- | AGT | asgsucucCfcAfCfCfuuuucuucuuL96 | 599 | asAfsgaaGfaAfAfagguGfgGfagacusgsg | 947 |
| 1684534 | | | | | |
| AD- | AGT | gsuscuccCfaCfCfUfuuucuucuauL96 | 600 | asUfsagaAfgAfAfaaggUfgGfgagacsusg | 948 |
| 1633946 | | | | | |
| AD-67328 | AGT | uscsucccAfcCfUfUfuucuucuaauL96 | 5 | asUfsuagAfaGfAfaaagGfuGfggagascsu | 949 |
| AD- | AGT | csuscccaCfcUfUfUfucuucuaauuL96 | 601 | asAfsuuaGfaAfGfaaaaGfgUfgggagsasc | 950 |
| 1633947 | | | | | |
| AD- | AGT | uscsccacCfuUfUfUfcuucuaauguL96 | 602 | asCfsauuAfgAfAfgaaaAfgGfugggasgsa | 951 |
| 1633948 | | | | | |
| AD- | AGT | cscscaccUfuUfUfCfuucuaaugauL96 | 603 | asUfscauUfaGfAfagaaAfaGfgugggsasg | 952 |
| 1633949 | | | | | |
| AD-84700 | AGT | cscsaccuUfuUfCfUfucuaaugaguL96 | 604 | asCfsucaUfuAfGfaagaAfaAfgguggsgsa | 953 |
| AD- | AGT | csasccuuUfuCfUfUfcuaaugaguuL96 | 605 | asAfscucAfuUfAfgaagAfaAfaggugsgsg | 954 |
| 1633950 | | | | | |
| AD- | AGT | ascscuuuUfcUfUfCfuaaugagucuL96 | 606 | asGfsacuCfaUfUfagaaGfaAfaaggusgsg | 955 |
| 1633951 | | | | | |
| AD- | AGT | cscsguuuCfuCfCfUfuggucuaaguL96 | 607 | asCfsuuaGfaCfCfaaggAfgAfaacggscsu | 956 |
| 1633991 | | | | | |
| AD- | AGT | csgsuuucUfcCfUfUfggucuaaguuL96 | 608 | asAfscuuAfgAfCfcaagGfaGfaaacgsgsc | 957 |
| 1633992 | | | | | |
| AD- | AGT | gsusuucuCfcUfUfGfgucuaaguguL96 | 609 | asCfsacuUfaGfAfccaaGfgAfgaaacsgsg | 958 |
| 1633993 | | | | | |
| AD- | AGT | gsusuugcUfgGfGfUfuuauuuuaguL96 | 610 | asCfsuaaAfaUfAfaaccCfaGfcaaacsusg | 959 |
| 1634065 | | | | | |
| AD- | AGT | ususugcuGfgGfUfUfuauuuuagauL96 | 611 | asUfscuaAfaAfUfaaacCfcAfgcaaascsu | 960 |
| 1634066 | | | | | |
| AD- | AGT | ususgcugGfgUfUfUfauuuuagaguL96 | 612 | asCfsucuAfaAfAfuaaaCfcCfagcaasasc | 961 |
| 1634067 | | | | | |
| AD- | AGT | usgscuggGfuUfUfAfuuuuagagauL96 | 613 | asUfscucUfaAfAfauaaAfcCfcagcasasa | 962 |
| 1634068 | | | | | |
| AD-84730 | AGT | gscsugggUfuUfAfUfuuuagagaauL96 | 614 | asUfsucuCfuAfAfaauaAfaCfccagcsasa | 963 |
| AD- | AGT | csusggguUfuAfUfUfuuagagaauuL96 | 615 | asAfsuucUfcUfAfaaauAfaAfcccagscsa | 964 |
| 1634069 | | | | | |
| AD- | AGT | usgsgguuUfaUfUfUfuagagaauguL96 | 616 | asCfsauuCfuCfUfaaaaUfaAfacccasgsc | 965 |
| 1634070 | | | | | |
| AD- | AGT | gsgsguuuAfuUfUfUfagagaaugguL96 | 617 | asCfscauUfcUfCfuaaaAfuAfaacccsasg | 966 |
| 1634071 | | | | | |
| AD- | AGT | gsgsuuuaUfuUfUfAfgagaauggguL96 | 618 | asCfsccaUfuCfUfcuaaAfaUfaaaccscsa | 967 |
| 1684536 | | | | | |
| AD- | AGT | gsusuuauUfuUfAfGfagaaugggguL96 | 619 | asCfscccAfuUfCfucuaAfaAfuaaacscsc | 968 |
| 1684538 | | | | | |
| AD- | AGT | ususuauuUfuAfGfAfgaauggggguL96 | 620 | asCfscccCfaUfUfcucuAfaAfauaaascsc | 969 |
| 1684540 | | | | | |
| AD- | AGT | asgsaaugGfgGfGfUfggggaggcauL96 | 621 | asUfsgccUfcCfCfcaccCfcCfauucuscsu | 970 |
| 1684542 | | | | | |
| AD- | AGT | gsasauggGfgGfUfGfgggaggcaauL96 | 622 | asUfsugcCfuCfCfccacCfcCfcauucsusc | 971 |
| 1684544 | | | | | |
| AD- | AGT | asasugggGfgUfGfGfggaggcaaguL96 | 623 | asCfsuugCfcUfCfcccaCfcCfccauuscsu | 972 |
| 1684546 | | | | | |
| AD- | AGT | asusggggGfuGfGfGfgaggcaagauL96 | 624 | asUfscuuGfcCfUfccccAfcCfcccaususc | 973 |
| 1684548 | | | | | |
| AD- | AGT | usgsggggUfgGfGfGfaggcaagaauL96 | 625 | asUfsucuUfgCfCfucccCfaCfccccasusu | 974 |
| 1684550 | | | | | |
| AD- | AGT | gsgsggguGfgGfGfAfggcaagaacuL96 | 626 | asGfsuucUfuGfCfcuccCfcAfcccccsasu | 975 |
| 1684552 | | | | | |
| AD- | AGT | gsusggggAfgGfCfAfagaaccaguuL96 | 627 | asAfscugGfuUfCfuugcCfuCfcccacscsc | 976 |
| 1684554 | | | | | |
| AD- | AGT | usgsgggaGfgCfAfAfgaaccaguguL96 | 628 | asCfsacuGfgUfUfcuugCfcUfccccascsc | 977 |
| 1684556 | | | | | |
| AD- | AGT | gsgsggagGfcAfAfGfaaccaguguuL96 | 629 | asAfscacUfgGfUfucuuGfcCfuccccsasc | 978 |
| 1684558 | | | | | |
| AD- | AGT | gsgsgaggCfaAfGfAfaccaguguuuL96 | 630 | asAfsacaCfuGfGfuucuUfgCfcucccscsa | 979 |
| 1634072 | | | | | |
| AD- | AGT | gsgsaggcAfaGfAfAfccaguguuuuL96 | 631 | asAfsaacAfcUfGfguucUfuGfccuccscsc | 980 |
| 1634073 | | | | | |
| AD- | AGT | gsasggcaAfgAfAfCfcaguguuuauL96 | 632 | asUfsaaaCfaCfUfgguuCfuUfgccucscsc | 981 |
| 1634074 | | | | | |
| AD- | AGT | asgsgcaaGfaAfCfCfaguguuuaguL96 | 633 | asCfsuaaAfcAfCfugguUfcUfugccuscsc | 982 |
| 1634075 | | | | | |
| AD- | AGT | gsgscaagAfaCfCfAfguguuuagcuL96 | 634 | asGfscuaAfaCfAfcuggUfuCfuugccsusc | 983 |
| 1634076 | | | | | |
| AD- | AGT | gscsaagaAfcCfAfGfuguuuagcguL96 | 635 | asCfsgcuAfaAfCfacugGfuUfcuugcscsu | 984 |
| 1634077 | | | | | |
| AD- | AGT | csasagaaCfcAfGfUfguuuagcgcuL96 | 636 | asGfscgcUfaAfAfcacuGfgUfucuugscsc | 985 |
| 1634078 | | | | | |
| AD- | AGT | asasgaacCfaGfUfGfuuuagcgcguL96 | 637 | asCfsgcgCfuAfAfacacUfgGfuucuusgsc | 986 |
| 1634079 | | | | | |
| AD- | AGT | asgsaaccAfgUfGfUfuuagcgcgguL96 | 638 | asCfscgcGfcUfAfaacaCfuGfguucususg | 987 |
| 1634080 | | | | | |
| AD- | AGT | gsasaccaGfuGfUfUfuagcgcggguL96 | 639 | asCfsccgCfgCfUfaaacAfcUfgguucsusu | 988 |
| 1634081 | | | | | |
| AD- | AGT | asasccagUfgUfUfUfagcgcgggauL96 | 640 | asUfscccGfcGfCfuaaaCfaCfugguuscsu | 989 |
| 1634082 | | | | | |
| AD- | AGT | csusguucCfaAfAfAfagaauuccauL96 | 641 | asUfsggaAfuUfCfuuuuUfgGfaacagsusa | 990 |
| 1634105 | | | | | |
| AD- | AGT | gsusuccaAfaAfAfGfaauuccaacuL96 | 642 | asGfsuugGfaAfUfucuuUfuUfggaacsasg | 991 |
| 1634107 | | | | | |
| AD- | AGT | uscscaaaAfaGfAfAfuuccaaccguL96 | 643 | asCfsgguUfgGfAfauucUfuUfuuggasasc | 992 |
| 1634109 | | | | | |
| AD- | AGT | cscsaaaaAfgAfAfUfuccaaccgauL96 | 644 | asUfscggUfuGfGfaauuCfuUfuuuggsasa | 993 |
| 1634110 | | | | | |
| AD- | AGT | csasaaaaGfaAfUfUfccaaccgacuL96 | 645 | asGfsucgGfuUfGfgaauUfcUfuuuugsgsa | 994 |
| 1634111 | | | | | |
| AD- | AGT | asasaaagAfaUfUfCfcaaccgaccuL96 | 646 | asGfsgucGfgUfUfggaaUfuCfuuuuusgsg | 995 |
| 1634112 | | | | | |
| AD- | AGT | asasaagaAfuUfCfCfaaccgaccauL96 | 647 | asUfsgguCfgGfUfuggaAfuUfcuuuususg | 996 |
| 1634113 | | | | | |
| AD- | AGT | asasagaaUfuCfCfAfaccgaccaguL96 | 648 | asCfsuggUfcGfGfuuggAfaUfucuuususu | 997 |
| 1634114 | | | | | |
| AD- | AGT | asasgaauUfcCfAfAfccgaccagcuL96 | 649 | asGfscugGfuCfGfguugGfaAfuucuususu | 998 |
| 1634115 | | | | | |
| AD-84724 | AGT | asgsaauuCfcAfAfCfcgaccagcuuL96 | 650 | asAfsgcuGfgUfCfgguuGfgAfauucususu | 999 |
| AD- | AGT | gsasauucCfaAfCfCfgaccagcuuuL96 | 651 | asAfsagcUfgGfUfcgguUfgGfaauucsusu | 1000 |
| 1634116 | | | | | |
| AD- | AGT | asasuuccAfaCfCfGfaccagcuuguL96 | 652 | asCfsaagCfuGfGfucggUfuGfgaauuscsu | 1001 |
| 1634117 | | | | | |
| AD- | AGT | asusuccaAfcCfGfAfccagcuuguuL96 | 653 | asAfscaaGfcUfGfgucgGfuUfggaaususc | 1002 |
| 1634118 | | | | | |
| AD-68579 | AGT | ususccaaCfcGfAfCfcagcuuguuuL96 | 654 | asAfsacaAfgCfUfggucGfgUfuggaasusu | 1003 |
| AD- | AGT | uscscaacCfgAfCfCfagcuuguuuuL96 | 655 | asAfsaacAfaGfCfugguCfgGfuuggasasu | 1004 |
| 1634119 | | | | | |
| AD- | AGT | cscsaaccGfaCfCfAfgcuuguuuguL96 | 656 | asCfsaaaCfaAfGfcuggUfcGfguuggsasa | 1005 |
| 1634120 | | | | | |
| AD- | AGT | csasaccgAfcCfAfGfcuuguuuguuL96 | 657 | asAfscaaAfcAfAfgcugGfuCfgguugsgsa | 1006 |
| 1634121 | | | | | |
| AD- | AGT | asasccgaCfcAfGfCfuuguuuguguL96 | 658 | asCfsacaAfaCfAfagcuGfgUfcgguusgsg | 1007 |
| 1634122 | | | | | |
| AD- | AGT | ascscgacCfaGfCfUfuguuugugauL96 | 659 | asUfscacAfaAfCfaagcUfgGfucggususg | 1008 |
| 1634123 | | | | | |
| AD- | AGT | cscsgaccAfgCfUfUfguuugugaauL96 | 660 | asUfsucaCfaAfAfcaagCfuGfgucggsusu | 1009 |
| 1634124 | | | | | |
| AD- | AGT | csgsaccaGfcUfUfGfuuugugaaauL96 | 661 | asUfsuucAfcAfAfacaaGfcUfggucgsgsu | 1010 |
| 1634125 | | | | | |
| AD- | AGT | gsasccagCfuUfGfUfuugugaaacuL96 | 662 | asGfsuuuCfaCfAfaacaAfgCfuggucsgsg | 1011 |
| 1634126 | | | | | |
| AD- | AGT | ascscagcUfuGfUfUfugugaaacauL96 | 663 | asUfsguuUfcAfCfaaacAfaGfcugguscsg | 1012 |
| 1634127 | | | | | |
| AD- | AGT | cscsagcuUfgUfUfUfgugaaacaauL96 | 664 | asUfsuguUfuCfAfcaaaCfaAfgcuggsusc | 1013 |
| 1634128 | | | | | |
| AD- | AGT | csasgcuuGfuUfUfGfugaaacaaauL96 | 665 | asUfsuugUfuUfCfacaaAfcAfagcugsgsu | 1014 |
| 1634129 | | | | | |
| AD- | AGT | asgscuugUfuUfGfUfgaaacaaaauL96 | 666 | asUfsuuuGfuUfUfcacaAfaCfaagcusgsg | 1015 |
| 1634130 | | | | | |
| AD- | AGT | gscsuuguUfuGfUfGfaaacaaaaauL96 | 667 | asUfsuuuUfgUfUfucacAfaAfcaagcsusg | 1016 |
| 1684562 | | | | | |
| AD- | AGT | csusuguuUfgUfGfAfaacaaaaaauL96 | 668 | asUfsuuuUfuGfUfuucaCfaAfacaagscsu | 1017 |
| 1684563 | | | | | |
| AD- | AGT | ususguuuGfuGfAfAfacaaaaaaguL96 | 669 | asCfsuuuUfuUfGfuuucAfcAfaacaasgsc | 1018 |
| 1684564 | | | | | |
| AD- | AGT | usgsuuccCfuUfUfUfcaaguugaguL96 | 670 | asCfsucaAfcUfUfgaaaAfgGfgaacascsu | 1019 |
| 1634135 | | | | | |
| AD- | AGT | gsusucccUfuUfUfCfaaguugagauL96 | 671 | asUfscucAfaCfUfugaaAfaGfggaacsasc | 1020 |
| 1634136 | | | | | |
| AD- | AGT | ususcccuUfuUfCfAfaguugagaauL96 | 672 | asUfsucuCfaAfCfuugaAfaAfgggaascsa | 1021 |
| 1634137 | | | | | |
| AD- | AGT | csasaguuGfaGfAfAfcaaaaauuguL96 | 673 | asCfsaauUfuUfUfguucUfcAfacuugsasa | 1022 |
| 1634146 | | | | | |
| AD- | AGT | asasguugAfgAfAfCfaaaaauugguL96 | 674 | asCfscaaUfuUfUfuguuCfuCfaacuusgsa | 1023 |
| 1634147 | | | | | |
| AD-68585 | AGT | asgsuugaGfaAfCfAfaaaauuggguL96 | 675 | asCfsccaAfuUfUfuuguUfcUfcaacususg | 1024 |
| AD- | AGT | gsusugagAfaCfAfAfaaauuggguuL96 | 676 | asAfscccAfaUfUfuuugUfuCfucaacsusu | 1025 |
| 1634148 | | | | | |
| AD- | AGT | ususgagaAfcAfAfAfaauuggguuuL96 | 677 | asAfsaccCfaAfUfuuuuGfuUfcucaascsu | 1026 |
| 1634149 | | | | | |
| AD- | AGT | usgsagaaCfaAfAfAfauuggguuuuL96 | 678 | asAfsaacCfcAfAfuuuuUfgUfucucasasc | 1027 |
| 1634150 | | | | | |
| AD- | AGT | gsasgaacAfaAfAfAfuuggguuuuuL96 | 679 | asAfsaaaCfcCfAfauuuUfuGfuucucsasa | 1028 |
| 1634151 | | | | | |
| AD- | AGT | asgsaacaAfaAfAfUfuggguuuuauL96 | 680 | asUfsaaaAfcCfCfaauuUfuUfguucuscsa | 1029 |
| 1634152 | | | | | |
| AD- | AGT | gsasacaaAfaAfUfUfggguuuuaauL96 | 681 | asUfsuaaAfaCfCfcaauUfuUfuguucsusc | 1030 |
| 1634153 | | | | | |
| AD- | AGT | asgsuauaCfaUfUfUfuugcauugcuL96 | 682 | asGfscaaUfgCfAfaaaaUfgUfauacususu | 1031 |
| 1634162 | | | | | |
| AD- | AGT | gsusauacAfuUfUfUfugcauugccuL96 | 683 | asGfsgcaAfuGfCfaaaaAfuGfuauacsusu | 1032 |
| 1634163 | | | | | |
| AD- | AGT | usasuacaUfuUfUfUfgcauugccuuL96 | 684 | asAfsggcAfaUfGfcaaaAfaUfguauascsu | 1033 |
| 1634164 | | | | | |
| AD- | AGT | asusacauUfuUfUfGfcauugccuuuL96 | 685 | asAfsaggCfaAfUfgcaaAfaAfuguausasc | 1034 |
| 1634165 | | | | | |
| AD- | AGT | asusuuuuGfcAfUfUfgccuucgguuL96 | 686 | asAfsccgAfaGfGfcaauGfcAfaaaausgsu | 1035 |
| 1634169 | | | | | |
| AD- | AGT | ususuuugCfaUfUfGfccuucgguuuL96 | 687 | asAfsaccGfaAfGfgcaaUfgCfaaaaasusg | 1036 |
| 1634170 | | | | | |
| AD- | AGT | ususuugcAfuUfGfCfcuucgguuuuL96 | 688 | asAfsaacCfgAfAfggcaAfuGfcaaaasasu | 1037 |
| 1634171 | | | | | |
| AD- | AGT | ususugcaUfuGfCfCfuucgguuuguL96 | 689 | asCfsaaaCfcGfAfaggcAfaUfgcaaasasa | 1038 |
| 1634172 | | | | | |
| AD- | AGT | ususgcauUfgCfCfUfucgguuuguuL96 | 690 | asAfscaaAfcCfGfaaggCfaAfugcaasasa | 1039 |
| 1634173 | | | | | |
| AD- | AGT | usgscauuGfcCfUfUfcgguuuguauL96 | 691 | asUfsacaAfaCfCfgaagGfcAfaugcasasa | 1040 |
| 1634174 | | | | | |
| AD- | AGT | gscsauugCfcUfUfCfgguuuguauuL96 | 692 | asAfsuacAfaAfCfcgaaGfgCfaaugcsasa | 1041 |
| 1634175 | | | | | |
| AD- | AGT | csasuugcCfuUfCfGfguuuguauuuL96 | 693 | asAfsauaCfaAfAfccgaAfgGfcaaugscsa | 1042 |
| 1634176 | | | | | |
| AD- | AGT | asusugccUfuCfGfGfuuuguauuuuL96 | 694 | asAfsaauAfcAfAfaccgAfaGfgcaausgsc | 1043 |
| 1634177 | | | | | |
| AD- | AGT | ususgccuUfcGfGfUfuuguauuuauL96 | 695 | asUfsaaaUfaCfAfaaccGfaAfggcaasusg | 1044 |
| 1634178 | | | | | |
| AD- | AGT | usgsccuuCfgGfUfUfuguauuuaguL96 | 696 | asCfsuaaAfuAfCfaaacCfgAfaggcasasu | 1045 |
| 1634179 | | | | | |
| AD- | AGT | gscscuucGfgUfUfUfguauuuaguuL96 | 697 | asAfscuaAfaUfAfcaaaCfcGfaaggcsasa | 1046 |
| 1634180 | | | | | |
| AD- | AGT | cscsuucgGfuUfUfGfuauuuaguguL96 | 698 | asCfsacuAfaAfUfacaaAfcCfgaaggscsa | 1047 |
| 1634181 | | | | | |
| AD- | AGT | csusucggUfuUfGfUfauuuaguguuL96 | 699 | asAfscacUfaAfAfuacaAfaCfcgaagsgsc | 1048 |
| 1634182 | | | | | |
| AD- | AGT | ususcgguUfuGfUfAfuuuagugucuL96 | 700 | asGfsacaCfuAfAfauacAfaAfccgaasgsg | 1049 |
| 1634183 | | | | | |
| AD- | AGT | uscsgguuUfgUfAfUfuuagugucuuL96 | 701 | asAfsgacAfcUfAfaauaCfaAfaccgasasg | 1050 |
| 1634184 | | | | | |
| AD- | AGT | csgsguuuGfuAfUfUfuagugucuuuL96 | 702 | asAfsagaCfaCfUfaaauAfcAfaaccgsasa | 1051 |
| 1634185 | | | | | |
| AD- | AGT | gsgsuuugUfaUfUfUfagugucuuguL96 | 703 | asCfsaagAfcAfCfuaaaUfaCfaaaccsgsa | 1052 |
| 1634186 | | | | | |
| AD- | AGT | gsusuuguAfuUfUfAfgugucuugauL96 | 704 | asUfscaaGfaCfAfcuaaAfuAfcaaacscsg | 1053 |
| 1634187 | | | | | |
| AD- | AGT | ususuguaUfuUfAfGfugucuugaauL96 | 705 | asUfsucaAfgAfCfacuaAfaUfacaaascsc | 1054 |
| 1634188 | | | | | |
| AD- | AGT | ususguauUfuAfGfUfgucuugaauuL96 | 706 | asAfsuucAfaGfAfcacuAfaAfuacaasasc | 1055 |
| 1634189 | | | | | |
| AD- | AGT | usgsuauuUfaGfUfGfucuugaauguL96 | 707 | asCfsauuCfaAfGfacacUfaAfauacasasa | 1056 |
| 1634190 | | | | | |
| AD- | AGT | gsusauuuAfgUfGfUfcuugaauguuL96 | 708 | asAfscauUfcAfAfgacaCfuAfaauacsasa | 1057 |
| 1634191 | | | | | |
| AD- | AGT | usasuuuaGfuGfUfCfuugaauguauL96 | 709 | asUfsacaUfuCfAfagacAfcUfaaauascsa | 1058 |
| 1634192 | | | | | |
| AD- | AGT | asusuuagUfgUfCfUfugaauguaauL96 | 710 | asUfsuacAfuUfCfaagaCfaCfuaaausasc | 1059 |
| 1634193 | | | | | |
| AD- | AGT | ususuaguGfuCfUfUfgaauguaaguL96 | 711 | asCfsuuaCfaUfUfcaagAfcAfcuaaasusa | 1060 |
| 1634194 | | | | | |
| AD- | AGT | ususagugUfcUfUfGfaauguaagauL96 | 712 | asUfscuuAfcAfUfucaaGfaCfacuaasasu | 1061 |
| 1634195 | | | | | |
| AD- | AGT | usasguguCfuUfGfAfauguaagaauL96 | 713 | asUfsucuUfaCfAfuucaAfgAfcacuasasa | 1062 |
| 1634196 | | | | | |
| AD- | AGT | asgsugucUfuGfAfAfuguaagaacuL96 | 714 | asGfsuucUfuAfCfauucAfaGfacacusasa | 1063 |
| 1634197 | | | | | |
| AD- | AGT | usgsucuuGfaAfUfGfuaagaacauuL96 | 715 | asAfsuguUfcUfUfacauUfcAfagacascsu | 1064 |
| 1634199 | | | | | |
| AD- | AGT | gsuscuugAfaUfGfUfaagaacauguL96 | 716 | asCfsaugUfuCfUfuacaUfuCfaagacsasc | 1065 |
| 1634200 | | | | | |
| AD- | AGT | ususgaauGfuAfAfGfaacaugaccuL96 | 717 | asGfsgucAfuGfUfucuuAfcAfuucaasgsa | 1066 |
| 1634203 | | | | | |
| AD- | AGT | gsusaagaAfcAfUfGfaccuccguguL96 | 718 | asCfsacgGfaGfGfucauGfuUfcuuacsasu | 1067 |
| 1634209 | | | | | |
| AD- | AGT | usasagaaCfaUfGfAfccuccguguuL96 | 719 | asAfscacGfgAfGfgucaUfgUfucuuascsa | 1068 |
| 1634210 | | | | | |
| AD- | AGT | asasgaacAfuGfAfCfcuccguguauL96 | 720 | asUfsacaCfgGfAfggucAfuGfuucuusasc | 1069 |
| 1634211 | | | | | |
| AD- | AGT | asgsaacaUfgAfCfCfuccguguaguL96 | 721 | asCfsuacAfcGfGfagguCfaUfguucususa | 1070 |
| 1634212 | | | | | |
| AD- | AGT | gsasacauGfaCfCfUfccguguaguuL96 | 722 | asAfscuaCfaCfGfgaggUfcAfuguucsusu | 1071 |
| 1634213 | | | | | |
| AD- | AGT | asascaugAfcCfUfCfcguguaguguL96 | 723 | asCfsacuAfcAfCfggagGfuCfauguuscsu | 1072 |
| 1634214 | | | | | |
| AD- | AGT | ascsaugaCfcUfCfCfguguaguguuL96 | 724 | asAfscacUfaCfAfcggaGfgUfcaugususc | 1073 |
| 1634215 | | | | | |
| AD- | AGT | csasugacCfuCfCfGfuguagugucuL96 | 725 | asGfsacaCfuAfCfacggAfgGfucaugsusu | 1074 |
| 1634216 | | | | | |
| AD- | AGT | asusgaccUfcCfGfUfguagugucuuL96 | 726 | asAfsgacAfcUfAfcacgGfaGfgucausgsu | 1075 |
| 1634217 | | | | | |
| AD- | AGT | csusuaguUfuUfUfUfccacagauguL96 | 727 | asCfsaucUfgUfGfgaaaAfaAfcuaagsgsu | 1076 |
| 1684565 | | | | | |
| AD- | AGT | ususaguuUfuUfUfCfcacagaugcuL96 | 728 | asGfscauCfuGfUfggaaAfaAfacuaasgsg | 1077 |
| 1684566 | | | | | |
| AD- | AGT | usasguuuUfuUfCfCfacagaugcuuL96 | 729 | asAfsgcaUfcUfGfuggaAfaAfaacuasasg | 1078 |
| 1684567 | | | | | |
| AD- | AGT | gsusuuuuUfcCfAfCfagaugcuuguL96 | 730 | asCfsaagCfaUfCfugugGfaAfaaaacsusa | 1079 |
| 1684568 | | | | | |
| AD- | AGT | ususuuucCfaCfAfGfaugcuuguguL96 | 731 | asCfsacaAfgCfAfucugUfgGfaaaaasasc | 1080 |
| 1684569 | | | | | |
| AD- | AGT | ususuuccAfcAfGfAfugcuugugauL96 | 732 | asUfscacAfaGfCfaucuGfuGfgaaaasasa | 1081 |
| 1684570 | | | | | |
| AD- | AGT | ususuccaCfaGfAfUfgcuugugauuL96 | 733 | asAfsucaCfaAfGfcaucUfgUfggaaasasa | 1082 |
| 1634234 | | | | | |
| AD- | AGT | ususccacAfgAfUfGfcuugugauuuL96 | 734 | asAfsaucAfcAfAfgcauCfuGfuggaasasa | 1083 |
| 1634235 | | | | | |
| AD- | AGT | uscscacaGfaUfGfCfuugugauuuuL96 | 735 | asAfsaauCfaCfAfagcaUfcUfguggasasa | 1084 |
| 1634236 | | | | | |
| AD- | AGT | cscsacagAfuGfCfUfugugauuuuuL96 | 736 | asAfsaaaUfcAfCfaagcAfuCfuguggsasa | 1085 |
| 1634237 | | | | | |
| AD- | AGT | csascagaUfgCfUfUfgugauuuuuuL96 | 737 | asAfsaaaAfuCfAfcaagCfaUfcugugsgsa | 1086 |
| 1634238 | | | | | |
| AD- | AGT | ascscugaAfuUfUfCfuguuugaauuL96 | 738 | asAfsuucAfaAfCfagaaAfuUfcaggusgsc | 1087 |
| 1634282 | | | | | |
| AD- | AGT | cscsugaaUfuUfCfUfguuugaauguL96 | 739 | asCfsauuCfaAfAfcagaAfaUfucaggsusg | 1088 |
| 1634283 | | | | | |
| AD- | AGT | gsgsaaccAfuAfGfCfugguuauuuuL96 | 740 | asAfsaauAfaCfCfagcuAfuGfguuccsgsc | 1089 |
| 1634304 | | | | | |
| AD- | AGT | gsasaccaUfaGfCfUfgguuauuucuL96 | 741 | asGfsaaaUfaAfCfcagcUfaUfgguucscsg | 1090 |
| 1634305 | | | | | |
| AD- | AGT | asasccauAfgCfUfGfguuauuucuuL96 | 742 | asAfsgaaAfuAfAfccagCfuAfugguuscsc | 1091 |
| 1634306 | | | | | |
| AD- | AGT | ascscauaGfcUfGfGfuuauuucucuL96 | 743 | asGfsagaAfaUfAfaccaGfcUfauggususc | 1092 |
| 1634307 | | | | | |
| AD- | AGT | cscsauagCfuGfGfUfuauuucuccuL96 | 744 | asGfsgagAfaAfUfaaccAfgCfuauggsusu | 1093 |
| 1634308 | | | | | |
| AD- | AGT | cscsuuguGfuUfAfGfuaauaaacguL96 | 745 | asCfsguuUfaUfUfacuaAfcAfcaaggsgsa | 1094 |
| 1634327 | | | | | |
| AD- | AGT | csusugugUfuAfGfUfaauaaacguuL96 | 746 | asAfscguUfuAfUfuacuAfaCfacaagsgsg | 1095 |
| 1634328 | | | | | |
| AD- | AGT | ususguguUfaGfUfAfauaaacgucuL96 | 747 | asGfsacgUfuUfAfuuacUfaAfcacaasgsg | 1096 |
| 1634329 | | | | | |
| AD- | AGT | usgsuguuAfgUfAfAfuaaacgucuuL96 | 748 | asAfsgacGfuUfUfauuaCfuAfacacasasg | 1097 |
| 1634330 | | | | | |
| AD- | AGT | gsusguuaGfuAfAfUfaaacgucuuuL96 | 749 | asAfsagaCfgUfUfuauuAfcUfaacacsasa | 1098 |
| 1634331 | | | | | |
| AD- | AGT | usgsuuagUfaAfUfAfaacgucuuguL96 | 750 | asCfsaagAfcGfUfuuauUfaCfuaacascsa | 1099 |
| 1634332 | | | | | |
| AD- | AGT | gsusuaguAfaUfAfAfacgucuugcuL96 | 751 | asGfscaaGfaCfGfuuuaUfuAfcuaacsasc | 1100 |
| 1634333 | | | | | |

TABLE 4
Abbreviations of nucleotide monomers used in nucleic acid sequence representation*

| Abbreviation | Nucleotide(s) |
|---|---|
| A | Adenosine-3′-phosphate |
| Ab | beta-L-adenosine-3′-phosphate |
| Abs | beta-L-adenosine-3′-phosphorothioate |
| Af | 2′-fluoroadenosine-3′-phosphate |
| Afs | 2′-fluoroadenosine-3′-phosphorothioate |
| As | adenosine-3′-phosphorothioate |
| C | cytidine-3′-phosphate |
| Cb | beta-L-cytidine-3′-phosphate |
| Cbs | beta-L-cytidine-3′-phosphorothioate |
| Cf | 2′-fluorocytidine-3′-phosphate |
| Cfs | 2′-fluorocytidine-3′-phosphorothioate |
| Cs | cytidine-3′-phosphorothioate |
| G | guanosine-3′-phosphate |
| Gb | beta-L-guanosine-3′-phosphate |
| Gbs | beta-L-guanosine-3′-phosphorothioate |
| Gf | 2′-fluoroguanosine-3′-phosphate |
| Gfs | 2′-fluoroguanosine-3′-phosphorothioate |
| Gs | guanosine-3′-phosphorothioate |
| T | 5′-methyluridine-3′-phosphate |
| Tf | 2′-fluoro-5-methyluridine-3′-phosphate |
| Tfs | 2′-fluoro-5-methyluridine-3′-phosphorothioate |
| Ts | 5-methyluridine-3′-phosphorothioate |
| U | Uridine-3′-phosphate |
| Uf | 2′-fluorouridine-3′-phosphate |
| Ufs | 2′-fluorouridine-3′-phosphorothioate |
| Us | uridine-3′-phosphorothioate |
| N | any nucleotide, modified or unmodified |
| a | 2′-O-methyladenosine-3′-phosphate |
| as | 2′-O-methyladenosine-3′-phosphorothioate |
| c | 2′-O-methylcytidine-3′-phosphate |
| CS | 2′-O-methylcytidine-3′-phosphorothioate |
| g | 2′-O-methylguanosine-3′-phosphate |
| gs | 2′-O-methylguanosine-3′-phosphorothioate |
| t | 2′-O-methyl-5-methyluridine-3′-phosphate |
| ts | 2′-O-methyl-5-methyluridine-3′-phosphorothioate |
| u | 2′-O-methyluridine-3′-phosphate |
| us | 2′-O-methyluridine-3′-phosphorothioate |
| s | phosphorothioate linkage |
| L96 | N-[tris(GalNAc-alkyl)-amido-dodecanoyl)]-4-hydroxyprolinol |
| | [Hyp-(GalNAc-alkyl)₃] |
| | 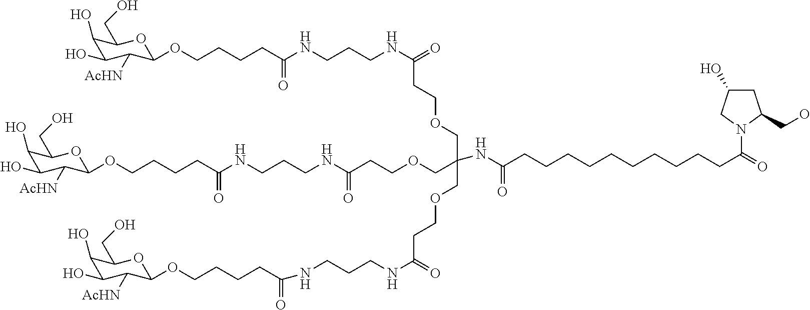 [SMILES: CC(=O)NC1C(OCCCCC(=O)NCCCNC(=O)CCOCC(COCCC(=O)NCCCNC(=O)CCCCOC2OC(CO)C(O)C(O)C2NC(C)=O)(COCCC(=O)NCCCNC(=O)CCCCOC2OC(CO)C(O)C(O)C2NC(C)=O)NC(=O)CCCCCCCCCCC(=O)N2C[C@H](O)C[C@H]2CO)OC(CO)C(O)C1O] |
| (Agn) | Adenosine-glycol nucleic acid (GNA) |
| (Cgn) | Cytidine-glycol nucleic acid (GNA) |
| (Ggn) | Guanosine-glycol nucleic acid (GNA) |
| (Tgn) | Thymidine-glycol nucleic acid (GNA) S-Isomer |
| P | Phosphate |
| VP | Vinyl-phosphonate (e.g., 5′-E-vinylphosphonate) |
| dA | 2′-deoxyadenosine-3′-phosphate |
| dAs | 2′-deoxyadenosine-3-phosphorothioate |
| dC | 2′-deoxycytidine-3-phosphate |
| dCs | 2′-deoxycytidine-3′-phosphorothioate |
| dG | 2′-deoxyguanosine-3′-phosphate |
| dGs | 2′-deoxyguanosine-3′-phosphorothioate |
| dT | 2′-deoxythymidine-3′-phosphate |
| dTs | 2′-deoxythymidine-3′-phosphorothioate |
| dU | 2′-deoxyuridine |
| dUs | 2′-deoxyuridine-3-phosphorothioate |
| (Ahd) | 2′-O-hexadecyl-adenosine-3′-phosphate |
| (Ahds) | 2′-O-hexadecyl-adenosine-3′-phosphorothioate |
| (Chd) | 2′-O-hexadecyl-cytidine-3′-phosphate |
| (Chds) | 2′-O-hexadecyl-cytidine-3′-phosphorothioate |
| (Ghd) | 2′-O-hexadecyl-guanosine-3′-phosphate |
| (Ghds) | 2′-O-hexadecyl-guanosine-3′-phosphorothioate |
| (Uhd) | 2′-O-hexadecyl-uridine-3′-phosphate |
| (Uhds) | 2′-O-hexadecyl-uridine-3′-phosphorothioate |

*It will be understood that these monomers, when present in an oligonucleotide, are mutually linked by 5′-3′-phosphodiester bonds; and it is understood that when the nucleotide contains a 2′-fluoro modification, then the fluoro replaces the hydroxy at that position of the parent nucleotide (i.e., it is a 2′-deoxy-2′-fluoronucleotide.

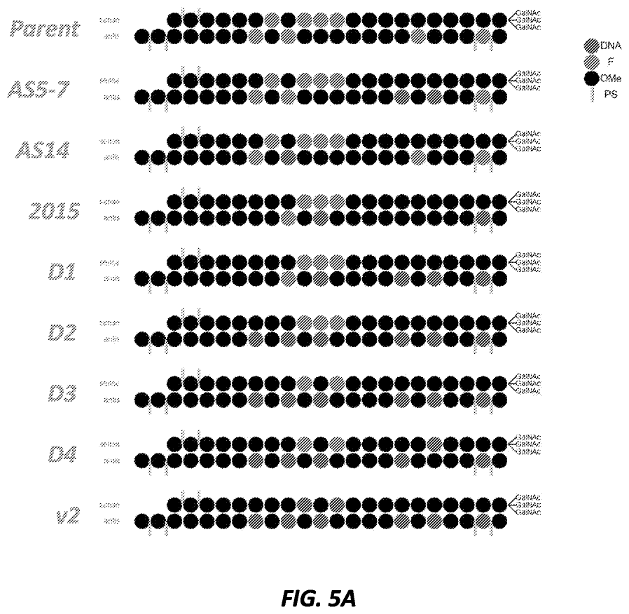

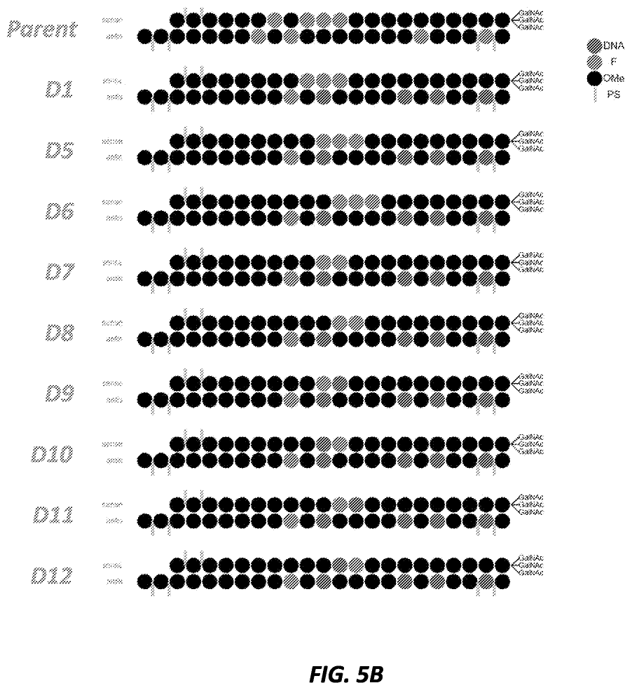

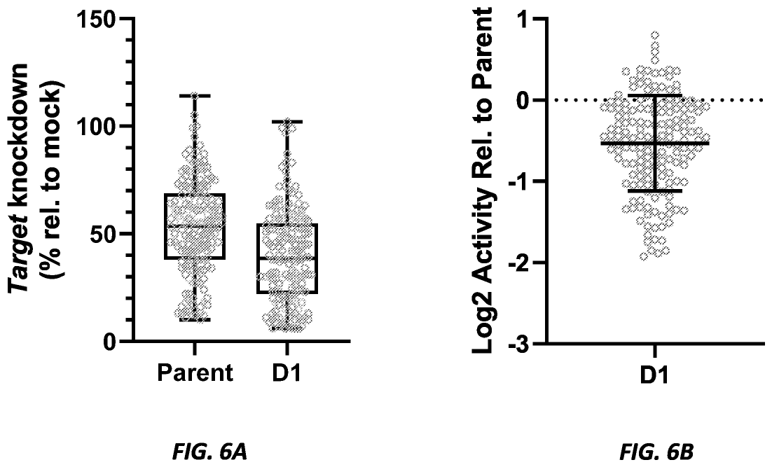

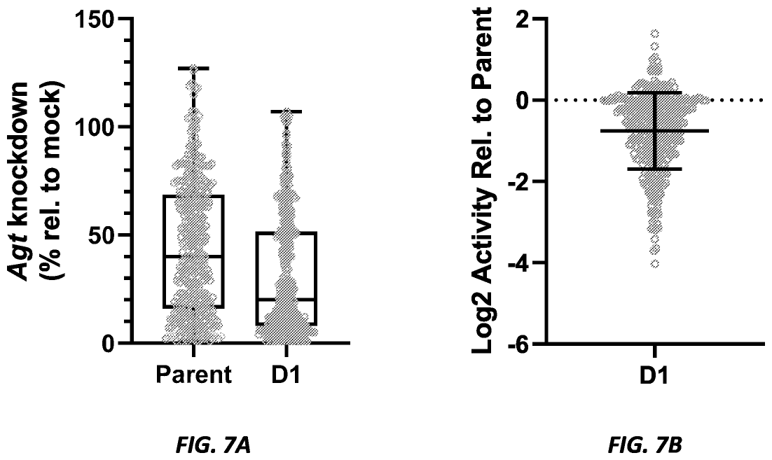

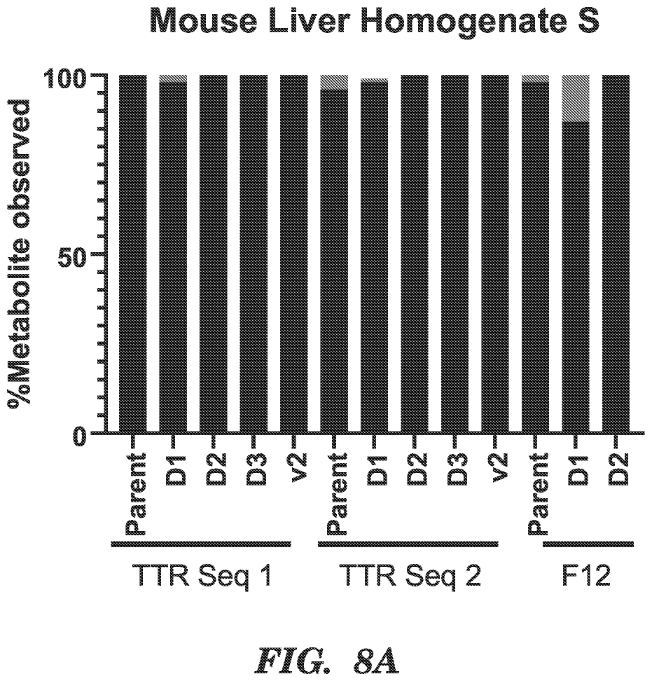

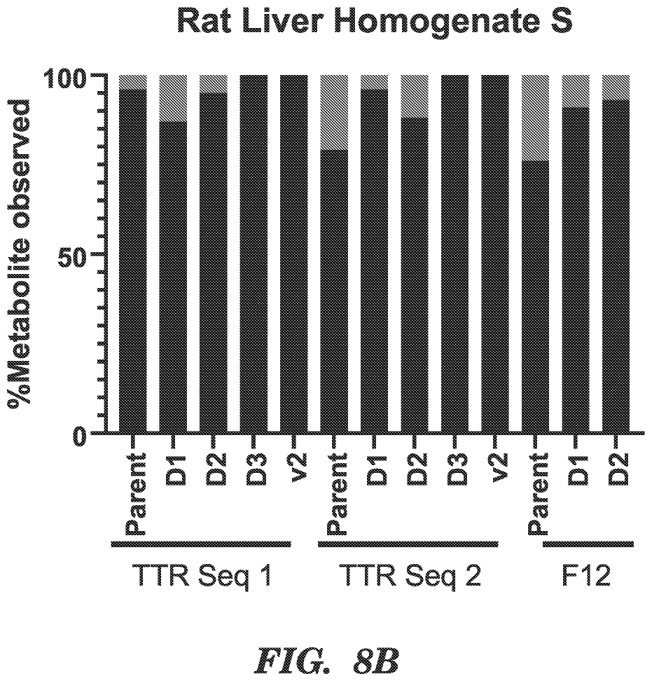

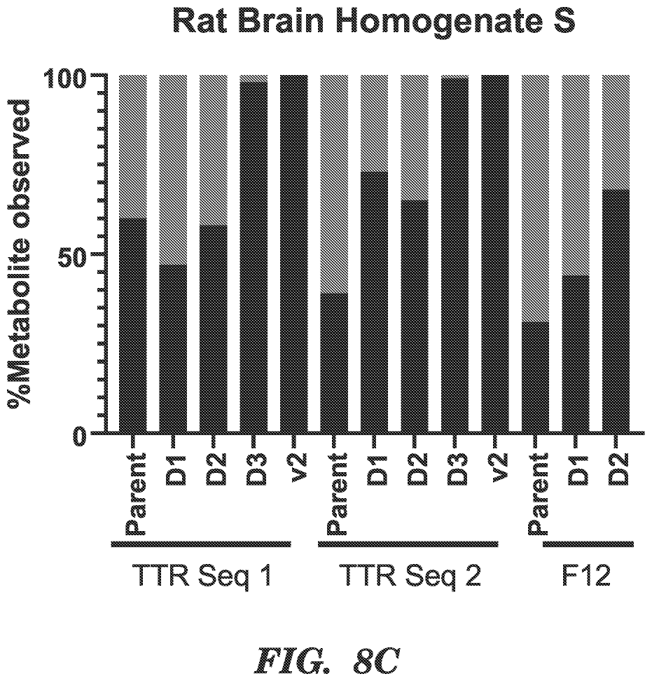

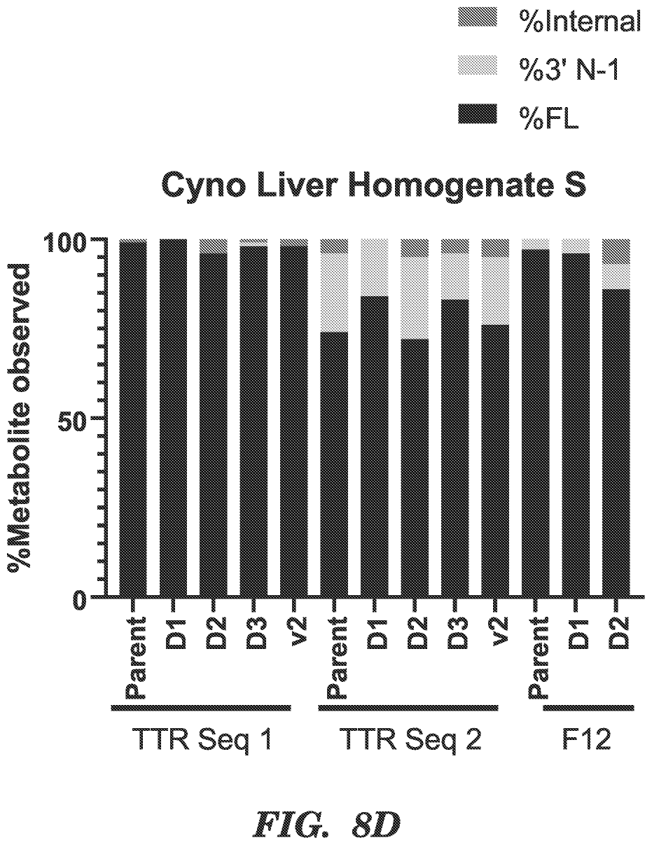

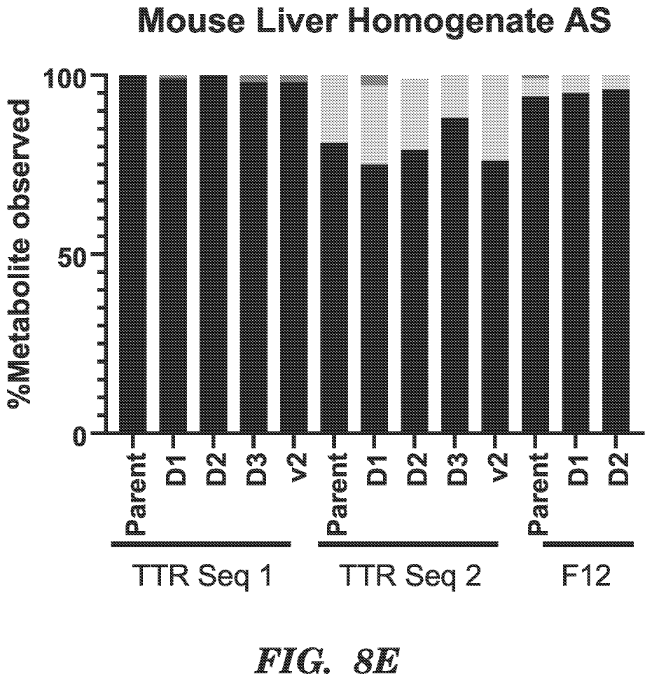

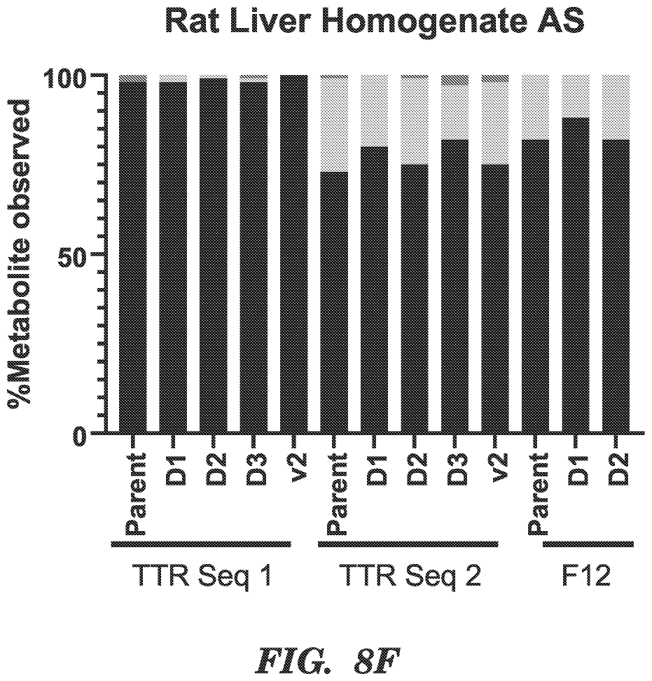

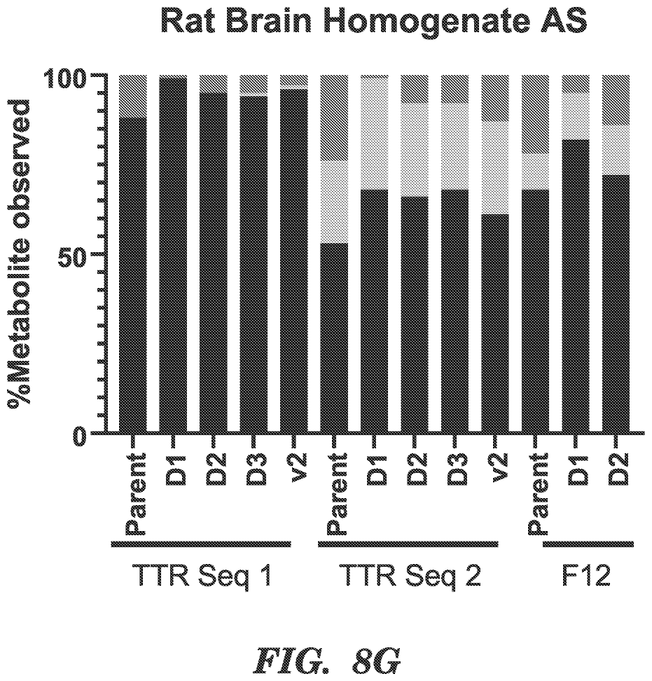

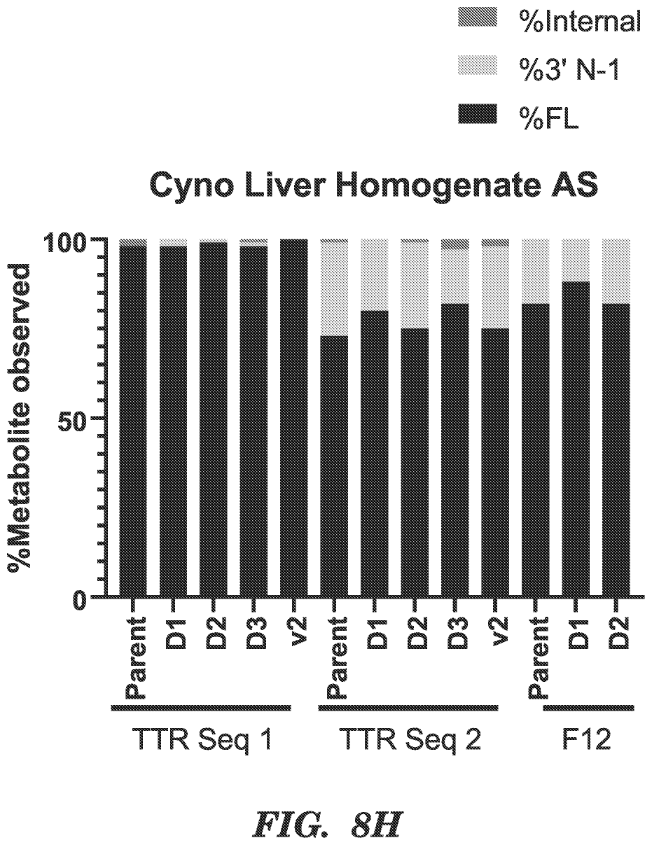

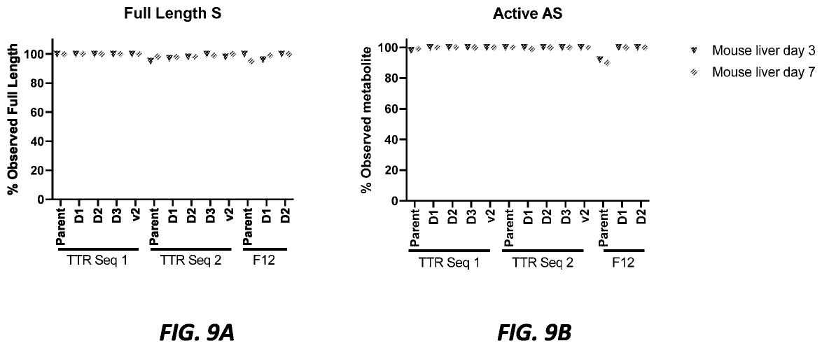

Some exemplary dsRNA molecule designs according to embodiments of the disclosure are shown schematically in FIGS. 5A and 5B. Exemplary dsRNA molecules according to some embodiments of the disclosure are listed in Table 5 and 6.

TABLE 5
Exemplary dsRNA molecule according to some embodiments of the disclosure

| | | | SEQ | | SEQ |
|---|---|---|---|---|---|
| Duplex | | | ID | | ID |
| ID | Target | Sense sequence (5′→3′) | NO | Antisense sequence (5′→3′) | NO: |
| AD- | Marc1 | asasaaaugu UfCfUfcaaaaaugaaL96 | 1101 | usdTscadTudTuugadGaAfcauuuuusasa | 1269 |
| 1636769 | | | | | |
| AD- | Marc1 | asasaaucacCfAfCfucuuugggcaL96 | 1102 | usdGsccdCadAagagdTgGfugauuuuscsc | 1270 |
| 1636805 | | | | | |
| AD- | Marc1 | asasagugggAfGfAfcccuguguaaL96 | 1103 | usdTsacdAcdAgggudCuCfccacuuusgsa | 1271 |
| 1636782 | | | | | |
| AD- | PNPLA3 | asasaugaaaGfAfCfaaagguggauL96 | 1104 | asdTsccdAcdCuuugdTcUfuucauuuscsu | 1272 |
| 1636754 | | | | | |
| AD- | C3 | asasaugaggGfUfUfucacagucaaL96 | 1105 | usdTsgadCudGugaadAcCfcucauuususc | 1273 |
| 1636695 | | | | | |
| AD- | PNPLA3 | asascuugcuAfCfCfcauuaggauaL96 | 1106 | usdAsucdCudAauggdGuAfgcaaguusgsc | 1274 |
| 1636733 | | | | | |
| AD- | Marc1 | asasucaccaCfUfCfuuugggcaguL96 | 1107 | asdCsugdCcdCaaagdAgUfggugauususu | 1275 |
| 1636798 | | | | | |
| AD- | C3 | ascsaugggcCfAfGfuggaagaucaL96 | 1108 | usdGsaudCudTccacdTgGfcccaugususg | 1276 |
| 1636720 | | | | | |
| AD- | C3 | ascscaggaaCfUfGfaaccuugauaL96 | 1109 | usdAsucdAadGguucdAgUfuccuggusgsg | 1277 |
| 1636701 | | | | | |
| AD- | C3 | ascscaggauGfCfCfacuaugucuaL96 | 1110 | usdAsgadCadTagugdGcAfuccugguscsu | 1278 |
| 1636716 | | | | | |
| AD- | PNPLA3 | ascscuaacuAfAfAfauaauguuuaL96 | 1111 | usdAsaadCadTuauudTuAfguuaggusgsa | 1279 |
| 1636729 | | | | | |
| AD- | Marc1 | ascscucgccUfGfGfuccugauuuaL96 | 1112 | usdAsaadTcdAggacdCaGfgcgaggususc | 1280 |
| 1636806 | | | | | |
| AD- | PNPLA3 | ascscuguugAfAfUfuuuguauuauL96 | 1113 | asdTsaadTadCaaaadTuCfaacaggusasa | 1281 |
| 1636735 | | | | | |
| AD- | C3 | ascsuacaugAfAfCfcuacagagauL96 | 1114 | asdTscudCudGuaggdTuCfauguagususg | 1282 |
| 1636714 | | | | | |
| AD- | Marc1 | ascsugauuaUfGfGfaauaguucuuL96 | 1115 | asdAsgadAcdTauucdCaUfaaucagususa | 1283 |
| 1636778 | | | | | |
| AD- | C3 | asgsaaauucUfAfCfuacaucuauuL96 | 1116 | asdAsuadGadTguagdTaGfaauuucuscsu | 1284 |
| 1479350 | | | | | |
| AD- | Marc1 | asgsacaggaUfUfCfugaaaacucaL96 | 1117 | usdGsagdTudTucagdAaUfccugucususg | 1285 |
| 1636785 | | | | | |
| AD- | C3 | asgsagcgggUfAfCfcucuucaucaL96 | 1118 | usdGsaudGadAgaggdTaCfccgcucusgsc | 1286 |
| 1636763 | | | | | |
| AD- | SCN9A | asgscaaaggUfCfAfcaauuuccuaL96 | 1119 | usdAsggdAadAuugudGaCfcuuugcuscsa | 1287 |
| 1636834 | | | | | |
| AD- | SCN9A | asgscauaaaUfGfUfuuucgaaauaL96 | 1120 | usdAsuudTcdGaaaadCaUfuuaugcususc | 1288 |
| 1636811 | | | | | |
| AD- | PNPLA3 | asgscaugagGfUfUfcuuagaauguL96 | 1121 | asdCsaudTcdTaagadAcCfucaugcusgsg | 1289 |
| 1636757 | | | | | |
| AD- | PNPLA3 | asgsgaagcaAfCfCfuuucgccuguL96 | 1122 | asdCsagdGcdGaaagdGuUfgcuuccusasg | 1290 |
| 1636758 | | | | | |
| AD- | Marc1 | asgsgaccagAfUfUfgcuuacucaaL96 | 1123 | usdTsgadGudAagcadAuCfugguccususg | 1291 |
| 1636791 | | | | | |
| AD- | Marc1 | asgsgagaagAfAfAfagugauucaaL96 | 1124 | usdTsgadAudCacuudTuCfuucuccuscsc | 1292 |
| 1636777 | | | | | |
| AD- | SCN9A | asgsucuucaAfGfUfuggcaaaauaL96 | 1125 | usdAsuudTudGccaadCuUfgaagacuscsg | 1293 |
| 1636838 | | | | | |
| AD- | C3 | asgsuggacuAfUfGfuguacaagauL96 | 1126 | asdTscudTgdTacacdAuAfguccacuscsc | 1294 |
| 1636703 | | | | | |
| AD- | SCN9A | asgsuuccuaUfCfUfccuuucagaaL96 | 1127 | usdTscudGadAaggadGaUfaggaacusasc | 1295 |
| 1636831 | | | | | |
| AD- | PNPLA3 | asusaaugucUfUfAfuguaaugcuuL96 | 1128 | asdAsgcdAudTacaudAaGfacauuauscsc | 1296 |
| 1636732 | | | | | |
| AD- | PNPLA3 | asusacaugaGfCfAfagauuugcaaL96 | 1129 | usdTsgcdAadAucuudGcUfcauguauscsc | 1297 |
| 1636737 | | | | | |
| AD- | Marc1 | asuscaaccaGfGfAfgggaaacauaL96 | 1130 | usdAsugdTudTcccudCcUfgguugauscsa | 1298 |
| 1636779 | | | | | |
| AD- | SCN9A | asuscaucuuUfGfGfgucauucuuuL96 | 1131 | asdAsagdAadTgaccdCaAfagaugausasa | 1299 |
| 1636843 | | | | | |
| AD- | Marc1 | asuscugaugAfAfGfuauauuuuuuL96 | 1132 | asdAsaadAadTauacdTuCfaucagauscsu | 1300 |
| 1636772 | | | | | |
| AD- | SCN9A | asuscuucuuUfGfUfcguagugauuL96 | 1133 | asdAsucdAcdTacgadCaAfagaagauscsa | 1301 |
| 1636836 | | | | | |
| AD- | SCN9A | asusgcugagAfAfAfuugucgaaaaL96 | 1134 | usdTsuudCgdAcaaudTuCfucagcauscsu | 1302 |
| 1636813 | | | | | |
| AD- | Marc1 | asusggcuugUfUfCfcagaugcauuL96 | 1135 | asdAsugdCadTcuggdAaCfaagccauscsa | 1303 |
| 1636783 | | | | | |
| AD- | C3 | asusguaccaUfGfCfuaaggccaaaL96 | 1136 | usdTsugdGcdCuuagdCaUfgguacaususg | 1304 |
| 1636692 | | | | | |
| AD- | SCN9A | asusgucgagUfAfCfacuuuuacuuL96 | 1137 | asdAsgudAadAagugdTaCfucgacaususu | 1305 |
| 1636844 | | | | | |
| AD- | PNPLA3 | asusguuaguAfGfAfauaagccuuaL96 | 1138 | usdAsagdGcdTuauudCuAfcuaacauscsu | 1306 |
| 1636726 | | | | | |
| AD- | PNPLA3 | asusuaggauAfAfUfgucuuauguaL96 | 1139 | usdAscadTadAgacadTuAfuccuaausgsg | 1307 |
| 1636760 | | | | | |
| AD- | SCN9A | asusucucuuCfGfUfucacagaugaL96 | 1140 | usdCsaudCudGugaadCgAfagagaauscsc | 1308 |
| 1636841 | | | | | |
| AD- | Marc1 | csasacacuuGfAfAfgcaugguguuL96 | 1141 | asdAscadCcdAugcudTcAfaguguugsusc | 1309 |
| 1636789 | | | | | |
| AD- | PNPLA3 | csasagauuuGfCfAfacuugcuacaL96 | 1142 | usdGsuadGcdAaguudGcAfaaucuugscsu | 1310 |
| 1636762 | | | | | |
| AD- | C3 | csasaggucuAfCfGfccuauuacauL96 | 1143 | asdTsgudAadTaggcdGuAfgaccuugsasc | 1311 |
| 1636766 | | | | | |
| AD- | C3 | csascccucaUfCfAfucuaccuggaL96 | 1144 | usdCscadGgdTagaudGaUfgagggugsusu | 1312 |
| 1636718 | | | | | |
| AD- | C3 | csasccguauCfCfAfcugggaaucuL96 | 1145 | asdGsaudTcdCcagudGgAfuacggugsgsg | 1313 |
| 1636707 | | | | | |
| AD- | PNPLA3 | csasccuuuuUfCfAfccuaacuaaaL96 | 1146 | usdTsuadGudTaggudGaAfaaaggugsusu | 1314 |
| 1636727 | | | | | |
| AD- | PNPLA3 | csasccuuuuUfCfAfccuaacuaauL96 | 1147 | asdTsuadGudTaggudGaAfaaaggugsusu | 1315 |
| 1636741 | | | | | |
| AD- | SCN9A | csascuccuuCfCfUfgauuguguuuL96 | 1148 | asdAsacdAcdAaucadGgAfaggagugsgsa | 1316 |
| 1636830 | | | | | |
| AD- | Marc1 | csasgaacgaAfAfGfuuauauggaaL96 | 1149 | usdTsccdAudAuaacdTuUfcguucugsasa | 1317 |
| 1636780 | | | | | |
| AD- | C3 | csasgagaaaUfUfCfuacuacaucuL96 | 1150 | asdGsaudGudAguagdAaUfuucucugsusa | 1318 |
| 1636689 | | | | | |
| AD- | SCN9A | csasugaacgAfCfUfucuuccacuuL96 | 1151 | asdAsgudGgdAagaadGuCfguucaugsusg | 1319 |
| 1636839 | | | | | |
| AD- | PNPLA3 | csasugagcaAfGfAfuuugcaacuuL96 | 1152 | asdAsgudTgdCaaaudCuUfgcucaugsusa | 1320 |
| 1636753 | | | | | |
| AD- | Marc1 | csasugguguUfUfCfagaacugagaL96 | 1153 | usdCsucdAgdTucugdAaAfcaccaugscsu | 1321 |
| 1636792 | | | | | |
| AD- | C3 | csasuugagaAfCfCfcggaaggcauL96 | 1154 | asdTsgcdCudTccggdGuUfcucaaugsusu | 1322 |
| 1636709 | | | | | |
| AD- | C3 | cscsacagccAfAfAfgauaagaacuL96 | 1155 | asdGsuudCudTaucudTuGfgcuguggsusc | 1323 |
| 1636721 | | | | | |
| AD- | C3 | cscsagauccAfCfUfucaccaagauL96 | 1156 | asdTscudTgdGugaadGuGfgaucuggsusa | 1324 |
| 1636719 | | | | | |
| AD- | PNPLA3 | cscsauuaggAfUfAfaugucuuauuL96 | 1157 | asdAsuadAgdAcauudAuCfcuaauggsgsu | 1325 |
| 1636765 | | | | | |
| AD- | C3 | cscscgucguGfCfGfuuggcucaauL96 | 1158 | asdTsugdAgdCcaacdGcAfcgacgggsasg | 1326 |
| 1636693 | | | | | |
| AD | Marc1 | cscsgacccaAfGfGfaccagauuguL96 | 1159 | asdCsaadTcdTggucdCuUfgggucggsasa | 1327 |
| 1636807 | | | | | |
| AD- | C3 | cscsgagccgUfUfCfucuacaauuuL96 | 1160 | asdAsaudTgdTagagdAaCfggcucggsasu | 1328 |
| 1479283 | | | | | |
| AD- | Marc1 | cscsguauguCfCfUfggaauauuauL96 | 1161 | asdTsaadTadTuccadGgAfcauacggsusu | 1329 |
| 1636774 | | | | | |
| AD- | PNPLA3 | cscsuaacuaAfAfAfuaauguuuaaL96 | 1162 | usdTsaadAcdAuuaudTuUfaguuaggsusg | 1330 |
| 1636725 | | | | | |
| AD- | C3 | cscsuugucuUfCfUfcagaaccagaL96 | 1163 | usdCsugdGudTcugadGaAfgacaaggsasg | 1331 |
| 1636715 | | | | | |
| AD- | PNPLA3 | csgsacaucuGfCfCfcuaaagucaaL96 | 1164 | usdTsgadCudTuaggdGcAfgaugucgsusa | 1332 |
| 1636750 | | | | | |
| AD- | C3 | csgsugcguuGfGfCfucaaugaacuL96 | 1165 | asdGsuudCadTugagdCcAfacgcacgsasc | 1333 |
| 1636702 | | | | | |
| AD- | C3 | csusacccuaCfUfCfuguuguucgaL96 | 1166 | usdCsgadAcdAacagdAgUfaggguagscsc | 1334 |
| 1636705 | | | | | |
| AD- | C3 | csusacugcaGfCfUfaaaagacuuuL96 | 1167 | asdAsagdTcdTuuuadGcUfgcaguagsgsg | 1335 |
| 1636690 | | | | | |
| AD- | C3 | csusagugcuGfUfCfcagugagaaaL96 | 1168 | usdTsucdTcdAcuggdAcAfgcacuagsusu | 1336 |
| 1636694 | | | | | |
| AD- | PNPLA3 | csusauuaauGfGfUfcagacuguuaL96 | 1169 | usdAsacdAgdTcugadCcAfuuaauagsgsg | 1337 |
| 1636743 | | | | | |
| AD | PNPLA3 | csusccauggCfGfGfggguaacaaaL96 | 1170 | usdTsugdTudAccccdCgCfcauggagsasc | 1338 |
| 1636756 | | | | | |
| AD- | PNPLA3 | csusgaguugGfUfUfuuaugaaaauL96 | 1171 | asdTsuudTcdAuaaadAcCfaacucagscsu | 1339 |
| 1636734 | | | | | |
| AD- | SCN9A | csusgcccaaAfAfUfacugauaauaL96 | 1172 | usdAsuudAudCaguadTuUfugggcagscsa | 1340 |
| 1636840 | | | | | |
| AD- | C3 | csusgggaggAfCfCfcugguaagcaL96 | 1173 | usdGscudTadCcaggdGuCfcucccagscsg | 1341 |
| 1636712 | | | | | |
| AD- | Marc1 | csusguggagGfAfGfaagaaaaguaL96 | 1174 | usdAscudTudTcuucdTcCfuccacagsasa | 1342 |
| 1636800 | | | | | |
| AD- | Marc1 | csusucuuauUfGfGfugacguggaaL96 | 1175 | usdTsccdAcdGucacdCaAfuaagaagscsu | 1343 |
| 1636790 | | | | | |
| AD- | Marc1 | csusuguuccAfGfAfugcauuuuaaL96 | 1176 | usdTsaadAadTgcaudCuGfgaacaagscsc | 1344 |
| 1636770 | | | | | |
| AD- | SCN9A | csusuugucgUfAfGfugauuuuccuL96 | 1177 | asdGsgadAadAucacdTaCfgacaaagsasa | 1345 |
| 1636837 | | | | | |
| AD- | C3 | gsascagacaAfGfAfccaucuacauL96 | 1178 | asdTsgudAgdAuggudCuUfgucugucsusg | 1346 |
| 1636699 | | | | | |
| AD- | C3 | gsasgaaauuCfUfAfcuacaucuauL96 | 1179 | asdTsagdAudGuagudAgAfauuucucsusg | 1347 |
| 1479341 | | | | | |
| AD- | C3 | gsasgaaccaGfAfAfacaaugccauL96 | 1180 | asdTsggdCadTuguudTcUfgguucucsusu | 1348 |
| 1636691 | | | | | |
| AD- | SCN9A | gsasggucaaGfAfCfaucuuuaugaL96 | 1181 | usdCsaudAadAgaugdTcUfugaccucscsa | 1349 |
| 1636815 | | | | | |
| AD- | Marc1 | gsasgugcucCfUfUfcuccaguucuL96 | 1182 | asdGsaadCudGgagadAgGfagcacucscsg | 1350 |
| 1636795 | | | | | |
| AD- | PNPLA3 | gsasuuugcaAfCfUfugcuacccauL96 | 1183 | asdTsggdGudAgcaadGuUfgcaaaucsusu | 1351 |
| 1636731 | | | | | |
| AD- | PNPLA3 | gscsacagggAfAfCfcucuaccuuaL96 | 1184 | usdAsagdGudAgaggdTuCfccugugcsasg | 1352 |
| 1636744 | | | | | |
| AD- | C3 | gscsaggcagAfGfCfgcagcgggauL96 | 1185 | asdTsccdCgdCugcgdCuCfugccugcsasc | 1353 |
| 1636717 | | | | | |
| AD- | Marc1 | gscscauuccCfCfUfcagcuaaugaL96 | 1186 | usdCsaudTadGcugadGgGfgaauggcsasa | 1354 |
| 1636796 | | | | | |
| AD- | C3 | gscscucuucUfUfAfacaaauuucuL96 | 1187 | asdGsaadAudTuguudAaGfaagaggcscsc | 1355 |
| 1636710 | | | | | |
| AD- | SCN9A | gscsguuguaGfUfUfccuaucuccuL96 | 1188 | asdGsgadGadTaggadAcUfacaacgcscsu | 1356 |
| 1636822 | | | | | |
| AD- | SCN9A | gscsucaucaUfGfUfgcacuauucuL96 | 1189 | asdGsaadTadGugcadCaUfgaugagcsasu | 1357 |
| 1636828 | | | | | |
| AD- | C3 | gscsugaggaGfAfAfuugcuucauuL96 | 1190 | asdAsugdAadGcaaudTcUfccucagcsasc | 1358 |
| 1636767 | | | | | |
| AD- | PNPLA3 | gscsugaguuGfGfUfuuuaugaaaaL96 | 1191 | usdTsuudCadTaaaadCcAfacucagcsusc | 1359 |
| 1636739 | | | | | |
| AD- | Marc1 | gscsuucucaGfAfCfagcauuggauL96 | 1192 | asdTsccdAadTgcugdTcUfgagaagcsasg | 1360 |
| 1636803 | | | | | |
| AD- | Marc1 | gsgsaccagaUfUfGfcuuacucagaL96 | 1193 | usdCsugdAgdTaagcdAaUfcugguccsusu | 1361 |
| 1636801 | | | | | |
| AD- | C3 | gsgsagguugUfGfCfugagccggaaL96 | 1194 | usdTsccdGgdCucagdCaCfaaccuccscsc | 1362 |
| 1636713 | | | | | |
| AD- | SCN9A | gsgscacaugAfAfCfgacuucuucaL96 | 1195 | usdGsaadGadAgucgdTuCfaugugccsasc | 1363 |
| 1636833 | | | | | |
| AD- | PNPLA3 | gsgsccuuauCfCfCfuccuuccuuaL96 | 1196 | usdAsagdGadAggagdGgAfuaaggccsasc | 1364 |
| 1636740 | | | | | |
| AD- | SCN9A | gsgscuggauUfUfCfcuaauuguuuL96 | 1197 | asdAsacdAadTuaggdAaAfuccagccsasa | 1365 |
| 1636823 | | | | | |
| AD- | SCN9A | gsgsgaaaacAfAfUfcuuccguuuaL96 | 1198 | usdAsaadCgdGaagadTuGfuuuucccsusu | 1366 |
| 1636827 | | | | | |
| AD- | PNPLA3 | gsgsgguaacAfAfGfaugauaaucuL96 | 1199 | asdGsaudTadTcaucdTuGfuuaccccscsg | 1367 |
| 1636749 | | | | | |
| AD- | Marc1 | gsgsugucucAfAfUfgcuucaauguL96 | 1200 | asdCsaudTgdAagcadTuGfagacaccsasg | 1368 |
| 1636799 | | | | | |
| AD- | C3 | gsusaaugcaGfGfAfcuucuucauuL96 | 1201 | asdAsugdAadGaagudCcUfgcauuacsusg | 1369 |
| 1636700 | | | | | |
| AD- | C3 | gsusaccucuUfCfAfuccagacaguL96 | 1202 | asdCsugdTcdTggaudGaAfgagguacscsc | 1370 |
| 1636706 | | | | | |
| AD- | Marc1 | gsusauaacuCfUfAfagaucugauuL96 | 1203 | asdAsucdAgdAucuudAgAfguuauacsasa | 1371 |
| 1636771 | | | | | |
| AD- | SCN9A | gsusccucuaAfGfAfagaauaucuaL96 | 1204 | usdAsgadTadTucuudCuUfagaggacsusg | 1372 |
| 1636826 | | | | | |
| AD- | Marc1 | gsusgacccuUfCfAfgaacgaaaguL96 | 1205 | asdCsuudTcdGuucudGaAfgggucacsasc | 1373 |
| 1636848 | | | | | |
| AD- | PNPLA3 | gsusgagugaCfAfAfcguacccuuaL96 | 1206 | usdAsagdGgdTacgudTgUfcacucacsusc | 1374 |
| 1636746 | | | | | |
| AD- | PNPLA3 | gsusgcuaaaGfUfUfucccaucuuuL96 | 1207 | asdAsagdAudGggaadAcUfuuagcacscsu | 1375 |
| 1636747 | | | | | |
| AD- | C3 | gsusgggagaAfGfUfucggccuagaL96 | 1208 | usdCsuadGgdCcgaadCuUfcucccacsusg | 1376 |
| 1636723 | | | | | |
| AD- | Marc1 | usasacucuaAfGfAfucugaugaauL96 | 1209 | asdTsucdAudCagaudCuUfagaguuasusa | 1377 |
| 1636847 | | | | | |
| AD- | SCN9A | usascaugauCfUfUfcuuugucguaL96 | 1210 | usdAscgdAcdAaagadAgAfucauguasgsg | 1378 |
| 1636824 | | | | | |
| AD- | PNPLA3 | usasccuguuGfAfAfuuuuguauuaL96 | 1211 | usdAsaudAcdAaaaudTcAfacagguasasc | 1379 |
| 1636748 | | | | | |
| AD- | C3 | usascgugcuGfCfCfcaguuucgaaL96 | 1212 | usdTscgdAadAcuggdGcAfgcacguascsu | 1380 |
| 1636704 | | | | | |
| AD- | PNPLA3 | usasggauaaUfGfUfcuuauguaauL96 | 1213 | asdTsuadCadTaagadCaUfuauccuasasu | 1381 |
| 1636724 | | | | | |
| AD- | SCN9A | usasugccaaAfAfUfccuuuuuauaL96 | 1214 | usdAsuadAadAaggadTuUfuggcauasgsa | 1382 |
| 1636832 | | | | | |
| AD- | SCN9A | usasugugaaAfCfAfaaccuuacgaL96 | 1215 | usdCsgudAadGguuudGuUfucacauasasu | 1383 |
| 1636835 | | | | | |
| AD- | Marc1 | usasuuguaaUfUfUfcaggaugcgaL96 | 1216 | usdCsgcdAudCcugadAaUfuacaauasusu | 1384 |
| 1636788 | | | | | |
| AD- | Marc1 | uscsaaugcuUfCfAfaugucccaguL96 | 1217 | asdCsugdGgdAcauudGaAfgcauugasgsa | 1385 |
| 1636787 | | | | | |
| AD- | PNPLA3 | uscsacuugaGfGfAfggcgagucuaL96 | 1218 | usdAsgadCudCgccudCcUfcaagugascsu | 1386 |
| 1636761 | | | | | |
| AD- | Marc1 | uscsagaacgAfAfAfguuauauggaL96 | 1219 | usdCscadTadTaacudTuCfguucugasasg | 1387 |
| 1636794 | | | | | |
| AD- | Marc1 | uscsaggaugCfGfAfugucuaugcaL96 | 1220 | usdGscadTadGacaudCgCfauccugasasa | 1388 |
| 1636804 | | | | | |
| AD- | Marc1 | uscscauagaUfCfUfggaucuggcaL96 | 1221 | usdGsccdAgdAuccadGaUfcuauggasasa | 1389 |
| 1636802 | | | | | |
| AD- | SCN9A | uscscauuguCfUfUfgacaucuuauL96 | 1222 | asdTsaadGadTgucadAgAfcaauggasusc | 1390 |
| 1636814 | | | | | |
| AD- | SCN9A | uscscucuaaGfAfAfgaauaucuauL96 | 1223 | asdTsagdAudAuucudTcUfuagaggascsu | 1391 |
| 1636812 | | | | | |
| AD- | SCN9A | uscscugcaaGfUfCfaaguuccaaaL96 | 1224 | usdTsugdGadAcuugdAcUfugcaggasasa | 1392 |
| 1636850 | | | | | |
| AD- | PNPLA3 | uscsugagcuGfAfGfuugguuuuauL96 | 1225 | asdTsaadAadCcaacdTcAfgcucagasgsg | 1393 |
| 1636738 | | | | | |
| AD- | Marc1 | usgsacccuuCfAfGfaacgaaaguuL96 | 1226 | asdAscudTudCguucdTgAfagggucascsa | 1394 |
| 1636797 | | | | | |
| AD- | PNPLA3 | usgsagugaaGfAfAfaugaaagacaL96 | 1227 | usdGsucdTudTcauudTcUfucacucasgsu | 1395 |
| 1636728 | | | | | |
| AD- | SCN9A | usgsauaguuAfCfCfuaguuugcaaL96 | 1228 | usdTsgcdAadAcuagdGuAfacuaucasasa | 1396 |
| 1636842 | | | | | |
| AD- | SCN9A | usgscagacaAfGfAfucuucacuuaL96 | 1229 | usdAsagdTgdAagaudCuUfgucugcasusa | 1397 |
| 1636852 | | | | | |
| AD- | PNPLA3 | usgsccaaaaCfAfAfccaucaccguL96 | 1230 | asdCsggdTgdAuggudTgUfuuuggcasusc | 1398 |
| 1636764 | | | | | |
| AD- | C3 | usgscgaucaGfAfAfgagaccaaguL96 | 1231 | asdCsuudGgdTcucudTcUfgaucgcasgsg | 1399 |
| 1636698 | | | | | |
| AD- | C3 | usgscuaaggCfCfAfaagaucaacuL96 | 1232 | asdGsuudGadTcuuudGgCfcuuagcasusg | 1400 |
| 1636697 | | | | | |
| AD- | SCN9A | usgscucuccAfUfAfuuggauaaaaL96 | 1233 | usdTsuudAudCcaaudAuGfgagagcasasu | 1401 |
| 1636816 | | | | | |
| AD- | SCN9A | usgscucuccUfUfUfgugguuucauL96 | 1234 | asdTsgadAadCcacadAaGfgagagcasusc | 1402 |
| 1636846 | | | | | |
| AD- | C3 | usgsgacuauGfUfGfuacaagaccuL96 | 1235 | asdGsgudCudTguacdAcAfuaguccascsu | 1403 |
| 1636711 | | | | | |
| AD- | Marc1 | usgsgaggagAfAfGfaaaagugauuL96 | 1236 | asdAsucdAcdTuuucdTuCfuccuccascsa | 1404 |
| 1636793 | | | | | |
| AD- | PNPLA3 | usgsgauacaUfGfAfgcaagauuuaL96 | 1237 | usdAsaadTcdTugcudCaUfguauccascsc | 1405 |
| 1636742 | | | | | |
| AD- | PNPLA3 | usgsggagagAfUfAfugccuucgaaL96 | 1238 | usdTscgdAadGgcaudAuCfucucccasgsc | 1406 |
| 1636755 | | | | | |
| AD- | C3 | usgsggcaacUfCfCfaacaauuacuL96 | 1239 | asdGsuadAudTguugdGaGfuugcccascsg | 1407 |
| 1636696 | | | | | |
| AD- | SCN9A | usgsggucauUfCfUfucacuuugaaL96 | 1240 | usdTscadAadGugaadGaAfugacccasasa | 1408 |
| 1636821 | | | | | |
| AD- | SCN9A | usgsgucuuuAfCfUfggaaucuuuuL96 | 1241 | asdAsaadGadTuccadGuAfaagaccasasa | 1409 |
| 1636851 | | | | | |
| AD- | PNPLA3 | usgsgugacaUfGfGfcuuccagauaL96 | 1242 | usdAsucdTgdGaagcdCaUfgucaccasgsu | 1410 |
| 1636751 | | | | | |
| AD- | SCN9A | usgsgugucaUfCfAfuagauaauuuL96 | 1243 | asdAsaudTadTcuaudGaUfgacaccasasu | 1411 |
| 1636818 | | | | | |
| AD- | Marc1 | usgsgugucuCfAfAfugcuucaauaL96 | 1244 | usdAsuudGadAgcaudTgAfgacaccasgsa | 1412 |
| 1636781 | | | | | |
| AD- | C3 | usgsuacaagAfCfCfcgacuggucaL96 | 1245 | usdGsacdCadGucggdGuCfuuguacascsa | 1413 |
| 1636722 | | | | | |
| AD- | SCN9A | usgsuaggagAfAfUfucacuuuucaL96 | 1246 | usdGsaadAadGugaadTuCfuccuacascsa | 1414 |
| 1636820 | | | | | |
| AD- | SCN9A | usgsuaggagAfAfUfucacuuuucuL96 | 1247 | asdGsaadAadGugaadTuCfuccuacascsa | 1415 |
| 1636817 | | | | | |
| AD- | SCN9A | usgsucgaguAfCfAfcuuuuacugaL96 | 1248 | usdCsagdTadAaagudGuAfcucgacasusu | 1416 |
| 1636825 | | | | | |
| AD- | C3 | usgsuucgugCfUfGfaauaagaaguL96 | 1249 | asdCsuudCudTauucdAgCfacgaacascsg | 1417 |
| 1636708 | | | | | |
| AD- | SCN9A | usgsuucuguCfUfGfaguguguuuaL96 | 1250 | usdAsaadCadCacucdAgAfcagaacascsa | 1418 |
| 1636819 | | | | | |
| AD- | Marc1 | usgsuuuaaaAfCfCfcaauaucuauL96 | 1251 | asdTsagdAudAuuggdGuUfuuaaacasasc | 1419 |
| 1636775 | | | | | |
| AD- | Marc1 | ususaaaacuGfUfGfaauaaauggaL96 | 1252 | usdCscadTudTauucdAcAfguuuuaasasa | 1420 |
| 1636784 | | | | | |
| AD- | SCN9A | ususaccuauCfUfCfugcuucaaguL96 | 1253 | asdCsuudGadAgcagdAgAfuagguaascsc | 1421 |
| 1636829 | | | | | |
| AD- | PNPLA3 | ususaccuguUfGfAfauuuuguauuL96 | 1254 | asdAsuadCadAaauudCaAfcagguaascsa | 1422 |
| 1636768 | | | | | |
| AD- | PNPLA3 | ususauguaaUfGfCfugcccuguaaL96 | 1255 | usdTsacdAgdGgcagdCaUfuacauaasgsa | 1423 |
| 1636745 | | | | | |
| AD- | SCN9A | ususccucaaGfGfAfaaaagauaaaL96 | 1256 | usdTsuadTcdTuuuudCcUfugaggaasasu | 1424 |
| 1636809 | | | | | |
| AD- | C3 | ususcgugcuGfAfAfuaagaagaauL96 | 1257 | asdTsucdTudCuuaudTcAfgcacgaascsa | 1425 |
| 1636688 | | | | | |
| AD- | Marc1 | ususgccauuUfUfGfuccuuugauuL96 | 1258 | asdAsucdAadAggacdAaAfauggcaasusa | 1426 |
| 1636773 | | | | | |
| AD- | PNPLA3 | ususgcuaccCfAfUfuaggauaauaL96 | 1259 | usdAsuudAudCcuaadTgGfguagcaasgsu | 1427 |
| 1636752 | | | | | |
| AD- | PNPLA3 | ususgguuuuAfUfGfaaaagcuagaL96 | 1260 | usdCsuadGcdTuuucdAuAfaaaccaascsu | 1428 |
| 1636759 | | | | | |
| AD- | Marc1 | ususguaauuUfCfAfggaugcgauaL96 | 1261 | usdAsucdGcdAuccudGaAfauuacaasusa | 1429 |
| 1636849 | | | | | |
| AD- | Marc1 | ususguuccaGfAfUfgcauuuuaauL96 | 1262 | asdTsuadAadAugcadTcUfggaacaasgsc | 1430 |
| 1636776 | | | | | |
| AD- | SCN9A | ususuaguacAfCfUfccuuauucauL96 | 1263 | asdTsgadAudAaggadGuGfuacuaaasasu | 1431 |
| 1636845 | | | | | |
| AD- | SCN9A | ususuaucauCfUfUfugggucauuaL96 | 1264 | usdAsaudGadCccaadAgAfugauaaasgsa | 1432 |
| 1636810 | | | | | |
| AD- | Marc1 | ususuccauaGfAfUfcuggaucugaL96 | 1265 | usdCsagdAudCcagadTcUfauggaaasasu | 1433 |
| 1636786 | | | | | |
| AD- | SCN9A | ususuguagaUfCfUfugcaauuacaL96 | 1266 | usdGsuadAudTgcaadGaUfcuacaaasasg | 1434 |
| 1636808 | | | | | |
| AD- | PNPLA3 | ususuuagaaCfAfCfcuuuuucacuL96 | 1267 | asdGsugdAadAaaggdTgUfucuaaaasusu | 1435 |
| 1636736 | | | | | |
| AD- | PNPLA3 | ususuuucacCfUfAfacuaaaauaaL96 | 1268 | usdTsaudTudTaguudAgGfugaaaaasgsg | 1436 |
| 1636730 | | | | | |

TABLE 6
More Exemplary dsRNA molecule according to some embodiments of the disclosure

| | | | SEQ | | SEQ |
|---|---|---|---|---|---|
| Duplex | | | ID | | ID |
| ID | Target | Sense sequence (5′→3′) | NO: | Antisense sequence (5′→3′) | NO: |
| AD- | AGT | asgsccugagGfGfCfcaccauccuuL96 | 1437 | asdAsggdAudGguggdCcCfucaggcuscsa | 1786 |
| 1657992 | | | | | |
| AD- | AGT | cscsugagggCfCfAfccauccucuuL96 | 1438 | asdAsgadGgdAuggudGgCfccucaggscsu | 1787 |
| 1657994 | | | | | |
| AD- | AGT | asgsggccacCfAfUfccucugccuuL96 | 1439 | asdAsggdCadGaggadTgGfuggcccuscsa | 1788 |
| 1657998 | | | | | |
| AD- | AGT | gsusgaccggGfUfGfuacauacacuL96 | 1440 | asdGsugdTadTguacdAcCfcggucacscsu | 1789 |
| 1658030 | | | | | |
| AD- | AGT | cscsggguguAfCfAfuacaccccuuL96 | 1441 | asdAsggdGgdTguaudGuAfcacccggsusc | 1790 |
| 1684491 | | | | | |
| AD- | AGT | csgsgguguaCfAfUfacaccccuuuL96 | 1442 | asdAsagdGgdGuguadTgUfacacccgsgsu | 1791 |
| 1684493 | | | | | |
| AD- | AGT | gsgsguguacAfUfAfcaccccuucuL96 | 1443 | asdGsaadGgdGgugudAuGfuacacccsgsg | 1792 |
| 1684495 | | | | | |
| AD- | AGT | gsgsuguacaUfAfCfaccccuuccuL96 | 1444 | asdGsgadAgdGggugdTaUfguacaccscsg | 1793 |
| 1684497 | | | | | |
| AD- | AGT | gsusguacauAfCfAfccccuuccauL96 | 1445 | asdTsggdAadGgggudGuAfuguacacscsc | 1794 |
| 1684499 | | | | | |
| AD- | AGT | usgsuacauaCfAfCfcccuuccacuL96 | 1446 | asdGsugdGadAggggdTgUfauguacascsc | 1795 |
| 1684501 | | | | | |
| AD- | AGT | gsusacauacAfCfCfccuuccaccuL96 | 1447 | asdGsgudGgdAagggdGuGfuauguacsasc | 1796 |
| 1684503 | | | | | |
| AD- | AGT | usascauacaCfCfCfcuuccaccuuL96 | 1448 | asdAsggdTgdGaaggdGgUfguauguascsa | 1797 |
| 1684505 | | | | | |
| AD- | AGT | ascsauacacCfCfCfuuccaccucuL96 | 1449 | asdGsagdGudGgaagdGgGfuguaugusasc | 1798 |
| 1684507 | | | | | |
| AD- | AGT | csasuacaccCfCfUfuccaccucguL96 | 1450 | asdCsgadGgdTggaadGgGfguguaugsusa | 1799 |
| 1684509 | | | | | |
| AD- | AGT | asusacacccCfUfUfccaccucguuL96 | 1451 | asdAscgdAgdGuggadAgGfgguguausgsu | 1800 |
| 1684511 | | | | | |
| AD- | AGT | cscscuuccaCfCfUfcgucauccauL96 | 1452 | asdTsggdAudGacgadGgUfggaagggsgsu | 1801 |
| 1684513 | | | | | |
| AD- | AGT | cscsuuccacCfUfCfgucauccacuL96 | 1453 | asdGsugdGadTgacgdAgGfuggaaggsgsg | 1802 |
| 1684515 | | | | | |
| AD- | AGT | csusuccaccUfCfGfucauccacauL96 | 1454 | asdTsgudGgdAugacdGaGfguggaagsgsg | 1803 |
| 1658032 | | | | | |
| AD- | AGT | ususccaccuCfGfUfcauccacaauL96 | 1455 | asdTsugdTgdGaugadCgAfgguggaasgsg | 1804 |
| 1658033 | | | | | |
| AD- | AGT | uscscaccucGfUfCfauccacaauuL96 | 1456 | asdAsuudGudGgaugdAcGfagguggasasg | 1805 |
| 1658034 | | | | | |
| AD- | AGT | cscsaccucgUfCfAfuccacaauguL96 | 1457 | asdCsaudTgdTggaudGaCfgagguggsasa | 1806 |
| 1658035 | | | | | |
| AD- | AGT | csasccucguCfAfUfccacaaugauL96 | 1458 | asdTscadTudGuggadTgAfcgaggugsgsa | 1807 |
| 1658036 | | | | | |
| AD- | AGT | cscsucgucaUfCfCfacaaugagauL96 | 1459 | asdTscudCadTugugdGaUfgacgaggsusg | 1808 |
| 1658038 | | | | | |
| AD- | AGT | csuscgucauCfCfAfcaaugagaguL96 | 1460 | asdCsucdTcdAuugudGgAfugacgagsgsu | 1809 |
| 1658039 | | | | | |
| AD- | AGT | uscsgucaucCfAfCfaaugagaguuL96 | 1461 | asdAscudCudCauugdTgGfaugacgasgsg | 1810 |
| 1658040 | | | | | |
| AD- | AGT | csgsucauccAfCfAfaugagaguauL96 | 1462 | asdTsacdTcdTcauudGuGfgaugacgsasg | 1811 |
| 1658041 | | | | | |
| AD- | AGT | gsuscauccaCfAfAfugagaguacuL96 | 1463 | asdGsuadCudCucaudTgUfggaugacsgsa | 1812 |
| 1658042 | | | | | |
| AD- | AGT | uscsauccacAfAfUfgagaguaccuL96 | 1464 | asdGsgudAcdTcucadTuGfuggaugascsg | 1813 |
| 1658043 | | | | | |
| AD- | AGT | csasuccacaAfUfGfagaguaccuuL96 | 1465 | asdAsggdTadCucucdAuUfguggaugsasc | 1814 |
| 1658044 | | | | | |
| AD- | AGT | asusccacaaUfGfAfgaguaccuguL96 | 1466 | asdCsagdGudAcucudCaUfuguggausgsa | 1815 |
| 1658045 | | | | | |
| AD- | AGT | uscscacaauGfAfGfaguaccuguuL96 | 1467 | asdAscadGgdTacucdTcAfuuguggasusg | 1816 |
| 1658046 | | | | | |
| AD- | AGT | cscsacaaugAfGfAfguaccuguguL96 | 1468 | asdCsacdAgdGuacudCuCfauuguggsasu | 1817 |
| 1658047 | | | | | |
| AD- | AGT | csascaaugaGfAfGfuaccugugauL96 | 1469 | asdTscadCadGguacdTcUfcauugugsgsa | 1818 |
| 1658048 | | | | | |
| AD- | AGT | ascsaaugagAfGfUfaccugugaguL96 | 1470 | asdCsucdAcdAgguadCuCfucauugusgsg | 1819 |
| 1658049 | | | | | |
| AD- | AGT | csasaugagaGfUfAfccugugagcuL96 | 1471 | asdGscudCadCaggudAcUfcucauugsusg | 1820 |
| 1658050 | | | | | |
| AD- | AGT | asasugagagUfAfCfcugugagcauL96 | 1472 | asdTsgcdTcdAcaggdTaCfucucauusgsu | 1821 |
| 1658051 | | | | | |
| AD- | AGT | asusgagaguAfCfCfugugagcaguL96 | 1473 | asdCsugdCudCacagdGuAfcucucaususg | 1822 |
| 1658052 | | | | | |
| AD- | AGT | usgsagaguaCfCfUfgugagcagcuL96 | 1474 | asdGscudGcdTcacadGgUfacucucasusu | 1823 |
| 1658053 | | | | | |
| AD- | AGT | gsasgaguacCfUfGfugagcagcuuL96 | 1475 | asdAsgcdTgdCucacdAgGfuacucucsasu | 1824 |
| 1658054 | | | | | |
| AD- | AGT | asgsaguaccUfGfUfgagcagcuguL96 | 1476 | asdCsagdCudGcucadCaGfguacucuscsa | 1825 |
| 1658055 | | | | | |
| AD- | AGT | gsasguaccuGfUfGfagcagcugguL96 | 1477 | asdCscadGcdTgcucdAcAfgguacucsusc | 1826 |
| 1658056 | | | | | |
| AD- | AGT | asgsuaccugUfGfAfgcagcuggcuL96 | 1478 | asdGsccdAgdCugcudCaCfagguacuscsu | 1827 |
| 1658057 | | | | | |
| AD- | AGT | gsusaccuguGfAfGfcagcuggcauL96 | 1479 | asdTsgcdCadGcugcdTcAfcagguacsusc | 1828 |
| 1658058 | | | | | |
| AD- | AGT | usasccugugAfGfCfagcuggcaauL96 | 1480 | asdTsugdCcdAgcugdCuCfacagguascsu | 1829 |
| 1658059 | | | | | |
| AD- | AGT | asasuggucgGfGfAfugcuggccauL96 | 1481 | asdTsggdCcdAgcaudCcCfgaccauusgsc | 1830 |
| 1658184 | | | | | |
| AD- | AGT | asusggucggGfAfUfgcuggccaauL96 | 1482 | asdTsugdGcdCagcadTcCfcgaccaususg | 1831 |
| 1658185 | | | | | |
| AD- | AGT | usgsgucgggAfUfGfcuggccaacuL96 | 1483 | asdGsuudGgdCcagcdAuCfccgaccasusu | 1832 |
| 1658186 | | | | | |
| AD- | AGT | gsgsucgggaUfGfCfuggccaacuuL96 | 1484 | asdAsgudTgdGccagdCaUfcccgaccsasu | 1833 |
| 1658187 | | | | | |
| AD- | AGT | gsuscgggauGfCfUfggccaacuuuL96 | 1485 | asdAsagdTudGgccadGcAfucccgacscsa | 1834 |
| 1658188 | | | | | |
| AD- | AGT | uscsgggaugCfUfGfgccaacuucuL96 | 1486 | asdGsaadGudTggccdAgCfaucccgascsc | 1835 |
| 1658189 | | | | | |
| AD- | AGT | csgsggaugcUfGfGfccaacuucuuL96 | 1487 | asdAsgadAgdTuggcdCaGfcaucccgsasc | 1836 |
| 1658190 | | | | | |
| AD- | AGT | gsgsgaugcuGfGfCfcaacuucuuuL96 | 1488 | asdAsagdAadGuuggdCcAfgcaucccsgsa | 1837 |
| 1658191 | | | | | |
| AD- | AGT | gsgsaugcugGfCfCfaacuucuuguL96 | 1489 | asdCsaadGadAguugdGcCfagcauccscsg | 1838 |
| 1658192 | | | | | |
| AD- | AGT | gsasugcuggCfCfAfacuucuugguL96 | 1490 | asdCscadAgdAaguudGgCfcagcaucscsc | 1839 |
| 1658193 | | | | | |
| AD- | AGT | gscsuggccaAfCfUfucuugggcuuL96 | 1491 | asdAsgcdCcdAagaadGuUfggccagcsasu | 1840 |
| 1658196 | | | | | |
| AD- | AGT | csusggccaaCfUfUfcuugggcuuuL96 | 1492 | asdAsagdCcdCaagadAgUfuggccagscsa | 1841 |
| 1658197 | | | | | |
| AD- | AGT | gscscaacuuCfUfUfgggcuuccguL96 | 1493 | asdCsggdAadGcccadAgAfaguuggcscsa | 1842 |
| 1658200 | | | | | |
| AD- | AGT | cscsaacuucUfUfGfggcuuccguuL96 | 1494 | asdAscgdGadAgcccdAaGfaaguuggscsc | 1843 |
| 1658201 | | | | | |
| AD- | AGT | csasacuucuUfGfGfgcuuccguauL96 | 1495 | asdTsacdGgdAagccdCaAfgaaguugsgsc | 1844 |
| 1658202 | | | | | |
| AD- | AGT | asascuucuuGfGfGfcuuccguauuL96 | 1496 | asdAsuadCgdGaagcdCcAfagaaguusgsg | 1845 |
| 1658203 | | | | | |
| AD- | AGT | ascsuucuugGfGfCfuuccguauauL96 | 1497 | asdTsaudAcdGgaagdCcCfaagaagususg | 1846 |
| 1658204 | | | | | |
| AD- | AGT | csusucuuggGfCfUfuccguauauuL96 | 1498 | asdAsuadTadCggaadGcCfcaagaagsusu | 1847 |
| 1658205 | | | | | |
| AD- | AGT | ususcuugggCfUfUfccguauauauL96 | 1499 | asdTsaudAudAcggadAgCfccaagaasgsu | 1848 |
| 1658206 | | | | | |
| AD- | AGT | uscsuugggcUfUfCfcguauauauuL96 | 1500 | asdAsuadTadTacggdAaGfcccaagasasg | 1849 |
| 1658207 | | | | | |
| AD- | AGT | csusugggcuUfCfCfguauauauguL96 | 1501 | asdCsaudAudAuacgdGaAfgcccaagsasa | 1850 |
| 1658208 | | | | | |
| AD- | AGT | ususgggcuuCfCfGfuauauaugguL96 | 1502 | asdCscadTadTauacdGgAfagcccaasgsa | 1851 |
| 1658209 | | | | | |
| AD- | AGT | gsgsgcuuccGfUfAfuauauggcauL96 | 1503 | asdTsgcdCadTauaudAcGfgaagcccsasa | 1852 |
| 1658211 | | | | | |
| AD- | AGT | gsgscuuccgUfAfUfauauggcauuL96 | 1504 | asdAsugdCcdAuauadTaCfggaagccscsa | 1853 |
| 1658212 | | | | | |
| AD- | AGT | gscsuuccguAfUfAfuauggcauguL96 | 1505 | asdCsaudGcdCauaudAuAfcggaagcscsc | 1854 |
| 1658213 | | | | | |
| AD- | AGT | usasuauaugGfCfAfugcacaguguL96 | 1506 | asdCsacdTgdTgcaudGcCfauauauascsg | 1855 |
| 1658220 | | | | | |
| AD- | AGT | asusauauggCfAfUfgcacagugauL96 | 1507 | asdTscadCudGugcadTgCfcauauausasc | 1856 |
| 1658221 | | | | | |
| AD- | AGT | usasuauggcAfUfGfcacagugaguL96 | 1508 | asdCsucdAcdTgugcdAuGfccauauasusa | 1857 |
| 1658222 | | | | | |
| AD- | AGT | asusauggcaUfGfCfacagugagcuL96 | 1509 | asdGscudCadCugugdCaUfgccauausasu | 1858 |
| 1658223 | | | | | |
| AD- | AGT | usasuggcauGfCfAfcagugagcuuL96 | 1510 | asdAsgcdTcdAcugudGcAfugccauasusa | 1859 |
| 1658224 | | | | | |
| AD- | AGT | asusggcaugCfAfCfagugagcuauL96 | 1511 | asdTsagdCudCacugdTgCfaugccausasu | 1860 |
| 1658225 | | | | | |
| AD- | AGT | usgsgcaugcAfCfAfgugagcuauuL96 | 1512 | asdAsuadGcdTcacudGuGfcaugccasusa | 1861 |
| 1658226 | | | | | |
| AD- | AGT | gsgscaugcaCfAfGfugagcuauguL96 | 1513 | asdCsaudAgdCucacdTgUfgcaugccsasu | 1862 |
| 1658227 | | | | | |
| AD- | AGT | gscsaugcacAfGfUfgagcuaugguL96 | 1514 | asdCscadTadGcucadCuGfugcaugcscsa | 1863 |
| 1658228 | | | | | |
| AD- | AGT | csasugcacaGfUfGfagcuauggguL96 | 1515 | asdCsccdAudAgcucdAcUfgugcaugscsc | 1864 |
| 1684517 | | | | | |
| AD- | AGT | asusgcacagUfGfAfgcuaugggguL96 | 1516 | asdCsccdCadTagcudCaCfugugcausgsc | 1865 |
| 1684519 | | | | | |
| AD- | AGT | cscsucucccCfAfAfcggcugucuuL96 | 1517 | asdAsgadCadGccgudTgGfggagaggsasc | 1866 |
| 1684521 | | | | | |
| AD- | AGT | usgsgcacccUfGfGfccucucucuuL96 | 1518 | asdAsgadGadGaggcdCaGfggugccasasa | 1867 |
| 1658242 | | | | | |
| AD- | AGT | gsgscacccuGfGfCfcucucucuauL96 | 1519 | asdTsagdAgdAgaggdCcAfgggugccsasa | 1868 |
| 1658243 | | | | | |
| AD- | AGT | gsascaggcuAfCfAfggcaauccuuL96 | 1520 | asdAsggdAudTgccudGuAfgccugucsasg | 1869 |
| 1658288 | | | | | |
| AD- | AGT | ascsaggcuaCfAfGfgcaauccuguL96 | 1521 | asdCsagdGadTugccdTgUfagccuguscsa | 1870 |
| 1658289 | | | | | |
| AD- | AGT | ususccuuggAfAfGfgacaagaacuL96 | 1522 | asdGsuudCudTguccdTuCfcaaggaascsa | 1871 |
| 1658313 | | | | | |
| AD- | AGT | cscsuuggaaGfGfAfcaagaacuguL96 | 1523 | asdCsagdTudCuugudCcUfuccaaggsasa | 1872 |
| 1658315 | | | | | |
| AD- | AGT | csusuggaagGfAfCfaagaacugcuL96 | 1524 | asdGscadGudTcuugdTcCfuuccaagsgsa | 1873 |
| 1658316 | | | | | |
| AD- | AGT | csasccugaaGfCfAfgccguuuguuL96 | 1525 | asdAscadAadCggcudGcUfucaggugscsa | 1874 |
| 1658448 | | | | | |
| AD- | AGT | csusgaagcaGfCfCfguuugugcauL96 | 1526 | asdTsgcdAcdAaacgdGcUfgcuucagsgsu | 1875 |
| 1658451 | | | | | |
| AD- | AGT | ususugugcaGfGfGfccuggcucuuL96 | 1527 | asdAsgadGcdCaggcdCcUfgcacaaascsg | 1876 |
| 1658463 | | | | | |
| AD- | AGT | ususgugcagGfGfCfcuggcucucuL96 | 1528 | asdGsagdAgdCcaggdCcCfugcacaasasc | 1877 |
| 1658464 | | | | | |
| AD- | AGT | usgsugcaggGfCfCfuggcucucuuL96 | 1529 | asdAsgadGadGccagdGcCfcugcacasasa | 1878 |
| 1658465 | | | | | |
| AD- | AGT | gsusgcagggCfCfUfggcucucuauL96 | 1530 | asdTsagdAgdAgccadGgCfccugcacsasa | 1879 |
| 1658466 | | | | | |
| AD- | AGT | usgscagggcCfUfGfgcucucuauuL96 | 1531 | asdAsuadGadGagccdAgGfcccugcascsa | 1880 |
| 1658467 | | | | | |
| AD- | AGT | ascsgcucucUfGfGfacuucacaguL96 | 1532 | asdCsugdTgdAagucdCaGfagagcgusgsg | 1881 |
| 1658484 | | | | | |
| AD- | AGT | csgscucucuGfGfAfcuucacagauL96 | 1533 | asdTscudGudGaagudCcAfgagagcgsusg | 1882 |
| 1658485 | | | | | |
| AD- | AGT | csusgagaagAfUfUfgacagguucuL96 | 1534 | asdGsaadCcdTgucadAuCfuucucagscsa | 1883 |
| 1658519 | | | | | |
| AD- | AGT | usgsagaagaUfUfGfacagguucauL96 | 1535 | asdTsgadAcdCugucdAaUfcuucucasgsc | 1884 |
| 1658520 | | | | | |
| AD- | AGT | gsasgaagauUfGfAfcagguucauuL96 | 1536 | asdAsugdAadCcugudCaAfucuucucsasg | 1885 |
| 1658521 | | | | | |
| AD- | AGT | asgsaagauuGfAfCfagguucauguL96 | 1537 | asdCsaudGadAccugdTcAfaucuucuscsa | 1886 |
| 1658522 | | | | | |
| AD- | AGT | gsasagauugAfCfAfgguucaugcuL96 | 1538 | asdGscadTgdAaccudGuCfaaucuucsusc | 1887 |
| 1658523 | | | | | |
| AD- | AGT | asasgauugaCfAfGfguucaugcauL96 | 1539 | asdTsgcdAudGaaccdTgUfcaaucuuscsu | 1888 |
| 1658524 | | | | | |
| AD- | AGT | asgsauugacAfGfGfuucaugcaguL96 | 1540 | asdCsugdCadTgaacdCuGfucaaucususc | 1889 |
| 1658525 | | | | | |
| AD- | AGT | gsasuugacaGfGfUfucaugcagguL96 | 1541 | asdCscudGcdAugaadCcUfgucaaucsusu | 1890 |
| 1658526 | | | | | |
| AD- | AGT | asusugacagGfUfUfcaugcaggcuL96 | 1542 | asdGsccdTgdCaugadAcCfugucaauscsu | 1891 |
| 1658527 | | | | | |
| AD- | AGT | ususgacaggUfUfCfaugcaggcuuL96 | 1543 | asdAsgcdCudGcaugdAaCfcugucaasusc | 1892 |
| 1658528 | | | | | |
| AD- | AGT | usgsacagguUfCfAfugcaggcuguL96 | 1544 | asdCsagdCcdTgcaudGaAfccugucasasu | 1893 |
| 1658529 | | | | | |
| AD- | AGT | gsascagguuCfAfUfgcaggcuguuL96 | 1545 | asdAscadGcdCugcadTgAfaccugucsasa | 1894 |
| 1658530 | | | | | |
| AD- | AGT | ascsagguucAfUfGfcaggcuguguL96 | 1546 | asdCsacdAgdCcugcdAuGfaaccuguscsa | 1895 |
| 1658531 | | | | | |
| AD- | AGT | asusgcaggcUfGfUfgacaggauguL96 | 1547 | asdCsaudCcdTgucadCaGfccugcausgsa | 1896 |
| 1658539 | | | | | |
| AD- | AGT | gscsaggcugUfGfAfcaggauggauL96 | 1548 | asdTsccdAudCcugudCaCfagccugcsasu | 1897 |
| 1658541 | | | | | |
| AD- | AGT | csasggcuguGfAfCfaggauggaauL96 | 1549 | asdTsucdCadTccugdTcAfcagccugscsa | 1898 |
| 1658542 | | | | | |
| AD- | AGT | gscsuuucaaCfAfCfcuacguccauL96 | 1550 | asdTsggdAcdGuaggdTgUfugaaagcscsa | 1899 |
| 1658605 | | | | | |
| AD- | AGT | gsasguucugGfGfUfggacaacaguL96 | 1551 | asdCsugdTudGuccadCcCfagaacucscsu | 1900 |
| 1658650 | | | | | |
| AD- | AGT | gsgsacaacaGfCfAfccucaguguuL96 | 1552 | asdAscadCudGaggudGcUfguuguccsasc | 1901 |
| 1658661 | | | | | |
| AD- | AGT | gsascaacagCfAfCfcucagugucuL96 | 1553 | asdGsacdAcdTgaggdTgCfuguugucscsa | 1902 |
| 1658662 | | | | | |
| AD- | AGT | ascsaacagcAfCfCfucagugucuuL96 | 1554 | asdAsgadCadCugagdGuGfcuguuguscsc | 1903 |
| 1658663 | | | | | |
| AD- | AGT | csasacagcaCfCfUfcagugucuguL96 | 1555 | asdCsagdAcdAcugadGgUfgcuguugsusc | 1904 |
| 1658664 | | | | | |
| AD | AGT | asascagcacCfUfCfagugucuguuL96 | 1556 | asdAscadGadCacugdAgGfugcuguusgsu | 1905 |
| 1658665 | | | | | |
| AD- | AGT | asusgccucuGfAfCfcuggacaaguL96 | 1557 | asdCsuudGudCcaggdTcAfgaggcausasg | 1906 |
| 1658801 | | | | | |
| AD- | AGT | asasgguggaGfGfGfucucacuuuuL96 | 1558 | asdAsaadGudGagacdCcUfccaccuusgsu | 1907 |
| 1658818 | | | | | |
| AD- | AGT | asgsguggagGfGfUfcucacuuucuL96 | 1559 | asdGsaadAgdTgagadCcCfuccaccususg | 1908 |
| 1658819 | | | | | |
| AD- | AGT | gsgsuggaggGfUfCfucacuuuccuL96 | 1560 | asdGsgadAadGugagdAcCfcuccaccsusu | 1909 |
| 1658820 | | | | | |
| AD- | AGT | gsasgggucuCfAfCfuuuccagcauL96 | 1561 | asdTsgcdTgdGaaagdTgAfgacccucscsa | 1910 |
| 1658824 | | | | | |
| AD- | AGT | asgsggucucAfCfUfuuccagcaauL96 | 1562 | asdTsugdCudGgaaadGuGfagacccuscsc | 1911 |
| 1658825 | | | | | |
| AD- | AGT | gsgsucucacUfUfUfccagcaaaauL96 | 1563 | asdTsuudTgdCuggadAaGfugagaccscsu | 1912 |
| 1658827 | | | | | |
| AD- | AGT | gsuscucacuUfUfCfcagcaaaacuL96 | 1564 | asdGsuudTudGcuggdAaAfgugagacscsc | 1913 |
| 1658828 | | | | | |
| AD- | AGT | uscsucacuuUfCfCfagcaaaacuuL96 | 1565 | asdAsgudTudTgcugdGaAfagugagascsc | 1914 |
| 1658829 | | | | | |
| AD- | AGT | csuscacuuuCfCfAfgcaaaacucuL96 | 1566 | asdGsagdTudTugcudGgAfaagugagsasc | 1915 |
| 1658830 | | | | | |
| AD- | AGT | uscsacuuucCfAfGfcaaaacuccuL96 | 1567 | asdGsgadGudTuugcdTgGfaaagugasgsa | 1916 |
| 1658831 | | | | | |
| AD- | AGT | csascuuuccAfGfCfaaaacucccuL96 | 1568 | asdGsggdAgdTuuugdCuGfgaaagugsasg | 1917 |
| 1658832 | | | | | |
| AD- | AGT | ascsuuuccaGfCfAfaaacucccuuL96 | 1569 | asdAsggdGadGuuuudGcUfggaaagusgsa | 1918 |
| 1658833 | | | | | |
| AD- | AGT | csusuuccagCfAfAfaacucccucuL96 | 1570 | asdGsagdGgdAguuudTgCfuggaaagsusg | 1919 |
| 1658834 | | | | | |
| AD- | AGT | ususuccagcAfAfAfacucccucauL96 | 1571 | asdTsgadGgdGaguudTuGfcuggaaasgsu | 1920 |
| 1658835 | | | | | |
| AD- | AGT | ususccagcaAfAfAfcucccucaauL96 | 1572 | asdTsugdAgdGgagudTuUfgcuggaasasg | 1921 |
| 1658836 | | | | | |
| AD- | AGT | uscscagcaaAfAfCfucccucaacuL96 | 1573 | asdGsuudGadGggagdTuUfugcuggasasa | 1922 |
| 1658837 | | | | | |
| AD- | AGT | cscsagcaaaAfCfUfcccucaacuuL96 | 1574 | asdAsgudTgdAgggadGuUfuugcuggsasa | 1923 |
| 1658838 | | | | | |
| AD- | AGT | csasgcaaaaCfUfCfccucaacuguL96 | 1575 | asdCsagdTudGagggdAgUfuuugcugsgsa | 1924 |
| 1658839 | | | | | |
| AD- | AGT | asgscaaaacUfCfCfcucaacugguL96 | 1576 | asdCscadGudTgaggdGaGfuuuugcusgsg | 1925 |
| 1658840 | | | | | |
| AD- | AGT | gscsaaaacuCfCfCfucaacuggauL96 | 1577 | asdTsccdAgdTugagdGgAfguuuugcsusg | 1926 |
| 1658841 | | | | | |
| AD- | AGT | csasaaacucCfCfUfcaacuggauuL96 | 1578 | asdAsucdCadGuugadGgGfaguuuugscsu | 1927 |
| 1658842 | | | | | |
| AD- | AGT | asasaacuccCfUfCfaacuggauguL96 | 1579 | asdCsaudCcdAguugdAgGfgaguuuusgsc | 1928 |
| 1658843 | | | | | |
| AD- | AGT | asasacucccUfCfAfacuggaugauL96 | 1580 | asdTscadTcdCaguudGaGfggaguuususg | 1929 |
| 1658844 | | | | | |
| AD- | AGT | asascucccuCfAfAfcuggaugaauL96 | 1581 | asdTsucdAudCcagudTgAfgggaguususu | 1930 |
| 1658845 | | | | | |
| AD- | AGT | ascsucccucAfAfCfuggaugaaguL96 | 1582 | asdCsuudCadTccagdTuGfagggagususu | 1931 |
| 1658846 | | | | | |
| AD- | AGT | csuscccucaAfCfUfggaugaagauL96 | 1583 | asdTscudTcdAuccadGuUfgagggagsusu | 1932 |
| 1658847 | | | | | |
| AD- | AGT | uscsccucaaCfUfGfgaugaagaauL96 | 1584 | asdTsucdTudCauccdAgUfugagggasgsu | 1933 |
| 1658848 | | | | | |
| AD- | AGT | cscscucaacUfGfGfaugaagaaauL96 | 1585 | asdTsuudCudTcaucdCaGfuugagggsasg | 1934 |
| 1658849 | | | | | |
| AD- | AGT | cscsucaacuGfGfAfugaagaaacuL96 | 1586 | asdGsuudTcdTucaudCcAfguugaggsgsa | 1935 |
| 1321390 | | | | | |
| AD- | AGT | csuscaacugGfAfUfgaagaaacuuL96 | 1587 | asdAsgudTudCuucadTcCfaguugagsgsg | 1936 |
| 1658850 | | | | | |
| AD- | AGT | asgsgaucuuAfUfGfaccugcagguL96 | 1588 | asdCscudGcdAggucdAuAfagauccususg | 1937 |
| 1658875 | | | | | |
| AD- | AGT | csusuaugacCfUfGfcaggaccuguL96 | 1589 | asdCsagdGudCcugcdAgGfucauaagsasu | 1938 |
| 1658880 | | | | | |
| AD- | AGT | csgsagcugaAfCfCfugcaaaaauuL96 | 1590 | asdAsuudTudTgcagdGuUfcagcucgsgsu | 1939 |
| 1658929 | | | | | |
| AD- | AGT | gsasgcugaaCfCfUfgcaaaaauuuL96 | 1591 | asdAsaudTudTugcadGgUfucagcucsgsg | 1940 |
| 1658930 | | | | | |
| AD- | AGT | asgscugaacCfUfGfcaaaaauuguL96 | 1592 | asdCsaadTudTuugcdAgGfuucagcuscsg | 1941 |
| 1658931 | | | | | |
| AD- | AGT | gscsugaaccUfGfCfaaaaauugauL96 | 1593 | asdTscadAudTuuugdCaGfguucagcsusc | 1942 |
| 1658932 | | | | | |
| AD- | AGT | csusgaaccuGfCfAfaaaauugaguL96 | 1594 | asdCsucdAadTuuuudGcAfgguucagscsu | 1943 |
| 1658933 | | | | | |
| AD- | AGT | usgsaaccugCfAfAfaaauugagcuL96 | 1595 | asdGscudCadAuuuudTgCfagguucasgsc | 1944 |
| 1658934 | | | | | |
| AD- | AGT | gsasaccugcAfAfAfaauugagcauL96 | 1596 | asdTsgcdTcdAauuudTuGfcagguucsasg | 1945 |
| 1658935 | | | | | |
| AD- | AGT | asasccugcaAfAfAfauugagcaauL96 | 1597 | asdTsugdCudCaauudTuUfgcagguuscsa | 1946 |
| 1658936 | | | | | |
| AD- | AGT | ascscugcaaAfAfAfuugagcaauuL96 | 1598 | asdAsuudGcdTcaaudTuUfugcaggususc | 1947 |
| 1658937 | | | | | |
| AD- | AGT | cscsugcaaaAfAfUfugagcaauguL96 | 1599 | asdCsaudTgdCucaadTuUfuugcaggsusu | 1948 |
| 1658938 | | | | | |
| AD- | AGT | csusgcaaaaAfUfUfgagcaaugauL96 | 1600 | asdTscadTudGcucadAuUfuuugcagsgsu | 1949 |
| 1658939 | | | | | |
| AD- | AGT | usgscaaaaaUfUfGfagcaaugacuL96 | 1601 | asdGsucdAudTgcucdAaUfuuuugcasgsg | 1950 |
| 1658940 | | | | | |
| AD- | AGT | gsasggugcuGfAfAfcagcauuuuuL96 | 1602 | asdAsaadAudGcugudTcAfgcaccucscsc | 1951 |
| 1658954 | | | | | |
| AD- | AGT | asgsgugcugAfAfCfagcauuuuuuL96 | 1603 | asdAsaadAadTgcugdTuCfagcaccuscsc | 1952 |
| 1658955 | | | | | |
| AD- | AGT | gsgsugcugaAfCfAfgcauuuuuuuL96 | 1604 | asdAsaadAadAugcudGuUfcagcaccsusc | 1953 |
| 1658956 | | | | | |
| AD- | AGT | gsusgcugaaCfAfGfcauuuuuuuuL96 | 1605 | asdAsaadAadAaugcdTgUfucagcacscsu | 1954 |
| 1658957 | | | | | |
| AD- | AGT | usgscugaacAfGfCfauuuuuuuuuL96 | 1606 | asdAsaadAadAaaugdCuGfuucagcascsc | 1955 |
| 1658958 | | | | | |
| AD- | AGT | gscsugaacaGfCfAfuuuuuuuuguL96 | 1607 | asdCsaadAadAaaaudGcUfguucagcsasc | 1956 |
| 1658959 | | | | | |
| AD- | AGT | csusgaacagCfAfUfuuuuuuugauL96 | 1608 | asdTscadAadAaaaadTgCfuguucagscsa | 1957 |
| 1658960 | | | | | |
| AD- | AGT | usgsagagagAfGfCfccacagaguuL96 | 1609 | asdAscudCudGugggdCuCfucucucasusc | 1958 |
| 1658992 | | | | | |
| AD- | AGT | asgsagagagCfCfCfacagagucuuL96 | 1610 | asdAsgadCudCugugdGgCfucucucuscsa | 1959 |
| 1658994 | | | | | |
| AD- | AGT | gsasgagagcCfCfAfcagagucuauL96 | 1611 | asdTsagdAcdTcugudGgGfcucucucsusc | 1960 |
| 1658995 | | | | | |
| AD- | AGT | gsasaccgccCfAfUfuccuguuuguL96 | 1612 | asdCsaadAcdAggaadTgGfgcgguucsasg | 1961 |
| 1659055 | | | | | |
| AD- | AGT | asasccgcccAfUfUfccuguuugcuL96 | 1613 | asdGscadAadCaggadAuGfggcgguuscsa | 1962 |
| 1659056 | | | | | |
| AD- | AGT | ascscgcccaUfUfCfcuguuugcuuL96 | 1614 | asdAsgcdAadAcaggdAaUfgggcggususc | 1963 |
| 1659057 | | | | | |
| AD- | AGT | cscsgcccauUfCfCfuguuugcuguL96 | 1615 | asdCsagdCadAacagdGaAfugggcggsusu | 1964 |
| 1659058 | | | | | |
| AD- | AGT | csgscccauuCfCfUfguuugcuguuL96 | 1616 | asdAscadGcdAaacadGgAfaugggcgsgsu | 1965 |
| 1659059 | | | | | |
| AD- | AGT | gscsccauucCfUfGfuuugcuguguL96 | 1617 | asdCsacdAgdCaaacdAgGfaaugggcsgsg | 1966 |
| 1659060 | | | | | |
| AD- | AGT | cscscauuccUfGfUfuugcuguguuL96 | 1618 | asdAscadCadGcaaadCaGfgaaugggscsg | 1967 |
| 1659061 | | | | | |
| AD- | AGT | cscsauuccuGfUfUfugcuguguauL96 | 1619 | asdTsacdAcdAgcaadAcAfggaauggsgsc | 1968 |
| 1659062 | | | | | |
| AD- | AGT | csasuuccugUfUfUfgcuguguauuL96 | 1620 | asdAsuadCadCagcadAaCfaggaaugsgsg | 1969 |
| 1659063 | | | | | |
| AD- | AGT | asusuccuguUfUfGfcuguguauguL96 | 1621 | asdCsaudAcdAcagcdAaAfcaggaausgsg | 1970 |
| 1659064 | | | | | |
| AD- | AGT | ususccuguuUfGfCfuguguaugauL96 | 1622 | asdTscadTadCacagdCaAfacaggaasusg | 1971 |
| 1659065 | | | | | |
| AD- | AGT | uscscuguuuGfCfUfguguaugauuL96 | 1623 | asdAsucdAudAcacadGcAfaacaggasasu | 1972 |
| 1659066 | | | | | |
| AD- | AGT | cscsuguuugCfUfGfuguaugaucuL96 | 1624 | asdGsaudCadTacacdAgCfaaacaggsasa | 1973 |
| 1659067 | | | | | |
| AD- | AGT | csusguuugcUfGfUfguaugaucauL96 | 1625 | asdTsgadTcdAuacadCaGfcaaacagsgsa | 1974 |
| 1659068 | | | | | |
| AD- | AGT | usgsuuugcuGfUfGfuaugaucaauL96 | 1626 | asdTsugdAudCauacdAcAfgcaaacasgsg | 1975 |
| 1659069 | | | | | |
| AD- | AGT | gsusuugcugUfGfUfaugaucaaauL96 | 1627 | asdTsuudGadTcauadCaCfagcaaacsasg | 1976 |
| 1659070 | | | | | |
| AD- | AGT | ususugcuguGfUfAfugaucaaaguL96 | 1628 | asdCsuudTgdAucaudAcAfcagcaaascsa | 1977 |
| 1659071 | | | | | |
| AD- | AGT | cscsccagucUfCfCfcaccuuuucuL96 | 1629 | asdGsaadAadGguggdGaGfacuggggsgsu | 1978 |
| 1684529 | | | | | |
| AD- | AGT | cscscagucuCfCfCfaccuuuucuuL96 | 1630 | asdAsgadAadAggugdGgAfgacugggsgsg | 1979 |
| 1684531 | | | | | |
| AD- | AGT | cscsagucucCfCfAfccuuuucuuuL96 | 1631 | asdAsagdAadAaggudGgGfagacuggsgsg | 1980 |
| 1684533 | | | | | |
| AD- | AGT | asgsucucccAfCfCfuuuucuucuuL96 | 1632 | asdAsgadAgdAaaagdGuGfggagacusgsg | 1981 |
| 1684535 | | | | | |
| AD- | AGT | gsuscucccaCfCfUfuuucuucuauL96 | 1633 | asdTsagdAadGaaaadGgUfgggagacsusg | 1982 |
| 1659162 | | | | | |
| AD- | hAGT | uscsucccacCfUfUfuucuucuaauL96 | 1634 | asdTsuadGadAgaaadAgGfugggagascsu | 1983 |
| 1321384 | | | | | |
| AD- | AGT | csuscccaccUfUfUfucuucuaauuL96 | 1635 | asdAsuudAgdAagaadAaGfgugggagsasc | 1984 |
| 1659163 | | | | | |
| AD- | AGT | uscsccaccuUfUfUfcuucuaauguL96 | 1636 | asdCsaudTadGaagadAaAfggugggasgsa | 1985 |
| 1659164 | | | | | |
| AD- | AGT | cscscaccuuUfUfCfuucuaaugauL96 | 1637 | asdTscadTudAgaagdAaAfaggugggsasg | 1986 |
| 1659165 | | | | | |
| AD- | AGT | cscsaccuuuUfCfUfucuaaugaguL96 | 1638 | asdCsucdAudTagaadGaAfaagguggsgsa | 1987 |
| 1659166 | | | | | |
| AD- | AGT | csasccuuuuCfUfUfcuaaugaguuL96 | 1639 | asdAscudCadTuagadAgAfaaaggugsgsg | 1988 |
| 1659167 | | | | | |
| AD- | AGT | ascscuuuucUfUfCfuaaugagucuL96 | 1640 | asdGsacdTcdAuuagdAaGfaaaaggusgsg | 1989 |
| 1659168 | | | | | |
| AD- | AGT | cscsguuucuCfCfUfuggucuaaguL96 | 1641 | asdCsuudAgdAccaadGgAfgaaacggscsu | 1990 |
| 1659208 | | | | | |
| AD- | AGT | csgsuuucucCfUfUfggucuaaguuL96 | 1642 | asdAscudTadGaccadAgGfagaaacgsgsc | 1991 |
| 1659209 | | | | | |
| AD- | AGT | gsusuucuccUfUfGfgucuaaguguL96 | 1643 | asdCsacdTudAgaccdAaGfgagaaacsgsg | 1992 |
| 1659210 | | | | | |
| AD- | AGT | gsusuugcugGfGfUfuuauuuuaguL96 | 1644 | asdCsuadAadAuaaadCcCfagcaaacsusg | 1993 |
| 1659282 | | | | | |
| AD- | AGT | ususugcuggGfUfUfuauuuuagauL96 | 1645 | asdTscudAadAauaadAcCfcagcaaascsu | 1994 |
| 1659283 | | | | | |
| AD- | AGT | ususgcugggUfUfUfauuuuagaguL96 | 1646 | asdCsucdTadAaauadAaCfccagcaasasc | 1995 |
| 1659284 | | | | | |
| AD- | AGT | usgscuggguUfUfAfuuuuagagauL96 | 1647 | asdTscudCudAaaaudAaAfcccagcasasa | 1996 |
| 1659285 | | | | | |
| AD- | AGT | gscsuggguuUfAfUfuuuagagaauL96 | 1648 | asdTsucdTcdTaaaadTaAfacccagcsasa | 1997 |
| 1659286 | | | | | |
| AD- | AGT | csusggguuuAfUfUfuuagagaauuL96 | 1649 | asdAsuudCudCuaaadAuAfaacccagscsa | 1998 |
| 1659287 | | | | | |
| AD- | AGT | usgsgguuuaUfUfUfuagagaauguL96 | 1650 | asdCsaudTcdTcuaadAaUfaaacccasgsc | 1999 |
| 1659288 | | | | | |
| AD- | AGT | gsgsguuuauUfUfUfagagaaugguL96 | 1651 | asdCscadTudCucuadAaAfuaaacccsasg | 2000 |
| 1659289 | | | | | |
| AD- | AGT | gsgsuuuauuUfUfAfgagaauggguL96 | 1652 | asdCsccdAudTcucudAaAfauaaaccscsa | 2001 |
| 1684537 | | | | | |
| AD- | AGT | gsusuuauuuUfAfGfagaaugggguL96 | 1653 | asdCsccdCadTucucdTaAfaauaaacscsc | 2002 |
| 1684539 | | | | | |
| AD- | AGT | ususuauuuuAfGfAfgaauggggguL96 | 1654 | asdCsccdCcdAuucudCuAfaaauaaascsc | 2003 |
| 1684541 | | | | | |
| AD- | AGT | asgsaaugggGfGfUfggggaggcauL96 | 1655 | asdTsgcdCudCcccadCcCfccauucuscsu | 2004 |
| 1684543 | | | | | |
| AD- | AGT | gsasauggggGfUfGfgggaggcaauL96 | 1656 | asdTsugdCcdTccccdAcCfcccauucsusc | 2005 |
| 1684545 | | | | | |
| AD- | AGT | asasugggggUfGfGfggaggcaaguL96 | 1657 | asdCsuudGcdCucccdCaCfccccauuscsu | 2006 |
| 1684547 | | | | | |
| AD- | AGT | asusggggguGfGfGfgaggcaagauL96 | 1658 | asdTscudTgdCcuccdCcAfcccccaususc | 2007 |
| 1684549 | | | | | |
| AD- | AGT | usgsggggugGfGfGfaggcaagaauL96 | 1659 | asdTsucdTudGccucdCcCfacccccasusu | 2008 |
| 1684551 | | | | | |
| AD- | AGT | gsgsggguggGfGfAfggcaagaacuL96 | 1660 | asdGsuudCudTgccudCcCfcacccccsasu | 2009 |
| 1684553 | | | | | |
| AD- | AGT | gsusggggagGfCfAfagaaccaguuL96 | 1661 | asdAscudGgdTucuudGcCfuccccacscsc | 2010 |
| 1684555 | | | | | |
| AD- | AGT | usgsgggaggCfAfAfgaaccaguguL96 | 1662 | asdCsacdTgdGuucudTgCfcuccccascsc | 2011 |
| 1684557 | | | | | |
| AD- | AGT | gsgsggaggcAfAfGfaaccaguguuL96 | 1663 | asdAscadCudGguucdTuGfccuccccsasc | 2012 |
| 1684559 | | | | | |
| AD- | AGT | gsgsgaggcaAfGfAfaccaguguuuL96 | 1664 | asdAsacdAcdTgguudCuUfgccucccscsa | 2013 |
| 1684560 | | | | | |
| AD- | AGT | gsgsaggcaaGfAfAfccaguguuuuL96 | 1665 | asdAsaadCadCuggudTcUfugccuccscsc | 2014 |
| 1684561 | | | | | |
| AD- | AGT | gsasggcaagAfAfCfcaguguuuauL96 | 1666 | asdTsaadAcdAcuggdTuCfuugccucscsc | 2015 |
| 1659290 | | | | | |
| AD- | AGT | asgsgcaagaAfCfCfaguguuuaguL96 | 1667 | asdCsuadAadCacugdGuUfcuugccuscsc | 2016 |
| 1659291 | | | | | |
| AD- | AGT | gsgscaagaaCfCfAfguguuuagcuL96 | 1668 | asdGscudAadAcacudGgUfucuugccsusc | 2017 |
| 1659292 | | | | | |
| AD- | AGT | gscsaagaacCfAfGfuguuuagcguL96 | 1669 | asdCsgcdTadAacacdTgGfuucuugcscsu | 2018 |
| 1659293 | | | | | |
| AD- | AGT | csasagaaccAfGfUfguuuagcgcuL96 | 1670 | asdGscgdCudAaacadCuGfguucuugscsc | 2019 |
| 1659294 | | | | | |
| AD- | AGT | asasgaaccaGfUfGfuuuagcgcguL96 | 1671 | asdCsgcdGcdTaaacdAcUfgguucuusgsc | 2020 |
| 1659295 | | | | | |
| AD- | AGT | asgsaaccagUfGfUfuuagcgcgguL96 | 1672 | asdCscgdCgdCuaaadCaCfugguucususg | 2021 |
| 1659296 | | | | | |
| AD- | AGT | gsasaccaguGfUfUfuagcgcggguL96 | 1673 | asdCsccdGcdGcuaadAcAfcugguucsusu | 2022 |
| 1659297 | | | | | |
| AD- | AGT | asasccagugUfUfUfagcgcgggauL96 | 1674 | asdTsccdCgdCgcuadAaCfacugguuscsu | 2023 |
| 1659298 | | | | | |
| AD- | AGT | csusguuccaAfAfAfagaauuccauL96 | 1675 | asdTsggdAadTucuudTuUfggaacagsusa | 2024 |
| 1659321 | | | | | |
| AD- | AGT | gsusuccaaaAfAfGfaauuccaacuL96 | 1676 | asdGsuudGgdAauucdTuUfuuggaacsasg | 2025 |
| 1659323 | | | | | |
| AD- | AGT | uscscaaaaaGfAfAfuuccaaccguL96 | 1677 | asdCsggdTudGgaaudTcUfuuuuggasasc | 2026 |
| 1659325 | | | | | |
| AD- | AGT | cscsaaaaagAfAfUfuccaaccgauL96 | 1678 | asdTscgdGudTggaadTuCfuuuuuggsasa | 2027 |
| 1659326 | | | | | |
| AD- | AGT | csasaaaagaAfUfUfccaaccgacuL96 | 1679 | asdGsucdGgdTuggadAuUfcuuuuugsgsa | 2028 |
| 1659327 | | | | | |
| AD- | AGT | asasaaagaaUfUfCfcaaccgaccuL96 | 1680 | asdGsgudCgdGuuggdAaUfucuuuuusgsg | 2029 |
| 1659328 | | | | | |
| AD- | AGT | asasaagaauUfCfCfaaccgaccauL96 | 1681 | asdTsggdTcdGguugdGaAfuucuuuususg | 2030 |
| 1659329 | | | | | |
| AD- | AGT | asasagaauuCfCfAfaccgaccaguL96 | 1682 | asdCsugdGudCgguudGgAfauucuuususu | 2031 |
| 1659330 | | | | | |
| AD- | AGT | asasgaauucCfAfAfccgaccagcuL96 | 1683 | asdGscudGgdTcggudTgGfaauucuususu | 2032 |
| 1659331 | | | | | |
| AD- | AGT | asgsaauuccAfAfCfcgaccagcuuL96 | 1684 | asdAsgcdTgdGucggdTuGfgaauucususu | 2033 |
| 1659332 | | | | | |
| AD- | AGT | gsasauuccaAfCfCfgaccagcuuuL96 | 1685 | asdAsagdCudGgucgdGuUfggaauucsusu | 2034 |
| 1659333 | | | | | |
| AD- | AGT | asasuuccaaCfCfGfaccagcuuguL96 | 1686 | asdCsaadGcdTggucdGgUfuggaauuscsu | 2035 |
| 1659334 | | | | | |
| AD- | AGT | asusuccaacCfGfAfccagcuuguuL96 | 1687 | asdAscadAgdCuggudCgGfuuggaaususc | 2036 |
| 1659335 | | | | | |
| AD- | AGT | ususccaaccGfAfCfcagcuuguuuL96 | 1688 | asdAsacdAadGcuggdTcGfguuggaasusu | 2037 |
| 1659336 | | | | | |
| AD- | AGT | uscscaaccgAfCfCfagcuuguuuuL96 | 1689 | asdAsaadCadAgcugdGuCfgguuggasasu | 2038 |
| 1659337 | | | | | |
| AD- | AGT | cscsaaccgaCfCfAfgcuuguuuguL96 | 1690 | asdCsaadAcdAagcudGgUfcgguuggsasa | 2039 |
| 1659338 | | | | | |
| AD- | AGT | csasaccgacCfAfGfcuuguuuguuL96 | 1691 | asdAscadAadCaagcdTgGfucgguugsgsa | 2040 |
| 1659339 | | | | | |
| AD- | AGT | asasccgaccAfGfCfuuguuuguguL96 | 1692 | asdCsacdAadAcaagdCuGfgucgguusgsg | 2041 |
| 1659340 | | | | | |
| AD- | AGT | ascscgaccaGfCfUfuguuugugauL96 | 1693 | asdTscadCadAacaadGcUfggucggususg | 2042 |
| 1659341 | | | | | |
| AD- | AGT | cscsgaccagCfUfUfguuugugaauL96 | 1694 | asdTsucdAcdAaacadAgCfuggucggsusu | 2043 |
| 1659342 | | | | | |
| AD- | AGT | csgsaccagcUfUfGfuuugugaaauL96 | 1695 | asdTsuudCadCaaacdAaGfcuggucgsgsu | 2044 |
| 1659343 | | | | | |
| AD- | AGT | gsasccagcuUfGfUfuugugaaacuL96 | 1696 | asdGsuudTcdAcaaadCaAfgcuggucsgsg | 2045 |
| 1659344 | | | | | |
| AD- | AGT | ascscagcuuGfUfUfugugaaacauL96 | 1697 | asdTsgudTudCacaadAcAfagcugguscsg | 2046 |
| 1659345 | | | | | |
| AD- | AGT | cscsagcuugUfUfUfgugaaacaauL96 | 1698 | asdTsugdTudTcacadAaCfaagcuggsusc | 2047 |
| 1659346 | | | | | |
| AD- | AGT | csasgcuuguUfUfGfugaaacaaauL96 | 1699 | asdTsuudGudTucacdAaAfcaagcugsgsu | 2048 |
| 1659347 | | | | | |
| AD- | AGT | asgscuuguuUfGfUfgaaacaaaauL96 | 1700 | asdTsuudTgdTuucadCaAfacaagcusgsg | 2049 |
| 1659348 | | | | | |
| AD- | AGT | gscsuuguuuGfUfGfaaacaaaaauL96 | 1701 | asdTsuudTudGuuucdAcAfaacaagcsusg | 2050 |
| 1659349 | | | | | |
| AD- | AGT | csusuguuugUfGfAfaacaaaaaauL96 | 1702 | asdTsuudTudTguuudCaCfaaacaagscsu | 2051 |
| 1659350 | | | | | |
| AD- | AGT | ususguuuguGfAfAfacaaaaaaguL96 | 1703 | asdCsuudTudTuguudTcAfcaaacaasgsc | 2052 |
| 1659351 | | | | | |
| AD- | AGT | usgsuucccuUfUfUfcaaguugaguL96 | 1704 | asdCsucdAadCuugadAaAfgggaacascsu | 2053 |
| 1659371 | | | | | |
| AD- | AGT | gsusucccuuUfUfCfaaguugagauL96 | 1705 | asdTscudCadAcuugdAaAfagggaacsasc | 2054 |
| 1659372 | | | | | |
| AD- | AGT | ususcccuuuUfCfAfaguugagaauL96 | 1706 | asdTsucdTcdAacuudGaAfaagggaascsa | 2055 |
| 1659373 | | | | | |
| AD- | AGT | csasaguugaGfAfAfcaaaaauuguL96 | 1707 | asdCsaadTudTuugudTcUfcaacuugsasa | 2056 |
| 1659382 | | | | | |
| AD- | AGT | asasguugagAfAfCfaaaaauugguL96 | 1708 | asdCscadAudTuuugdTuCfucaacuusgsa | 2057 |
| 1659383 | | | | | |
| AD- | AGT | asgsuugagaAfCfAfaaaauuggguL96 | 1709 | asdCsccdAadTuuuudGuUfcucaacususg | 2058 |
| 1659384 | | | | | |
| AD- | AGT | gsusugagaaCfAfAfaaauuggguuL96 | 1710 | asdAsccdCadAuuuudTgUfucucaacsusu | 2059 |
| 1659385 | | | | | |
| AD- | AGT | ususgagaacAfAfAfaauuggguuuL96 | 1711 | asdAsacdCcdAauuudTuGfuucucaascsu | 2060 |
| 1659386 | | | | | |
| AD- | AGT | usgsagaacaAfAfAfauuggguuuuL96 | 1712 | asdAsaadCcdCaauudTuUfguucucasasc | 2061 |
| 1659387 | | | | | |
| AD- | AGT | gsasgaacaaAfAfAfuuggguuuuuL96 | 1713 | asdAsaadAcdCcaaudTuUfuguucucsasa | 2062 |
| 1659388 | | | | | |
| AD- | AGT | asgsaacaaaAfAfUfuggguuuuauL96 | 1714 | asdTsaadAadCccaadTuUfuuguucuscsa | 2063 |
| 1659389 | | | | | |
| AD- | AGT | gsasacaaaaAfUfUfggguuuuaauL96 | 1715 | asdTsuadAadAcccadAuUfuuuguucsusc | 2064 |
| 1659390 | | | | | |
| AD- | AGT | asgsuauacaUfUfUfuugcauugcuL96 | 1716 | asdGscadAudGcaaadAaUfguauacususu | 2065 |
| 1659399 | | | | | |
| AD- | AGT | gsusauacauUfUfUfugcauugccuL96 | 1717 | asdGsgcdAadTgcaadAaAfuguauacsusu | 2066 |
| 1659400 | | | | | |
| AD- | AGT | usasuacauuUfUfUfgcauugccuuL96 | 1718 | asdAsggdCadAugcadAaAfauguauascsu | 2067 |
| 1659401 | | | | | |
| AD- | AGT | asusacauuuUfUfGfcauugccuuuL96 | 1719 | asdAsagdGcdAaugcdAaAfaauguausasc | 2068 |
| 1659402 | | | | | |
| AD- | AGT | asusuuuugcAfUfUfgccuucgguuL96 | 1720 | asdAsccdGadAggcadAuGfcaaaaausgsu | 2069 |
| 1659406 | | | | | |
| AD- | AGT | ususuuugcaUfUfGfccuucgguuuL96 | 1721 | asdAsacdCgdAaggcdAaUfgcaaaaasusg | 2070 |
| 1659407 | | | | | |
| AD- | AGT | ususuugcauUfGfCfcuucgguuuuL96 | 1722 | asdAsaadCcdGaaggdCaAfugcaaaasasu | 2071 |
| 1659408 | | | | | |
| AD- | AGT | ususugcauuGfCfCfuucgguuuguL96 | 1723 | asdCsaadAcdCgaagdGcAfaugcaaasasa | 2072 |
| 1659409 | | | | | |
| AD- | AGT | ususgcauugCfCfUfucgguuuguuL96 | 1724 | asdAscadAadCcgaadGgCfaaugcaasasa | 2073 |
| 1659410 | | | | | |
| AD- | AGT | usgscauugcCfUfUfcgguuuguauL96 | 1725 | asdTsacdAadAccgadAgGfcaaugcasasa | 2074 |
| 1659411 | | | | | |
| AD- | AGT | gscsauugccUfUfCfgguuuguauuL96 | 1726 | asdAsuadCadAaccgdAaGfgcaaugcsasa | 2075 |
| 1659412 | | | | | |
| AD- | AGT | csasuugccuUfCfGfguuuguauuuL96 | 1727 | asdAsaudAcdAaaccdGaAfggcaaugscsa | 2076 |
| 1659413 | | | | | |
| AD- | AGT | asusugccuuCfGfGfuuuguauuuuL96 | 1728 | asdAsaadTadCaaacdCgAfaggcaausgsc | 2077 |
| 1659414 | | | | | |
| AD- | AGT | ususgccuucGfGfUfuuguauuuauL96 | 1729 | asdTsaadAudAcaaadCcGfaaggcaasusg | 2078 |
| 1659415 | | | | | |
| AD- | AGT | usgsccuucgGfUfUfuguauuuaguL96 | 1730 | asdCsuadAadTacaadAcCfgaaggcasasu | 2079 |
| 1659416 | | | | | |
| AD- | AGT | gscscuucggUfUfUfguauuuaguuL96 | 1731 | asdAscudAadAuacadAaCfcgaaggcsasa | 2080 |
| 1659417 | | | | | |
| AD- | AGT | cscsuucgguUfUfGfuauuuaguguL96 | 1732 | asdCsacdTadAauacdAaAfccgaaggscsa | 2081 |
| 1659418 | | | | | |
| AD- | AGT | csusucgguuUfGfUfauuuaguguuL96 | 1733 | asdAscadCudAaauadCaAfaccgaagsgsc | 2082 |
| 1659419 | | | | | |
| AD- | AGT | ususcgguuuGfUfAfuuuagugucuL96 | 1734 | asdGsacdAcdTaaaudAcAfaaccgaasgsg | 2083 |
| 1659420 | | | | | |
| AD- | AGT | uscsgguuugUfAfUfuuagugucuuL96 | 1735 | asdAsgadCadCuaaadTaCfaaaccgasasg | 2084 |
| 1659421 | | | | | |
| AD- | AGT | csgsguuuguAfUfUfuagugucuuuL96 | 1736 | asdAsagdAcdAcuaadAuAfcaaaccgsasa | 2085 |
| 1659422 | | | | | |
| AD- | AGT | gsgsuuuguaUfUfUfagugucuuguL96 | 1737 | asdCsaadGadCacuadAaUfacaaaccsgsa | 2086 |
| 1659423 | | | | | |
| AD- | AGT | gsusuuguauUfUfAfgugucuugauL96 | 1738 | asdTscadAgdAcacudAaAfuacaaacscsg | 2087 |
| 1659424 | | | | | |
| AD- | AGT | ususuguauuUfAfGfugucuugaauL96 | 1739 | asdTsucdAadGacacdTaAfauacaaascsc | 2088 |
| 1659425 | | | | | |
| AD- | AGT | ususguauuuAfGfUfgucuugaauuL96 | 1740 | asdAsuudCadAgacadCuAfaauacaasasc | 2089 |
| 1659426 | | | | | |
| AD- | AGT | usgsuauuuaGfUfGfucuugaauguL96 | 1741 | asdCsaudTcdAagacdAcUfaaauacasasa | 2090 |
| 1659427 | | | | | |
| AD- | AGT | gsusauuuagUfGfUfcuugaauguuL96 | 1742 | asdAscadTudCaagadCaCfuaaauacsasa | 2091 |
| 1659428 | | | | | |
| AD- | AGT | usasuuuaguGfUfCfuugaauguauL96 | 1743 | asdTsacdAudTcaagdAcAfcuaaauascsa | 2092 |
| 1659429 | | | | | |
| AD- | AGT | asusuuagugUfCfUfugaauguaauL96 | 1744 | asdTsuadCadTucaadGaCfacuaaausasc | 2093 |
| 1659430 | | | | | |
| AD- | AGT | ususuaguguCfUfUfgaauguaaguL96 | 1745 | asdCsuudAcdAuucadAgAfcacuaaasusa | 2094 |
| 1659431 | | | | | |
| AD- | AGT | ususagugucUfUfGfaauguaagauL96 | 1746 | asdTscudTadCauucdAaGfacacuaasasu | 2095 |
| 1659432 | | | | | |
| AD- | AGT | usasgugucuUfGfAfauguaagaauL96 | 1747 | asdTsucdTudAcauudCaAfgacacuasasa | 2096 |
| 1659433 | | | | | |
| AD- | AGT | asgsugucuuGfAfAfuguaagaacuL96 | 1748 | asdGsuudCudTacaudTcAfagacacusasa | 2097 |
| 1659434 | | | | | |
| AD- | AGT | usgsucuugaAfUfGfuaagaacauuL96 | 1749 | asdAsugdTudCuuacdAuUfcaagacascsu | 2098 |
| 1659436 | | | | | |
| AD- | AGT | gsuscuugaaUfGfUfaagaacauguL96 | 1750 | asdCsaudGudTcuuadCaUfucaagacsasc | 2099 |
| 1659437 | | | | | |
| AD- | AGT | ususgaauguAfAfGfaacaugaccuL96 | 1751 | asdGsgudCadTguucdTuAfcauucaasgsa | 2100 |
| 1659440 | | | | | |
| AD- | AGT | gsusaagaacAfUfGfaccuccguguL96 | 1752 | asdCsacdGgdAggucdAuGfuucuuacsasu | 2101 |
| 1659446 | | | | | |
| AD- | AGT | usasagaacaUfGfAfccuccguguuL96 | 1753 | asdAscadCgdGaggudCaUfguucuuascsa | 2102 |
| 1659447 | | | | | |
| AD- | AGT | asasgaacauGfAfCfcuccguguauL96 | 1754 | asdTsacdAcdGgaggdTcAfuguucuusasc | 2103 |
| 1659448 | | | | | |
| AD- | AGT | asgsaacaugAfCfCfuccguguaguL96 | 1755 | asdCsuadCadCggagdGuCfauguucususa | 2104 |
| 1659449 | | | | | |
| AD- | AGT | gsasacaugaCfCfUfccguguaguuL96 | 1756 | asdAscudAcdAcggadGgUfcauguucsusu | 2105 |
| 1659450 | | | | | |
| AD- | AGT | asascaugacCfUfCfcguguaguguL96 | 1757 | asdCsacdTadCacggdAgGfucauguuscsu | 2106 |
| 1659451 | | | | | |
| AD- | AGT | ascsaugaccUfCfCfguguaguguuL96 | 1758 | asdAscadCudAcacgdGaGfgucaugususc | 2107 |
| 1659452 | | | | | |
| AD- | AGT | csasugaccuCfCfGfuguagugucuL96 | 1759 | asdGsacdAcdTacacdGgAfggucaugsusu | 2108 |
| 1659453 | | | | | |
| AD- | AGT | asusgaccucCfGfUfguagugucuuL96 | 1760 | asdAsgadCadCuacadCgGfaggucausgsu | 2109 |
| 1659454 | | | | | |
| AD- | AGT | csusuaguuuUfUfUfccacagauguL96 | 1761 | asdCsaudCudGuggadAaAfaacuaagsgsu | 2110 |
| 1659481 | | | | | |
| AD- | AGT | ususaguuuuUfUfCfcacagaugcuL96 | 1762 | asdGscadTcdTguggdAaAfaaacuaasgsg | 2111 |
| 1659482 | | | | | |
| AD- | AGT | usasguuuuuUfCfCfacagaugcuuL96 | 1763 | asdAsgcdAudCugugdGaAfaaaacuasasg | 2112 |
| 1659483 | | | | | |
| AD- | AGT | gsusuuuuucCfAfCfagaugcuuguL96 | 1764 | asdCsaadGcdAucugdTgGfaaaaaacsusa | 2113 |
| 1659485 | | | | | |
| AD- | AGT | ususuuuccaCfAfGfaugcuuguguL96 | 1765 | asdCsacdAadGcaucdTgUfggaaaaasasc | 2114 |
| 1659487 | | | | | |
| AD- | AGT | ususuuccacAfGfAfugcuugugauL96 | 1766 | asdTscadCadAgcaudCuGfuggaaaasasa | 2115 |
| 1659488 | | | | | |
| AD- | AGT | ususuccacaGfAfUfgcuugugauuL96 | 1767 | asdAsucdAcdAagcadTcUfguggaaasasa | 2116 |
| 1659489 | | | | | |
| AD- | AGT | ususccacagAfUfGfcuugugauuuL96 | 1768 | asdAsaudCadCaagcdAuCfuguggaasasa | 2117 |
| 1659490 | | | | | |
| AD- | AGT | uscscacagaUfGfCfuugugauuuuL96 | 1769 | asdAsaadTcdAcaagdCaUfcuguggasasa | 2118 |
| 1659491 | | | | | |
| AD- | AGT | cscsacagauGfCfUfugugauuuuuL96 | 1770 | asdAsaadAudCacaadGcAfucuguggsasa | 2119 |
| 1659492 | | | | | |
| AD- | AGT | csascagaugCfUfUfgugauuuuuuL96 | 1771 | asdAsaadAadTcacadAgCfaucugugsgsa | 2120 |
| 1659493 | | | | | |
| AD- | AGT | ascscugaauUfUfCfuguuugaauuL96 | 1772 | asdAsuudCadAacagdAaAfuucaggusgsc | 2121 |
| 1659537 | | | | | |
| AD- | AGT | cscsugaauuUfCfUfguuugaauguL96 | 1773 | asdCsaudTcdAaacadGaAfauucaggsusg | 2122 |
| 1659538 | | | | | |
| AD- | AGT | gsgsaaccauAfGfCfugguuauuuuL96 | 1774 | asdAsaadTadAccagdCuAfugguuccsgsc | 2123 |
| 1659559 | | | | | |
| AD- | AGT | gsasaccauaGfCfUfgguuauuucuL96 | 1775 | asdGsaadAudAaccadGcUfaugguucscsg | 2124 |
| 1659560 | | | | | |
| AD- | AGT | asasccauagCfUfGfguuauuucuuL96 | 1776 | asdAsgadAadTaaccdAgCfuaugguuscsc | 2125 |
| 1659561 | | | | | |
| AD- | AGT | ascscauagcUfGfGfuuauuucucuL96 | 1777 | asdGsagdAadAuaacdCaGfcuauggususc | 2126 |
| 1659562 | | | | | |
| AD- | AGT | cscsauagcuGfGfUfuauuucuccuL96 | 1778 | asdGsgadGadAauaadCcAfgcuauggsusu | 2127 |
| 1659563 | | | | | |
| AD- | AGT | cscsuuguguUfAfGfuaauaaacguL96 | 1779 | asdCsgudTudAuuacdTaAfcacaaggsgsa | 2128 |
| 1659582 | | | | | |
| AD- | AGT | csusuguguuAfGfUfaauaaacguuL96 | 1780 | asdAscgdTudTauuadCuAfacacaagsgsg | 2129 |
| 1659583 | | | | | |
| AD- | AGT | ususguguuaGfUfAfauaaacgucuL96 | 1781 | asdGsacdGudTuauudAcUfaacacaasgsg | 2130 |
| 1659584 | | | | | |
| AD- | AGT | usgsuguuagUfAfAfuaaacgucuuL96 | 1782 | asdAsgadCgdTuuaudTaCfuaacacasasg | 2131 |
| 1659585 | | | | | |
| AD- | AGT | gsusguuaguAfAfUfaaacgucuuuL96 | 1783 | asdAsagdAcdGuuuadTuAfcuaacacsasa | 2132 |
| 1659586 | | | | | |
| AD- | AGT | usgsuuaguaAfUfAfaacgucuuguL96 | 1784 | asdCsaadGadCguuudAuUfacuaacascsa | 2133 |
| 1659587 | | | | | |
| AD- | AGT | gsusuaguaaUfAfAfacgucuugcuL96 | 1785 | asdGscadAgdAcguudTaUfuacuaacsasc | 2134 |
| 1659588 | | | | | |

In some embodiments, the dsRNA molecule is not a dsRNA molecule listed in Table 7.

TABLE 7
Additional dsRNA molecules

| | | SEQ | | SEQ |
|---|---|---|---|---|
| Duplex | | ID | | ID |
| ID | Sense sequence (5′→3′) | NO: | Antisense sequence (5′→3′) | NO: |
| AD- | cscsgcu(Chd)agGfUfUfcugcuuuuaaL96 | 2135 | VPusdTsaadAadGcagadAcCfudGagcggscsc | 2136 |
| 1289862.1 | | | | |
| AD- | uscsugagCfuGfAfGfuugguuuuauL96 | 193 | asdTsaadAadCcaacdTcdAgdCucagasgsg | 2137 |
| 1193318.1 | | | | |
| AD- | uscsugagCfuGfAfGfuugguuuuauL96 | 193 | asdTsaadAadCcaacdTcdAgdCucagascsc | 2138 |
| 1193320.1 | | | | |
| AD- | ususuuagAfaCfAfCfcuuuuucacuL96 | 235 | asdGsugdAadAaaggdTgUfuCfuaaaasusu | 2139 |
| 1193410.1 | | | | |
| AD- | ususuuagAfaCfAfCfcuuuuucacuL96 | 235 | asdGsugdAadAaaggdTgUfuCfuaaaascsc | 2140 |
| 1193413.1 | | | | |

Total RNA isolation using DYNABEADS mRNA Isolation Kit (Invitrogen™, part #: 610-12)

Cells were lysed in 75 μL of Lysis/Binding Buffer containing 3 μL of beads per well and mixed for 10 minutes on an electrostatic shaker. The washing steps were automated on a Biotek EL406, using a magnetic plate support. Beads were washed (in 90 μL) once in Buffer A, once in Buffer B, and twice in Buffer E, with aspiration steps in between. Following a final aspiration, complete 10 μL RT mixture was added to each well, as described below.

Cdna Synthesis Using ABI High Capacity cDNA Reverse Transcription Kit (Applied Biosystems, Foster City, CA, Cat #4368813)

A master mix of 1 μL 10× Buffer, 0.4 μL 25× dNTPs, 1 μL Random primers, 0.5 L Reverse Transcriptase, 0.5 μL RNase inhibitor and 6.6 μL of H₂O per reaction were added per well. Plates were sealed, agitated for 10 minutes on an electrostatic shaker, and then incubated at 37° C. for 2 hours. Following this, the plates were agitated at 80° C. for 8 minutes

### Real Time PCR

Two μL of cDNA were added to a master mix containing 0.5 μL of human GAPDH TaqMan Probe (4326317E), 0.5 μL human AGT (HsO0174854 ml), 2 μL nuclease-free water and 5 μL Lightcycler 480 probe master mix (Roche Cat #O4887301001) per well in a 384 well plates (Roche cat #O4887301001). Real time PCR was done in a LightCycler480 Real Time PCR system (Roche).

To calculate relative fold change, data were analyzed using the ΔΔCt method and normalized to assays performed with cells transfected with 10 nM AD-1955, or mock transfected cells.

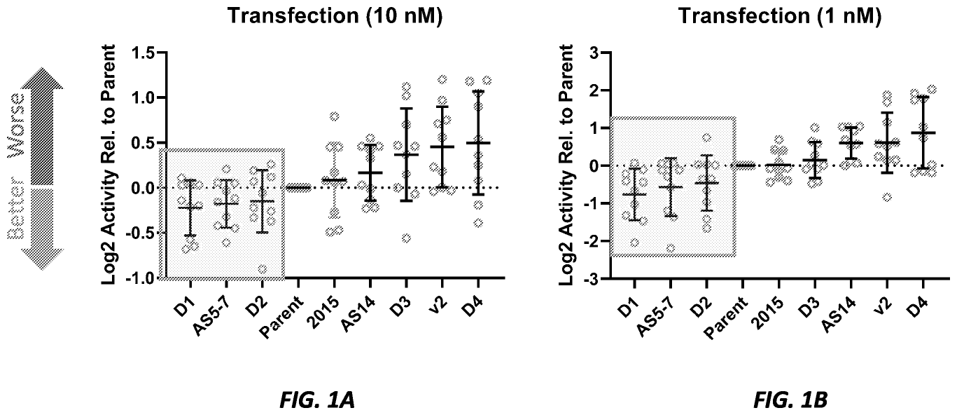

Results are shown in FIGS. 1A-1B and 3A-4C and summarized in Tables 8-14. The results show a consistently improved in vitro activity relative to the parent design across several exemplary designs and across a large set of exemplary sequences, targets, and cell lines.

TABLE 8
In vitro activity for various designs in primary mouse hepatocytes

| Parent | | | | | | | | | | | | |
|---|---|---|---|---|---|---|---|---|---|---|---|---|
| Duplex ID | Dose | | Parent | StDev | AS5-7 | StDev | AS14 | StDev | 2015 | StDev | D1 | StDev |
| AD-1181392 | 10 | nM | 4.2 | 0.5 | 4.9 | 0.3 | 5.6 | 0.5 | 4.7 | 0.6 | 4.4 | 0.3 |
| AD-1181401 | | | 7.4 | 1.0 | 5.9 | 1.2 | 10.9 | 3.2 | 6.1 | 0.5 | 4.6 | 0.2 |
| AD-1181410 | | | 5.8 | 0.3 | 4.4 | 0.5 | 5.8 | 0.9 | 6.1 | 1.1 | 6.0 | 0.7 |
| AD-1181417 | | | 5.8 | 0.8 | 6.1 | 1.8 | 8.1 | 1.9 | 10.1 | 2.3 | 6.3 | 1.4 |
| AD-1181426 | | | 6.2 | 1.5 | 6.4 | 0.2 | 5.3 | 0.7 | 6.4 | 0.6 | 4.2 | 0.7 |
| AD-64972 | | | 5.6 | 0.9 | 5.2 | 0.6 | 5.7 | 1.7 | 7.7 | 0.7 | 4.9 | 1.1 |
| AD-1181443 | | | 5.3 | 0.4 | 4.7 | 1.0 | 6.6 | 0.4 | 3.8 | 0.7 | 4.6 | 0.6 |
| AD-1181451 | | | 6.3 | 1.0 | 4.2 | 0.2 | 5.4 | 0.4 | 6.7 | 1.0 | 5.8 | 0.7 |
| AD-1181460 | | | 5.5 | 0.7 | 5.4 | 0.5 | 7.5 | 0.4 | 7.5 | 0.5 | 5.6 | 0.8 |
| AD-1181469 | | | 11.2 | 1.8 | 8.2 | 1.8 | 10.3 | 2.8 | 8.0 | 1.3 | 7.1 | 0.8 |
| AD-674282 | | | 5.3 | 4.9 | 10.9 | 0.9 | | | | | 27.0 | 10.3 |
| AD-674283 | | | 1.3 | 0.4 | 2.2 | 0.3 | | | | | 1.4 | 0.4 |
| AD-674287 | | | 4.1 | 1.2 | 4.5 | 0.7 | | | | | 12.4 | 2.0 |
| AD-674289 | | | 9.2 | 6.3 | 11.8 | 2.0 | | | | | 14.0 | 2.8 |
| AD-674293 | | | 3.1 | 3.4 | 7.7 | 1.7 | | | | | 11.0 | 3.8 |
| AD-674299 | | | 1.5 | 1.0 | 2.4 | 1.4 | | | | | 3.2 | 0.7 |
| AD-674303 | | | 29.5 | 6.4 | 26.3 | 4.4 | | | | | 29.3 | 4.6 |
| AD-674305 | | | 2.1 | 1.7 | 5.5 | 2.3 | | | | | 6.0 | 2.7 |
| AD-674307 | | | 2.9 | 3.0 | 2.3 | 1.0 | | | | | 3.1 | 0.2 |
| AD-674310 | | | 4.8 | 0.9 | 2.9 | 1.0 | | | | | 5.5 | 2.6 |
| AD-674312 | | | 6.3 | 3.7 | 4.0 | 1.4 | | | | | 5.0 | 2.3 |
| AD-674314 | | | 16.8 | 2.9 | 19.8 | 6.0 | | | | | 21.1 | 9.3 |
| AD-674323 | | | 21.4 | 5.9 | 16.0 | 1.9 | | | | | 30.8 | 6.8 |
| AD-674325 | | | 14.4 | 2.6 | 13.8 | 0.7 | | | | | 17.9 | 2.8 |
| AD-68435 | | | 4.2 | 2.0 | 1.9 | 0.5 | | | | | 4.9 | 1.4 |
| AD-1181392 | 1 | nM | 2.8 | 0.2 | 2.9 | 0.3 | 3.0 | 0.2 | 2.3 | 0.2 | 3.0 | 0.2 |
| AD-1181401 | | | 8.9 | 1.6 | 3.9 | 0.3 | 18.3 | 3.8 | 6.5 | 0.6 | 3.6 | 0.1 |
| AD-1181410 | | | 3.8 | 0.4 | 3.5 | 0.2 | 3.8 | 0.1 | 3.7 | 0.5 | 3.6 | 0.3 |
| AD-1181417 | | | 11.5 | 2.0 | 7.7 | 0.8 | 17.1 | 1.9 | 10.5 | 1.4 | 4.2 | 1.0 |
| AD-1181426 | | | 3.6 | 0.4 | 3.7 | 0.6 | 3.9 | 0.4 | 4.7 | 0.6 | 2.7 | 0.3 |
| AD-64972 | | | 5.3 | 0.5 | 4.3 | 0.4 | 10.8 | 2.5 | 8.6 | 2.2 | 4.6 | 0.7 |
| AD-1181443 | | | 4.5 | 0.5 | 4.7 | 1.1 | 6.5 | 1.3 | 3.4 | 0.5 | 3.3 | 0.4 |
| AD-1181451 | | | 10.8 | 1.9 | 9.9 | 1.5 | 17.2 | 3.0 | 10.2 | 2.3 | 6.8 | 1.1 |
| AD-1181460 | | | 18.7 | 2.0 | 4.1 | 0.5 | 35.5 | 1.9 | 19.9 | 2.0 | 4.5 | 0.6 |
| AD-1181469 | | | 33.5 | 3.1 | 13.1 | 2.4 | 68.6 | 2.5 | 44.9 | 1.6 | 16.5 | 1.5 |
| AD-674282 | | | 11.5 | 11.6 | 15.9 | 6.3 | | | | | 16.5 | 4.0 |
| AD-674283 | | | 11.8 | 6.2 | 5.2 | 1.6 | | | | | 3.5 | 0.4 |
| AD-674287 | | | 14.8 | 2.2 | 7.3 | 2.1 | | | | | 12.4 | 2.4 |
| AD-674289 | | | 67.0 | 11.6 | 29.3 | 6.8 | | | | | 19.0 | 4.5 |
| AD-674293 | | | 30.4 | 9.4 | 17.7 | 2.8 | | | | | 9.1 | 1.0 |
| AD-674299 | | | 16.0 | 4.4 | 7.7 | 1.4 | | | | | 4.4 | 1.2 |
| AD-674303 | | | 87.2 | 4.5 | 42.6 | 5.9 | | | | | 39.1 | 4.4 |
| AD-674305 | | | 28.8 | 8.2 | 8.5 | 1.5 | | | | | 6.7 | 1.4 |
| AD-674307 | | | 15.9 | 2.0 | 3.5 | 1.4 | | | | | 2.4 | 1.3 |
| AD-674310 | | | 18.7 | 4.5 | 5.9 | 1.9 | | | | | 4.8 | 2.4 |
| AD-674312 | | | 29.1 | 5.0 | 15.1 | 5.2 | | | | | 6.3 | 2.0 |
| AD-674314 | | | 36.5 | 9.1 | 20.1 | 6.3 | | | | | 15.9 | 3.2 |
| AD-674323 | | | 63.6 | 7.5 | 18.7 | 4.2 | | | | | 14.5 | 4.2 |
| AD-674325 | | | 36.9 | 6.4 | 16.1 | 2.5 | | | | | 11.4 | 1.7 |
| AD-68435 | | | 14.9 | 1.7 | 4.5 | 0.8 | | | | | 4.0 | 0.9 |
| AD-674282 | 0.1 | nM | 3.5 | 2.0 | 17.6 | 3.4 | | | | | 31.5 | 8.3 |
| AD-674283 | | | 5.9 | 2.9 | 9.7 | 3.5 | | | | | 7.5 | 1.5 |
| AD-674287 | | | 13.4 | 7.8 | 14.7 | 4.8 | | | | | 34.3 | 15.5 |
| AD-674289 | | | 53.5 | 6.6 | 42.0 | 7.3 | | | | | 52.0 | 16.3 |
| AD-674293 | | | 22.9 | 1.1 | 30.2 | 4.6 | | | | | 42.9 | 5.5 |
| AD-674299 | | | 13.5 | 4.3 | 9.3 | 1.3 | | | | | 17.9 | 7.1 |
| AD-674303 | | | 57.4 | 5.9 | 64.5 | 11.7 | | | | | 67.7 | 11.8 |
| AD-674305 | | | 25.7 | 4.9 | 15.2 | 7.6 | | | | | 32.2 | 11.3 |
| AD-674307 | | | 14.8 | 1.4 | 5.9 | 0.4 | | | | | 17.2 | 2.0 |
| AD-674310 | | | 23.3 | 11.8 | 13.7 | 3.8 | | | | | 33.6 | 7.0 |
| AD-674312 | | | 35.9 | 12.0 | 27.0 | 4.5 | | | | | 29.8 | 9.4 |
| AD-674314 | | | 34.9 | 2.5 | 41.3 | 10.2 | | | | | 60.8 | 9.8 |
| AD-674323 | | | 70.3 | 6.8 | 46.4 | 13.8 | | | | | 77.4 | 1.8 |
| AD-674325 | | | 28.8 | 2.4 | 32.1 | 11.7 | | | | | 51.6 | 10.9 |
| AD-68435 | | | 15.8 | 2.4 | 9.2 | 4.8 | | | | | 46.3 | 16.4 |

TABLE 9
In vitro activity for various designs in primary mouse hepatocytes

| Parent | | | | | | | | | | | | |
|---|---|---|---|---|---|---|---|---|---|---|---|---|
| Duplex ID | Dose | | Parent | StDev | D2 | StDev | D3 | StDev | D4 | StDev | v2 | StDev |
| AD-1181392 | 10 | nM | 4.2 | 0.5 | 5.0 | 0.1 | 9.1 | 1.2 | 9.6 | 0.3 | 7.0 | 0.4 |
| AD-1181401 | | | 7.4 | 1.0 | 6.2 | 1.8 | 8.2 | 0.8 | 7.8 | 1.2 | 7.4 | 0.5 |
| AD-1181410 | | | 5.8 | 0.3 | 4.7 | 0.2 | 5.8 | 0.8 | 7.5 | 0.2 | 5.7 | 1.5 |
| AD-1181417 | | | 5.8 | 0.8 | 5.0 | 1.4 | 11.8 | 2.1 | 12.1 | 1.5 | 8.6 | 0.6 |
| AD-1181426 | | | 6.2 | 1.5 | 5.9 | 0.8 | 5.9 | 0.8 | 9.0 | 2.1 | 7.2 | 1.8 |
| AD-64972 | | | 5.6 | 0.9 | 5.9 | 1.1 | 7.8 | 1.7 | 10.5 | 0.8 | 13.0 | 0.5 |
| AD-1181443 | | | 5.3 | 0.4 | 6.0 | 1.4 | 6.8 | 0.5 | 4.0 | 0.5 | 5.2 | 0.3 |
| AD-1181451 | | | 6.3 | 1.0 | 4.9 | 0.5 | 8.8 | 4.9 | 7.6 | 0.6 | 7.2 | 0.3 |
| AD-1181460 | | | 5.5 | 0.7 | 6.0 | 0.7 | 8.9 | 1.1 | 12.4 | 3.3 | 10.7 | 2.0 |
| AD-1181469 | | | 11.2 | 1.8 | 6.0 | 1.2 | 7.6 | 1.2 | 9.9 | 2.3 | 18.3 | 5.2 |
| AD-674282 | | | 5.3 | 4.9 | | | | | | | | |
| AD-674283 | | | 1.3 | 0.4 | | | | | | | | |
| AD-674287 | | | 4.1 | 1.2 | | | | | | | | |
| AD-674289 | | | 9.2 | 6.3 | | | | | | | | |
| AD-674293 | | | 3.1 | 3.4 | | | | | | | | |
| AD-674299 | | | 1.5 | 1.0 | | | | | | | | |
| AD-674303 | | | 29.5 | 6.4 | | | | | | | | |
| AD-674305 | | | 2.1 | 1.7 | | | | | | | | |
| AD-674307 | | | 2.9 | 3.0 | | | | | | | | |
| AD-674310 | | | 4.8 | 0.9 | | | | | | | | |
| AD-674312 | | | 6.3 | 3.7 | | | | | | | | |
| AD-674314 | | | 16.8 | 2.9 | | | | | | | | |
| AD-674323 | | | 21.4 | 5.9 | | | | | | | | |
| AD-674325 | | | 14.4 | 2.6 | | | | | | | | |
| AD-68435 | | | 4.2 | 2.0 | | | | | | | | |
| AD-1181392 | 1 | nM | 2.8 | 0.2 | 2.9 | 0.1 | 5.7 | 0.7 | 9.7 | 0.6 | 4.1 | 0.4 |
| AD-1181401 | | | 8.9 | 1.6 | 4.6 | 0.8 | 6.6 | 0.5 | 7.8 | 0.4 | 5.0 | 0.2 |
| AD-1181410 | | | 3.8 | 0.4 | 3.0 | 0.2 | 3.6 | 0.7 | 3.4 | 0.9 | 4.2 | 0.4 |
| AD-1181417 | | | 11.5 | 2.0 | 4.3 | 0.5 | 8.3 | 1.2 | 43.7 | 5.6 | 17.7 | 4.3 |
| AD-1181426 | | | 3.6 | 0.4 | 2.8 | 0.5 | 5.3 | 0.6 | 14.5 | 3.2 | 5.3 | 0.4 |
| AD-64972 | | | 5.3 | 0.5 | 5.4 | 2.3 | 8.3 | 0.8 | 17.7 | 1.1 | 19.3 | 1.5 |
| AD-1181443 | | | 4.5 | 0.5 | 4.5 | 0.5 | 5.4 | 0.3 | 4.0 | 0.5 | 4.9 | 0.8 |
| AD-1181451 | | | 10.8 | 1.9 | 18.0 | 5.2 | 11.1 | 0.2 | 11.0 | 1.0 | 34.9 | 3.8 |
| AD-1181460 | | | 18.7 | 2.0 | 5.9 | 0.7 | 17.6 | 2.0 | 38.2 | 6.6 | 22.1 | 2.6 |
| AD-1181469 | | | 33.5 | 3.1 | 21.2 | 0.9 | 33.8 | 0.8 | 56.1 | 2.2 | 74.3 | 5.8 |
| AD-674282 | | | 11.5 | 11.6 | | | | | | | | |
| AD-674283 | | | 11.8 | 6.2 | | | | | | | | |
| AD-674287 | | | 14.8 | 2.2 | | | | | | | | |
| AD-674289 | | | 67.0 | 11.6 | | | | | | | | |
| AD-674293 | | | 30.4 | 9.4 | | | | | | | | |
| AD-674299 | | | 16.0 | 4.4 | | | | | | | | |
| AD-674303 | | | 87.2 | 4.5 | | | | | | | | |
| AD-674305 | | | 28.8 | 8.2 | | | | | | | | |
| AD-674307 | | | 15.9 | 2.0 | | | | | | | | |
| AD-674310 | | | 18.7 | 4.5 | | | | | | | | |
| AD-674312 | | | 29.1 | 5.0 | | | | | | | | |
| AD-674314 | | | 36.5 | 9.1 | | | | | | | | |
| AD-674323 | | | 63.6 | 7.5 | | | | | | | | |
| AD-674325 | | | 36.9 | 6.4 | | | | | | | | |
| AD-68435 | | | 14.9 | 1.7 | | | | | | | | |
| AD-674282 | 0.1 | nM | 3.5 | 2.0 | | | | | | | | |
| AD-674283 | | | 5.9 | 2.9 | | | | | | | | |
| AD-674287 | | | 13.4 | 7.8 | | | | | | | | |
| AD-674289 | | | 53.5 | 6.6 | | | | | | | | |
| AD-674293 | | | 22.9 | 1.1 | | | | | | | | |
| AD-674299 | | | 13.5 | 4.3 | | | | | | | | |
| AD-674303 | | | 57.4 | 5.9 | | | | | | | | |
| AD-674305 | | | 25.7 | 4.9 | | | | | | | | |
| AD-674307 | | | 14.8 | 1.4 | | | | | | | | |
| AD-674310 | | | 23.3 | 11.8 | | | | | | | | |
| AD-674312 | | | 35.9 | 12.0 | | | | | | | | |
| AD-674314 | | | 34.9 | 2.5 | | | | | | | | |
| AD-674323 | | | 70.3 | 6.8 | | | | | | | | |
| AD-674325 | | | 28.8 | 2.4 | | | | | | | | |
| AD-68435 | | | 15.8 | 2.4 | | | | | | | | |

TABLE 10
In vitro activity for various designs in primary mouse hepatocytes

| Parent | | | | | | | | | | | | |
|---|---|---|---|---|---|---|---|---|---|---|---|---|
| Duplex ID | Dose | | Parent | StDev | D1 | StDev | D5 | StDev | D6 | StDev | D7 | StDev |
| AD-1181392 | 10 | nM | 1.2 | 0.1 | 0.9 | 0.1 | 1.8 | 0.3 | 8.6 | 1.2 | 2.8 | 0.3 |
| AD-1181401 | | | 24.2 | 2.3 | 4.5 | 1.1 | 7.4 | 1.1 | 19.1 | 1.1 | 8.5 | 2.1 |
| AD-1181410 | | | 5.3 | 1.9 | 1.7 | 0.4 | 2.2 | 0.5 | 3.5 | 1.2 | 2.5 | 0.3 |
| AD-1181417 | | | 15.4 | 3.6 | 5.5 | 1.6 | 9.8 | 2.6 | 59.8 | 21.7 | 13.6 | 4.3 |
| AD-1181426 | | | 2.6 | 0.6 | 1.4 | 0.3 | 4.5 | 0.7 | 9.7 | 1.5 | 4.7 | 1.1 |
| AD-64972 | | | 12.1 | 2.9 | 5.0 | 1.4 | 9.4 | 2.5 | 15.0 | 5.1 | 11.8 | 1.8 |
| AD-1181443 | | | 4.0 | 0.5 | 1.5 | 0.6 | 5.1 | 1.0 | 10.8 | 2.0 | 5.5 | 0.3 |
| AD-1181451 | | | 13.1 | 0.7 | 6.3 | 1.4 | 13.9 | 1.8 | 17.2 | 2.0 | 14.8 | 1.9 |
| AD-1181460 | | | 22.6 | 4.7 | 3.0 | 0.5 | 4.9 | 0.2 | 16.4 | 1.8 | 7.7 | 1.1 |
| AD-1181392 | 1 | nM | 5.5 | 0.5 | 3.7 | 0.2 | 7.0 | 2.1 | 22.7 | 4.9 | 10.9 | 2.5 |
| AD-1181401 | | | 47.8 | 13.8 | 15.0 | 2.9 | 18.6 | 4.6 | 39.1 | 2.5 | 17.9 | 1.3 |
| AD-1181410 | | | 16.7 | 2.9 | 9.2 | 1.3 | 8.9 | 0.9 | 12.0 | 3.2 | 14.0 | 2.3 |
| AD-1181417 | | | 49.0 | 14.9 | 27.9 | 3.2 | 42.6 | 9.8 | 88.7 | 11.6 | 34.8 | 7.4 |
| AD-1181426 | | | 15.8 | 3.8 | 8.1 | 0.8 | 8.6 | 3.5 | 20.5 | 2.6 | 12.0 | 2.9 |
| AD-64972 | | | 39.9 | 6.4 | 17.9 | 2.7 | 32.0 | 8.5 | 36.2 | 8.5 | 31.2 | 6.0 |
| AD-1181443 | | | 16.9 | 2.3 | 9.5 | 4.1 | 21.4 | 4.0 | 28.3 | 4.3 | 20.8 | 4.2 |
| AD-1181451 | | | 44.9 | 3.3 | 31.9 | 9.0 | 49.0 | 6.0 | 53.4 | 6.9 | 43.3 | 4.4 |
| AD-1181460 | | | 74.8 | 15.4 | 23.9 | 0.7 | 23.7 | 0.8 | 46.7 | 6.7 | 28.4 | 6.8 |
| AD-1181392 | 0.1 | nM | 23.5 | 1.4 | 18.7 | 1.4 | 26.1 | 7.9 | 56.7 | 7.6 | 27.7 | 3.2 |
| AD-1181401 | | | 61.8 | 6.4 | 33.9 | 3.7 | 37.7 | 4.1 | 75.4 | 15.2 | 45.9 | 8.7 |
| AD-1181410 | | | 48.7 | 4.0 | 28.3 | 3.9 | 20.9 | 1.8 | 32.0 | 2.3 | 35.5 | 7.8 |
| AD-1181417 | | | 62.5 | 7.4 | 56.2 | 14.9 | 88.6 | 13.4 | 132.8 | 33.8 | 84.7 | 22.3 |
| AD-1181426 | | | 71.0 | 16.7 | 53.9 | 4.2 | 46.1 | 17.4 | 136.9 | 20.8 | 69.9 | 16.6 |
| AD-64972 | | | 153.9 | 12.2 | 94.2 | 14.1 | 108.8 | 22.1 | 56.3 | 2.6 | 54.8 | 13.3 |
| AD-1181443 | | | 80.2 | 5.8 | 20.2 | 2.4 | 42.0 | 2.4 | 45.0 | 9.6 | 34.3 | 5.8 |
| AD-1181451 | | | 69.6 | 6.4 | 58.7 | 7.8 | 71.2 | 9.0 | 80.8 | 25.3 | 65.2 | 10.0 |
| AD-1181460 | | | 72.3 | 4.0 | 44.3 | 5.7 | 48.3 | 2.9 | 53.6 | 7.2 | 51.1 | 1.9 |

TABLE 11
In vitro activity for various designs in primary mouse hepatocytes

| Parent | | | | | | | | | | | | | | |
|---|---|---|---|---|---|---|---|---|---|---|---|---|---|---|
| Duplex ID | Dose | | Parent | StDev | D8 | StDev | D9 | StDev | D10 | StDev | D11 | StDev | D12 | StDev |
| AD-1181392 | 10 | nM | 1.2 | 0.1 | 11.0 | 2.4 | 9.3 | 1.7 | 4.7 | 0.7 | 10.5 | 3.0 | 14.7 | 2.5 |
| AD-1181401 | | | 24.2 | 2.3 | 21.5 | 3.2 | 13.6 | 2.6 | 11.4 | 2.0 | 24.1 | 3.0 | 23.4 | 3.6 |
| AD-1181410 | | | 5.3 | 1.9 | 3.0 | 0.3 | 4.3 | 0.6 | 3.6 | 0.7 | 10.5 | 3.6 | 5.7 | 1.7 |
| AD-1181417 | | | 15.4 | 3.6 | 48.9 | 10.6 | 48.8 | 13.3 | 23.4 | 11.8 | 54.9 | 16.4 | 53.8 | 6.7 |
| AD-1181426 | | | 2.6 | 0.6 | 10.7 | 3.6 | 6.1 | 1.3 | 6.9 | 2.2 | 21.0 | 5.4 | 18.3 | 4.6 |
| AD-64972 | | | 12.1 | 2.9 | 13.7 | 3.1 | 13.4 | 4.4 | 15.1 | 5.6 | 27.0 | 5.7 | 30.5 | 3.6 |
| AD-1181443 | | | 4.0 | 0.5 | 11.4 | 3.1 | 9.2 | 1.8 | 6.9 | 1.0 | 16.8 | 3.9 | 14.9 | 3.3 |
| AD-1181451 | | | 13.1 | 0.7 | 19.6 | 3.2 | 20.4 | 4.1 | 15.2 | 3.3 | 30.9 | 3.4 | 39.0 | 6.9 |
| AD-1181460 | | | 22.6 | 4.7 | 24.3 | 5.2 | 23.0 | 2.8 | 15.0 | 1.8 | 35.8 | 3.9 | 47.1 | 2.6 |
| AD-1181392 | 1 | nM | 5.5 | 0.5 | 29.8 | 1.0 | 27.1 | 3.3 | 12.6 | 0.9 | 28.1 | 8.1 | 32.2 | 3.7 |
| AD-1181401 | | | 47.8 | 13.8 | 44.7 | 5.3 | 30.3 | 4.6 | 29.8 | 6.7 | 43.3 | 0.4 | 48.2 | 11.0 |
| AD-1181410 | | | 16.7 | 2.9 | 13.2 | 1.4 | 15.2 | 4.2 | 12.4 | 3.0 | 24.2 | 6.3 | 16.2 | 2.3 |
| AD-1181417 | | | 49.0 | 14.9 | 68.4 | 6.6 | 74.2 | 16.5 | 40.2 | 5.1 | 76.1 | 7.5 | 109.0 | 13.3 |
| AD-1181426 | | | 15.8 | 3.8 | 30.7 | 9.4 | 20.1 | 2.9 | 19.6 | 6.0 | 41.7 | 3.8 | 30.4 | 7.2 |
| AD-64972 | | | 39.9 | 6.4 | 33.8 | 5.9 | 41.3 | 5.1 | 34.7 | 8.0 | 62.8 | 3.6 | 62.5 | 7.1 |
| AD-1181443 | | | 16.9 | 2.3 | 34.9 | 4.2 | 28.8 | 5.8 | 23.2 | 6.5 | 45.6 | 11.2 | 39.0 | 3.7 |
| AD-1181451 | | | 44.9 | 3.3 | 66.8 | 4.3 | 62.9 | 15.2 | 55.0 | 13.0 | 76.2 | 10.5 | 82.0 | 19.4 |
| AD-1181460 | | | 74.8 | 15.4 | 70.3 | 13.8 | 49.4 | 6.2 | 50.2 | 7.3 | 77.5 | 9.4 | 79.8 | 13.4 |
| AD-1181392 | 0.1 | nM | 23.5 | 1.4 | 54.1 | 10.4 | 47.7 | 2.3 | 28.9 | 1.1 | 49.2 | 3.2 | 54.4 | 2.2 |
| AD-1181401 | | | 61.8 | 6.4 | 60.0 | 5.6 | 47.9 | 5.6 | 51.8 | 7.7 | 66.5 | 6.4 | 68.6 | 2.0 |
| AD-1181410 | | | 48.7 | 4.0 | 36.9 | 4.3 | 34.1 | 7.6 | 33.4 | 1.8 | 62.0 | 9.3 | 43.6 | 6.5 |
| AD-1181417 | | | 62.5 | 7.4 | 160.5 | 30.4 | 98.3 | 27.8 | 118.4 | 35.9 | 76.4 | 7.7 | 110.2 | 13.7 |
| AD-1181426 | | | 71.0 | 16.7 | 46.9 | 6.8 | 48.4 | 12.4 | 77.7 | 25.4 | 107.5 | 28.7 | 88.3 | 36.8 |
| AD-64972 | | | 153.9 | 12.2 | 55.6 | 11.8 | 68.1 | 24.6 | 69.6 | 22.2 | 130.8 | 26.1 | 123.2 | 18.6 |
| AD-1181443 | | | 80.2 | 5.8 | 60.7 | 9.7 | 63.6 | 8.4 | 46.4 | 5.7 | 56.6 | 7.5 | 58.8 | 1.6 |
| AD-1181451 | | | 69.6 | 6.4 | 53.8 | 2.3 | 60.8 | 1.8 | 51.2 | 1.0 | 68.4 | 8.3 | 77.0 | 4.5 |
| AD-1181460 | | | 72.3 | 4.0 | 64.1 | 9.0 | 71.2 | 3.6 | 64.4 | 3.5 | 68.4 | 3.2 | 68.6 | 3.3 |

TABLE 12
In vitro activity for various designs in primary cynomolgus hepatocytes

| Parent Duplex ID | Dose | | Parent | StDev | D1 | StDev | D2 | StDev |
|---|---|---|---|---|---|---|---|---|
| AD-157464.8 | 10 | nM | 1.0 | 0.0 | 1.0 | 0.3 | 1.2 | 0.3 |
| AD-157448.7 | | | 1.5 | 0.3 | 2.4 | 0.3 | 3.0 | 1.5 |
| AD-157468.11 | | | 2.2 | 1.2 | 1.6 | 0.2 | 1.8 | 0.1 |
| AD-67327.28 | | | 8.3 | 11.8 | 1.7 | 0.5 | 1.8 | 0.1 |
| AD-85446.5 | | | 1.9 | 0.3 | 1.7 | 0.7 | 1.8 | 0.5 |
| AD-85435.13 | | | 2.9 | 0.1 | 2.1 | 0.2 | 1.7 | 0.2 |
| AD-85438.5 | | | 1.7 | 0.3 | 1.2 | 0.1 | 1.8 | 0.1 |
| AD-157464.8 | 1 | nM | 3.0 | 0.6 | 1.4 | 0.4 | 1.6 | 0.4 |
| AD-157448.7 | | | 3.7 | 0.7 | 3.5 | 0.6 | 2.8 | 0.8 |
| AD-157468.11 | | | 5.7 | 0.6 | 4.4 | 0.2 | 3.6 | 0.4 |
| AD-67327.28 | | | 1.6 | 0.0 | 1.9 | 0.1 | 2.2 | 0.2 |
| AD-85446.5 | | | 4.5 | 0.6 | 2.6 | 0.7 | 2.7 | 0.7 |
| AD-85435.13 | | | 6.7 | 0.5 | 2.8 | 0.3 | 2.8 | 0.2 |
| AD-85438.5 | | | 3.5 | 0.1 | 3.2 | 0.5 | 5.9 | 1.0 |
| AD-157464.8 | 0.1 | nM | 10.2 | 0.1 | 1.9 | 0.5 | 2.9 | 0.9 |
| AD-157448.7 | | | 9.0 | 1.0 | 8.9 | 1.7 | 6.9 | 1.0 |
| AD-157468.11 | | | 16.9 | 3.9 | 13.1 | 1.5 | 10.0 | 1.3 |
| AD-67327.28 | | | 4.6 | 0.7 | 3.9 | 0.4 | 4.8 | 0.4 |
| AD-85446.5 | | | 15.9 | 1.5 | 5.9 | 1.1 | 6.8 | 0.6 |
| AD-85435.13 | | | 18.4 | 2.2 | 5.4 | 1.3 | 7.2 | 0.1 |
| AD-85438.5 | | | 10.8 | 1.7 | 10.2 | 2.1 | 14.2 | 2.5 |

TABLE 13
In vitro activity for various designs in Hep3B

| | Parent | | | | D1 | | | |
|---|---|---|---|---|---|---|---|---|
| Target | Duplex ID | 10 nM | 1 nM | 0.1 nM | Duplex ID | 10 nM | 1 nM | 0.1 nM |
| Marc1 | AD-1531684.2 | 8 ± 1.2 | 18.4 ± 3.7 | 12 ± 1.8 | AD-1636769.1 | 6.6 ± 0.8 | 11.4 ± 1.4 | 16.4 ± 1.3 |
| Marc1 | AD-1531719.2 | 26.8 ± 2.8 | 61.9 ± 6.6 | 69 ± 8.4 | AD-1636805.1 | 10.1 ± 1.7 | 32.9 ± 5.2 | 53.2 ± 3.4 |
| Marc1 | AD-1531682.2 | 16.4 ± 2.8 | 42.4 ± 7.2 | 27.3 ± 2.4 | AD-1636782.1 | 11.9 ± 1.1 | 25.9 ± 1.8 | 38.2 ± 5.1 |
| PNPLA3 | AD-75247.4 | 53.6 ± 9 | 88.8 ± 9.5 | 90.3 ± 10.4 | AD-1636754.1 | 52.2 ± 3.3 | 71.2 ± 17.2 | 69.6 ± 13.7 |
| C3 | AD-1531655.2 | 14.3 ± 2.4 | 46 ± 2.2 | 54.8 ± 11.1 | AD-1636695.1 | 14.6 ± 2.4 | 26.6 ± 4.6 | 30.5 ± 3.1 |
| PNPLA3 | AD-67589.6 | 40.1 ± 10.3 | 68.2 ± 16.9 | 57.6 ± 7.6 | AD-1636733.1 | 29.9 ± 4.7 | 31 ± 5 | 37.9 ± 1.7 |
| Marc1 | AD-1531703.2 | 15.4 ± 1.5 | 47.9 ± 2.3 | 54.5 ± 6.6 | AD-1636798.1 | 9 ± 1.8 | 16.6 ± 3.2 | 34.3 ± 3.5 |
| C3 | AD-1531665.2 | 46.4 ± 5.3 | 83.4 ± 12.2 | 94.3 ± 7 | AD-1636720.1 | 35.7 ± 5 | 52.4 ± 3.4 | 71.4 ± 3.5 |
| C3 | AD-1531660.2 | 14.8 ± 4.1 | 39.2 ± 8.6 | 54.6 ± 8.7 | AD-1636701.1 | 13.9 ± 4.4 | 30.3 ± 6.8 | 41.1 ± 4.1 |
| C3 | AD-1531672.2 | 25.6 ± 4.4 | 62.5 ± 3.8 | 82 ± 9.7 | AD-1636716.1 | 19.2 ± 2.9 | 38.5 ± 5.4 | 55.1 ± 2.6 |
| PNPLA3 | AD-1010735.4 | 35.2 ± 8.8 | 55.1 ± 21.7 | 52.6 ± 7.4 | AD-1636729.1 | 48.4 ± 3.4 | 45 ± 4.2 | 56.2 ± 11 |
| Marc1 | AD-1531721.2 | 38.2 ± 4.3 | 61.6 ± 5.7 | 69.9 ± 10.8 | AD-1636806.1 | 16.7 ± 1.1 | 30.4 ± 2 | 53.6 ± 9.8 |
| PNPLA3 | AD-67605.10 | 30.7 ± 3.2 | 44.9 ± 3.4 | 42.2 ± 2.6 | AD-1636735.1 | 41.9 ± 2.3 | 51.9 ± 11.3 | 64.6 ± 11.6 |
| C3 | AD-571552.3 | 33.6 ± 7.3 | 73.4 ± 4.7 | 87.3 ± 7.4 | AD-1636714.1 | 25.1 ± 2.2 | 42.2 ± 7.6 | 50.4 ± 1.3 |
| Marc1 | AD-1531716.2 | 8 ± 1.4 | 20.3 ± 4.7 | 15.6 ± 2.3 | AD-1636778.1 | 7.5 ± 0.8 | 10.5 ± 1.5 | 13.2 ± 1.1 |
| C3 | AD-569269.6 | 7.3 ± 1 | 12.3 ± 1.4 | 12.3 ± 3.2 | AD-1479350.2 | 8.7 ± 1.2 | 10.5 ± 1.2 | 14.4 ± 1.7 |
| Marc1 | AD-1531718.2 | 9.1 ± 1.4 | 29.1 ± 5.1 | 23.1 ± 2.7 | AD-1636785.1 | 7.6 ± 1.3 | 12.6 ± 1.9 | 24.1 ± 2.7 |
| C3 | AD-1531663.3 | 14.9 ± 1.6 | 26.9 ± 4.6 | 56.5 ± 2.7 | AD-1636763.1 | 12.6 ± 2 | 14.5 ± 4.5 | 27.4 ± 3.6 |
| SCN9A | AD-1531765.2 | 40.2 ± 2.8 | 55.3 ± 7.9 | 68.2 ± 1.9 | AD-1636834.1 | 34.7 ± 3.3 | 38.6 ± 2.2 | 47.7 ± 10.4 |
| SCN9A | AD-1531735.2 | 33.2 ± 4 | 46.1 ± 5.2 | 34.9 ± 4 | AD-1636811.1 | 28.7 ± 3 | 42.5 ± 5.4 | 52.8 ± 4.3 |
| PNPLA3 | AD-519933.3 | 54.6 ± 8 | 99.2 ± 17.3 | 91.4 ± 18.6 | AD-1636757.1 | 52 ± 6.2 | 56.2 ± 4.3 | 60.4 ± 2.7 |
| PNPLA3 | AD-519780.2 | 50.1 ± 3.9 | 80.1 ± 12.2 | 79.5 ± 10.2 | AD-1636758.1 | 44.8 ± 7.9 | 55.5 ± 8 | 74.1 ± 6.4 |
| Marc1 | AD-1531679.2 | 28.2 ± 2.9 | 60.8 ± 10.5 | 61.4 ± 4.4 | AD-1636791.1 | 16.4 ± 1.4 | 46.6 ± 8.1 | 59 ± 7.7 |
| Marc1 | AD-1531692.2 | 13.5 ± 0.7 | 23.7 ± 1.7 | 17.9 ± 2.9 | AD-1636777.1 | 9.8 ± 2.7 | 20 ± 1.4 | 28.7 ± 3.8 |
| SCN9A | AD-1531741.2 | 72.7 ± 1.3 | 83.5 ± 8.9 | 69.8 ± 2.9 | AD-1636838.1 | 47.3 ± 10.9 | 76.9 ± 4 | 106.3 ± 11.5 |
| C3 | AD-572575.3 | 20.8 ± 4.2 | 57.3 ± 9.8 | 68.9 ± 11.1 | AD-1636703.1 | 26.4 ± 6 | 38 ± 10.6 | 58.9 ± 14.5 |
| SCN9A | AD-1531744.2 | 49.2 ± 3.9 | 65.5 ± 8.5 | 63.3 ± 7.5 | AD-1636831.1 | 34.5 ± 5.7 | 66.5 ± 5.5 | 89.5 ± 7.2 |
| PNPLA3 | AD-519354.4 | 62.3 ± 16.1 | 77.2 ± 22.9 | 84.6 ± 15.6 | AD-1636732.1 | 37.5 ± 7.3 | 44.5 ± 6.7 | 64.5 ± 13.5 |
| PNPLA3 | AD-67551.7 | 22.5 ± 1.9 | 38.4 ± 12 | 41 ± 4.2 | AD-1636737.1 | 30.5 ± 4.4 | 39.2 ± 8.3 | 60 ± 7.1 |
| Marc1 | AD-1531687.2 | 14.7 ± 2.6 | 34.6 ± 3.9 | 21.1 ± 1.9 | AD-1636779.1 | 14.3 ± 2.8 | 23.6 ± 1.9 | 32.5 ± 7.6 |
| SCN9A | AD-1531736.2 | 73.3 ± 10.3 | 78.8 ± 7.7 | 68.3 ± 6.1 | AD-1636843.1 | 82.1 ± 9.5 | 95.3 ± 12.7 | 107.5 ± 11.9 |
| Marc1 | AD-1531688.2 | 5 ± 1.1 | 13 ± 2.3 | 9.2 ± 1.3 | AD-1636772.1 | 6.5 ± 1 | 10.1 ± 0.5 | 11.2 ± 1.7 |
| SCN9A | AD-1531747.2 | 69.4 ± 2 | 77.8 ± 3.6 | 80 ± 5.3 | AD-1636836.1 | 45.7 ± 2.9 | 60.4 ± 5.5 | 93.2 ± 3.3 |
| SCN9A | AD-1531756.2 | 38.3 ± 4.9 | 46.5 ± 2.8 | 46.4 ± 5.2 | AD-1636813.1 | 29.8 ± 1.1 | 46.7 ± 4.7 | 53.2 ± 2.7 |
| Marc1 | AD-1531696.2 | 9.4 ± 0.8 | 22.3 ± 4.3 | 18.5 ± 4.2 | AD-1636783.1 | 7.5 ± 0.7 | 12.7 ± 1.3 | 25 ± 2.2 |
| C3 | AD-1531658.2 | 13.3 ± 1.8 | 30.7 ± 0.8 | 39.7 ± 3.7 | AD-1636692.1 | 12.1 ± 1.2 | 24.2 ± 5.8 | 40.9 ± 10.6 |
| SCN9A | AD-1531757.2 | 64.6 ± 4 | 75.8 ± 7.1 | 70.3 ± 3.8 | AD-1636844.1 | 30.1 ± 2.4 | 46.6 ± 5.4 | 59.5 ± 3.3 |
| PNPLA3 | AD-67565.3 | 62.4 ± 17.9 | 69.5 ± 15 | 68.4 ± 8.1 | AD-1636726.1 | 71.3 ± 5.6 | 50 ± 2.7 | 76.6 ± 18.7 |
| PNPLA3 | AD-1010714.3 | 52.6 ± 19 | 99.7 ± 26.3 | 85.9 ± 13 | AD-1636760.1 | 28.4 ± 5.4 | 40 ± 6 | 54.8 ± 4.3 |
| SCN9A | AD-1531728.2 | 60.8 ± 4.2 | 69.8 ± 9.1 | 76.8 ± 4.7 | AD-1636841.1 | 33.6 ± 5.1 | 53.3 ± 8.2 | 65.3 ± 8.5 |
| Marc1 | AD-1531686.2 | 16.4 ± 2.6 | 52.5 ± 3.5 | 50.2 ± 8.3 | AD-1636789.1 | 12.3 ± 2.3 | 25.2 ± 4.6 | 45.5 ± 4.1 |
| PNPLA3 | AD-67583.7 | 45.2 ± 4.6 | 81.4 ± 24.3 | 64.5 ± 13.5 | AD-1636762.1 | 46.4 ± 12.4 | 48.8 ± 8.4 | 48.8 ± 10.2 |
| C3 | AD-572388.7 | 20.2 ± 1.8 | 49.7 ± 7.8 | 68.7 ± 5.5 | AD-1636766.1 | 71 ± 9.6 | 81.7 ± 9.4 | 90.1 ± 12.4 |
| C3 | AD-1531662.2 | 37.3 ± 5.1 | 83.1 ± 8.3 | 91.9 ± 5.3 | AD-1636718.1 | 31.5 ± 1.6 | 68.9 ± 9.3 | 77.7 ± 5.9 |
| C3 | AD-571901.2 | 26.2 ± 7.2 | 62.6 ± 5.1 | 78.2 ± 7.4 | AD-1636707.1 | 32.1 ± 7.9 | 60 ± 7.6 | 61 ± 6.7 |
| PNPLA3 | AD-1010732.4 | 30.3 ± 6.7 | 63.6 ± 15 | 52.1 ± 4.7 | AD-1636727.1 | 39.3 ± 3.8 | 55 ± 13.3 | 59.5 ± 11 |
| PNPLA3 | AD-520061.6 | 32.7 ± 8.1 | 49.5 ± 8.4 | 51.3 ± 5.3 | AD-1636741.1 | 42.2 ± 5.7 | 44.4 ± 2.4 | 57.3 ± 4.9 |
| SCN9A | AD-1531770.2 | 50.6 ± 8.6 | 74.1 ± 0.9 | 76.6 ± 3.5 | AD-1636830.1 | 38.3 ± 1.7 | 59.3 ± 7.3 | 72.6 ± 7.7 |
| Marc1 | AD-1531705.2 | 5.8 ± 0.7 | 21.9 ± 2.7 | 11.9 ± 2.1 | AD-1636780.1 | 6 ± 0.7 | 14.2 ± 5.2 | 27.9 ± 2.5 |
| C3 | AD-569266.3 | 7.5 ± 1.3 | 19 ± 4.3 | 24.5 ± 5.1 | AD-1636689.1 | 12.2 ± 2.1 | 18.5 ± 3.2 | 22.2 ± 4.3 |
| SCN9A | AD-1531740.2 | 68.1 ± 10.5 | 84.9 ± 5.5 | 66.9 ± 2.4 | AD-1636839.1 | 41.3 ± 6 | 56.5 ± 9.4 | 72 ± 4.9 |
| PNPLA3 | AD-75265.5 | 48.9 ± 5.5 | 83.9 ± 20.7 | 82.7 ± 8.8 | AD-1636753.1 | 36.1 ± 10.1 | 55.8 ± 8.4 | 67.9 ± 6.8 |
| Marc1 | AD-1531709.2 | 13.9 ± 3.1 | 37.2 ± 7.5 | 46.9 ± 13.3 | AD-1636792.1 | 10.5 ± 0.7 | 20.9 ± 2.8 | 30.7 ± 6.5 |
| C3 | AD-569048.2 | 20.9 ± 4 | 56.4 ± 11.3 | 64.2 ± 11.2 | AD-1636709.1 | 34.3 ± 0.8 | 50.6 ± 10 | 75.3 ± 7.9 |
| C3 | AD-571633.3 | 34.2 ± 5.7 | 72.1 ± 7.6 | 81.3 ± 7 | AD-1636721.1 | 29.7 ± 2.1 | 51.3 ± 3 | 78.2 ± 9.8 |
| C3 | AD-569516.3 | 28.8 ± 2.2 | 76.2 ± 9.6 | 91.9 ± 10.4 | AD-1636719.1 | 32.5 ± 5.2 | 51.9 ± 12.9 | 72.2 ± 11.7 |
| PNPLA3 | AD-519346.5 | 26.2 ± 4.5 | 53 ± 6.2 | 51 ± 4.9 | AD-1636765.1 | 27 ± 5.3 | 28.3 ± 3.6 | 41.8 ± 5.1 |
| C3 | AD-571748.3 | 8.2 ± 2.8 | 26.5 ± 2.2 | 38.3 ± 5.8 | AD-1636693.1 | 13.5 ± 2.4 | 32.2 ± 7.1 | 37.7 ± 6.8 |
| Marc1 | AD-1531708.2 | 27.5 ± 1.5 | 61.3 ± 6.1 | 70.9 ± 12.7 | AD-1636807.1 | 18.9 ± 2.9 | 26.2 ± 4.7 | 49.5 ± 5.9 |
| C3 | AD-570712.7 | 7.3 ± 0.5 | 17.3 ± 1.6 | 28.1 ± 3.6 | AD-1479283.2 | 10.2 ± 0.7 | 16.5 ± 1.3 | 27.9 ± 4.1 |
| Marc1 | AD-1531706.2 | 5.4 ± 0.4 | 14.1 ± 1.1 | 8.5 ± 1.1 | AD-1636774.1 | 5.7 ± 0.9 | 10.8 ± 2.4 | 13.8 ± 1.1 |
| PNPLA3 | AD-67584.8 | 49.1 ± 8.5 | 57.2 ± 5.6 | 49.8 ± 13.5 | AD-1636725.1 | 57.4 ± 13 | 57.4 ± 16.8 | 67.4 ± 9.5 |
| C3 | AD-1531668.2 | 17.2 ± 2.9 | 49.8 ± 8.7 | 61.8 ± 9.7 | AD-1636715.1 | 13.8 ± 1.8 | 28.2 ± 4.4 | 45.9 ± 4.9 |
| PNPLA3 | AD-67577.7 | 27.2 ± 3.3 | 41.7 ± 6.5 | 44.1 ± 7.5 | AD-1636750.1 | 30.7 ± 7.3 | 38.2 ± 4.1 | 58.8 ± 7.1 |
| C3 | AD-571753.4 | 11.7 ± 2.3 | 30 ± 6.6 | 47.8 ± 10 | AD-1636702.1 | 12.3 ± 0.9 | 17.9 ± 3.4 | 31.6 ± 3.1 |
| C3 | AD-1531657.2 | 20.6 ± 5.8 | 58.1 ± 9.5 | 73 ± 7.9 | AD-1636705.1 | 16 ± 2 | 30.2 ± 5.1 | 42 ± 4.2 |
| C3 | AD-571715.5 | 5.7 ± 0.6 | 11.7 ± 0.9 | 24.5 ± 4.2 | AD-1636690.1 | 6.4 ± 1.5 | 13.3 ± 2.2 | 19.1 ± 2.2 |
| C3 | AD-1531659.2 | 12.1 ± 1.9 | 29.4 ± 3.5 | 38.2 ± 7.2 | AD-1636694.1 | 12.8 ± 1.8 | 22.3 ± 6.6 | 25.6 ± 2.1 |
| PNPLA3 | AD-67560.7 | 52.7 ± 14.7 | 63.9 ± 17.1 | 68.5 ± 10 | AD-1636743.1 | 43.9 ± 6.2 | 46.6 ± 7.3 | 51.7 ± 9.1 |
| PNPLA3 | AD-1531674.2 | 51.8 ± 5.6 | 55.2 ± 5.4 | 47.2 ± 8.1 | AD-1636756.1 | 47.3 ± 8.3 | 61.1 ± 10.9 | 60.3 ± 6.7 |
| PNPLA3 | AD-519757.6 | 48.2 ± 8.9 | 43.1 ± 14.7 | 59.6 ± 5.1 | AD-1636734.1 | 36.9 ± 8.1 | 65.6 ± 10.7 | 51.3 ± 10.8 |
| SCN9A | AD-1531732.2 | 65.4 ± 1.9 | 43.7 ± 8.3 | 57.1 ± 4.6 | AD-1636840.1 | 54.3 ± 6.5 | 63.4 ± 5.7 | 56.7 ± 12.5 |
| C3 | AD-1531656.2 | 57.5 ± 10.1 | 104.8 ± 19.6 | 90.9 ± 15.1 | AD-1636712.1 | 44.6 ± 2.5 | 59.1 ± 11.9 | 94.7 ± 8.9 |
| Marc1 | AD-1531678.2 | 36.1 ± 3.9 | 66 ± 4.3 | 58.6 ± 11.2 | AD-1636800.1 | 15.4 ± 3.1 | 22.7 ± 1.6 | 38.5 ± 5.7 |
| Marc1 | AD-1531701.2 | 15.3 ± 1.7 | 32.8 ± 2.4 | 31.4 ± 3.2 | AD-1636790.1 | 16 ± 2.9 | 31.8 ± 3.5 | 49.4 ± 7.7 |
| Marc1 | AD-1531680.2 | 5.1 ± 0.6 | 16.4 ± 1.8 | 9.7 ± 1.1 | AD-1636770.1 | 6.5 ± 0.8 | 14.5 ± 3.6 | 19.7 ± 5 |
| SCN9A | AD-1531734.2 | 56.4 ± 4.3 | 67.5 ± 10.7 | 82 ± 4 | AD-1636837.1 | 48.4 ± 6.4 | 78.6 ± 3.6 | 107.1 ± 13.3 |
| C3 | AD-568977.3 | 14.4 ± 1.7 | 38 ± 4.8 | 41.4 ± 11.1 | AD-1636699.1 | 17.7 ± 1.1 | 24.9 ± 3.3 | 39.3 ± 2.8 |
| C3 | AD-569268.10 | 6.8 ± 0.4 | 10.2 ± 1.9 | 14.2 ± 1.9 | AD-1479341.2 | 8.7 ± 1.6 | 12 ± 0.7 | 16.6 ± 2.5 |
| C3 | AD-572818.2 | 10.5 ± 1.5 | 27.2 ± 4.4 | 42 ± 7.4 | AD-1636691.1 | 13.1 ± 1.8 | 23.3 ± 2.2 | 36.9 ± 6.1 |
| SCN9A | AD-1531729.2 | 45 ± 3.7 | 60.9 ± 4.8 | 49.6 ± 7.2 | AD-1636815.1 | 35.8 ± 5.6 | 49.7 ± 6 | 51.2 ± 1.5 |
| Marc1 | AD-1531689.2 | 36.6 ± 7.1 | 61.4 ± 7.9 | 69.7 ± 4.9 | AD-1636795.1 | 22.5 ± 3.4 | 33.4 ± 1.5 | 51.1 ± 5.9 |
| PNPLA3 | AD-75275.3 | 56 ± 8.3 | 60.8 ± 11.8 | 44.6 ± 2.6 | AD-1636731.1 | 48.6 ± 4.2 | 61 ± 8.4 | 70.7 ± 11.9 |
| PNPLA3 | AD-67568.3 | 33.8 ± 2.8 | 35 ± 4.9 | 50.8 ± 8.1 | AD-1636744.1 | 36.2 ± 6.1 | 43.9 ± 5.5 | 48.5 ± 2.2 |
| C3 | AD-569494.2 | 98.1 ± 8 | 113.9 ± 3.9 | 105.2 ± 8.5 | AD-1636717.1 | 105.6 ± 13.2 | 92.8 ± 7.2 | 90.9 ± 14.1 |
| Marc1 | AD-1531722.2 | 26.5 ± 0.9 | 54.4 ± 5 | 54.4 ± 6.6 | AD-1636796.1 | 12.3 ± 2.2 | 21.8 ± 0.4 | 27.7 ± 3.4 |
| C3 | AD-571610.3 | 21.1 ± 7.8 | 49 ± 4.8 | 69.1 ± 9.5 | AD-1636710.1 | 25.4 ± 1.7 | 45.3 ± 5.6 | 61.5 ± 8.6 |
| SCN9A | AD-1531753.2 | 42.9 ± 1.7 | 59.3 ± 4.7 | 54.2 ± 5.1 | AD-1636822.1 | 33 ± 2.8 | 53.9 ± 6.7 | 62 ± 7.7 |
| SCN9A | AD-1531759.2 | 46.3 ± 3.4 | 50.6 ± 5.4 | 70.6 ± 2.3 | AD-1636828.1 | 30.8 ± 2.4 | 41.1 ± 4.7 | 52.9 ± 7.6 |
| C3 | AD-572495.3 | 8.7 ± 0.9 | 10.7 ± 1.7 | 17 ± 2.5 | AD-1636767.1 | 11.5 ± 1.2 | 15 ± 1.5 | 21.2 ± 5.2 |
| PNPLA3 | AD-1010719.3 | 30 ± 4.2 | 79.4 ± 24.3 | 28.9 ± 4 | AD-1636739.1 | 20.9 ± 2.1 | 24.4 ± 5.8 | 33.7 ± 8.2 |
| Marc1 | AD-1531683.2 | 28.6 ± 5.8 | 64.7 ± 6.8 | 54.8 ± 3 | AD-1636803.1 | 32.4 ± 4.6 | 58.5 ± 9.1 | 85.2 ± 5 |
| Marc1 | AD-1531711.2 | 22.6 ± 6.1 | 46.2 ± 9.7 | 41.4 ± 4.7 | AD-1636801.1 | 11.7 ± 1 | 20.4 ± 5.1 | 40.3 ± 4.2 |
| C3 | AD-1531664.2 | 19.1 ± 5.3 | 50.6 ± 8.8 | 59.4 ± 2.3 | AD-1636713.1 | 32.7 ± 5.9 | 47.5 ± 4.9 | 53.4 ± 2.6 |
| SCN9A | AD-1531762.2 | 57.9 ± 4 | 75.4 ± 6.4 | 73.8 ± 5.9 | AD-1636833.1 | 35.9 ± 3.5 | 49.3 ± 5.4 | 78.1 ± 13.9 |
| PNPLA3 | AD-67526.5 | 26.9 ± 8.8 | 41.8 ± 13.4 | 22 ± 2.2 | AD-1636740.1 | 19.7 ± 3.7 | 19.4 ± 1.9 | 32.9 ± 4.9 |
| SCN9A | AD-1531725.2 | 26.8 ± 4.4 | 32.5 ± 3.4 | 37.6 ± 3.4 | AD-1636823.1 | 23.8 ± 2.4 | 32.5 ± 5.5 | 36.7 ± 4.8 |
| SCN9A | AD-1531737.2 | 40.1 ± 2.1 | 43.3 ± 3.6 | 57.4 ± 10 | AD-1636827.1 | 33.5 ± 2.8 | 38 ± 4.2 | 54.5 ± 6.9 |
| PNPLA3 | AD-1531673.2 | 42 ± 4.6 | 78.6 ± 23.6 | 65.1 ± 4.5 | AD-1636749.1 | 40.7 ± 3.3 | 31.6 ± 5.5 | 35.8 ± 3.3 |
| Marc1 | AD-1531717.2 | 16.1 ± 2.2 | 38.5 ± 2.8 | 46.9 ± 5.2 | AD-1636799.1 | 11 ± 1.2 | 13.8 ± 2.7 | 25.9 ± 7.2 |
| C3 | AD-570644.2 | 15.1 ± 3.2 | 43.5 ± 6.8 | 61.4 ± 10.6 | AD-1636700.1 | 12.8 ± 1.7 | 30.7 ± 6 | 35.8 ± 7.3 |
| C3 | AD-568962.3 | 24 ± 4.2 | 57.1 ± 12.1 | 69.8 ± 7.9 | AD-1636706.1 | 21.1 ± 4.3 | 35.1 ± 2.9 | 50.5 ± 4.3 |
| Marc1 | AD-1531690.2 | 6.7 ± 0.9 | 14.2 ± 2.8 | 8.7 ± 1.3 | AD-1636771.1 | 6 ± 1.1 | 10.5 ± 1 | 10.8 ± 1.3 |
| SCN9A | AD-1531750.2 | 46.9 ± 2.7 | 58.3 ± 6.8 | 55.3 ± 4.3 | AD-1636826.1 | 41 ± 5.9 | 52.9 ± 9.3 | 66.5 ± 9.4 |
| Marc1 | AD-1531710.3 | 14.9 ± 2.2 | 34.3 ± 6.1 | 40.1 ± 7.6 | AD-1636848.1 | 8.2 ± 1.1 | 25 ± 6 | 44.4 ± 3.3 |
| PNPLA3 | AD-67578.3 | 61 ± 15.4 | 54.9 ± 4.2 | 66.5 ± 4.9 | AD-1636746.1 | 58.6 ± 4.5 | 60.5 ± 16.3 | 68.6 ± 14.1 |
| PNPLA3 | AD-67582.6 | 47.8 ± 7 | 71.9 ± 12.8 | 80.3 ± 12.1 | AD-1636747.1 | 39.9 ± 1.7 | 51.8 ± 5.2 | 66.7 ± 12.8 |
| C3 | AD-1531666.2 | 39.7 ± 10.3 | 71.9 ± 4.8 | 85.9 ± 11.1 | AD-1636723.1 | 15 ± 2.2 | 31.9 ± 6.2 | 67.3 ± 12.3 |
| Marc1 | AD-1531695.3 | 6.7 ± 0.6 | 11.8 ± 3.2 | 11 ± 1.4 | AD-1636847.1 | 7.5 ± 0.5 | 13.4 ± 2.7 | 19.9 ± 3.2 |
| SCN9A | AD-1531723.2 | 29.7 ± 5.8 | 37.8 ± 5.7 | 43.1 ± 3.3 | AD-1636824.1 | 19.3 ± 1.5 | 28.9 ± 6.7 | 32.1 ± 3 |
| PNPLA3 | AD-75270.4 | 46.6 ± 12 | 62.6 ± 15.2 | 65.6 ± 12.2 | AD-1636748.1 | 42.2 ± 1.7 | 39.2 ± 9.5 | 54.7 ± 2.5 |
| C3 | AD-1531671.2 | 11.8 ± 2.7 | 33.2 ± 4.2 | 49.2 ± 7.8 | AD-1636704.1 | 24.1 ± 3.8 | 37 ± 8.3 | 56.6 ± 10.9 |
| PNPLA3 | AD-519350.6 | 35.8 ± 6.4 | 50.4 ± 4.8 | 49.4 ± 5 | AD-1636724.1 | 45.8 ± 8.2 | 55 ± 8.8 | 50.7 ± 9 |
| SCN9A | AD-1531764.2 | 63.2 ± 4.6 | 66 ± 3.1 | 57 ± 10 | AD-1636832.1 | 57.3 ± 4.8 | 69 ± 7.9 | 62.6 ± 5.9 |
| SCN9A | AD-1531739.2 | 39.7 ± 5.3 | 58.5 ± 5.4 | 60.7 ± 7.9 | AD-1636835.1 | 24.5 ± 5.3 | 38.9 ± 1.9 | 57 ± 9.2 |
| Marc1 | AD-1531681.2 | 17.3 ± 3.6 | 55.2 ± 7.2 | 60.8 ± 11.6 | AD-1636788.1 | 11.3 ± 2.6 | 21.4 ± 5.8 | 32.2 ± 9.2 |
| Marc1 | AD-1531704.2 | 7.4 ± 0.7 | 36.8 ± 5 | 26.5 ± 2.2 | AD-1636787.1 | 8.6 ± 2.2 | 14.5 ± 3.1 | 23.5 ± 3.8 |
| PNPLA3 | AD-67564.3 | 71.1 ± 5.4 | 74.7 ± 9 | 76.7 ± 7.6 | AD-1636761.1 | 56.3 ± 2.7 | 55.7 ± 4.5 | 63.4 ± 6.7 |
| Marc1 | AD-1531676.2 | 18.8 ± 4.6 | 57.6 ± 10 | 80 ± 12.9 | AD-1636794.1 | 9.6 ± 1.9 | 22.2 ± 3.4 | 34.6 ± 7.4 |
| Marc1 | AD-1531700.2 | 30.1 ± 5.2 | 71.1 ± 6.8 | 76.9 ± 7.3 | AD-1636804.1 | 12.8 ± 2.6 | 28.6 ± 2.7 | 51.1 ± 9.4 |
| Marc1 | AD-1531702.2 | 37.9 ± 6.4 | 72.7 ± 4.3 | 75.3 ± 5.5 | AD-1636802.1 | 30.3 ± 4.9 | 55 ± 9.5 | 71.9 ± 20.4 |
| SCN9A | AD-1531730.2 | 39.4 ± 2.8 | 47.3 ± 1.2 | 42.4 ± 6.1 | AD-1636814.1 | 30.6 ± 1.6 | 46.2 ± 2.2 | 57.3 ± 0.4 |
| SCN9A | AD-1531738.2 | 32.3 ± 5.3 | 39.4 ± 1.9 | 45 ± 5.5 | AD-1636812.1 | 30.4 ± 2.2 | 38.5 ± 7.1 | 43.3 ± 8 |
| SCN9A | AD-1531733.3 | 45.3 ± 2 | 53.3 ± 10.4 | 35.1 ± 6.9 | AD-1636850.1 | 29.7 ± 3.6 | 44.5 ± 13.3 | 59.6 ± 9.9 |
| PNPLA3 | AD-67554.11 | 39.8 ± 4.4 | 45.5 ± 10 | 55.4 ± 7 | AD-1636738.1 | 48.8 ± 4.6 | 56.6 ± 13.9 | 56 ± 9.1 |
| Marc1 | AD-1531685.2 | 25.3 ± 1.4 | 41.9 ± 6.6 | 51.9 ± 2.1 | AD-1636797.1 | 6 ± 0.9 | 14.9 ± 2.9 | 23.6 ± 1.9 |
| PNPLA3 | AD-75269.3 | 49.5 ± 10.5 | 66.5 ± 13.9 | 44.8 ± 8.1 | AD-1636728.1 | 44.3 ± 2.7 | 40.4 ± 4 | 57.7 ± 11.3 |
| SCN9A | AD-1531752.2 | 87.9 ± 13.1 | 64.7 ± 10.1 | 70 ± 9.8 | AD-1636842.1 | 62.9 ± 1.5 | 78.6 ± 8.9 | 87.1 ± 5.6 |
| SCN9A | AD-1531754.3 | 42.1 ± 1.8 | 46.6 ± 3.8 | 39.8 ± 6.3 | AD-1636852.1 | 25.4 ± 4 | 46 ± 2.2 | 52.6 ± 2.1 |
| PNPLA3 | AD-518942.2 | 59 ± 6.6 | 90.6 ± 20.3 | 106.6 ± 15.5 | AD-1636764.1 | 37.6 ± 5.1 | 33.4 ± 4.9 | 40.6 ± 6.9 |
| C3 | AD-571932.2 | 22.1 ± 2.2 | 45.8 ± 6.9 | 62 ± 6.7 | AD-1636698.1 | 17.8 ± 1.9 | 31 ± 5.9 | 49.4 ± 11.2 |
| C3 | AD-572022.2 | 14.4 ± 2.8 | 34 ± 3.8 | 44.4 ± 6.9 | AD-1636697.1 | 15.9 ± 1.1 | 32.2 ± 5.4 | 52.1 ± 7.3 |
| SCN9A | AD-1531760.2 | 46.7 ± 3.2 | 68.7 ± 6.3 | 58.3 ± 1.6 | AD-1636816.1 | 29.1 ± 5.4 | 51.5 ± 7.6 | 60.9 ± 8.7 |
| SCN9A | AD-1531766.2 | 50.2 ± 8.7 | 55.5 ± 5.8 | 48.9 ± 4.1 | AD-1636846.1 | 55.2 ± 4.6 | 67.2 ± 14.9 | 87.1 ± 18.8 |
| C3 | AD-572577.3 | 35.8 ± 7.2 | 79.9 ± 14.7 | 95.7 ± 6.3 | AD-1636711.1 | 51 ± 12.3 | 74.5 ± 14.2 | 89.2 ± 5.4 |
| Marc1 | AD-1531697.2 | 21.5 ± 4.6 | 41 ± 1.2 | 26.5 ± 0.5 | AD-1636793.1 | 13.1 ± 1.9 | 17.1 ± 3.5 | 31.4 ± 5.3 |
| PNPLA3 | AD-67561.3 | 71.2 ± 14.1 | 81.5 ± 16.5 | 101.6 ± 7.1 | AD-1636742.1 | 65.7 ± 4.6 | 93.1 ± 24.5 | 74.2 ± 12.7 |
| PNPLA3 | AD-67586.3 | 73.6 ± 11.5 | 87.6 ± 12.8 | 86.7 ± 6.3 | AD-1636755.1 | 57.5 ± 5.8 | 61.7 ± 12.1 | 64.6 ± 9.3 |
| C3 | AD-569763.5 | 18.2 ± 4.8 | 45.4 ± 3.6 | 52.7 ± 2.7 | AD-1636696.1 | 19.2 ± 6.9 | 34.1 ± 5.9 | 43.5 ± 5.2 |
| SCN9A | AD-1531726.2 | 32.2 ± 2.5 | 42 ± 3.8 | 46.7 ± 4.1 | AD-1636821.1 | 24.9 ± 1.7 | 39.4 ± 4.5 | 54.4 ± 2.4 |
| SCN9A | AD-1531742.3 | 32.4 ± 5.7 | 52.4 ± 4.3 | 43.4 ± 4.2 | AD-1636851.1 | 23.8 ± 4.1 | 56.9 ± 3.2 | 75.8 ± 13.1 |
| PNPLA3 | AD-67573.3 | 62.3 ± 10.5 | 74.5 ± 8.6 | 77.5 ± 10.2 | AD-1636751.1 | 42.6 ± 2.7 | 41.8 ± 5.2 | 69.3 ± 10.7 |
| SCN9A | AD-1531761.2 | 58.2 ± 5.9 | 72.5 ± 5.2 | 60.6 ± 8.9 | AD-1636818.1 | 26.3 ± 4.7 | 47.6 ± 10 | 56.8 ± 6.5 |
| Marc1 | AD-1531712.2 | 8 ± 0.2 | 19.6 ± 2 | 14.5 ± 0.5 | AD-1636781.1 | 11 ± 0.1 | 13.5 ± 0.7 | 19.5 ± 3.7 |
| C3 | AD-1531669.2 | 48.1 ± 3.2 | 87 ± 9.8 | 80.1 ± 5.5 | AD-1636722.1 | 29.7 ± 3.1 | 50.1 ± 3.9 | 59.9 ± 5.4 |
| SCN9A | AD-1531745.2 | 41.3 ± 3.1 | 50.1 ± 2.4 | 42 ± 1.9 | AD-1636820.1 | 29.3 ± 2.2 | 38.5 ± 2.8 | 47.9 ± 3.8 |
| SCN9A | AD-1531724.2 | 48.3 ± 2.7 | 55.4 ± 4.6 | 47.9 ± 0.9 | AD-1636817.1 | 38 ± 6.1 | 50.9 ± 5 | 67.9 ± 5.4 |
| SCN9A | AD-1531731.2 | 43.6 ± 5.2 | 52.8 ± 1.2 | 66.9 ± 7 | AD-1636825.1 | 33.6 ± 4.3 | 49.6 ± 5.4 | 56.3 ± 2 |
| C3 | AD-570132.3 | 26.7 ± 3.7 | 60.8 ± 9.6 | 87.8 ± 11.1 | AD-1636708.1 | 19.5 ± 3.6 | 31 ± 3.8 | 45.3 ± 5.5 |
| SCN9A | AD-1531769.2 | 38.3 ± 4.6 | 53.1 ± 6.1 | 48.9 ± 3.6 | AD-1636819.1 | 32.6 ± 2.1 | 43.7 ± 5.3 | 54.3 ± 1.1 |
| Marc1 | AD-1531694.2 | 6.4 ± 0.6 | 17.3 ± 2 | 11.6 ± 1.7 | AD-1636775.1 | 8 ± 1.9 | 15.7 ± 2.8 | 19 ± 3.1 |
| Marc1 | AD-1531677.2 | 31.7 ± 3.8 | 59.5 ± 7.4 | 60.1 ± 15.2 | AD-1636784.1 | 16.3 ± 1.8 | 25.4 ± 6.1 | 33 ± 4.8 |
| SCN9A | AD-1531727.2 | 50.1 ± 2.8 | 61.8 ± 5.6 | 74.4 ± 6.6 | AD-1636829.1 | 42.8 ± 3.9 | 59.8 ± 7.2 | 68.7 ± 9 |
| PNPLA3 | AD-67575.11 | 62.6 ± 13.6 | 56.2 ± 5.8 | 58.4 ± 4.3 | AD-1636768.1 | 49.4 ± 9.5 | 50.9 ± 6.4 | 48.9 ± 7.4 |
| PNPLA3 | AD-75274.6 | 72.6 ± 6 | 74.9 ± 13.3 | 96.4 ± 13.1 | AD-1636745.1 | 66 ± 8.7 | 68.9 ± 7.1 | 86.5 ± 7.2 |
| SCN9A | AD-1531768.2 | 32.2 ± 1.8 | 46.7 ± 4.3 | 54.9 ± 5.9 | AD-1636809.1 | 20 ± 0.9 | 30.9 ± 4.4 | 43.4 ± 4.9 |
| C3 | AD-570134.4 | 7.7 ± 1.1 | 12.8 ± 0.6 | 24.1 ± 7 | AD-1636688.1 | 9 ± 1 | 20.9 ± 5.7 | 31.8 ± 8.4 |
| Marc1 | AD-1531707.2 | 6.6 ± 1.2 | 15.8 ± 0.7 | 9.2 ± 0.8 | AD-1636773.1 | 7.9 ± 1 | 10 ± 0.4 | 12.1 ± 0.6 |
| PNPLA3 | AD-75272.3 | 56.9 ± 19.1 | 73.2 ± 12 | 68.6 ± 13.7 | AD-1636752.1 | 41 ± 6 | 42 ± 7.5 | 69.6 ± 9.1 |
| PNPLA3 | AD-67567.3 | 82.7 ± 22.8 | 95.3 ± 15.3 | 85.4 ± 9.9 | AD-1636759.1 | 56.3 ± 6.1 | 62.9 ± 15.9 | 72.3 ± 8.3 |
| Marc1 | AD-1531714.3 | 6.4 ± 0.9 | 18.1 ± 2.3 | 23.7 ± 5.3 | AD-1636849.1 | 9 ± 1 | 16.6 ± 1.8 | 24.5 ± 1.2 |
| Marc1 | AD-1531720.2 | 5.4 ± 0.7 | 12.6 ± 1.7 | 10.8 ± 1.4 | AD-1636776.1 | 5.3 ± 0.6 | 10.9 ± 1.2 | 12.6 ± 1.2 |
| SCN9A | AD-1531746.2 | 73.3 ± 4.7 | 86.7 ± 2.2 | 71.3 ± 10.6 | AD-1636845.1 | 43.2 ± 5.8 | 91.2 ± 8.6 | 86.4 ± 19.1 |
| SCN9A | AD-1531755.2 | 37.8 ± 6.1 | 49.1 ± 5.4 | 46.9 ± 7.4 | AD-1636810.1 | 36.2 ± 2.1 | 61.4 ± 3.2 | 73.5 ± 5.5 |
| Marc1 | AD-1531675.2 | 16.9 ± 3.3 | 32.5 ± 4 | 33 ± 5.7 | AD-1636786.1 | 10.1 ± 1.6 | 16.9 ± 4.3 | 27 ± 4.3 |
| SCN9A | AD-1531767.2 | 28.3 ± 1.2 | 44 ± 2.7 | 56.2 ± 8.9 | AD-1636808.1 | 20.5 ± 1.4 | 32 ± 3.3 | 39.4 ± 5.1 |
| PNPLA3 | AD-520053.7 | 30.9 ± 5.9 | 34.7 ± 8.4 | 52.9 ± 8.6 | AD-1636736.1 | 31.1 ± 6.6 | 44 ± 4.2 | 52.5 ± 4 |
| PNPLA3 | AD-1010734.3 | 42.8 ± 16.5 | 58.8 ± 17.2 | 49.6 ± 9.8 | AD-1636730.1 | 50.8 ± 10.1 | 38.6 ± 7.4 | 53.7 ± 6.5 |

TABLE 14
In vitro activity for various designs targeting Agt in Hep3B

| Parent | | | | D1 | | | |
|---|---|---|---|---|---|---|---|
| Duplex ID | 10 nM | 1 nM | 0.1 nM | Duplex ID | 10 nM | 1 nM | 0.1 nM |
| AD-1632799.1 | 88 ± 15 | 105 ± 23 | 90 ± 21 | AD-1657992.1 | 55 ± 11 | 91 ± 9 | 73 ± 8 |
| AD-1632801.1 | 6 ± 1 | 23 ± 4 | 33 ± 3 | AD-1657994.1 | 2 ± 1 | 12 ± 3 | 15 ± 4 |
| AD-1632805.1 | 82 ± 7 | 95 ± 7 | 83 ± 12 | AD-1657998.1 | 43 ± 4 | 65 ± 16 | 66 ± 6 |
| AD-1632838.1 | 58 ± 5 | 94 ± 7 | 92 ± 10 | AD-1658030.1 | 64 ± 5 | 84 ± 7 | 82 ± 8 |
| AD-1684490.1 | 50 ± 11 | 83 ± 26 | 80 ± 18 | AD-1684491.1 | 38 ± 12 | 67 ± 14 | 73 ± 17 |
| AD-1684492.1 | 28 ± 4 | 68 ± 7 | 71 ± 20 | AD-1684493.1 | 19 ± 2 | 47 ± 12 | 70 ± 11 |
| AD-1684494.1 | 92 ± 19 | 101 ± 10 | 116 ± 6 | AD-1684495.1 | 18 ± 4 | 54 ± 9 | 60 ± 6 |
| AD-1684496.1 | 24 ± 5 | 72 ± 8 | 99 ± 10 | AD-1684497.1 | 4 ± 1 | 15 ± 3 | 42 ± 7 |
| AD-1684498.1 | 8 ± 2 | 31 ± 9 | 66 ± 9 | AD-1684499.1 | 34 ± 8 | 53 ± 10 | 79 ± 8 |
| AD-1684500.1 | 4 ± 1 | 16 ± 5 | 33 ± 6 | AD-1684501.1 | 2 ± 0 | 7 ±2 | 24 ± 2 |
| AD-1684502.1 | 98 ± 12 | 107 ± 8 | 98 ± 3 | AD-1684503.1 | 78 ± 15 | 102 ± 10 | 112 ± 14 |
| AD-1684504.1 | 12 ± 2 | 56 ± 6 | 78 ± 10 | AD-1684505.1 | 1 ± 0 | 7 ± 3 | 18 ± 5 |
| AD-1684506.1 | 16 ± 5 | 53 ± 12 | 58 ± 11 | AD-1684507.1 | 10 ± 3 | 46 ± 9 | 54 ± 6 |
| AD-1684508.1 | 31 ± 8 | 87 ± 10 | 101 ± 15 | AD-1684509.1 | 5 ± 1 | 17 ± 2 | 52 ± 3 |
| AD-1684510.1 | 95 ± 8 | 119 ± 9 | 131 ± 11 | AD-1684511.1 | 30 ± 6 | 67 ± 5 | 96 ± 13 |
| AD-1684512.1 | 2 ± 1 | 9 ± 3 | 24 ± 3 | AD-1684513.1 | 4 ± 2 | 28 ± 3 | 49 ± 9 |
| AD-1684514.1 | 39 ± 12 | 67 ± 8 | 82 ± 10 | AD-1684515.1 | 44 ± 9 | 88 ± 10 | 100 ± 4 |
| AD-1632840.1 | 17 ± 4 | 62 ± 5 | 77 ± 7 | AD-1658032.1 | 7 ± 2 | 34 ± 3 | 52 ± 3 |
| AD-1632841.1 | 9 ± 2 | 45 ± 8 | 70 ± 7 | AD-1658033.1 | 2 ± 0 | 21 ± 4 | 32 ± 6 |
| AD-1632842.1 | 62 ± 11 | 60 ± 4 | 67 ± 13 | AD-1658034.1 | 29 ± 6 | 77 ± 17 | 72 ± 12 |
| AD-1632843.1 | 6 ± 3 | 38 ± 6 | 45 ± 5 | AD-1658035.1 | 3 ± 1 | 9 ± 1 | 18 ± 3 |
| AD-1632844.1 | 7 ± 1 | 28 ± 5 | 45 ± 6 | AD-1658036.1 | 3 ± 0 | 12 ± 2 | 20 ± 4 |
| AD-1632846.1 | 7 ± 3 | 30 ± 7 | 54 ± 10 | AD-1658038.1 | 5 ± 2 | 18 ± 2 | 38 ± 1 |
| AD-1632847.1 | 9 ± 4 | 35 ± 7 | 47 ± 4 | AD-1658039.1 | 2 ± 0 | 5 ± 1 | 15 ± 7 |
| AD-1632848.1 | 67 ± 11 | 92 ± 8 | 108 ± 8 | AD-1658040.1 | 5 ± 1 | 18 ± 2 | 34 ± 5 |
| AD-1632849.1 | 4 ± 1 | 13 ± 2 | 22 ± 1 | AD-1658041.1 | 7 ± 2 | 16 ± 0 | 31 ± 4 |
| AD-1632836.1 | 1 ± 0 | 3 ± 2 | 7 ± 1 | AD-1658042.1 | 1 ± 0 | 1 ± 0 | 3 ± 1 |
| AD-1632850.1 | 26 ± 2 | 62 ± 7 | 70 ± 9 | AD-1658043.1 | 2 ± 0 | 7 ± 1 | 14 ± 2 |
| AD-1632851.1 | 17 ± 5 | 53 ± 8 | 74 ± 5 | AD-1658044.1 | 1 ± 0 | 6 ± 2 | 12 ± 2 |
| AD-1632852.1 | 4 ± 2 | 23 ± 4 | 46 ± 10 | AD-1658045.1 | 2 ± 1 | 6 ± 2 | 16 ± 2 |
| AD-1632853.1 | 16 ± 7 | 55 ± 14 | 62 ± 7 | AD-1658046.1 | 2 ± 1 | 6 ± 1 | 19 ± 5 |
| AD-1632854.1 | 40 ± 12 | 68 ± 11 | 74 ± 9 | AD-1658047.1 | 6 ± 3 | 17 ± 1 | 30 ± 7 |
| AD-1632855.1 | 2 ± 1 | 9 ± 1 | 13 ± 1 | AD-1658048.1 | 3 ± 1 | 9 ± 3 | 18 ± 4 |
| AD-1632856.1 | 21 ± 6 | 47 ± 8 | 75 ± 9 | AD-1658049.1 | 1 ± 0 | 7 ± 1 | 16 ± 3 |
| AD-1632857.1 | 8 ± 3 | 40 ± 5 | 43 ± 11 | AD-1658050.1 | 5 ± 1 | 20 ± 5 | 29 ± 4 |
| AD-1632858.1 | 6 ± 1 | 23 ± 6 | 48 ± 4 | AD-1658051.1 | 6 ± 2 | 14 ± 1 | 31 ± 2 |
| AD-1632859.1 | 3 ± 2 | 12 ± 3 | 43 ± 8 | AD-1658052.1 | 2 ± 0 | 6 ± 0 | 17 ± 3 |
| AD-1632860.1 | 9 ± 3 | 27 ± 4 | 57 ± 5 | AD-1658053.1 | 6 ± 1 | 22 ± 2 | 51 ± 4 |
| AD-1632861.1 | 10 ± 2 | 38 ± 5 | 63 ± 8 | AD-1658054.1 | 2 ± 1 | 8 ± 4 | 22 ± 3 |
| AD-1632862.1 | 53 ± 10 | 69 ± 10 | 100 ± 11 | AD-1658055.1 | 12 ± 4 | 40 ± 5 | 69 ± 6 |
| AD-1632863.1 | 13 ± 2 | 29 ± 5 | 75 ± 14 | AD-1658056.1 | 18 ± 5 | 51 ± 1 | 80 ± 5 |
| AD-1632864.1 | 61 ± 11 | 74 ± 9 | 113 ± 24 | AD-1658057.1 | 67 ± 6 | 66 ± 16 | 96 ± 18 |
| AD-1632865.1 | 37 ± 4 | 63 ± 10 | 86 ± 17 | AD-1658058.1 | 19 ± 5 | 40 ± 6 | 56 ± 12 |
| AD-1632866.1 | 17 ± 1 | 42 ± 8 | 62 ± 8 | AD-1658059.1 | 12 ± 4 | 27 ± 6 | 41 ± 7 |
| AD-1632991.1 | 18 ± 5 | 50 ± 2 | 74 ± 10 | AD-1658184.1 | 10 ± 2 | 38 ± 9 | 58 ± 9 |
| AD-1632992.1 | 63 ± 9 | 100 ± 20 | 90 ± 11 | AD-1658185.1 | 38 ± 6 | 67 ± 12 | 86 ± 13 |
| AD-1632993.1 | 50 ± 4 | 87 ± 8 | 102 ± 20 | AD-1658186.1 | 5 ± 1 | 29 ± 1 | 55 ± 9 |
| AD-1632994.1 | 6 ± 1 | 28 ± 2 | 65 ± 14 | AD-1658187.1 | 3 ± 0 | 11 ± 1 | 32 ± 4 |
| AD-1632995.1 | 35 ± 9 | 47 ± 4 | 75 ± 17 | AD-1658188.1 | 4 ± 2 | 11 ± 3 | 26 ± 4 |
| AD-1632996.1 | 13 ± 2 | 42 ± 8 | 76 ± 6 | AD-1658189.1 | 7 ± 2 | 27 ± 4 | 56 ± 12 |
| AD-1632997.1 | 5 ± 1 | 23 ± 4 | 50 ± 6 | AD-1658190.1 | 4 ± 1 | 19 ± 6 | 31 ± 6 |
| AD-1632998.1 | 8 ± 2 | 23 ± 2 | 58 ± 4 | AD-1658191.1 | 13 ± 4 | 23 ± 3 | 48 ± 11 |
| AD-1632999.1 | 41 ± 5 | 75 ± 4 | 83 ± 16 | AD-1658192.1 | 21 ± 6 | 46 ± 10 | 77 ± 9 |
| AD-1633000.1 | 106 ± 9 | 126 ± 8 | 96 ± 10 | AD-1658193.1 | 25 ± 3 | 60 ± 5 | 83 ± 11 |
| AD-1633003.1 | 4 ± 1 | 33 ± 7 | 64 ± 9 | AD-1658196.1 | 2 ± 0 | 6 ± 2 | 22 ± 6 |
| AD-1633004.1 | 55 ± 10 | 83 ± 11 | 122 ± 18 | AD-1658197.1 | 8 ± 1 | 24 ± 5 | 32 ± 3 |
| AD-1633007.1 | 6 ± 1 | 19 ± 2 | 69 ± 8 | AD-1658200.1 | 2 ± 0 | 7 ± 1 | 22 ± 4 |
| AD-1633008.1 | 26 ± 7 | 53 ± 8 | 53 ± 3 | AD-1658201.1 | 19 ± 3 | 63 ± 12 | 89 ± 9 |
| AD-1633009.1 | 5 ± 1 | 12 ± 3 | 30 ± 5 | AD-1658202.1 | 10 ± 1 | 25 ± 5 | 50 ± 9 |
| AD-1633010.1 | 5 ± 0 | 9 ± 3 | 13 ± 2 | AD-1658203.1 | 1 ± 0 | 1 ± 0 | 4 ± 1 |
| AD-1633011.1 | 3 ± 1 | 10 ± 2 | 26 ± 1 | AD-1658204.1 | 1 ± 0 | 5 ± 1 | 16 ± 3 |
| AD-1633012.1 | 5 ± 1 | 33 ± 4 | 64 ± 9 | AD-1658205.1 | 2 ± 0 | 4 ± 1 | 10 ± 2 |
| AD-1633013.1 | 3 ± 1 | 10 ± 3 | 25 ± 5 | AD-1658206.1 | 1 ± 0 | 5 ± 1 | 14 ± 3 |
| AD-1633014.1 | 3 ± 1 | 23 ± 4 | 35 ± 5 | AD-1658207.1 | 4 ± 1 | 12 ± 3 | 22 ± 5 |
| AD-1633015.1 | 16 ± 4 | 58 ± 5 | 72 ± 7 | AD-1658208.1 | 4 ± 0 | 26 ± 6 | 39 ± 6 |
| AD-1633016.1 | 21 ± 5 | 90 ± 5 | 104 ± 13 | AD-1658209.1 | 14 ± 3 | 51 ± 4 | 71 ± 4 |
| AD-1633018.1 | 84 ± 5 | 118 ± 17 | 109 ± 7 | AD-1658211.1 | 84 ± 18 | 105 ± 10 | 102 ± 18 |
| AD-1633019.1 | 8 ± 1 | 28 ± 0 | 50 ± 5 | AD-1658212.1 | 2 ± 0 | 4 ± 1 | 8 ± 3 |
| AD-1633020.1 | 27 ± 5 | 70 ± 6 | 96 ± 13 | AD-1658213.1 | 9 ± 2 | 28 ± 6 | 56 ± 6 |
| AD-1633027.1 | 17 ± 2 | 46 ± 7 | 65 ± 12 | AD-1658220.1 | 4 ± 1 | 6 ± 1 | 15 ± 4 |
| AD-1633028.1 | 15 ± 2 | 49 ± 8 | 63 ± 7 | AD-1658221.1 | 5 ± 1 | 28 ± 4 | 49 ± 7 |
| AD-1633029.1 | 113 ± 4 | 120 ± 14 | 108 ± 15 | AD-1658222.1 | 28 ± 2 | 47 ± 4 | 58 ± 9 |
| AD-1633030.1 | 19 ± 3 | 70 ± 13 | 86 ± 7 | AD-1658223.1 | 4 ± 1 | 25 ± 7 | 36 ± 3 |
| AD-1633031.1 | 6 ± 3 | 14 ± 2 | 40 ± 7 | AD-1658224.1 | 4 ± 1 | 4 ± 0 | 10 ± 2 |
| AD-84731.2 | 8 ± 1 | 16 ± 1 | 28 ± 7 | AD-1658225.1 | 3 ± 1 | 6 ± 1 | 12 ± 2 |
| AD-1633032.1 | 6 ± 2 | 14 ± 4 | 29 ± 7 | AD-1658226.1 | 4 ± 1 | 6 ± 1 | 19 ± 4 |
| AD-1633033.1 | 6 ± 1 | 12 ± 2 | 29 ± 3 | AD-1658227.1 | 3 ± 1 | 5 ± 1 | 10 ± 3 |
| AD-1633034.1 | 82 ± 12 | 92 ± 11 | 95 ± 16 | AD-1658228.1 | 42 ± 8 | 75 ± 17 | 95 ± 13 |
| AD-1684516.1 | 42 ± 11 | 83 ± 18 | 77 ± 23 | AD-1684517.1 | 10 ± 2 | 30 ± 9 | 70 ± 6 |
| AD-1684518.1 | 80 ± 10 | 107 ± 22 | 108 ± 20 | AD-1684519.1 | 100 ± 7 | 107 ± 21 | 111 ± 21 |
| AD-1684520.1 | 24 ± 6 | 75 ± 13 | 87 ± 8 | AD-1684521.1 | 6 ± 1 | 15 ± 5 | 21 ± 2 |
| AD-1633048.1 | 46 ± 9 | 82 ± 8 | 98 ± 10 | AD-1658242.1 | 20 ± 6 | 43 ± 6 | 65 ± 10 |
| AD-1633049.1 | 10 ± 2 | 28 ± 6 | 39 ± 10 | AD-1658243.1 | 9 ± 1 | 19 ± 2 | 45 ± 4 |
| AD-1633094.1 | 19 ± 7 | 31 ± 3 | 56 ± 7 | AD-1658288.1 | 24 ± 4 | 53 ± 10 | 63 ± 13 |
| AD-1633095.1 | 48 ± 15 | 63 ± 13 | 66 ± 11 | AD-1658289.1 | 16 ± 5 | 36 ± 7 | 55 ± 7 |
| AD-1633119.1 | 43 ± 8 | 72 ± 17 | 96 ± 16 | AD-1658313.1 | 25 ± 3 | 49 ± 8 | 54 ± 5 |
| AD-1633121.1 | 18 ± 5 | 53 ± 10 | 74 ± 13 | AD-1658315.1 | 3 ± 1 | 11 ± 2 | 34 ± 9 |
| AD-1633122.1 | 66 ± 18 | 85 ± 17 | 96 ± 15 | AD-1658316.1 | 13 ± 4 | 40 ± 8 | 63 ± 8 |
| AD-1633254.2 | 11 ± 4 | 21 ± 6 | 32 ± 5 | AD-1658448.2 | 7 ± 1 | 16 ± 4 | 21 ± 9 |
| AD-1633257.2 | 10 ± 2 | 27 ± 4 | 39 ± 7 | AD-1658451.2 | 9 ± 1 | 23 ± 3 | 28 ± 3 |
| AD-1633269.2 | 10 ± 0 | 18 ± 4 | 22 ± 2 | AD-1658463.2 | 7 ± 1 | 9 ± 2 | 11 ± 1 |
| AD-1633270.2 | 47 ± 3 | 68 ± 2 | 75 ± 12 | AD-1658464.2 | 48 ± 4 | 69 ± 8 | 81 ± 13 |
| AD-1633271.2 | 12 ± 3 | 29 ± 8 | 44 ± 10 | AD-1658465.2 | 8 ± 3 | 17 ± 3 | 22 ± 4 |
| AD-1633272.2 | 27 ± 6 | 35 ± 6 | 57 ± 4 | AD-1658466.2 | 20 ± 3 | 27 ± 4 | 43 ± 7 |
| AD-1633273.2 | 76 ± 2 | 78 ± 2 | 98 ± 17 | AD-1658467.2 | 83 ± 6 | 76 ± 11 | 101 ± 11 |
| AD-1633290.2 | 42 ± 4 | 68 ± 11 | 75 ± 9 | AD-1658484.2 | 5 ± 1 | 16 ± 6 | 28 ± 3 |
| AD-1633291.2 | 30 ± 5 | 50 ± 13 | 64 ± 11 | AD-1658485.2 | 73 ± 15 | 97 ± 8 | 93 ± 20 |
| AD-1633324.2 | 6 ± 2 | 14 ± 3 | 22 ± 5 | AD-1658519.2 | 2 ± 0 | 4 ± 2 | 4 ± 1 |
| AD-84739.5 | 2 ± 0 | 3 ± 1 | 3 ± 1 | AD-1658520.2 | 2 ± 0 | 4 ± 1 | 8 ± 1 |
| AD-1633325.2 | 2 ± 0 | 3 ± 1 | 5 ± 2 | AD-1658521.2 | 1 ± 1 | 4 ± 1 | 5 ± 1 |
| AD-1633326.2 | 3 ± 1 | 7 ± 1 | 17 ± 1 | AD-1658522.2 | 1 ± 0 | 2 ± 0 | 3 ± 1 |
| AD-1633327.2 | 20 ± 4 | 38 ± 5 | 49 ± 3 | AD-1658523.2 | 12 ± 1 | 29 ± 6 | 58 ± 3 |
| AD-1633328.2 | 7 ± 1 | 16 ± 2 | 27 ± 3 | AD-1658524.2 | 4 ± 1 | 11 ± 1 | 28 ± 2 |
| AD-1633329.2 | 7 ± 1 | 21 ± 4 | 44 ± 7 | AD-1658525.2 | 2 ± 1 | 6 ± 1 | 12 ± 4 |
| AD-1633330.2 | 14 ± 3 | 34 ± 2 | 58 ± 1 | AD-1658526.2 | 6 ± 1 | 24 ± 7 | 33 ± 6 |
| AD-1633331.2 | 112 ± 5 | 115 ± 6 | 123 ± 19 | AD-1658527.2 | 31 ± 2 | 59 ± 5 | 80 ± 9 |
| AD-1633332.2 | 12 ± 3 | 40 ± 8 | 61 ± 6 | AD-1658528.2 | 8 ± 2 | 25 ± 3 | 33 ± 3 |
| AD-1633333.2 | 2 ± 0 | 8 ± 2 | 15 ± 1 | AD-1658529.2 | 3 ± 0 | 6 ± 1 | 12 ± 2 |
| AD-1633334.2 | 9 ± 1 | 24 ± 4 | 50 ± 3 | AD-1658530.2 | 6 ± 1 | 11 ± 2 | 18 ± 4 |
| AD-1633335.2 | 105 ± 18 | 127 ± 10 | 140 ± 12 | AD-1658531.2 | 27 ± 2 | 50 ± 5 | 73 ± 8 |
| AD-1633343.2 | 63 ± 5 | 87 ± 7 | 100 ± 14 | AD-1658539.2 | 32 ± 4 | 44 ± 5 | 69 ± 4 |
| AD-1633345.2 | 29 ± 3 | 56 ± 5 | 87 ± 11 | AD-1658541.2 | 13 ± 3 | 27 ± 3 | 49 ± 5 |
| AD-1633346.2 | 5 ± 1 | 13 ± 3 | 27 ± 6 | AD-1658542.2 | 5 ± 1 | 16 ± 5 | 20 ± 4 |
| AD-1633409.2 | 4 ± 0 | 12 ± 4 | 21 ± 4 | AD-1658605.2 | 4 ± 1 | 13 ± 5 | 23 ± 1 |
| AD-1633453.2 | 67 ± 7 | 81 ± 13 | 102 ± 6 | AD-1658650.2 | 25 ± 1 | 53 ± 3 | 82 ± 6 |
| AD-1633464.2 | 14 ± 1 | 38 ± 4 | 67 ± 3 | AD-1658661.2 | 5 ± 1 | 14 ± 2 | 27 ± 3 |
| AD-1633465.2 | 5 ± 1 | 15 ± 3 | 39 ± 3 | AD-1658662.2 | 4 ± 3 | 10 ± 6 | 12 ± 2 |
| AD-1633466.2 | 11 ± 2 | 34 ± 10 | 61 ± 5 | AD-1658663.2 | 3 ± 1 | 10 ± 1 | 19 ± 0 |
| AD-1633467.2 | 6 ± 2 | 22 ± 1 | 39 ± 6 | AD-1658664.2 | 3 ± 1 | 8 ± 2 | 13 ± 2 |
| AD-1633468.2 | 85 ± 8 | 104 ± 9 | 114 ± 3 | AD-1658665.2 | 52 ± 6 | 67 ± 5 | 89 ± 7 |
| AD-1633604.2 | 82 ± 13 | 103 ± 14 | 118 ± 17 | AD-1658801.2 | 51 ± 6 | 75 ± 6 | 106 ± 12 |
| AD-1633621.2 | 4 ± 1 | 8 ± 1 | 21 ± 2 | AD-1658818.2 | 3 ± 1 | 7 ± 2 | 16 ± 2 |
| AD-1633622.2 | 24 ± 5 | 53 ± 5 | 84 ± 10 | AD-1658819.2 | 25 ± 4 | 48 ± 13 | 75 ± 7 |
| AD-1633623.2 | 46 ± 4 | 75 ± 9 | 118 ± 6 | AD-1658820.2 | 10 ± 2 | 35 ± 4 | 58 ± 6 |
| AD-1633627.2 | 6 ± 1 | 20 ± 4 | 34 ± 1 | AD-1658824.2 | 4 ± 1 | 8 ± 2 | 19 ± 3 |
| AD-1633628.2 | 4 ± 1 | 11 ± 2 | 19 ± 2 | AD-1658825.2 | 2 ± 1 | 7 ± 1 | 14 ± 3 |
| AD-1633630.2 | 41 ± 7 | 56 ± 5 | 67 ± 15 | AD-1658827.2 | 15 ± 2 | 20 ± 5 | 53 ± 9 |
| AD-1633631.2 | 5 ± 1 | 11 ± 2 | 22 ± 4 | AD-1658828.2 | 3 ± 1 | 6 ± 1 | 10 ± 2 |
| AD-1633632.2 | 3 ± 1 | 8 ± 1 | 17 ± 2 | AD-1658829.2 | 3 ± 1 | 9 ± 3 | 17 ± 5 |
| AD-1633633.1 | 4 ± 1 | 9 ± 1 | 33 ± 9 | AD-1658830.1 | 3 ± 1 | 9 ± 2 | 20 ± 6 |
| AD-1633634.1 | 3 ± 1 | 5 ± 1 | 19 ± 4 | AD-1658831.1 | 1 ± 0 | 2 ± 0 | 6 ± 2 |
| AD-1633635.1 | 20 ± 3 | 36 ± 6 | 71 ± 8 | AD-1658832.1 | 4 ± 1 | 10 ± 2 | 29 ± 4 |
| AD-1633636.1 | 1 ± 0 | 2 ± 0 | 6 ± 1 | AD-1658833.1 | 1 ± 0 | 1 ± 0 | 4 ± 1 |
| AD-1633637.1 | 9 ± 1 | 25 ± 7 | 54 ± 17 | AD-1658834.1 | 2 ± 1 | 2 ± 0 | 5 ± 2 |
| AD-1633638.1 | 24 ± 5 | 36 ± 9 | 71 ± 10 | AD-1658835.1 | 7 ± 1 | 13 ± 1 | 30 ± 6 |
| AD-1633639.1 | 1 ± 0 | 2 ± 0 | 5 ± 1 | AD-1658836.1 | 3 ± 1 | 5 ± 2 | 12 ± 4 |
| AD-1633640.1 | 4 ± 1 | 6 ± 1 | 17 ± 1 | AD-1658837.1 | 3 ± 0 | 5 ± 1 | 18 ± 5 |
| AD-1633641.1 | 7 ± 3 | 17 ± 3 | 31 ± 8 | AD-1658838.1 | 2 ± 0 | 3 ± 1 | 8 ± 1 |
| AD-1633642.1 | 39 ± 7 | 65 ± 11 | 71 ± 12 | AD-1658839.1 | 2 ± 1 | 4 ± 1 | 10 ± 2 |
| AD-1633643.1 | 23 ± 1 | 39 ± 9 | 70 ± 14 | AD-1658840.1 | 8 ± 2 | 10 ± 2 | 34 ± 8 |
| AD-1633644.1 | 3 ± 0 | 8 ± 2 | 25 ± 4 | AD-1658841.1 | 2 ± 1 | 8 ± 1 | 16 ± 4 |
| AD-1633645.1 | 9 ± 2 | 20 ± 3 | 37 ± 5 | AD-1658842.1 | 2 ± 1 | 3 ± 1 | 8 ± 2 |
| AD-1633646.1 | 42 ± 6 | 48 ± 8 | 55 ± 8 | AD-1658843.1 | 19 ± 5 | 28 ± 3 | 48 ± 4 |
| AD-1633647.1 | 87 ± 9 | 78 ± 10 | 89 ± 9 | AD-1658844.1 | 66 ± 12 | 81 ± 11 | 93 ± 8 |
| AD-1633648.1 | 8 ± 3 | 13 ± 3 | 32 ± 4 | AD-1658845.1 | 2 ± 0 | 5 ± 0 | 14 ± 1 |
| AD-1633649.1 | 27 ± 3 | 48 ± 8 | 87 ± 3 | AD-1658846.1 | 2 ± 1 | 7 ± 3 | 19 ± 5 |
| AD-1633650.1 | 31 ± 8 | 54 ± 7 | 82 ± 8 | AD-1658847.1 | 17 ± 8 | 30 ± 5 | 53 ± 4 |
| AD-1633651.1 | 44 ± 6 | 63 ± 4 | 75 ± 8 | AD-1658848.1 | 19 ± 3 | 32 ± 5 | 53 ± 4 |
| AD-1633652.1 | 10 ± 1 | 18 ± 5 | 28 ± 3 | AD-1658849.1 | 17 ± 3 | 25 ± 4 | 46 ± 6 |
| AD-84707.2 | 2 ± 1 | 5 ± 2 | 12 ± 2 | AD-1321390.2 | 2 ± 0 | 3 ± 1 | 8 ± 2 |
| AD-1633653.1 | 6 ± 1 | 15 ± 3 | 28 ± 7 | AD-1658850.1 | 5 ± 1 | 10 ± 3 | 16 ± 4 |
| AD-1633678.1 | 113 ± 6 | 99 ± 13 | 96 ± 9 | AD-1658875.1 | 67 ± 7 | 103 ± 12 | 95 ± 2 |
| AD-1633683.1 | 114 ± 14 | 118 ± 4 | 110 ± 8 | AD-1658880.1 | 88 ± 8 | 96 ± 11 | 98 ± 10 |
| AD-1633732.1 | 1 ± 0 | 3 ± 1 | 5 ± 2 | AD-1658929.1 | 2 ± 1 | 5 ± 1 | 6 ± 2 |
| AD-1633733.1 | 6 ± 2 | 10 ± 2 | 20 ± 2 | AD-1658930.1 | 4 ± 1 | 8 ± 3 | 15 ± 2 |
| AD-1633734.1 | 20 ± 3 | 32 ± 4 | 51 ± 6 | AD-1658931.1 | 2 ± 0 | 3 ± 0 | 5 ± 2 |
| AD-1633735.1 | 12 ± 4 | 21 ± 1 | 38 ± 6 | AD-1658932.1 | 3 ± 1 | 9 ± 1 | 21 ± 5 |
| AD-1633736.1 | 5 ± 1 | 8 ± 1 | 20 ± 2 | AD-1658933.1 | 1 ± 0 | 2 ± 1 | 4 ± 1 |
| AD-1633737.1 | 27 ± 3 | 41 ± 2 | 63 ± 5 | AD-1658934.1 | 3 ± 1 | 8 ± 2 | 19 ± 3 |
| AD-1633738.1 | 36 ± 3 | 47 ± 2 | 66 ± 3 | AD-1658935.1 | 9 ± 2 | 15 ± 2 | 31 ± 5 |
| AD-1633739.1 | 2 ± 1 | 4 ± 1 | 8 ± 2 | AD-1658936.1 | 1 ± 0 | 2 ± 1 | 4 ± 1 |
| AD-1633740.1 | 1 ± 0 | 2 ± 0 | 4 ± 1 | AD-1658937.1 | 2 ± 0 | 2 ± 0 | 3 ± 1 |
| AD-1633741.1 | 5 ± 1 | 10 ± 2 | 22 ± 2 | AD-1658938.1 | 2 ± 0 | 4 ± 0 | 6 ± 1 |
| AD-1633742.1 | 12 ± 1 | 22 ± 2 | 37 ± 8 | AD-1658939.1 | 5 ± 1 | 8 ± 1 | 16 ± 1 |
| AD-1633743.1 | 5 ± 2 | 6 ± 2 | 14 ± 3 | AD-1658940.1 | 4 ± 1 | 6 ± 1 | 8 ± 2 |
| AD-1633759.1 | 1 ± 0 | 2 ± 1 | 4 ± 1 | AD-1658954.1 | 1 ± 0 | 2 ± 1 | 3 ± 2 |
| AD-1684522.1 | 4 ± 2 | 7 ± 2 | 13 ± 3 | AD-1658955.1 | 3 ± 1 | 6 ± 1 | 9 ± 2 |
| AD-1684523.1 | 3 ± 1 | 6 ± 1 | 7 ± 2 | AD-1658956.1 | 3 ± 1 | 5 ± 1 | 7 ± 1 |
| AD-1684524.1 | 2 ± 1 | 3 ± 1 | 8 ± 2 | AD-1658957.1 | 2 ± 0 | 3 ± 1 | 5 ± 1 |
| AD-1684525.1 | 15 ± 5 | 18 ± 4 | 34 ± 5 | AD-1658958.1 | 2 ± 1 | 4 ± 2 | 5 ± 3 |
| AD-1684526.1 | 2 ± 1 | 3 ± 1 | 7 ± 1 | AD-1658959.1 | 1 ± 0 | 1 ± 0 | 2 ± 1 |
| AD-1684527.1 | 1 ± 1 | 3 ± 1 | 4 ± 1 | AD-1658960.1 | 1 ± 1 | 2 ± 1 | 3 ± 1 |
| AD-1633777.1 | 14 ± 3 | 18 ± 3 | 38 ± 5 | AD-1658992.1 | 2 ± 1 | 5 ± 1 | 16 ± 2 |
| AD-1633779.1 | 11 ± 4 | 27 ± 7 | 41 ± 7 | AD-1658994.1 | 3 ± 1 | 6 ± 1 | 19 ± 4 |
| AD-1633780.1 | 30 ± 5 | 52 ± 8 | 61 ± 11 | AD-1658995.1 | 51 ± 6 | 52 ± 7 | 55 ± 9 |
| AD-1633840.1 | 66 ± 13 | 91 ± 7 | 99 ± 13 | AD-1659055.1 | 7 ± 1 | 19 ± 3 | 25 ± 3 |
| AD-1633841.1 | 96 ± 13 | 99 ± 25 | 88 ± 9 | AD-1659056.1 | 93 ± 10 | 103 ± 9 | 92 ± 11 |
| AD-1633842.1 | 4 ± 2 | 22 ± 6 | 49 ± 10 | AD-1659057.1 | 2 ± 1 | 13 ± 3 | 31 ± 5 |
| AD-1633843.1 | 6 ± 1 | 36 ± 2 | 55 ± 13 | AD-1659058.1 | 3 ± 1 | 12 ± 2 | 37 ± 7 |
| AD-1633844.1 | 22 ± 3 | 47 ± 10 | 81 ± 9 | AD-1659059.1 | 5 ± 1 | 27 ± 6 | 54 ± 8 |
| AD-1633845.1 | 28 ± 4 | 83 ± 9 | 71 ± 7 | AD-1659060.1 | 14 ± 2 | 41 ± 5 | 65 ± 13 |
| AD-1633846.1 | 10 ± 2 | 32 ± 6 | 58 ± 14 | AD-1659061.1 | 1 ± 0 | 12 ± 3 | 22 ± 8 |
| AD-84712.2 | 2 ± 0 | 8 ± 1 | 17 ± 4 | AD-1659062.1 | 1 ± 0 | 6 ± 2 | 12 ± 3 |
| AD-1633847.1 | 14 ± 3 | 69 ± 10 | 86 ± 5 | AD-1659063.1 | 5 ± 1 | 28 ± 8 | 54 ± 4 |
| AD-1633848.1 | 12 ± 4 | 40 ± 2 | 80 ± 20 | AD-1659064.1 | 3 ± 1 | 22 ± 6 | 31 ± 1 |
| AD-1633849.1 | 8 ± 1 | 34 ± 3 | 64 ± 6 | AD-1659065.1 | 6 ± 2 | 21 ± 5 | 50 ± 12 |
| AD-1633850.1 | 2 ± 0 | 12 ± 3 | 23 ± 1 | AD-1659066.1 | 2 ± 1 | 9 ± 1 | 27 ± 2 |
| AD-1633851.1 | 6 ± 2 | 37 ± 5 | 52 ± 7 | AD-1659067.1 | 3 ± 0 | 24 ± 5 | 46 ± 8 |
| AD-1633852.1 | 8 ± 2 | 39 ± 5 | 62 ± 8 | AD-1659068.1 | 7 ± 1 | 20 ± 6 | 39 ± 4 |
| AD-1633853.1 | 1 ± 0 | 7 ± 2 | 17 ± 4 | AD-1659069.1 | 1 ± 0 | 3 ± 1 | 14 ± 3 |
| AD-1633854.1 | 22 ± 3 | 59 ± 9 | 85 ± 9 | AD-1659070.1 | 7 ± 2 | 27 ± 4 | 52 ± 12 |
| AD-1633855.1 | 21 ± 3 | 82 ± 14 | 91 ± 12 | AD-1659071.1 | 2 ± 0 | 13 ± 3 | 35 ± 5 |
| AD-1684528.1 | 7 ± 2 | 30 ± 5 | 61 ± 8 | AD-1684529.1 | 3 ± 0 | 12 ± 3 | 34 ± 7 |
| AD-1684530.1 | 3 ± 1 | 18 ± 1 | 45 ± 4 | AD-1684531.1 | 2 ± 0 | 9 ± 1 | 33 ± 5 |
| AD-1684532.1 | 7 ± 1 | 28 ± 3 | 44 ± 5 | AD-1684533.1 | 3 ± 1 | 19 ± 8 | 47 ± 11 |
| AD-1684534.1 | 3 ± 1 | 15 ± 1 | 30 ± 3 | AD-1684535.1 | 4 ± 3 | 4 ± 0 | 12 ± 2 |
| AD-1633946.1 | 3 ± 1 | 10 ± 3 | 22 ± 3 | AD-1659162.1 | 1 ± 0 | 6 ± 2 | 19 ± 4 |
| AD-67328.2 | 1 ± 0 | 7 ± 1 | 20 ± 3 | AD-1321384.2 | 3 ± 0 | 9 ± 1 | 22 ± 4 |
| AD-1633947.1 | 3 ± 1 | 10 ± 2 | 23 ± 1 | AD-1659163.1 | 2 ± 1 | 10 ± 1 | 22 ± 3 |
| AD-1633948.1 | 2 ± 1 | 17 ± 9 | 32 ± 5 | AD-1659164.1 | 1 ± 0 | 4 ± 1 | 11 ± 3 |
| AD-1633949.1 | 3 ± 1 | 21 ± 1 | 51 ± 8 | AD-1659165.1 | 3 ± 0 | 12 ± 2 | 23 ± 2 |
| AD-84700.2 | 11 ± 2 | 37 ± 3 | 66 ± 4 | AD-1659166.1 | 2 ± 0 | 12 ± 1 | 25 ± 4 |
| AD-1633950.1 | 1 ± 0 | 9 ± 2 | 29 ± 5 | AD-1659167.1 | 1 ± 0 | 6 ± 2 | 18 ± 2 |
| AD-1633951.1 | 5 ± 1 | 29 ± 6 | 60 ± 10 | AD-1659168.1 | 4 ± 1 | 15 ± 1 | 33 ± 7 |
| AD-1633991.1 | 3 ± 1 | 19 ± 3 | 46 ± 4 | AD-1659208.1 | 2 ± 0 | 11 ± 2 | 26 ± 4 |
| AD-1633992.1 | 14 ± 1 | 45 ± 5 | 77 ± 9 | AD-1659209.1 | 3 ± 1 | 8 ± 2 | 23 ± 5 |
| AD-1633993.1 | 27 ± 5 | 57 ± 10 | 73 ± 3 | AD-1659210.1 | 11 ± 1 | 43 ± 5 | 70 ± 8 |
| AD-1634065.1 | 13 ± 3 | 48 ± 6 | 79 ± 1 | AD-1659282.1 | 4 ± 1 | 18 ± 4 | 44 ± 6 |
| AD-1634066.1 | 45 ± 5 | 77 ± 9 | 98 ± 12 | AD-1659283.1 | 30 ± 2 | 69 ± 14 | 80 ± 11 |
| AD-1634067.1 | 25 ± 5 | 68 ± 6 | 87 ± 11 | AD-1659284.1 | 24 ± 4 | 60 ± 2 | 71 ± 3 |
| AD-1634068.1 | 10 ± 2 | 38 ± 4 | 58 ± 3 | AD-1659285.1 | 11 ± 1 | 38 ± 2 | 64 ± 5 |
| AD-84730.2 | 7 ± 1 | 19 ± 4 | 30 ± 8 | AD-1659286.1 | 5 ± 1 | 21 ± 7 | 41 ± 6 |
| AD-1634069.1 | 5 ± 1 | 19 ± 4 | 34 ± 6 | AD-1659287.1 | 2 ± 1 | 12 ± 2 | 22 ± 5 |
| AD-1634070.1 | 2 ± 1 | 12 ± 4 | 24 ± 4 | AD-1659288.1 | 3 ± 1 | 13 ± 3 | 22 ± 2 |
| AD-1634071.1 | 29 ± 3 | 65 ± 6 | 98 ± 16 | AD-1659289.1 | 24 ± 6 | 55 ± 8 | 79 ± 12 |
| AD-1684536.1 | 54 ± 6 | 83 ± 5 | 84 ± 5 | AD-1684537.1 | 75 ± 4 | 105 ± 5 | 109 ± 19 |
| AD-1684538.1 | 64 ± 6 | 103 ± 6 | 102 ± 20 | AD-1684539.1 | 61 ± 5 | 105 ± 8 | 83 ± 8 |
| AD-1684540.1 | 73 ± 5 | 65 ± 4 | 87 ± 9 | AD-1684541.1 | 57 ± 9 | 67 ± 10 | 96 ± 15 |
| AD-1684542.1 | 89 ± 22 | 65 ± 5 | 97 ± 9 | AD-1684543.1 | 85 ± 10 | 78 ± 1 | 104 ± 19 |
| AD-1684544.1 | 98 ± 15 | 87 ± 8 | 117 ± 19 | AD-1684545.1 | 56 ± 9 | 56 ± 6 | 94 ± 6 |
| AD-1684546.1 | 79 ± 9 | 84 ± 9 | 94 ± 12 | AD-1684547.1 | 23 ± 3 | 45 ± 3 | 64 ± 8 |
| AD-1684548.1 | 58 ± 9 | 62 ± 12 | 86 ± 18 | AD-1684549.1 | 16 ± 1 | 44 ± 8 | 57 ± 8 |
| AD-1684550.1 | 47 ± 7 | 63 ± 15 | 100 ± 17 | AD-1684551.1 | 37 ± 3 | 50 ± 7 | 80 ± 3 |
| AD-1684552.1 | 55 ± 5 | 74 ± 14 | 112 ± 20 | AD-1684553.1 | 31 ± 5 | 54 ± 7 | 76 ± 9 |
| AD-1684554.1 | 75 ± 8 | 98 ± 17 | 107 ± 18 | AD-1684555.1 | 25 ± 2 | 50 ± 9 | 53 ± 5 |
| AD-1684556.1 | 76 ± 10 | 82 ± 12 | 115 ± 26 | AD-1684557.1 | 26 ± 7 | 45 ± 8 | 75 ± 20 |
| AD-1684558.1 | 57 ± 9 | 65 ± 7 | 101 ± 14 | AD-1684559.1 | 21 ± 6 | 41 ± 5 | 69 ± 6 |
| AD-1634072.1 | 8 ± 1 | 19 ± 3 | 32 ± 8 | AD-1684560.1 | 6 ± 0 | 15 ± 3 | 25 ± 4 |
| AD-1634073.1 | 5 ± 1 | 13 ± 3 | 18 ± 3 | AD-1684561.1 | 3 ± 1 | 10 ± 1 | 11 ± 3 |
| AD-1634074.1 | 3 ± 0 | 8 ± 1 | 15 ± 3 | AD-1659290.1 | 3 ± 1 | 9 ± 1 | 15 ± 1 |
| AD-1634075.1 | 5 ± 1 | 15 ± 2 | 32 ± 9 | AD-1659291.1 | 5 ± 1 | 15 ± 2 | 19 ± 3 |
| AD-1634076.1 | 11 ± 2 | 36 ± 5 | 45 ± 4 | AD-1659292.1 | 9 ± 1 | 25 ± 5 | 37 ± 9 |
| AD-1634077.1 | 12 ± 2 | 30 ± 5 | 59 ± 8 | AD-1659293.1 | 3 ± 2 | 10 ± 2 | 16 ± 1 |
| AD-1634078.1 | 11 ± 3 | 29 ± 7 | 55 ± 8 | AD-1659294.1 | 5 ± 1 | 13 ± 3 | 24 ± 4 |
| AD-1634079.1 | 3 ± 1 | 10 ± 3 | 21 ± 4 | AD-1659295.1 | 2 ± 0 | 4 ± 0 | 11 ± 1 |
| AD-1634080.1 | 10 ± 3 | 34 ± 2 | 48 ± 7 | AD-1659296.1 | 6 ± 2 | 15 ± 2 | 35 ± 7 |
| AD-1634081.1 | 5 ± 1 | 20 ± 2 | 38 ± 8 | AD-1659297.1 | 14 ± 2 | 26 ± 2 | 52 ± 7 |
| AD-1634082.1 | 27 ± 3 | 60 ± 4 | 79 ± 11 | AD-1659298.1 | 17 ± 3 | 34 ± 6 | 60 ± 10 |
| AD-1634105.1 | 2 ± 0 | 4 ± 1 | 12 ± 2 | AD-1659321.1 | 2 ± 1 | 5 ± 0 | 15 ± 2 |
| AD-1634107.1 | 6 ± 2 | 21 ± 2 | 37 ± 9 | AD-1659323.1 | 6 ± 1 | 13 ± 3 | 25 ± 2 |
| AD-1634109.1 | 17 ± 2 | 40 ± 5 | 66 ± 8 | AD-1659325.1 | 7 ± 2 | 17 ± 3 | 39 ± 6 |
| AD-1634110.1 | 9 ± 2 | 26 ± 5 | 42 ± 2 | AD-1659326.1 | 6 ± 1 | 19 ± 1 | 41 ± 6 |
| AD-1634111.1 | 5 ± 2 | 11 ± 3 | 25 ± 3 | AD-1659327.1 | 3 ± 1 | 14 ± 1 | 18 ± 2 |
| AD-1634112.1 | 10 ± 1 | 32 ± 2 | 50 ± 9 | AD-1659328.1 | 3 ± 1 | 10 ± 1 | 24 ± 6 |
| AD-1634113.1 | 7 ± 2 | 17 ± 3 | 37 ± 4 | AD-1659329.1 | 4 ± 1 | 15 ± 1 | 29 ± 6 |
| AD-1634114.1 | 17 ± 5 | 33 ± 1 | 68 ± 8 | AD-1659330.1 | 2 ± 1 | 5 ± 1 | 11 ± 2 |
| AD-1634115.1 | 35 ± 5 | 63 ± 11 | 110 ± 7 | AD-1659331.1 | 7 ± 1 | 24 ± 4 | 43 ± 7 |
| AD-84724.2 | 24 ± 2 | 45 ± 6 | 61 ± 8 | AD-1659332.1 | 5 ± 1 | 11 ± 1 | 24 ± 2 |
| AD-1634116.1 | 5 ± 2 | 15 ± 3 | 33 ± 5 | AD-1659333.1 | 3 ± 2 | 9 ± 1 | 17 ± 3 |
| AD-1634117.1 | 9 ± 1 | 32 ± 3 | 45 ± 6 | AD-1659334.1 | 10 ± 3 | 29 ± 1 | 42 ± 6 |
| AD-1634118.1 | 25 ± 4 | 46 ± 5 | 79 ± 4 | AD-1659335.1 | 6 ± 1 | 20 ± 3 | 31 ± 4 |
| AD-68579.2 | 17 ± 5 | 41 ± 5 | 69 ± 6 | AD-1659336.1 | 3 ± 1 | 10 ± 2 | 17 ± 3 |
| AD-1634119.1 | 4 ± 1 | 14 ± 1 | 33 ± 4 | AD-1659337.1 | 3 ± 0 | 10 ± 2 | 21 ± 2 |
| AD-1634120.1 | 11 ± 3 | 33 ± 6 | 80 ± 5 | AD-1659338.1 | 3 ± 1 | 8 ± 3 | 20 ± 1 |
| AD-1634121.1 | 8 ± 2 | 22 ± 6 | 44 ± 4 | AD-1659339.1 | 2 ± 1 | 6 ± 1 | 18 ± 3 |
| AD-1634122.1 | 18 ± 13 | 29 ± 2 | 60 ± 7 | AD-1659340.1 | 2 ± 0 | 15 ± 3 | 24 ± 4 |
| AD-1634123.1 | 4 ± 1 | 14 ± 3 | 41 ± 1 | AD-1659341.1 | 6 ± 1 | 23 ± 3 | 46 ± 8 |
| AD-1634124.1 | 5 ± 1 | 17 ± 6 | 32 ± 5 | AD-1659342.1 | 3 ± 0 | 12 ± 2 | 19 ± 3 |
| AD-1634125.1 | 4 ± 1 | 11 ± 2 | 22 ± 2 | AD-1659343.1 | 4 ± 1 | 10 ± 3 | 26 ± 5 |
| AD-1634126.1 | 6 ± 1 | 16 ± 4 | 30 ± 4 | AD-1659344.1 | 2 ± 0 | 8 ± 1 | 16 ± 3 |
| AD-1634127.1 | 3 ± 0 | 7 ± 2 | 16 ± 1 | AD-1659345.1 | 3 ± 0 | 6 ± 1 | 12 ± 2 |
| AD-1634128.1 | 1 ± 0 | 1 ± 1 | 3 ± 2 | AD-1659346.1 | 1 ± 0 | 2 ± 1 | 5 ± 1 |
| AD-1634129.1 | 2 ± 1 | 6 ± 3 | 15 ± 2 | AD-1659347.1 | 1 ± 0 | 1 ± 0 | 5 ± 2 |
| AD-1634130.1 | 4 ± 1 | 13 ± 1 | 24 ± 4 | AD-1659348.1 | 1 ± 1 | 1 ± 0 | 3 ± 1 |
| AD-1684562.1 | 16 ± 4 | 33 ± 3 | 54 ± 7 | AD-1659349.1 | 9 ± 2 | 23 ± 2 | 40 ± 1 |
| AD-1684563.1 | 1 ± 0 | 1 ± 1 | 3 ± 0 | AD-1659350.1 | 1 ± 0 | 1 ± 1 | 2 ± 1 |
| AD-1684564.1 | 1 ± 0 | 2 ± 1 | 2 ± 1 | AD-1659351.1 | 1 ± 0 | 1 ± 0 | 1 ± 0 |
| AD-1634135.1 | 2 ± 1 | 7 ± 1 | 13 ± 4 | AD-1659371.1 | 1 ± 0 | 2 ± 1 | 4 ± 1 |
| AD-1634136.1 | 2 ± 1 | 7 ± 3 | 12 ± 1 | AD-1659372.1 | 3 ± 1 | 9 ± 1 | 21 ± 4 |
| AD-1634137.1 | 1 ± 0 | 2 ± 0 | 2 ± 1 | AD-1659373.1 | 1 ± 1 | 2 ± 1 | 3 ± 1 |
| AD-1634146.1 | 1 ± 0 | 2 ± 0 | 3 ± 1 | AD-1659382.1 | 1 ± 0 | 1 ± 0 | 1 ± 0 |
| AD-1634147.1 | 1 ± 0 | 4 ± 1 | 10 ± 3 | AD-1659383.1 | 1 ± 1 | 2 ± 0 | 2 ± 0 |
| AD-68585.2 | 5 ± 2 | 20 ± 2 | 29 ± 4 | AD-1659384.1 | 2 ± 1 | 7 ± 1 | 11 ± 5 |
| AD-1634148.1 | 2 ± 0 | 5 ± 1 | 9 ± 2 | AD-1659385.1 | 1 ± 0 | 1 ± 0 | 1 ± 0 |
| AD-1634149.1 | 1 ± 0 | 1 ± 0 | 3 ± 1 | AD-1659386.1 | 1 ± 0 | 1 ± 0 | 3 ± 0 |
| AD-1634150.1 | 1 ± 0 | 1 ± 0 | 4 ± 1 | AD-1659387.1 | 1 ± 0 | 1 ± 0 | 2 ± 1 |
| AD-1634151.1 | 0 ± 0 | 1 ± 0 | 2 ± 0 | AD-1659388.1 | 0 ± 0 | 1 ± 0 | 1 ± 0 |
| AD-1634152.1 | 1 ± 0 | 1 ± 0 | 2 ± 1 | AD-1659389.1 | 1 ± 0 | 1 ± 0 | 2 ± 0 |
| AD-1634153.1 | 2 ± 1 | 2 ± 1 | 5 ± 1 | AD-1659390.1 | 1 ± 0 | 2 ± 0 | 5 ± 2 |
| AD-1634162.1 | 3 ± 0 | 5 ± 2 | 12 ± 2 | AD-1659399.1 | 3 ± 1 | 5 ± 1 | 8 ± 1 |
| AD-1634163.1 | 4 ± 0 | 11 ± 2 | 18 ± 3 | AD-1659400.1 | 9 ± 2 | 18 ± 5 | 30 ± 3 |
| AD-1634164.1 | 29 ± 6 | 35 ± 3 | 48 ± 7 | AD-1659401.1 | 26 ± 1 | 23 ± 3 | 33 ± 4 |
| AD-1634165.1 | 19 ± 3 | 19 ± 2 | 30 ± 5 | AD-1659402.1 | 20 ± 3 | 20 ± 5 | 24 ± 3 |
| AD-1634169.1 | 65 ± 4 | 70 ± 7 | 79 ± 5 | AD-1659406.1 | 63 ± 18 | 59 ± 11 | 53 ± 6 |
| AD-1634170.1 | 84 ± 13 | 74 ± 9 | 72 ± 1 | AD-1659407.1 | 58 ± 5 | 69 ± 8 | 68 ± 6 |
| AD-1634171.1 | 81 ± 15 | 75 ± 8 | 85 ± 7 | AD-1659408.1 | 86 ± 25 | 91 ± 9 | 81 ± 5 |
| AD-1634172.1 | 50 ± 13 | 53 ± 7 | 67 ± 8 | AD-1659409.1 | 58 ± 4 | 68 ± 6 | 65 ± 7 |
| AD-1634173.1 | 60 ± 5 | 61 ± 5 | 56 ± 7 | AD-1659410.1 | 49 ± 6 | 49 ± 2 | 52 ± 7 |
| AD-1634174.1 | 69 ± 2 | 73 ± 5 | 77 ± 9 | AD-1659411.1 | 36 ± 3 | 37 ± 4 | 39 ± 2 |
| AD-1634175.1 | 61 ± 9 | 70 ± 9 | 81 ± 8 | AD-1659412.1 | 36 ± 4 | 41 ± 7 | 42 ± 5 |
| AD-1634176.1 | 70 ± 5 | 82 ± 5 | 88 ± 7 | AD-1659413.1 | 64 ± 9 | 68 ± 4 | 74 ± 6 |
| AD-1634177.1 | 57 ± 12 | 65 ± 9 | 67 ± 4 | AD-1659414.1 | 52 ± 10 | 67 ± 3 | 67 ± 3 |
| AD-1634178.1 | 63 ± 8 | 76 ± 6 | 74 ± 4 | AD-1659415.1 | 58 ± 5 | 76 ± 4 | 67 ± 9 |
| AD-1634179.1 | 79 ± 11 | 83 ± 18 | 103 ± 9 | AD-1659416.1 | 65 ± 6 | 86 ± 7 | 92 ± 7 |
| AD-1634180.1 | 47 ± 7 | 67 ± 10 | 72 ± 14 | AD-1659417.1 | 58 ± 11 | 74 ± 7 | 73 ± 15 |
| AD-1634181.1 | 71 ± 11 | 79 ± 6 | 72 ± 16 | AD-1659418.1 | 54 ± 9 | 69 ± 7 | 56 ± 13 |
| AD-1634182.1 | 62 ± 5 | 77 ± 3 | 86 ± 4 | AD-1659419.1 | 57 ± 17 | 68 ± 7 | 76 ± 7 |
| AD-1634183.1 | 61 ± 11 | 70 ± 11 | 81 ± 11 | AD-1659420.1 | 63 ± 8 | 79 ± 11 | 86 ± 5 |
| AD-1634184.1 | 64 ± 12 | 74 ± 9 | 80 ± 16 | AD-1659421.1 | 33 ± 3 | 56 ± 8 | 62 ± 10 |
| AD-1634185.1 | 58 ± 9 | 71 ± 9 | 76 ± 12 | AD-1659422.1 | 58 ± 7 | 72 ± 18 | 61 ± 6 |
| AD-1634186.1 | 60 ± 9 | 86 ± 12 | 86 ± 10 | AD-1659423.1 | 62 ± 11 | 81 ± 15 | 82 ± 9 |
| AD-1634187.1 | 43 ± 10 | 51 ± 9 | 42 ± 8 | AD-1659424.1 | 81 ± 9 | 92 ± 11 | 82 ± 27 |
| AD-1634188.1 | 37 ± 5 | 53 ± 8 | 48 ± 10 | AD-1659425.1 | 42 ± 8 | 52 ± 14 | 73 ± 4 |
| AD-1634189.1 | 49 ± 5 | 70 ± 17 | 87 ± 8 | AD-1659426.1 | 47 ± 9 | 51 ± 9 | 67 ± 5 |
| AD-1634190.1 | 50 ± 9 | 56 ± 10 | 72 ± 5 | AD-1659427.1 | 46 ± 6 | 59 ± 16 | 76 ± 9 |
| AD-1634191.1 | 44 ± 3 | 56 ± 7 | 65 ± 7 | AD-1659428.1 | 56 ± 6 | 50 ± 8 | 80 ± 15 |
| AD-1634192.1 | 45 ± 10 | 49 ± 5 | 61 ± 5 | AD-1659429.1 | 34 ± 5 | 42 ± 7 | 50 ± 5 |
| AD-1634193.1 | 47 ± 10 | 42 ± 8 | 62 ± 10 | AD-1659430.1 | 63 ± 6 | 47 ± 7 | 69 ± 11 |
| AD-1634194.1 | 49 ± 2 | 53 ± 2 | 62 ± 7 | AD-1659431.1 | 49 ± 8 | 50 ± 7 | 63 ± 11 |
| AD-1634195.1 | 52 ± 6 | 59 ± 11 | 80 ± 7 | AD-1659432.1 | 59 ± 5 | 73 ± 7 | 82 ± 13 |
| AD-1634196.1 | 57 ± 7 | 49 ± 3 | 71 ± 11 | AD-1659433.1 | 50 ± 12 | 43 ± 7 | 68 ± 11 |
| AD-1634197.1 | 52 ± 3 | 62 ± 4 | 70 ± 11 | AD-1659434.1 | 52 ± 5 | 63 ± 5 | 72 ± 8 |
| AD-1634199.1 | 61 ± 8 | 51 ± 5 | 60 ± 13 | AD-1659436.1 | 65 ± 5 | 53 ± 3 | 78 ± 8 |
| AD-1634200.1 | 62 ± 8 | 50 ± 5 | 73 ± 15 | AD-1659437.1 | 57 ± 4 | 61 ± 3 | 55 ± 5 |
| AD-1634203.1 | 77 ± 3 | 63 ± 7 | 86 ± 6 | AD-1659440.1 | 65 ± 4 | 54 ± 6 | 64 ± 13 |
| AD-1634209.1 | 91 ± 15 | 74 ± 8 | 112 ± 17 | AD-1659446.1 | 78 ± 8 | 69 ± 3 | 88 ± 12 |
| AD-1634210.1 | 73 ± 6 | 65 ± 6 | 85 ± 9 | AD-1659447.1 | 72 ± 5 | 68 ± 6 | 93 ± 10 |
| AD-1634211.1 | 74 ± 13 | 69 ± 8 | 85 ± 8 | AD-1659448.1 | 66 ± 12 | 50 ± 9 | 78 ± 9 |
| AD-1634212.1 | 43 ± 3 | 45 ± 3 | 68 ± 8 | AD-1659449.1 | 68 ± 3 | 61 ± 5 | 91 ± 9 |
| AD-1634213.1 | 71 ± 2 | 77 ± 17 | 98 ± 21 | AD-1659450.1 | 73 ± 2 | 61 ± 6 | 103 ± 10 |
| AD-1634214.1 | 85 ± 7 | 59 ± 5 | 85 ± 15 | AD-1659451.1 | 52 ± 9 | 49 ± 9 | 56 ± 7 |
| AD-1634215.1 | 82 ± 5 | 79 ± 9 | 108 ± 9 | AD-1659452.1 | 67 ± 8 | 59 ± 6 | 83 ± 6 |
| AD-1634216.1 | 76 ± 9 | 70 ± 6 | 113 ± 7 | AD-1659453.1 | 77 ± 10 | 64 ± 3 | 90 ± 12 |
| AD-1634217.1 | 92 ± 6 | 83 ± 1 | 116 ± 15 | AD-1659454.1 | 75 ± 4 | 70 ± 6 | 101 ± 6 |
| AD-1684565.1 | 85 ± 7 | 104 ± 15 | 84 ± 14 | AD-1659481.1 | 49 ± 5 | 49 ± 5 | 61 ± 5 |
| AD-1684566.1 | 122 ± 27 | 74 ± 9 | 98 ± 5 | AD-1659482.1 | 67 ± 6 | 51 ± 4 | 108 ± 15 |
| AD-1684567.1 | 85 ± 10 | 74 ± 7 | 98 ± 7 | AD-1659483.1 | 89 ± 2 | 77 ± 9 | 102 ± 5 |
| AD-1684568.1 | 71 ± 6 | 82 ± 12 | 107 ± 14 | AD-1659485.1 | 67 ± 4 | 60 ± 6 | 91 ± 11 |
| AD-1684569.1 | 68 ± 2 | 80 ± 4 | 100 ± 5 | AD-1659487.1 | 68 ± 7 | 88 ± 15 | 91 ± 13 |
| AD-1684570.1 | 78 ± 7 | 83 ± 6 | 123 ± 12 | AD-1659488.1 | 79 ± 7 | 87 ± 13 | 111 ± 9 |
| AD-1634234.1 | 45 ± 8 | 73 ± 12 | 91 ± 5 | AD-1659489.1 | 81 ± 5 | 75 ± 10 | 97 ± 7 |
| AD-1634235.1 | 65 ± 9 | 72 ± 6 | 91 ± 4 | AD-1659490.1 | 47 ± 2 | 68 ± 4 | 89 ± 15 |
| AD-1634236.1 | 70 ± 8 | 75 ± 9 | 96 ± 15 | AD-1659491.1 | 32 ± 6 | 39 ± 3 | 48 ± 5 |
| AD-1634237.1 | 75 ± 7 | 86 ± 14 | 113 ± 11 | AD-1659492.1 | 36 ± 7 | 40 ± 4 | 48 ± 4 |
| AD-1634238.1 | 79 ± 13 | 91 ± 11 | 96 ± 12 | AD-1659493.1 | 74 ± 3 | 78 ± 4 | 104 ± 5 |
| AD-1634282.1 | 64 ± 7 | 58 ± 8 | 72 ± 6 | AD-1659537.1 | 63 ± 9 | 51 ± 8 | 61 ± 6 |
| AD-1634283.1 | 76 ± 8 | 66 ± 3 | 97 ± 18 | AD-1659538.1 | 59 ± 2 | 55 ± 5 | 71 ± 4 |
| AD-1634304.1 | 69 ± 7 | 68 ± 8 | 102 ± 16 | AD-1659559.1 | 70 ± 7 | 61 ± 11 | 85 ± 14 |
| AD-1634305.1 | 51 ± 3 | 44 ± 4 | 50 ± 12 | AD-1659560.1 | 49 ± 3 | 59 ± 3 | 55 ± 4 |
| AD-1634306.1 | 61 ± 5 | 65 ± 5 | 74 ± 4 | AD-1659561.1 | 51 ± 5 | 39 ± 5 | 73 ± 5 |
| AD-1634307.1 | 83 ± 7 | 80 ± 7 | 103 ± 2 | AD-1659562.1 | 74 ± 5 | 66 ± 8 | 93 ± 7 |
| AD-1634308.1 | 61 ± 4 | 73 ± 7 | 82 ± 7 | AD-1659563.1 | 69 ± 3 | 72 ± 4 | 98 ± 8 |
| AD-1634327.1 | 61 ± 8 | 64 ± 7 | 80 ± 15 | AD-1659582.1 | 49 ± 9 | 52 ± 2 | 74 ± 10 |
| AD-1634328.1 | 64 ± 6 | 64 ± 9 | 91 ± 4 | AD-1659583.1 | 44 ± 3 | 56 ± 4 | 70 ± 5 |
| AD-1634329.1 | 65 ± 3 | 64 ± 7 | 85 ± 5 | AD-1659584.1 | 60 ± 5 | 61 ± 9 | 88 ± 11 |
| AD-1634330.1 | 56 ± 2 | 50 ± 6 | 54 ± 11 | AD-1659585.1 | 72 ± 2 | 65 ± 7 | 81 ± 8 |
| AD-1634331.1 | 44 ± 10 | 31 ± 6 | 49 ± 9 | AD-1659586.1 | 53 ± 7 | 51 ± 9 | 72 ± 7 |
| AD-1634332.1 | 46 ± 10 | 49 ± 13 | 81 ± 3 | AD-1659587.1 | 34 ± 6 | 57 ± 1 | 52 ± 17 |
| AD-1634333.1 | 67 ± 10 | 59 ± 15 | 84 ± 13 | AD-1659588.1 | 64 ± 3 | 62 ± 11 | 97 ± 13 |

### In Vivo Mouse and Cyno Studies

All studies were conducted using protocols consistent with local, state and federal regulations as applicable and approved by the Institutional Animal Care and Use Committees (IACUCs) at Alnylam Pharmaceuticals.

In mouse pharmacodynamic studies, female C57BL/6 mice (Charles River Laboratories) were administered a single dose of a vehicle control (1× PBS or 0.9% sodium chloride) or siRNA subcutaneously in the upper back. Bleeds were collected by retro-orbital bleeding. Serum were separated by centrifuging at 13000 rpm at room temperature for 10 mins. Mouse livers were collected and immediately snap frozen in liquid nitrogen, and stored at −80° C. for mRNA and siRNA analysis.

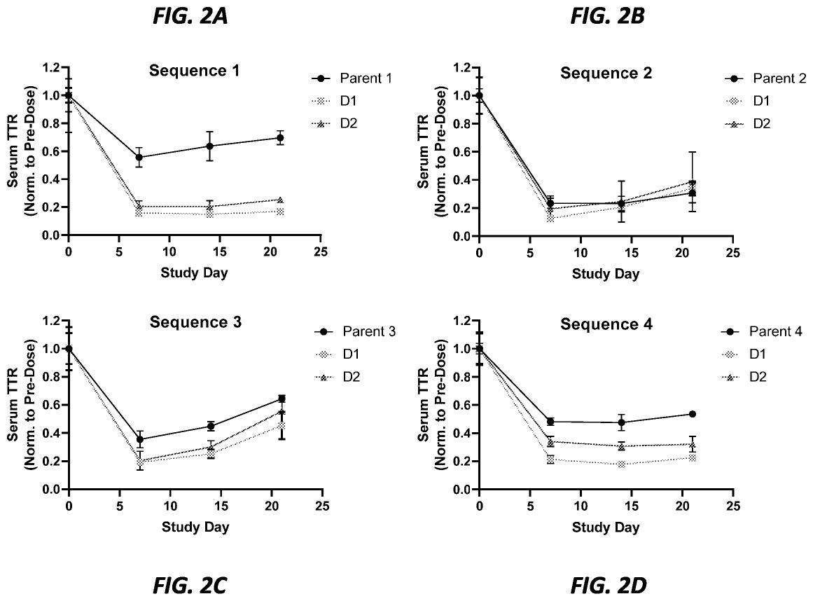

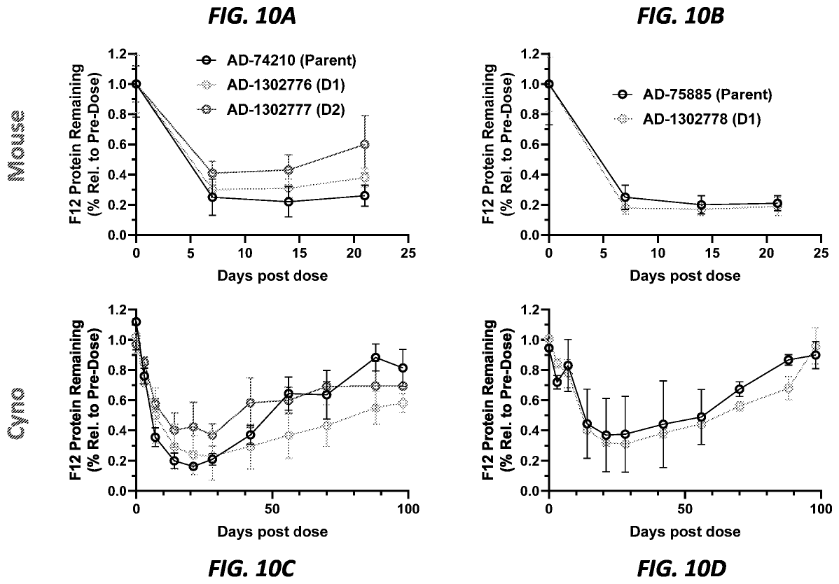

As shown in FIGS. 2A-2D and 10A-10D, the results demonstrate an improved or similar target knockdown in mice and an improved or similar target knockdown and duration of silencing in cyno.

### Serum Protein Quantification

TTR protein was quantified by ELISA from serum isolated from whole blood. ELISA was performed according to manufacturer protocol (ALPCO, 41-PALMS-EO1) after a 3025-fold dilution of the serum samples. Data were normalized to pre-bleed TTR levels. All samples were assayed in duplicate and each data point is the average of all the mice within each cohort (n=3).

In some embodiments, the dsRNA molecule is not a dsRNA molecule listed in any one of Tables 15-25.

TABLE 15
Exemplary dsRNA molecules

| | | SEQ | | SEQ |
|---|---|---|---|---|
| | | ID | | ID |
| Duplex ID | Sense sequence (5′→3′) | NO: | Antisense sequence (5′→3′) | NO: |
| AD-1353484.1 | asasaag(Ahd)gaAfAfGfuguuuuausasa | 2141 | VPusdTsaudAadAacacdTuUfcucuuuuscsu | 2166 |
| AD-1353468.1 | asasaga(Chd)ugAfUfAfcagaacgasusa | 2142 | VPusdAsucdGudTcugudAuCfagucuuuscsc | 2167 |
| AD-1353485.1 | asasagagaaAfGfUfguuu(Uhd)auasusa | 2143 | VPusdAsuadTadAaacadCuUfucucuuususc | 2168 |
| AD-1353483.1 | asascag(Uhd)gcUfAfAfuguuauugsgsa | 2144 | VPusdCscadAudAacaudTaGfcacuguusgsg | 2169 |
| AD-1353486.1 | asasgag(Ahd)aaGfUfGfuuuuauausasa | 2145 | VPusdTsaudAudAaaacdAcUfuucucuususu | 2170 |
| AD-1353479.1 | asasuuggauUfCfGfccau(Uhd)uuasusa | 2146 | VPusdAsuadAadAuggcdGaAfuccaauuscsc | 2171 |
| AD-1353473.1 | ascsaga(Ahd)caGfUfCfcuuaauccsasa | 2147 | VPusdTsggdAudTaaggdAcUfguucuguscsg | 2172 |
| AD-1353467.1 | ascscaggaaAfGfAfcuga(Uhd)acasgsa | 2148 | VPusdCsugdTadTcagudCuUfuccuggusgsc | 2173 |
| AD-1353482.1 | asgsauu(Ahd)gaGfAfGfuuuuauuuscsa | 2149 | VPusdGsaadAudAaaacdTcUfcuaaucususc | 2174 |
| AD-1353480.1 | asusugg(Ahd)uuCfGfCfcauuuuaususa | 2150 | VPusdAsaudAadAauggdCgAfauccaaususc | 2175 |
| AD-1353463.1 | csasuca(Chd)caUfGfCfagauuaugscsa | 2151 | VPusdGscadTadAucugdCaUfggugaugsusu | 2176 |
| AD-1353478.1 | cscsucu(Uhd)ggAfAfUfuggauucgscsa | 2152 | VPusdGscgdAadTccaadTuCfcaagaggsgsc | 2177 |
| AD-1353466.1 | csusacagcaCfAfAfcaaa(Uhd)gugsasa | 2153 | VPusdTscadCadTuugudTgUfgcuguagsgsg | 2178 |
| AD-1353488.1 | gsasaag(Uhd)guUfUfUfauauacggsusa | 2154 | VPusdAsccdGudAuauadAaAfcacuuucsusc | 2179 |
| AD-1353487.1 | gsasgaa(Ahd)guGfUfUfuuauauacsgsa | 2155 | VPusdCsgudAudAuaaadAcAfcuuucucsusu | 2180 |
| AD-1353481.1 | gsasuucgccAfUfUfuuau(Uhd)uuuscsa | 2156 | VPusdGsaadAadAuaaadAuGfgcgaaucscsg | 2181 |
| AD-1353477.1 | gsusccu(Uhd)aaUfCfCfagaaaccusgsa | 2157 | VPusdCsagdGudTucugdGaUfuaaggacsusg | 2182 |
| AD-1353489.1 | gsusguu(Uhd)uaUfAfUfacgguacususa | 2158 | VPusdAsagdTadCcguadTaUfaaaacacsusu | 2183 |
| AD-1353491.1 | usasgac(Ahd)uuGfCfUfauucuguususa | 2159 | VPusdAsaadCadGaauadGcAfaugucuasusu | 2184 |
| AD-1397058 | csusacagcaCfAfAfcaaa(Uhd)gugaaL96 | 2160 | VPusdTscadCadTuugudTgUfgcuguagsgsg | 2178 |
| AD-1397059 | asasaga(Chd)ugAfUfAfcagaacgauaL96 | 2161 | VPusdAsucdGudTcugudAuCfagucuuuscsc | 2167 |
| AD-1397064 | asusugg(Ahd)uuCfGfCfcauuuuauuaL96 | 2162 | VPusdAsaudAadAauggdCgAfauccaaususc | 2175 |
| AD-1397065 | gsasuucgccAfUfUfuuau(Uhd)uuucaL96 | 2163 | VPusdGsaadAadAuaaadAuGfgcgaaucscsg | 2181 |
| AD-1397066 | gsasgaa(Ahd)guGfUfUfuuauauacgaL96 | 2164 | VPusdCsgudAudAuaaadAcAfcuuucucsusu | 2180 |
| AD-1397067 | gsusguu(Uhd)uaUfAfUfacgguacuuaL96 | 2165 | VPusdAsagdTadCcguadTaUfaaaacacsusu | 2183 |

TABLE 16
Exemplary dsRNA Molecules

| | | SEQ | | SEQ |
|---|---|---|---|---|
| | | ID | | ID |
| Duplex ID | Sense sequence (5′→3′)) | NO | Antisense sequence (5′→3′) | NO: |
| AD-1397070.1 | ascsgug(Ahd)ccCfAfAfgcucgcaugaL96 | 2185 | VPusdCsaudGcdGagcudTgGfgucacgusgsa | 2290 |
| AD-1397073.1 | usgsacc(Chd)aaGfCfUfcgcauggucaL96 | 2186 | VPusdGsacdCadTgcgadGcUfugggucascsg | 2291 |
| AD-1397075.1 | ascscca(Ahd)gcUfCfGfcauggucagaL96 | 2187 | VPusdCsugdAcdCaugcdGaGfcuuggguscsa | 2292 |
| AD-1397090.1 | usgsgca(Ghd)caAfCfAfaaggauuugaL96 | 2188 | VPusdCsaadAudCcuuudGuUfgcugccascsu | 2293 |
| AD-1397091.1 | gsgscag(Chd)aaCfAfAfaggauuugaaL96 | 2189 | VPusdTscadAadTccuudTgUfugcugccsasc | 2294 |
| AD-1397094.1 | asascaa(Ahd)ggAfUfUfugaaacuugaL96 | 2190 | VPusdCsaadGudTucaadAuCfcuuuguusgsc | 2295 |
| AD-1397095.1 | ascsaaa(Ghd)gaUfUfUfgaaacuuggaL96 | 2191 | VPusdCscadAgdTuucadAaUfccuuugususg | 2296 |
| AD-1397096.1 | csasaag(Ghd)auUfUfGfaaacuugguaL96 | 2192 | VPusdAsccdAadGuuucdAaAfuccuuugsus | 2297 |
| | | | u | |
| AD-1397097.1 | asasagg(Ahd)uuUfGfAfaacuuggugaL96 | 2193 | VPusdCsacdCadAguuudCaAfauccuuusgsu | 2298 |
| AD-1397098.1 | asasgga(Uhd)uuGfAfAfacuugguguaL96 | 2194 | VPusdAscadCcdAaguudTcAfaauccuususg | 2299 |
| AD-1397102.1 | asgsacg(Ahd)ugUfCfAfaccuuguguaL96 | 2195 | VPusdAscadCadAgguudGaCfaucgucusgsc | 2300 |
| AD-1397107.1 | usasggg(Chd)uaAfCfCfaguucucuuaL96 | 2196 | VPusdAsagdAgdAacugdGuUfagcccuasasa | 2301 |
| AD-1397108.1 | gsgsgcu(Ahd)acCfAfGfuucucuuugaL96 | 2197 | VPusdCsaadAgdAgaacdTgGfuuagcccsusa | 2302 |
| AD-1397109.1 | gsgscua(Ahd)ccAfGfUfucucuuuguaL96 | 2198 | VPusdAscadAadGagaadCuGfguuagccscsu | 2303 |
| AD-1397110.1 | asascca(Ghd)uuCfUfCfuuuguaaggaL96 | 2199 | VPusdCscudTadCaaagdAgAfacugguusasg | 2304 |
| AD-1397116.1 | csuscuu(Uhd)guAfAfGfgacuugugcaL96 | 2200 | VPusdGscadCadAguccdTuAfcaaagagsasa | 2305 |
| AD-1397118.1 | asusacu(Ghd)agGfGfUfgaaauuaagaL96 | 2201 | VPusdCsuudAadTuucadCcCfucaguausgsg | 2306 |
| AD-1397119.1 | ascsuga(Ghd)ggUfGfAfaauuaagggaL96 | 2202 | VPusdCsccdTudAauuudCaCfccucagusasu | 2307 |
| AD-1397120.1 | csusgag(Ghd)guGfAfAfauuaagggaaL96 | 2203 | VPusdTsccdCudTaauudTcAfcccucagsusa | 2308 |
| AD-1397126.1 | usasggu(Ghd)uuUfCfUfgccuuguugaL96 | 2204 | VPusdCsaadCadAggcadGaAfacaccuasgsg | 2309 |
| AD-1397127.1 | asgsgug(Uhd)uuCfUfGfccuuguugaaL96 | 2205 | VPusdTscadAcdAaggcdAgAfaacaccusasg | 2310 |
| AD-1397132.1 | asgscug(Ahd)acAfUfAfuacauagauaL96 | 2206 | VPusdAsucdTadTguaudAuGfuucagcusgsc | 2311 |
| AD-1397133.1 | gscsuga(Ahd)caUfAfUfacauagaugaL96 | 2207 | VPusdCsaudCudAuguadTaUfguucagcsusg | 2312 |
| AD-1397135.1 | gsasaca(Uhd)auAfCfAfuagauguugaL96 | 2208 | VPusdCsaadCadTcuaudGuAfuauguucsasg | 2313 |
| AD-1397138.1 | gsasguu(Ghd)uaGfUfUfggauuugucaL96 | 2209 | VPusdGsacdAadAuccadAcUfacaacucsasa | 2314 |
| AD-1397139.1 | asgsuug(Uhd)agUfUfGfgauuugucuaL96 | 2210 | VPusdAsgadCadAauccdAaCfuacaacuscsa | 2315 |
| AD-1397140.1 | gsusugu(Ahd)guUfGfGfauuugucugaL96 | 2211 | VPusdCsagdAcdAaaucdCaAfcuacaacsusc | 2316 |
| AD-1397144.1 | asgsuug(Ghd)auUfUfGfucuguuuauaL96 | 2212 | VPusdAsuadAadCagacdAaAfuccaacusasc | 2317 |
| AD-1397145.1 | ususgga(Uhd)uuGfUfCfuguuuaugcaL96 | 2213 | VPusdGscadTadAacagdAcAfaauccaascsu | 2318 |
| AD-1397150.1 | ususguc(Uhd)guUfUfAfugcuuggauaL96 | 2214 | VPusdAsucdCadAgcaudAaAfcagacaasasu | 2319 |
| AD-1397159.1 | cscsaga(Ghd)ugAfCfUfaugauagugaL96 | 2215 | VPusdCsacdTadTcauadGuCfacucuggsusg | 2320 |
| AD-1397160.1 | csasgag(Uhd)gaCfUfAfugauagugaaL96 | 2216 | VPusdCsaudGcdGagcudTgGfgucacgusgsa | 2290 |
| AD-1397163.1 | csgscau(Ghd)uaUfCfUfugaaaugcuaL96 | 2217 | VPusdGsacdCadTgcgadGcUfugggucascsg | 2291 |
| AD-1397165.1 | csasugu(Ahd)ucUfUfGfaaaugcuugaL96 | 2218 | VPusdCsugdAcdCaugcdGaGfcuuggguscsa | 2292 |
| AD-1397180.1 | gsgsuuu(Ghd)ggUfAfCfaguuaaaggaL96 | 2219 | VPusdCsaadAudCcuuudGuUfgcugccascsu | 2293 |
| AD-1397181.1 | ususugg(Ghd)uaCfAfGfuuaaaggcaaL96 | 2220 | VPusdTscadAadTccuudTgUfugcugccsasc | 2294 |
| AD-1397184.1 | csasuua(Chd)ugCfCfAfacaguuucgaL96 | 2221 | VPusdCsaadGudTucaadAuCfcuuuguusgsc | 2295 |
| AD-1397185.1 | usascug(Chd)caAfCfAfguuucggcuaL96 | 2222 | VPusdCscadAgdTuucadAaUfccuuugususg | 2296 |
| AD-1397186.1 | gsusucc(Uhd)cuUfCfCfugaaguucuaL96 | 2223 | VPusdAsccdAadGuuucdAaAfuccuuugsus | 2297 |
| | | | u | |
| AD-1397187.1 | ususccu(Chd)uuCfCfUfgaaguucuuaL96 | 2224 | VPusdCsacdCadAguuudCaAfauccuuusgsu | 2298 |
| AD-1397188.1 | uscscuc(Uhd)ucCfUfGfaaguucuugaL96 | 2225 | VPusdAscadCcdAaguudTcAfaauccuususg | 2299 |
| AD-1397191.1 | uscsuuc(Chd)ugAfAfGfuucuugugcaL96 | 2226 | VPusdAscadCadAgguudGaCfaucgucusgsc | 2300 |
| AD-1397196.1 | csascgc(Uhd)ggCfUfUfgugaucuuaaL96 | 2227 | VPusdAsagdAgdAacugdGuUfagcccuasasa | 2301 |
| AD-1397197.1 | usgsggc(Uhd)agAfUfAfggauauacuaL96 | 2228 | VPusdTscadCudAucaudAgUfcacucugsgsu | 2321 |
| AD-1397200.1 | usasgau(Ahd)ggAfUfAfuacuguaugaL96 | 2229 | VPusdAsgcdAudTucaadGaUfacaugcgsusc | 2322 |
| AD-1397205.1 | ascsuuu(Ahd)ucAfAfUfaguuccauuaL96 | 2230 | VPusdCsucdTudTacaadGcAfuuucaagsasu | 2323 |
| AD-1397206.1 | asusagu(Uhd)ccAfUfUfuaaauugacaL96 | 2231 | VPusdCscudCudTuacadAgCfauuucaasgsa | 2324 |
| AD-1397211.1 | asgsacu(Ghd)uaUfCfCfuguuugcuaaL96 | 2232 | VPusdAsgadAadCcucudTuAfcaagcaususu | 2325 |
| AD-1397212.1 | csusgua(Uhd)ccUfGfUfuugcuauugaL96 | 2233 | VPusdTsagdAadAccucdTuUfacaagcasusu | 2326 |
| AD-1397217.1 | asusgga(Chd)auCfUfGfguugcuuugaL96 | 2234 | VPusdCscudTudAacugdTaCfccaaaccsasg | 2327 |
| AD-1397219.1 | csusucu(Ghd)auUfUfCfucuucagcuaL96 | 2235 | VPusdTscudCudAaccadCcAfccaaaucsusa | 2328 |
| AD-1397221.1 | usgsauu(Uhd)cuCfUfUfcagcuuugaaL96 | 2236 | VPusdCsgadAadCuguudGgCfaguaaugsas | 2329 |
| | | | g | |
| AD-1397222.1 | gsasuuu(Chd)ucUfUfCfagcuuugaaaL96 | 2237 | VPusdAsgcdCgdAaacudGuUfggcaguasasu | 2330 |
| AD-1397224.1 | csusugc(Ahd)agUfCfCfcaugauuucaL96 | 2238 | VPusdAsagdAadCuucadGgAfagaggaascsc | 2331 |
| AD-1397225.1 | usgscaa(Ghd)ucCfCfAfugauuucuuaL96 | 2239 | VPusdCsaadGadAcuucdAgGfaagaggasasc | 2332 |
| AD-1397226.1 | csasagu(Chd)ccAfUfGfauuucuucgaL96 | 2240 | VPusdAscadAgdAacuudCaGfgaagaggsasa | 2333 |
| AD-1397227.1 | asgsucc(Chd)auGfAfUfuucuucgguaL96 | 2241 | VPusdCsacdAadGaacudTcAfggaagagsgsa | 2334 |
| AD-1397228.1 | gsusccc(Ahd)ugAfUfUfucuucgguaaL96 | 2242 | VPusdGscadCadAgaacdTuCfaggaagasgsg | 2335 |
| AD-1397230.1 | cscscau(Ghd)auUfUfCfuucgguaauaL96 | 2243 | VPusdCsuadAadCcaugdAuCfuuaggcusgsg | 2336 |
| AD-1397231.1 | cscsaug(Ahd)uuUfCfUfucgguaauuaL96 | 2244 | VPusdCscudAadAccaudGaUfcuuaggcsusg | 2337 |
| AD-1397232.1 | asgsgga(Chd)auGfAfAfaucaucuuaaL96 | 2245 | VPusdAsaudTudAucugdCcAfgcacugasusc | 2338 |
| AD-1397234.1 | gsgsaca(Uhd)gaAfAfUfcaucuuagcaL96 | 2246 | VPusdAsgudAudAuccudAuCfuagcccascsc | 2339 |
| AD-1397235.1 | gsascau(Ghd)aaAfUfCfaucuuagcuaL96 | 2247 | VPusdCsagdTadTauccdTaUfcuagcccsasc | 2340 |
| AD-1397237.1 | asusgaa(Ahd)ucAfUfCfuuagcuuagaL96 | 2248 | VPusdCsaudAcdAguaudAuCfcuaucuasgsc | 2341 |
| AD-1397238.1 | gsasaau(Chd)auCfUfUfagcuuagcuaL96 | 2249 | VPusdGsaadCudAuugadTaAfagugaguscsa | 2342 |
| AD-1397243.1 | gsuscua(Uhd)auAfGfUfguauuguguaL96 | 2250 | VPusdGsucdAadTuuaadAuGfgaacuausus | 2343 |
| | | | g | |
| AD-1397245.1 | usasuau(Ahd)guGfUfAfuuguguguuaL96 | 2251 | VPusdCsaadAcdAggaudAcAfgucucacscsa | 2344 |
| AD-1397246.1 | asusaua(Ghd)ugUfAfUfuguguguuuaL96 | 2252 | VPusdGscadAadCaggadTaCfagucucascsc | 2345 |
| AD-1397247.1 | csasaau(Ghd)auUfUfAfcacugacugaL96 | 2253 | VPusdAsgcdAadAcaggdAuAfcagucucsasc | 2346 |
| AD-1397248.1 | asasuga(Uhd)uuAfCfAfcugacuguuaL96 | 2254 | VPusdTsagdCadAacagdGaUfacagucuscsa | 2347 |
| AD-1397249.1 | gsasaau(Ahd)aaGfUfUfauuacucugaL96 | 2255 | VPusdCsaadTadGcaaadCaGfgauacagsusc | 2348 |
| AD-1397070.2 | ascsgug(Ahd)ccCfAfAfgcucgcaugaL96 | 2185 | VPusdCsaudGcdGagcudTgGfgucacgusgsa | 2290 |
| AD-1397073.2 | usgsacc(Chd)aaGfCfUfcgcauggucaL96 | 2186 | VPusdGsacdCadTgcgadGcUfugggucascsg | 2291 |
| AD-1397075.2 | ascscca(Ahd)gcUfCfGfcauggucagaL96 | 2187 | VPusdCsugdAcdCaugcdGaGfcuuggguscsa | 2292 |
| AD-1397252.1 | gscsucg(Chd)auGfGfUfcaguaaaagaL96 | 2256 | VPusdCsuudTudAcugadCcAfugcgagcsusu | 2349 |
| AD-1397253.1 | csuscgc(Ahd)ugGfUfCfaguaaaagcaL96 | 2257 | VPusdGscudTudTacugdAcCfaugcgagscsu | 2350 |
| AD-1397261.1 | gsuscag(Uhd)aaAfAfGfcaaagacggaL96 | 2258 | VPusdCscgdTcdTuugcdTuUfuacugacscsa | 2351 |
| AD-1397262.1 | uscsagu(Ahd)aaAfGfCfaaagacgggaL96 | 2259 | VPusdCsccdGudCuuugdCuUfuuacugascsc | 2352 |
| AD-1397263.1 | csasgua(Ahd)aaGfCfAfaagacgggaaL96 | 2260 | VPusdTsccdCgdTcuuudGcUfuuuacugsasc | 2353 |
| AD-1397266.1 | asusaau(Ahd)ucAfAfAfcacgucccgaL96 | 2261 | VPusdCsggdGadCgugudTuGfauauuauscs | 2354 |
| | | | c | |
| AD-1397268.1 | asasuau(Chd)aaAfCfAfcgucccgggaL96 | 2262 | VPusdCsccdGgdGacgudGuUfugauauusas | 2355 |
| | | | u | |
| AD-1397270.1 | usasuca(Ahd)acAfCfGfucccgggagaL96 | 2263 | VPusdCsucdCcdGggacdGuGfuuugauasus | 2356 |
| | | | u | |
| AD-1397271.1 | asuscaa(Ahd)caCfGfUfcccgggaggaL96 | 2264 | VPusdCscudCcdCgggadCgUfguuugausasu | 2357 |
| AD-1397277.1 | csascgu(Chd)ccGfGfGfaggcggcagaL96 | 2265 | VPusdCsugdCcdGccucdCcGfggacgugsusu | 2358 |
| AD-1397281.1 | uscsccg(Ghd)gaGfGfCfggcagugugaL96 | 2266 | VPusdCsacdAcdTgccgdCcUfcccgggascsg | 2359 |
| AD-1397291.1 | gscsaaa(Uhd)agUfCfUfacaaaccagaL96 | 2267 | VPusdCsugdGudTuguadGaCfuauuugcsas | 2360 |
| | | | c | |
| AD-1397294.1 | asasuag(Uhd)cuAfCfAfaaccaguugaL96 | 2268 | VPusdCsaadCudGguuudGuAfgacuauusus | 2361 |
| | | | g | |
| AD-1397295.1 | usasguc(Uhd)acAfAfAfccaguugacaL96 | 2269 | VPusdGsucdAadCuggudTuGfuagacuasus | 2362 |
| | | | u | |
| AD-1397303.1 | csasaca(Uhd)ccAfUfCfauaaaccagaL96 | 2270 | VPusdCsugdGudTuaugdAuGfgauguugscs | 2363 |
| | | | c | |
| AD-1397306.1 | asuscca(Uhd)caUfAfAfaccaggaggaL96 | 2271 | VPusdCscudCcdTgguudTaUfgauggausgsu | 2364 |
| AD-1397308.1 | cscsauc(Ahd)uaAfAfCfcaggaggugaL96 | 2272 | VPusdCsacdCudCcuggdTuUfaugauggsasu | 2365 |
| AD-1397309.1 | csasuca(Uhd)aaAfCfCfaggagguggaL96 | 2273 | VPusdCscadCcdTccugdGuUfuaugaugsgsa | 2366 |
| AD-1397317.1 | ascscag(Ghd)agGfUfGfgccagguggaL96 | 2274 | VPusdCscadCcdTggccdAcCfuccuggususu | 2367 |
| AD-1423244.1 | asgsaua(Ahd)uuAfAfUfaagaagcugaL96 | 2275 | VPusdCsagdCudTcuuadTuAfauuaucusgsc | 2368 |
| AD-1423250.1 | ususaau(Ahd)agAfAfGfcuggaucuuaL96 | 2276 | VPusdAsagdAudCcagcdTuCfuuauuaasusu | 2369 |
| AD-1423253.1 | asusaag(Ahd)agCfUfGfgaucuuagcaL96 | 2277 | VPusdGscudAadGauccdAgCfuucuuausus | 2370 |
| | | | a | |
| AD-1423259.1 | asgscug(Ghd)auCfUfUfagcaacgucaL96 | 2278 | PusdGsacdGudTgcuadAgAfuccagcususc | 2371 |
| AD-1423260.1 | gscsugg(Ahd)ucUfUfAfgcaacguccaL96 | 2279 | VPusdGsgadCgdTugcudAaGfauccagcsusu | 2372 |
| AD-1423262.1 | usgsgau(Chd)uuAfGfCfaacguccagaL96 | 2280 | VPusdCsugdGadCguugdCuAfagauccasgsc | 2373 |
| AD-1423271.1 | gscsaac(Ghd)ucCfAfGfuccaaguguaL96 | 2281 | VPusdAscadCudTggacdTgGfacguugcsusa | 2374 |
| AD-1423272.1 | csasacg(Uhd)ccAfGfUfccaagugugaL96 | 2282 | VPusdCsacdAcdTuggadCuGfgacguugscsu | 2375 |
| AD-1423273.1 | asascgu(Chd)caGfUfCfcaaguguggaL96 | 2283 | VPusdCscadCadCuuggdAcUfggacguusgsc | 2376 |
| AD-1423281.1 | gsuscca(Ahd)guGfUfGfgcucaaaggaL96 | 2284 | VPusdCscudTudGagccdAcAfcuuggacsusg | 2377 |
| AD-1423282.1 | uscscaa(Ghd)ugUfGfGfcucaaaggaaL96 | 2285 | VPusdTsccdTudTgagcdCaCfacuuggascsu | 2378 |
| AD-1423283.1 | cscsaag(Uhd)guGfGfCfucaaaggauaL96 | 2286 | VPusdAsucdCudTugagdCcAfcacuuggsasc | 2379 |
| AD-1423289.1 | gsusggc(Uhd)caAfAfGfgauaauaucaL96 | 2287 | VPusdGsaudAudTauccdTuUfgagccacsasc | 2380 |
| AD-1423291.1 | gsgscuc(Ahd)aaGfGfAfuaauaucaaaL96 | 2288 | VPusdTsugdAudAuuaudCcUfuugagccsasc | 2381 |
| AD-1423299.1 | gsgsaua(Ahd)uaUfCfAfaacacguccaL96 | 2289 | VPusdGsgadCgdTguuudGaUfauuauccsus | 2382 |
| | | | u | |
| AD-1397266.2 | asusaau(Ahd)ucAfAfAfcacgucccgaL96 | 2261 | VPusdCsggdGadCgugudTuGfauauuauscs | 2354 |
| | | | c | |
| AD-1397268.2 | asasuau(Chd)aaAfCfAfcgucccgggaL96 | 2262 | VPusdCsccdGgdGacgudGuUfugauauusas | 2355 |
| | | | u | |
| AD-1397270.2 | usasuca(Ahd)acAfCfGfucccgggagaL96 | 2263 | VPusdCsucdCcdGggacdGuGfuuugauasus | 2356 |
| | | | u | |
| AD-1397271.2 | asuscaa(Ahd)caCfGfUfcccgggaggaL96 | 2264 | VPusdCscudCcdCgggadCgUfguuugausasu | 2357 |
| AD-1397277.2 | csascgu(Chd)ccGfGfGfaggcggcagaL96 | 2265 | VPusdCsugdCcdGccucdCcGfggacgugsusu | 2358 |
| AD-1397294.2 | asasuag(Uhd)cuAfCfAfaaccaguugaL96 | 2268 | VPusdCsaadCudGguuudGuAfgacuauusus | 2361 |
| | | | g | |
| AD-1397306.2 | asuscca(Uhd)caUfAfAfaccaggaggaL96 | 2271 | VPusdCscudCcdTgguudTaUfgauggausgsu | 2364 |
| AD-1397308.2 | cscsauc(Ahd)uaAfAfCfcaggaggugaL96 | 2272 | VPusdCsacdCudCcuggdTuUfaugauggsasu | 2365 |
| AD-1397073.3 | usgsacc(Chd)aaGfCfUfcgcauggucaL96 | 2186 | VPusdGsacdCadTgcgadGcUfugggucascsg | 2291 |
| AD-1397252.2 | gscsucg(Chd)auGfGfUfcaguaaaagaL96 | 2256 | VPusdCsuudTudAcugadCcAfugcgagcsusu | 2349 |
| AD-1397263.2 | csasgua(Ahd)aaGfCfAfaagacgggaaL96 | 2260 | VPusdTsccdCgdTcuuudGcUfuuuacugsasc | 2353 |
| AD-1397309.2 | csasuca(Uhd)aaAfCfCfaggagguggaL96 | 2273 | VPusdCscadCcdTccugdGuUfuaugaugsgsa | 2366 |

TABLE 17
Exemplary dsRNA molecules

| | | SEQ | | SEQ |
|---|---|---|---|---|
| | | ID | | ID |
| Duplex ID | Sense sequence (5′→3′) | NO: | Antisense sequence (5′→3′) | NO: |
| AD-1251268.1 | csasuga(Uhd)cuUfCfUfuugucguagaL96 | 2383 | VPusdCsuadCgdAcaaadGadAgdAuc | 2470 |
| | | | augsusg | |
| AD-1251270.1 | csasuga(Uhd)cuUfCfUfuugucguagaL96 | 2383 | VPusdCsuadCgdAcaaadGadAgdAuc | 2471 |
| | | | augscsc | |
| AD-1251275.1 | csasuga(Uhd)cuUfCfUfuugucguagaL96 | 2383 | VPudCuadCgdAcaaadGadAgdAucau | 2472 |
| | | | gsusg | |
| AD-1251275.2 | csasuga(Uhd)cuUfCfUfuugucguagaL96 | 2383 | VPudCuadCgdAcaaadGadAgdAucau | 2472 |
| | | | gsusg | |
| AD-1251282.1 | usgsucgaguAfCfAfcuuu(Uhd)acugaL96 | 2384 | VPusdCsagdTadAaagudGudAcdTcga | 2473 |
| | | | casusu | |
| AD-1251357.1 | gsasgaa(Uhd)UfcaCfUfuuucuucguaL96 | 2385 | VPudAcgdAadGaaaadGudGadAuuc | 2474 |
| | | | ucscsu | |
| AD-1251385.1 | asusgau(Chd)UfuCfUfUfugucguaguaL96 | 2386 | VPudAcudAcdGacaadAgdAadGauca | 2475 |
| | | | usgsu | |
| AD-1251386.1 | usgsauc(Uhd)UfcUfUfUfgucguagugaL96 | 2387 | VPudCacdTadCgacadAadGadAgauc | 2476 |
| | | | asusg | |
| AD-1251459.1 | asusaua(Uhd)UfuUfaCfaacauccguaL96 | 2388 | VPudAcgdGadTguugdTadAadAuaua | 2477 |
| | | | uscsg | |
| AD-1331348 | asusgau(Chd)UfuCfUfUfugucguagsusa | 2389 | VPudAcudAcdGacaadAgdAadGauca | 2475 |
| | | | usgsu | |
| AD-1479607 | usgsauc(Uhd)UfcUfUfUfgucguagusgsa | 2390 | VPudCacdTadCgacadAadGadAgauc | 2476 |
| | | | asusg | |
| AD-1481956 | asusgau(Chd)UfuCfUfUfugucguagsusa | 2389 | VPusdAscudAcdGacaadAgdAadGau | 2478 |
| | | | causgsu | |
| AD-1481957 | usgsauc(Uhd)UfcUfUfUfgucguagusgsa | 2390 | VPusdCsacdTadCgacadAadGadAgau | 2479 |
| | | | casusg | |
| AD-1251472.1 | usasuuu(Uhd)acdAaCfauccguuauaL96 | 2391 | VPudAuadAcdGgaugdTudGudAaaa | 2480 |
| | | | uasusg | |
| AD-1251272.1 | usgsaucuUfCfUfuugu(Chd)guagaL96 | 2392 | VPusdCsuadCgdAcaaadGadAgdAuc | 2481 |
| | | | asusg | |
| AD-1251358.1 | cscsugaagcAfUfAfaaug(Uhd)uuucaL96 | 2393 | VPusdGsaadAadCauuudAudGcUfuc | 2482 |
| | | | aggsusu | |
| AD-1251378.1 | usasaaugUfuUfuCfgaaa(Uhd)ucacaL96 | 2394 | VPudGugdAadTuucgdAadAaCfauuu | 2483 |
| | | | asusg | |
| AD-1251405.1 | uscsuuugUfcgUfAfguga(Uhd)uuucaL96 | 2395 | VPudGaadAadTcacudAcdGaCfaaaga | 2484 |
| | | | sgsg | |
| AD-1251476.1 | ususua(Chd)aacaUfCfcguuauuacaL96 | 2396 | VPudGuadAudAacggdAudGuUfguaa | 2485 |
| | | | asgsu | |
| AD-1251266.1 | ususgauaguUfaCfcuag(Uhd)uugcaL96 | 2397 | VPudGcadAadCuaggdTadAcUfauca | 2486 |
| | | | asgsg | |
| AD-1251259.1 | ususgauaguUfaCfcuag(Uhd)uugcaL96 | 2397 | VPusdGscadAadCuaggdTadAcUfauc | 2487 |
| | | | aasgsg | |
| AD-1479603 | usasaaugUfuUfuCfgaaa(Uhd)ucascsa | 2398 | VPudGugdAadTuucgdAadAaCfauuu | 2483 |
| | | | asusg | |
| AD-1479612 | uscsuuugUfcgUfAfguga(Uhd)uuuscsa | 2399 | VPudGaadAadTcacudAcdGaCfaaaga | 2484 |
| | | | sgsg | |
| AD-1479603 | usasaaugUfuUfuCfgaaa(Uhd)ucascsa | 2398 | VPudGugdAadTuucgdAadAaCfauuu | 2483 |
| | | | asusg | |
| AD-1479612 | uscsuuugUfcgUfAfguga(Uhd)uuuscsa | 2399 | VPudGaadAadTcacudAcdGaCfaaaga | 2484 |
| | | | sgsg | |
| AD-1481954 | usasaaugUfuUfuCfgaaa(Uhd)ucascsa | 2398 | VPusdGsugdAadTuucgdAadAaCfau | 2488 |
| | | | uuasusg | |
| AD-1481961 | uscsuuugUfcgUfAfguga(Uhd)uuuscsa | 2399 | VPusdGsaadAadTcacudAcdGaCfaaa | 2489 |
| | | | gasgsg | |
| AD-1251262.1 | gsasuaguUfaCfcuag(Uhd)uugcaL96 | 2400 | VPusdGscadAadCuaggdTadAcUfauc | 2490 |
| | | | sgsg | |
| AD-1251279.2 | csasaca(Chd)aaUfUfUfcuucuuagcaL96 | 2401 | VPudGcudAadGaagadAaUfudGugu | 2491 |
| | | | ugsusu | |
| AD-1251278.1 | ascsacaaUfUfUfcuuc(Uhd)uagcaL96 | 2402 | VPusdGscudAadGaagadAaUfudGug | 2492 |
| | | | ususg | |
| AD-1251319.1 | asgsggaaaaCfadAucuu(Chd)cguuaL96 | 2403 | VPudAacdGgdAagaudTgUfuUfucccu | 2493 |
| | | | susu | |
| AD-1251348.1 | usgsuaggagdAaUfucau(Uhd)uuucaL96 | 2404 | VPusdGsaadAadAugaadTuCfuCfcua | 2494 |
| | | | cascsg | |
| AD-1251354.1 | asgsgagaauUfcdAcuuu(Uhd)cuucaL96 | 2405 | VPudGaadGadAaagudGaAfuUfcucc | 2495 |
| | | | usgsc | |
| AD-1251365.1 | asgsca(Uhd)aaaUfgUfuuucgaaauaL96 | 2406 | VPudAuudTcdGaaaadCaUfuUfaugc | 2496 |
| | | | ususc | |
| AD-1251419.1 | gsusaga(Uhd)CfuUfgCfaauuaccauaL96 | 2407 | VPudAugdGudAauugdCaAfgAfucua | 2497 |
| | | | csgsg | |
| AD-1251474.1 | asusuuuacadAcdAuccg(Uhd)uauuaL96 | 2408 | VPudAaudAadCggaudGuUfgUfaaaa | 2498 |
| | | | usgsu | |
| AD-1251419.2 | gsusaga(Uhd)CfuUfgCfaauuaccauaL96 | 2407 | VPudAugdGudAauugdCaAfgAfucua | 2497 |
| | | | csgsg | |
| AD-1479622 | gsusaga(Uhd)CfuUfgCfaauuaccasusa | 2409 | VPudAugdGudAauugdCaAfgAfucua | 2497 |
| | | | csgsg | |
| AD-1479593 | asgsca(Uhd)aaaUfgUfuuucgaaasusa | 2410 | VPudAuudTcdGaaaadCaUfuUfaugc | 2496 |
| | | | ususc | |
| AD-1479622 | gsusaga(Uhd)CfuUfgCfaauuaccasusa | 2409 | VPudAugdGudAauugdCaAfgAfucua | 2497 |
| | | | csgsg | |
| AD-1481938 | gsusaga(Uhd)CfuUfgCfaauuaccasusa | 2409 | VPusdAsugdGudAauugdCaAfgAfucu | 2499 |
| | | | acsgsg | |
| AD-1481952 | asgsca(Uhd)aaaUfgUfuuucgaaasusa | 2410 | VPusdAsuudTcdGaaaadCaUfuUfaug | 2500 |
| | | | cususc | |
| AD-1481938 | gsusaga(Uhd)CfuUfgCfaauuaccasusa | 2409 | VPusdAsugdGudAauugdCaAfgAfucu | 2499 |
| | | | acsgsg | |
| AD-1479586 | usgsuaggagdAaUfucau(Uhd)uuuscsa | 2411 | VPusdGsaadAadAugaadTuCfuCfcua | 2494 |
| | | | cascsg | |
| AD-1251304.1 | ascsaaagggAfAfAfacaa(Uhd)cuucaL96 | 2412 | VPudGaadGadTuguuuuCfcCfuuugus | 2501 |
| | | | gsu | |
| AD-1251305.1 | csasaagggaAfAfAfcaau(Chd)uuccaL96 | 2413 | VPudGgadAgdAuuguuuUfcCfcuuugs | 2502 |
| | | | usg | |
| AD-1251321.1 | gsgsaaaa(Chd)aaUfCfuuccguuucaL96 | 2414 | VPudGaadAcdGgaagauUfgUfuuuccs | 2503 |
| | | | csu | |
| AD-1251345.1 | usgsuaggAfgAfAfUfucau(Uhd)uuucaL96 | 2415 | VPusdGsaadAadAugaauuCfuCfcuac | 2504 |
| | | | ascsg | |
| AD-1251351.1 | gsusaggagaaUfUfcacu(Uhd)uucuaL96 | 2416 | VPudAgadAadAgugaauUfcUfccuacs | 2505 |
| | | | gsc | |
| AD-1251353.1 | usasggagaaUfUfCfacuu(Uhd)ucuuaL96 | 2417 | VPusdAsagdAadAagugaaUfuCfuccu | 2506 |
| | | | ascsg | |
| AD-1251381.1 | usasca(Uhd)gauCfUfUfcuuugucguaL96 | 2418 | VPudAscgdAcdAaagaagAfuCfaugua | 2507 |
| | | | scsc | |
| AD-1251265.1 | ususgauaguUfAfCfcuag(Uhd)uugcaL96 | 2419 | VPudGcadAadCuagguaAfcUfaucaas | 2508 |
| | | | gsg | |
| AD-1251257.1 | ususgau(Ahd)guUfAfCfcuaguuugcaL96 | 2420 | VPusdGscadAadCuagguaAfcUfauca | 2509 |
| | | | asgsg | |
| AD-1251264.1 | ususgauaguUfAfCfcuaa(Uhd)uugcaL96 | 2421 | VPusdGscadAadTuagguaAfcUfauca | 2510 |
| | | | asgsg | |
| AD-1251258.1 | ususgauaguUfAfCfcuag(Uhd)uugcaL96 | 2419 | VPusdGscadAadCuagguaAfcUfauca | 2509 |
| | | | asgsg | |
| AD-1251261.1 | gsasuaguUfAfCfcuag(Uhd)uugcaL96 | 2422 | VPusdGscadAadCuagguaAfcUfaucs | 2511 |
| | | | gsg | |
| AD-1479593 | asgsca(Uhd)aaaUfgUfuuucgaaasusa | 2410 | VPudAuudTcdGaaaadCaUfuUfaugc | 2496 |
| | | | ususc | |
| AD-1251283.1 | usgsucgaguAfCfAfcuuu(Uhd)acugaL96 | 2384 | VPusCfsagdTadAaagudGudAcdTcga | 2512 |
| | | | casusu | |
| AD-1251384.1 | csasuga(Uhd)CfuUfCfUfuugucguagaL96 | 2423 | VPuCfuadCgdAcaaadGadAgdAucau | 2513 |
| | | | gsusg | |
| AD-1251274.2 | csasuga(Uhd)cuUfCfUfuugucguagaL96 | 2383 | VPuCfuadCgdAcaaadGadAgdAucau | 2513 |
| | | | gsusg | |
| AD-1251269.1 | csasuga(Uhd)cuUfCfUfuugucguagaL96 | 2383 | VPusCfsuadCgdAcaaadGadAgdAuca | 2514 |
| | | | ugsusg | |
| AD-1251274.1 | csasuga(Uhd)cuUfCfUfuugucguagaL96 | 2383 | VPuCfuadCgdAcaaadGadAgdAucau | 2513 |
| | | | gsusg | |
| AD-1251271.1 | csasuga(Uhd)cuUfCfUfuugucguagaL96 | 2383 | VPusCfsuadCgdAcaaadGadAgdAuca | 2515 |
| | | | ugscsc | |
| AD-1251274.3 | csasuga(Uhd)cuUfCfUfuugucguagaL96 | 2383 | VPuCfuadCgdAcaaadGadAgdAucau | 2513 |
| | | | gsusg | |
| AD-1331354 | csasuga(Uhd)cuUfCfUfuugucguasgsa | 2424 | VPuCfuadCgdAcaaadGadAgdAucau | 2513 |
| | | | gsusg | |
| AD-1479606 | csasuga(Uhd)CfuUfCfUfuugucguasgsa | 2425 | VPuCfuadCgdAcaaadGadAgdAucau | 2513 |
| | | | gsusg | |
| AD-1331354 | csasuga(Uhd)cuUfCfUfuugucguasgsa | 2424 | VPuCfuadCgdAcaaadGadAgdAucau | 2513 |
| | | | gsusg | |
| AD-1479606 | csasuga(Uhd)CfuUfCfUfuugucguasgsa | 2425 | VPuCfuadCgdAcaaadGadAgdAucau | 2513 |
| | | | gsusg | |
| AD-1481942 | csasuga(Uhd)cuUfCfUfuugucguasgsa | 2424 | VPusCfsuadCgdAcaaadGadAgdAuca | 2514 |
| | | | ugsusg | |
| AD-1481955 | csasuga(Uhd)CfuUfCfUfuugucguasgsa | 2425 | VPusCfsuadCgdAcaaadGadAgdAuca | 2514 |
| | | | ugsusg | |
| AD-1251273.1 | usgsaucuUfCfUfuugu(Chd)guagaL96 | 2392 | VPusCfsuadCgdAcaaadGadAgdAuca | 2516 |
| | | | susg | |
| AD-1251320.1 | gsgsgaaaAfcAfaUfcuuc(Chd)guuuaL96 | 2426 | VPusAfsaadCgdGaagadTudGuUfuuc | 2517 |
| | | | ccsusu | |
| AD-1251325.1 | asasaacaauCfUfUfccgu(Uhd)ucaaaL96 | 2427 | VPuUfugdAadAcggadAgdAuUfguuu | 2518 |
| | | | uscsc | |
| AD-1251356.1 | gsgsagaaUfuCfaCfuuuu(Chd)uucgaL96 | 2428 | VPuCfgadAgdAaaagdTgdAaUfucucc | 2519 |
| | | | susg | |
| AD-1251418.1 | usgsuagaUfcUfudGcaau(Uhd)accaaL96 | 2429 | VPuUfggdTadAuugcdAadGaUfcuaca | 2520 |
| | | | sgsg | |
| AD-1251457.1 | gsasua(Uhd)aUfuUfudAcaacauccgaL96 | 2430 | VPuCfggdAudGuugudAadAaUfauau | 2521 |
| | | | csgsc | |
| AD-1251485.1 | csasagugUfuCfCfUfacug(Uhd)caugaL96 | 2431 | VPusCfsaudGadCaguadGgdAaCfacu | 2522 |
| | | | ugsgsg | |
| AD-1251325.1 | asasaacaauCfUfUfccgu(Uhd)ucaaaL96 | 2427 | VPuUfugdAadAcggadAgdAuUfguuu | 2518 |
| | | | uscsc | |
| AD-1331350 | asasaacaauCfUfUfccgu(Uhd)ucasasa | 2432 | VPuUfugdAadAcggadAgdAuUfguuu | 2518 |
| | | | uscsc | |
| AD-1331350 | asasaacaauCfUfUfccgu(Uhd)ucasasa | 2432 | VPuUfugdAadAcggadAgdAuUfguuu | 2518 |
| | | | uscsc | |
| AD-1479588 | gsgsagaaUfuCfaCfuuuu(Chd)uucsgsa | 2433 | VPuCfgadAgdAaaagdTgdAaUfucucc | 2519 |
| | | | susg | |
| AD-1481945 | asasaacaauCfUfUfccgu(Uhd)ucasasa | 2432 | VPusUfsugdAadAcggadAgdAuUfguu | 2523 |
| | | | uuscsc | |
| AD-1481948 | gsgsagaaUfuCfaCfuuuu(Chd)uucsgsa | 2433 | VPusCfsgadAgdAaaagdTgdAaUfucu | 2524 |
| | | | ccsusg | |
| AD-1251284.1 | usgsucgaguAfCfAfcuuu(Uhd)acugaL96 | 2384 | VPusCfsagdTadAaagudGuAfcdTcgac | 2525 |
| | | | asusu | |
| AD-1251286.1 | usgsucgaguAfCfAfcuuu(Uhd)acugaL96 | 2384 | VPusCfsagdTadAaagudGuAfcdTcgac | 2526 |
| | | | ascsc | |
| AD-1251288.1 | uscsgaguAfCfAfcuuu(Uhd)acugaL96 | 2434 | VPusCfsagdTadAaagudGuAfcdTcgas | 2527 |
| | | | csg | |
| AD-1251290.1 | usgsucgaguAfCfAfcuuu(Uhd)acugaL96 | 2384 | VPuCfagdTadAaagudGuAfcdTcgaca | 2528 |
| | | | susu | |
| AD-1251471.1 | asusauu(Uhd)UfaCfadAcauccguuaaL96 | 2435 | VPuUfaadCgdGaugudTgUfadAaaua | 2529 |
| | | | usgsu | |
| AD-1251475.1 | ususuua(Chd)aaCfaUfccguuauuaaL96 | 2436 | VPuUfaadTadAcggadTgUfudGuaaaa | 2530 |
| | | | susg | |
| AD-1251284.2 | usgsucgaguAfCfAfcuuu(Uhd)acugaL96 | 2384 | VPusCfsagdTadAaagudGuAfcdTcgac | 2525 |
| | | | asusu | |
| AD-1331352 | usgsucgaguAfCfAfcuuu(Uhd)acusgsa | 2437 | VPusCfsagdTadAaagudGuAfcdTcgac | 2525 |
| | | | asusu | |
| AD-1251322.1 | gsasaaa(Chd)aaUfCfUfuccauuucaaL96 | 2438 | VPuUfgadAadTggaagaUfudGuuuucs | 2531 |
| | | | csc | |
| AD-1479581 | gsasaaa(Chd)aaUfCfUfuccauuucsasa | 2439 | VPuUfgadAadTggaagaUfudGuuuucs | 2531 |
| | | | csc | |
| AD-1479581 | gsasaaa(Chd)aaUfCfUfuccauuucsasa | 2439 | VPuUfgadAadTggaagaUfudGuuuucs | 2531 |
| | | | csc | |
| AD-1481943 | gsasaaa(Chd)aaUfCfUfuccauuucsasa | 2439 | VPusUfsgadAadTggaagaUfudGuuuu | 2532 |
| | | | cscsc | |
| AD-1479588 | gsgsagaaUfuCfaCfuuuu(Chd)uucsgsa | 2433 | VPuCfgadAgdAaaagdTgdAaUfucucc | 2519 |
| | | | susg | |
| AD-1251303.1 | csascaaagggAfAfaacaa(Uhd)cuuaL96 | 2440 | VPusAfsagdAudTguuuucCfcUfuugu | 2533 |
| | | | gsusu | |
| AD-1251306.1 | asasagggAfaAfAfCfaauc(Uhd)uccgaL96 | 2441 | VPusCfsggdAadGauuguuUfuCfccuu | 2534 |
| | | | usgsu | |
| AD-1251307.1 | asasagggaadAaCfaauc(Uhd)uccgaL96 | 2442 | VPuCfggdAadGauugdTuUfuCfccuuu | 2535 |
| | | | sgsu | |
| AD-1251318.1 | asgsggaaAfaCfAfAfucuu(Chd)cguuaL96 | 2443 | VPusAfsacdGgdAagauugUfuUfuccc | 2536 |
| | | | ususu | |
| AD-1251324.1 | asasaacaAfuCfUfUfccgu(Uhd)ucaaaL96 | 2444 | VPusUfsugdAadAcggaagAfuUfguuu | 2537 |
| | | | uscsc | |
| AD-1251249.1 | usgsucgaguAfCfAfcuuu(Uhd)acugaL96 | 2384 | VPusCfsagdTadAaaguguAfcUfcgaca | 2538 |
| | | | susu | |
| AD-1251254.1 | usgsucgaguAfCfAfcuuu(Uhd)acugaL96 | 2384 | VPuCfagdTadAaaguguAfcUfcgacasc | 2539 |
| | | | sc | |
| AD-1251250.1 | usgsucgaguAfCfAfcuuu(Uhd)acugaL96 | 2384 | VPusCfsagdTadAaaguguAfcUfcgaca | 2540 |
| | | | scsc | |
| AD-1251251.1 | uscsgaguAfCfAfcuuu(Uhd)acugaL96 | 2434 | VPusCfsagdTadAaaguguAfcUfcgascs | 2541 |
| | | | g | |
| AD-1251350.1 | gsusaggagaAfUfUfcacu(Uhd)uucuaL96 | 2445 | VPusAfsgadAadAgugaauUfcUfccuac | 2542 |
| | | | sgsc | |
| AD-1251352.1 | usasggagAfaUfUfCfacuu(Uhd)ucuuaL96 | 2446 | VPusAfsagdAadAagugaaUfuCfuccua | 2543 |
| | | | scsg | |
| AD-1251355.1 | gsgsagaaUfuCfAfCfuuuu(Chd)uucgaL96 | 2447 | VPusCfsgadAgdAaaagugAfaUfucucc | 2544 |
| | | | susg | |
| AD-1251359.1 | csusgaagCfaUfAfAfaugu(Uhd)uucgaL96 | 2448 | VPusCfsgadAadAcauuuaUfgCfuucag | 2545 |
| | | | sgsu | |
| AD-1251363.1 | gsasagcauadAaUfguuu(Uhd)cgaaaL96 | 2449 | VPuUfucdGadAaacadTuUfaUfgcuuc | 2546 |
| | | | sasg | |
| AD-1251362.1 | gsasagcaUfaAfAfUfguuu(Uhd)cgaaaL96 | 2450 | VPusUfsucdGadAaacauuUfaUfgcuu | 2547 |
| | | | csasg | |
| AD-1251369.1 | asgscauaaaUfgUfuuu(Uhd)gaaauaL96 | 2451 | VPusAfsuudTcdAaaaadCaUfuUfaug | 2548 |
| | | | cususc | |
| AD-1251376.1 | asusaaa(Uhd)guUfUfUfcgaaauucaaL96 | 2452 | VPusUfsgadAudTucgaaaAfcAfuuuau | 2549 |
| | | | sgsc | |
| AD-1251377.1 | asusaaa(Uhd)guUfUfUfcgaaauucaaL96 | 2452 | VPusUfsgadAudTucgaaaAfcAfuuuau | 2550 |
| | | | sgsu | |
| AD-1251380.1 | usasca(Uhd)gAfuCfUfUfcuuugucguaL96 | 2453 | VPusAfscgdAcdAaagaagAfuCfaugua | 2551 |
| | | | sgsg | |
| AD-1251383.1 | csasuga(Uhd)CfuUfCfUfuugucguagaL96 | 2423 | VPusCfsuadCgdAcaaagaAfgAfucaug | 2552 |
| | | | susg | |
| AD-1251431.1 | usasugugAfaAfCfAfaacu(Uhd)uacgaL96 | 2454 | VPusCfsgudAadAguuuguUfuCfacau | 2553 |
| | | | asgsu | |
| AD-1251447.1 | gsusgaaaCfadAaCfcuua(Chd)gugaaL96 | 2455 | VPuUfcadCgdTaaggdTuUfgUfuucac | 2554 |
| | | | sgsu | |
| AD-1251446.1 | gsusgaaaCfaAfAfCfcuua(Chd)gugaaL96 | 2456 | VPusUfscadCgdTaagguuUfgUfuucac | 2555 |
| | | | sgsu | |
| AD-1251456.1 | gsasua(Uhd)aUfuUfUfAfcaacauccgaL96 | 2457 | VPusCfsggdAudGuuguaaAfaUfauau | 2556 |
| | | | csgsc | |
| AD-1251458.1 | asusaua(Uhd)UfuUfAfCfaacauccguaL96 | 2458 | VPusAfscgdGadTguuguaAfaAfuaua | 2557 |
| | | | uscsg | |
| AD-1251470.1 | asusauu(Uhd)UfaCfAfAfcauccguuaaL96 | 2459 | VPusUfsaadCgdGauguugUfaAfaaua | 2558 |
| | | | usgsu | |
| AD-1251473.1 | asusuuuaCfaAfCfAfuccg(Uhd)uauuaL96 | 2460 | VPusAfsaudAadCggauguUfgUfaaaa | 2559 |
| | | | usgsu | |
| AD-1251484.1 | csasagugUfuCfCfUfacug(Uhd)caugaL96 | 2431 | VPusCfsaudGadCaguaggAfaCfacuug | 2560 |
| | | | sgsg | |
| AD-1251249.1 | usgsucgaguAfCfAfcuuu(Uhd)acugaL96 | 2384 | VPusCfsagdTadAaaguguAfcUfcgaca | 2538 |
| | | | susu | |
| AD-1251318.1 | asgsggaaAfaCfAfAfucuu(Chd)cguuaL96 | 2443 | VPusAfsacdGgdAagauugUfuUfuccc | 2536 |
| | | | ususu | |
| AD-1251363.1 | gsasagcauadAaUfguuu(Uhd)cgaaaL96 | 2449 | VPuUfucdGadAaacadTuUfaUfgcuuc | 2546 |
| | | | sasg | |
| AD-1251377.2 | asusaaa(Uhd)guUfUfUfcgaaauucaaL96 | 2452 | VPusUfsgadAudTucgaaaAfcAfuuuau | 2550 |
| | | | sgsu | |
| AD-1331355 | usgsucgaguAfCfAfcuuu(Uhd)acusgsa | 2437 | VPusCfsagdTadAaaguguAfcUfcgaca | 2538 |
| | | | susu | |
| AD-1479582 | asasaacaAfuCfUfUfccgu(Uhd)ucasasa | 2461 | VPusUfsugdAadAcggaagAfuUfguuu | 2537 |
| | | | uscsc | |
| AD-1479587 | gsgsagaaUfuCfAfCfuuuu(Chd)uucsgsa | 2462 | VPusCfsgadAgdAaaagugAfaUfucucc | 2544 |
| | | | susg | |
| AD-1479589 | csusgaagCfaUfAfAfaugu(Uhd)uucsgsa | 2463 | VPusCfsgadAadAcauuuaUfgCfuucag | 2545 |
| | | | sgsu | |
| AD-1479591 | gsasagcaUfaAfAfUfguuu(Uhd)cgasasa | 2464 | VPusUfsucdGadAaacauuUfaUfgcuu | 2547 |
| | | | csasg | |
| AD-1479596 | asgscauaaaUfgUfuuu(Uhd)gaaasusa | 2465 | VPusAfsuudTcdAaaaadCaUfuUfaug | 2548 |
| | | | cususc | |
| AD-1479601 | asusaaa(Uhd)guUfUfUfcgaaauucsasa | 2466 | VPusUfsgadAudTucgaaaAfcAfuuuau | 2549 |
| | | | sgsc | |
| AD-1479602 | asusaaa(Uhd)guUfUfUfcgaaauucsasa | 2466 | VPusUfsgadAudTucgaaaAfcAfuuuau | 2550 |
| | | | sgsu | |
| AD-1479604 | usasca(Uhd)gAfuCfUfUfcuuugucgsusa | 2467 | VPusAfscgdAcdAaagaagAfuCfaugua | 2551 |
| | | | sgsg | |
| AD-1479605 | csasuga(Uhd)CfuUfCfUfuugucguasgsa | 2425 | VPusCfsuadCgdAcaaagaAfgAfucaug | 2552 |
| | | | susg | |
| AD-1331349 | gsasagcauadAaUfguuu(Uhd)cgasasa | 2468 | VPuUfucdGadAaacadTuUfaUfgcuuc | 2546 |
| | | | sasg | |
| AD-1481950 | gsasagcauadAaUfguuu(Uhd)cgasasa | 2468 | VPusUfsucdGadAaacadTuUfaUfgcu | 2561 |
| | | | ucsasg | |
| AD-1251251.1 | uscsgaguAfCfAfcuuu(Uhd)acugaL96 | 2434 | VPusCfsagdTadAaaguguAfcUfcgascs | 2541 |
| | | | g | |
| AD-1479580 | uscsgaguAfCfAfcuuu(Uhd)acusgsa | 2469 | VPusCfsagdTadAaaguguAfcUfcgascs | 2541 |
| | | | g | |
| AD-1331349 | gsasagcauadAaUfguuu(Uhd)cgasasa | 2468 | VPuUfucdGadAaacadTuUfaUfgcuuc | 2546 |
| | | | sasg | |

TABLE 18
Exemplary dsRNA molecules

| | | SEQ | | SEQ |
|---|---|---|---|---|
| | | ID | | ID |
| Duplex ID | Sense sequence (5′→3′) | NO: | Antisense sequence (5′→3′) | NO: |
| AD-1395794.1 | csuscuccaagUfAfUfgaucgucuuL96 | 2562 | asdAsgadCgdAucaudAcUfuggagagscsg | 2567 |
| AD-1395803.1 | csasgugaugCfUfCfuccaaguauuL96 | 2563 | asdAsuadCudTggagdAgCfaucacugsusg | 2568 |
| AD-1395805.1 | cscsaaguauGfAfUfcgucugcaguL96 | 2564 | asdCsugdCadGacgadTcAfuacuuggsasg | 2569 |
| AD-1395816.1 | gsgsguuuguUfudGUfuucauuucuL96 | 2565 | asdGsaadAudGaaacdAaAfcaaacccsusg | 2570 |
| AD-1395823.1 | gsasguauuuCfUfCfagcauucaauL96 | 2566 | asdTsugdAadTgcugdAgAfaauacucscsc | 2571 |

TABLE 19
Exemplary dsRNA molecules

| | | SEQ | | SEQ |
|---|---|---|---|---|
| | | ID | | ID |
| Duplex ID | Sense sequence (5′→3′) | NO: | Antisense sequence (5′→3′) | NO: |
| AD-1465901.1 | csasgcuaagGfCfAfguucuacguuL96 | 2572 | asdAscgdTadGaacudGcCfuuagcugsusg | 2690 |
| AD-1465906.1 | ususcuugggCfCfUfacuuuauauuL96 | 2573 | asdAsuadTadAaguadGgCfccaagaasgsu | 2691 |
| AD-1465907.1 | usascuuuauAfUfGfcugaagucguL96 | 2574 | asdCsgadCudTcagcdAuAfuaaaguasgsg | 2692 |
| AD-1465908.1 | ascsuuuauaUfGfCfugaagucgguL96 | 2575 | asdCscgdAcdTucagdCaUfauaaagusasg | 2693 |
| AD-1465909.1 | asgsuaaauuAfUfCfagaaggugcuL96 | 2576 | asdGscadCcdTucugdAuAfauuuacusgsu | 2694 |
| AD-1465911.1 | asuscagaagGfUfGfcuucuuaccuL96 | 2577 | asdGsgudAadGaagcdAcCfuucugausasa | 2695 |
| AD-1465912.1 | uscsagaaggUfGfCfuucuuaccuuL96 | 2578 | asdAsggdTadAgaagdCaCfcuucugasusa | 2696 |
| AD-1465913.1 | csasgaagguGfCfUfucuuaccuuuL96 | 2579 | asdAsagdGudAagaadGcAfccuucugsasu | 2697 |
| AD-1465915.1 | ascscuaugaAfUfGfgaguaucaguL96 | 2580 | asdCsugdAudAcuccdAuUfcauaggusgsu | 2698 |
| AD-1465916.1 | cscsuaugaaUfGfGfaguaucaguuL96 | 2581 | asdAscudGadTacucdCaUfucauaggsusg | 2699 |
| AD-1465917.1 | asusgaauggAfGfUfaucagugaguL96 | 2582 | asdCsucdAcdTgauadCuCfcauucausasg | 2700 |
| AD-1465918.1 | asusgccucaCfAfCfacaucuauuuL96 | 2583 | asdAsaudAgdAugugdTgUfgaggcausgsg | 2701 |
| AD-1465919.1 | usgsccucacAfCfAfcaucuauuauL96 | 2584 | asdTsaadTadGaugudGuGfugaggcasusg | 2702 |
| AD-1465920.1 | gscscucacaCfAfCfaucuauuacuL96 | 2585 | asdGsuadAudAgaugdTgUfgugaggcsasu | 2703 |
| AD-1465921.1 | cscsucacacAfCfAfucuauuacuuL96 | 2586 | asdAsgudAadTagaudGuGfugugaggscsa | 2704 |
| AD-1465922.1 | csuscacacaCfAfUfcuauuacucuL96 | 2587 | asdGsagdTadAuagadTgUfgugugagsgsc | 2705 |
| AD-1465927.1 | gsascguuugAfCfAfagcaaaucguL96 | 2588 | asdCsgadTudTgcuudGuCfaaacgucsusu | 2706 |
| AD-1465928.1 | gscscagucaUfCfAfucccuaauguL96 | 2589 | asdCsaudTadGggaudGaUfgacuggcsusc | 2707 |
| AD-1465932.1 | asasuguacaCfAfGfucaauggauuL96 | 2590 | asdAsucdCadTugacdTgUfguacauusasg | 2708 |
| AD-1465935.1 | gscscagauaUfAfAfcaguuuguguL96 | 2591 | asdCsacdAadAcugudTaUfaucuggcsasu | 2709 |
| AD-1465936.1 | cscsagauauAfAfCfaguuugugcuL96 | 2592 | asdGscadCadAacugdTuAfuaucuggscsa | 2710 |
| AD-1465937.1 | gsasgcagaaCfCfAfucauaagguuL96 | 2593 | asdAsccdTudAugaudGgUfucugcucscsa | 2711 |
| AD-1465942.1 | csasuccacuAfCfCfgcaaauauguL96 | 2594 | asdCsaudAudTugcgdGuAfguggaugsusa | 2712 |
| AD-1465945.1 | gscsugggauGfCfAfggcuuacauuL96 | 2595 | asdAsugdTadAgccudGcAfucccagcsusu | 2713 |
| AD-1465946.1 | csusgggaugCfAfGfgcuuacauuuL96 | 2596 | asdAsaudGudAagccdTgCfaucccagscsu | 2714 |
| AD-1465947.1 | usgsggaugcAfGfGfcuuacauuguL96 | 2597 | asdCsaadTgdTaagcdCuGfcaucccasgsc | 2715 |
| AD-1465949.1 | gsgsaugcagGfCfUfuacauugacuL96 | 2598 | asdGsucdAadTguaadGcCfugcauccscsa | 2716 |
| AD-1465951.1 | asusgcaggcUfUfAfcauugacauuL96 | 2599 | asdAsugdTcdAaugudAaGfccugcauscsc | 2717 |
| AD-1465955.1 | asgsgcuuacAfUfUfgacauuaaauL96 | 2600 | asdTsuudAadTgucadAuGfuaagccusgsc | 2718 |
| AD-1465956.1 | gsgscuuacaUfUfGfacauuaaaauL96 | 2601 | asdTsuudTadAugucdAaUfguaagccsusg | 2719 |
| AD-1465957.1 | gscsuuacauUfGfAfcauuaaaaauL96 | 2602 | asdTsuudTudAaugudCaAfuguaagcscsu | 2720 |
| AD-1465958.1 | csusuacauuGfAfCfauuaaaaacuL96 | 2603 | asdGsuudTudTaaugdTcAfauguaagscsc | 2721 |
| AD-1465959.1 | ususacauugAfCfAfuuaaaaacuuL96 | 2604 | asdAsgudTudTuaaudGuCfaauguaasgsc | 2722 |
| AD-1465960.1 | usascauugaCfAfUfuaaaaacuguL96 | 2605 | asdCsagdTudTuuaadTgUfcaauguasasg | 2723 |
| AD-1465961.1 | ascsauugacAfUfUfaaaaacugcuL96 | 2606 | asdGscadGudTuuuadAuGfucaaugusasa | 2724 |
| AD-1465968.1 | csasccuguaAfUfAfccagcgaauuL96 | 2607 | asdAsuudCgdCuggudAuUfacaggugscsa | 2725 |
| AD-1465969.1 | usgsuaauacCfAfGfcgaauaugguL96 | 2608 | asdCscadTadTucgcdTgGfuauuacasgsg | 2726 |
| AD-1465970.1 | gsusaauaccAfGfCfgaauauggauL96 | 2609 | asdTsccdAudAuucgdCuGfguauuacsasg | 2727 |
| AD-1465972.1 | gsusuauguaCfAfCfacaguacgauL96 | 2610 | asdTscgdTadCugugdTgUfacauaacsusu | 2728 |
| AD-1465973.1 | ususauguacAfCfAfcaguacgaauL96 | 2611 | asdTsucdGudAcugudGuGfuacauaascsu | 2729 |
| AD-1465976.1 | gsusacacacAfGfUfacgaagauguL96 | 2612 | asdCsaudCudTcguadCuGfuguguacsasu | 2730 |
| AD-1465986.1 | asasaccuauAfCfUfuauaagugguL96 | 2613 | asdCscadCudTauaadGuAfuagguuuscsc | 2731 |
| AD-1465992.1 | asusgaaucaAfAfCfaucaugagcuL96 | 2614 | asdGscudCadTgaugdTuUfgauucausasa | 2732 |
| AD-1465997.1 | cscsaguggcAfCfUfucuguaguguL96 | 2615 | asdCsacdTadCagaadGuGfccacuggsasc | 2733 |
| AD-1465998.1 | asgsuggcacUfUfCfuguaguguguL96 | 2616 | asdCsacdAcdTacagdAaGfugccacusgsg | 2734 |
| AD-1465999.1 | csusgggcacUfCfAfuucaucuauuL96 | 2617 | asdAsuadGadTgaaudGaGfugcccagsusg | 2735 |
| AD-1466002.1 | ususaacuucCfAfUfgaauucuaguL96 | 2618 | asdCsuadGadAuucadTgGfaaguuaascsa | 2736 |
| AD-1466004.1 | usgsaugaagAfCfUfcauaugagauL96 | 2619 | asdTscudCadTaugadGuCfuucaucasusc | 2737 |
| AD-1466005.1 | asasacucauCfAfUfugaaucagguL96 | 2620 | asdCscudGadTucaadTgAfugaguuuscsg | 2738 |
| AD-1466006.1 | asasacacagAfUfAfuaauuguuguL96 | 2621 | asdCsaadCadAuuaudAuCfuguguuusgsa | 2739 |
| AD-1466007.1 | csascagauaUfAfAfuuguugguuuL96 | 2622 | asdAsacdCadAcaaudTaUfaucugugsusu | 2740 |
| AD-1466009.1 | asusucugaaGfAfCfccuauagaguL96 | 2623 | asdCsucdTadTagggdTcUfucagaausasu | 2741 |
| AD-1466010.1 | csgsucuacuUfUfCfacuuggugcuL96 | 2624 | asdGscadCcdAagugdAaAfguagacgsusa | 2742 |
| AD-1466012.1 | asasuuacuaGfCfAfcauaaaguuuL96 | 2625 | asdAsacdTudTaugudGcUfaguaauususc | 2743 |
| AD-1466013.1 | usascuagcaCfAfUfaaaguuggguL96 | 2626 | asdCsccdAadCuuuadTgUfgcuaguasasu | 2744 |
| AD-1466014.1 | gsasgauggcAfUfUfuggcuucuguL96 | 2627 | asdCsagdAadGccaadAuGfccaucucscsc | 2745 |
| AD-1466019.1 | asasguuuccUfAfGfaguuagacauL96 | 2628 | asdTsgudCudAacucdTaGfgaaacuususg | 2746 |
| AD-1466020.1 | cscsuagaguUfAfGfacauaaaucuL96 | 2629 | asdGsaudTudAugucdTaAfcucuaggsasa | 2747 |
| AD-1466021.1 | usascaaguaAfGfAfcaggauggauL96 | 2630 | asdTsccdAudCcugudCuUfacuuguasgsa | 2748 |
| AD-1466024.1 | asgsgaccuuUfCfAfcccucuaaguL96 | 2631 | asdCsuudAgdAgggudGaAfagguccuscsg | 2749 |
| AD-1466026.1 | usgscuucauAfAfAfuccaaugaauL96 | 2632 | asdTsucdAudTggaudTuAfugaagcascsc | 2750 |
| AD-1466027.1 | cscsaaugaaAfCfAfucucuucccuL96 | 2633 | asdGsggdAadGagaudGuUfucauuggsasu | 2751 |
| AD-1466034.1 | gsasccgaagUfCfAfcaaguccuuuL96 | 2634 | asdAsagdGadCuugudGaCfuucggucsasu | 2752 |
| AD-1466039.1 | ususcuacccUfUfCfugaaucuaguL96 | 2635 | asdCsuadGadTucagdAaGfgguagaasusa | 2753 |
| AD-1466040.1 | csasucuccuAfCfUfcucaaugauuL96 | 2636 | asdAsucdAudTgagadGuAfggagaugsasa | 2754 |
| AD-1466043.1 | ususuaauccAfCfUfgguuauaguuL96 | 2637 | asdAscudAudAaccadGuGfgauuaaasusu | 2755 |
| AD-1466044.1 | ususaauccaCfUfGfguuauaguguL96 | 2638 | asdCsacdTadTaaccdAgUfggauuaasasu | 2756 |
| AD-1466046.1 | asusgguacaGfAfUfuacauugaguL96 | 2639 | asdCsucdAadTguaadTcUfguaccauscsu | 2757 |
| AD-1466047.1 | ascsugauguUfAfGfgacaaacauuL96 | 2640 | asdAsugdTudTguccdTaAfcaucagususu | 2758 |
| AD-1466050.1 | usgsaagacuCfUfGfaugauauucuL96 | 2641 | asdGsaadTadTcaucdAgAfgucuucasasu | 2759 |
| AD-1466051.1 | gsusaugaagAfGfCfaucucggaauL96 | 2642 | asdTsucdCgdAgaugdCuCfuucauacsusc | 2760 |
| AD-1466052.1 | asgsagcaucUfCfGfgaauucuuguL96 | 2643 | asdCsaadGadAuuccdGaGfaugcucususc | 2761 |
| AD-1466055.1 | asasuucuugGfUfCfcuauuaucauL96 | 2644 | asdTsgadTadAuaggdAcCfaagaauuscsc | 2762 |
| AD-1466058.1 | usgsaaguggAfUfGfauguuauccuL96 | 2645 | asdGsgadTadAcaucdAuCfcacuucasgsc | 2763 |
| AD-1466059.1 | gsasaguggaUfGfAfuguuauccauL96 | 2646 | asdTsggdAudAacaudCaUfccacuucsasg | 2764 |
| AD-1466060.1 | asuscagaggGfAfAfagacuuauguL96 | 2647 | asdCsaudAadGucuudTcCfcucugausgsa | 2765 |
| AD-1466062.1 | asgsccaaauAfGfCfaguuauaccuL96 | 2648 | asdGsgudAudAacugdCuAfuuuggcusgsa | 2766 |
| AD-1466063.1 | asgscaguuaUfAfCfcuacguauguL96 | 2649 | asdCsaudAcdGuaggdTaUfaacugcusasu | 2767 |
| AD-1466070.1 | gsasgagaauUfUfGfucuuacuauuL96 | 2650 | asdAsuadGudAagacdAaAfuucucucsasu | 2768 |
| AD-1466081.1 | gsasaaagaaGfAfGfcugguacuauL96 | 2651 | asdTsagdTadCcagcdTcUfucuuuucsasu | 2769 |
| AD-1466083.1 | asasagaagaGfCfUfgguacuauguL96 | 2652 | asdCsaudAgdTaccadGcUfcuucuuususc | 2770 |
| AD-1466085.1 | asgsaagagcUfGfGfuacuaugaauL96 | 2653 | asdTsucdAudAguacdCaGfcucuucususu | 2771 |
| AD-1466089.1 | gsasgcugguAfCfUfaugaaaagauL96 | 2654 | asdTscudTudTcauadGuAfccagcucsusu | 2772 |
| AD-1466091.1 | gscsugguacUfAfUfgaaaagaaguL96 | 2655 | asdCsuudCudTuucadTaGfuaccagcsusc | 2773 |
| AD-1466095.1 | ususucacgcCfAfUfuaaugggauuL96 | 2656 | asdAsucdCcdAuuaadTgGfcgugaaascsu | 2774 |
| AD-1466096.1 | asusuaauggGfAfUfgaucuacaguL96 | 2657 | asdCsugdTadGaucadTcCfcauuaausgsg | 2775 |
| AD-1466097.1 | gscsucccaaGfAfCfauucacguguL96 | 2658 | asdCsacdGudGaaugdTcUfugggagcscsg | 2776 |
| AD-1466100.1 | asusgcaaacGfCfCfauuucuuauuL96 | 2659 | asdAsuadAgdAaaugdGcGfuuugcauscsc | 2777 |
| AD-1466101.1 | gscsaaacgcCfAfUfuucuuaucauL96 | 2660 | asdTsgadTadAgaaadTgGfcguuugcsasu | 2778 |
| AD-1466104.1 | usasagcacuGfGfUfaucauaucuuL96 | 2661 | asdAsgadTadTgauadCcAfgugcuuasgsu | 2779 |
| AD-1466108.1 | asasacaaugGfUfGfgaucuuauauL96 | 2662 | asdTsaudAadGauccdAcCfauuguuusasa | 2780 |
| AD-1466109.1 | csasauggugGfAfUfcuuauaauguL96 | 2663 | asdCsaudTadTaagadTcCfaccauugsusu | 2781 |
| AD-1466110.1 | gsgsuggaucUfUfAfuaaugcuuguL96 | 2664 | asdCsaadGcdAuuaudAaGfauccaccsasu | 2782 |
| AD-1466111.1 | asuscuuauaAfUfGfcuuggaguguL96 | 2665 | asdCsacdTcdCaagcdAuUfauaagauscsc | 2783 |
| AD-1466115.1 | usasuaccacAfGfAfguucuauguuL96 | 2666 | asdAscadTadGaacudCuGfugguauasgsc | 2784 |
| AD-1466116.1 | usasccacagAfGfUfucuauguaguL96 | 2667 | asdCsuadCadTagaadCuCfugugguasusa | 2785 |
| AD-1466118.1 | csascagaguUfCfUfauguagcuuuL96 | 2668 | asdAsagdCudAcauadGaAfcucugugsgsu | 2786 |
| AD-1466120.1 | asgsuucuauGfUfAfgcuuacaguuL96 | 2669 | asdAscudGudAagcudAcAfuagaacuscsu | 2787 |
| AD-1466126.1 | uscsaguuugAfCfCfcaccuauuguL96 | 2670 | asdCsaadTadGguggdGuCfaaacugasusu | 2788 |
| AD-1466127.1 | csasguuugaCfCfCfaccuauuguuL96 | 2671 | asdAscadAudAggugdGgUfcaaacugsasu | 2789 |
| AD-1466128.1 | csusauugugGfCfUfagauauauuuL96 | 2672 | asdAsaudAudAucuadGcCfacaauagsgsu | 2790 |
| AD-1466131.1 | asgscaaaucAfCfAfgcuucuucguL96 | 2673 | asdCsgadAgdAagcudGuGfauuugcususg | 2791 |
| AD-1466133.1 | asasauugauCfUfAfcucaagaucuL96 | 2674 | asdGsaudCudTgagudAgAfucaauuuscsu | 2792 |
| AD-1466139.1 | uscscaugguGfGfAfcaagauuuuuL96 | 2675 | asdAsaadAudCuugudCcAfccauggasgsg | 2793 |
| AD-1466140.1 | cscsauggugGfAfCfaagauuuuuuL96 | 2676 | asdAsaadAadTcuugdTcCfaccauggsasg | 2794 |
| AD-1466141.1 | csasugguggAfCfAfagauuuuuguL96 | 2677 | asdCsaadAadAucuudGuCfcaccaugsgsa | 2795 |
| AD-1466142.1 | asusgguggaCfAfAfgauuuuugauL96 | 2678 | asdTscadAadAaucudTgUfccaccausgsg | 2796 |
| AD-1466143.1 | usgsguggacAfAfGfauuuuugaauL96 | 2679 | asdTsucdAadAaaucdTuGfuccaccasusg | 2797 |
| AD-1466144.1 | gsgsuggacaAfGfAfuuuuugaaguL96 | 2680 | asdCsuudCadAaaaudCuUfguccaccsasu | 2798 |
| AD-1466145.1 | gsusggacaaGfAfUfuuuugaagguL96 | 2681 | asdCscudTcdAaaaadTcUfuguccacscsa | 2799 |
| AD-1466152.1 | asgsauuuuuGfAfAfggaaauacuuL96 | 2682 | asdAsgudAudTuccudTcAfaaaaucususg | 2800 |
| AD-1466153.1 | gsasuuuuugAfAfGfgaaauacuauL96 | 2683 | asdTsagdTadTuuccdTuCfaaaaaucsusu | 2801 |
| AD-1466157.1 | ususugaaggAfAfAfuacuaauacuL96 | 2684 | asdGsuadTudAguaudTuCfcuucaaasasa | 2802 |
| AD-1466158.1 | ususgaaggaAfAfUfacuaauaccuL96 | 2685 | asdGsgudAudTaguadTuUfccuucaasasa | 2803 |
| AD-1466160.1 | csusaauaccAfAfAfggacauguguL96 | 2686 | asdCsacdAudGuccudTuGfguauuagsusa | 2804 |
| AD-1466161.1 | usasauaccaAfAfGfgacaugugauL96 | 2687 | asdTscadCadTguccdTuUfgguauuasgsu | 2805 |
| AD-1466162.1 | csasaucauuUfCfCfagguuuaucuL96 | 2688 | asdGsaudAadAccugdGaAfaugauugsgsg | 2806 |
| AD-1466163.1 | asuscauuucCfAfGfguuuauccguL96 | 2689 | asdCsggdAudAaaccdTgGfaaaugaususg | 2807 |

TABLE 20
Exemplary dsRNA molecules

| | | SEQ | | SEQ |
|---|---|---|---|---|
| | | ID | | ID |
| Duplex ID | Sense sequence (5′→3′) | NO: | Antisense sequence (5′→3′) | NO: |
| AD-1395757.1 | csasggu(Chd)cuCfAfCfuuuaauccsusa | 2808 | VPusdAsggdAudTaaagdTgdAgdGaccugscsg | 2811 |
| AD-1395758.1 | csasggu(Chd)cuCfaCfUfuuaauccsusa | 2809 | VPusdAsggdAudTaaagdTgdAgdGaccugscsg | 2811 |
| AD-1395763.1 | csascuu(Uhd)aaUfCfCfucuauccasgsa | 2810 | VPusdCsugdGadTagagdGadTudAaagugsasg | 2812 |
| AD-1395756.1 | csasggu(Chd)cuCfAfCfuuuaauccsusa | 2808 | VPusdAsggdAudTaaagdTgAfggaccugscsg | 2813 |
| AD-1395762.1 | csascuu(Uhd)aaUfCfCfucuauccasgsa | 2810 | VPusdCsugdGadTagagdGaUfuaaagugsasg | 2814 |

TABLE 21
Exemplary dsRNA molecules

| | | SEQ | | SEQ |
|---|---|---|---|---|
| | | ID | | ID |
| Duplex ID | Sense sequence (5′→3′) | NO: | Antisense sequence (5′→3′) | NO: |
| AD-1331279.1 | asasuguucaCfAfAfuuaagcuccuL96 | 2815 | asdGsgadGcdTuaaudTgUfgaacauususu | 2839 |
| AD-1331280.1 | asusuugcuaUfGfUfuagacgauguL96 | 2816 | asdCsaudCgdTcuaadCaUfagcaaauscsu | 2840 |
| AD-1331281.1 | ususgcuaugUfUfAfgacgauguaaL96 | 2817 | usdTsacdAudCgucudAaCfauagcaasasu | 2841 |
| AD-1331282.1 | usgscuauguUfAfGfacgauguaaaL96 | 2818 | usdTsuadCadTcgucdTaAfcauagcasasa | 2842 |
| AD-1331283.1 | asascugagaAfGfAfacuacauauaL96 | 2819 | usdAsuadTgdTaguudCuUfcucaguuscsc | 2843 |
| AD-1331284.1 | asasccaacaGfCfAfuagucaaauaL96 | 2820 | usdAsuudTgdAcuaudGcUfguugguususa | 2844 |
| AD-1331285.1 | cscscacagaAfAfUfuucucuaucuL96 | 2821 | asdGsaudAgdAgaaadTuUfcugugggsusu | 2845 |
| AD-1331286.1 | csasgguaguCfCfAfuggacauuaaL96 | 2822 | usdTsaadTgdTccaudGgAfcuaccugsasu | 2846 |
| AD-1331287.1 | gsgsuaguccAfUfGfgacauuaauuL96 | 2823 | asdAsuudAadTguccdAuGfgacuaccsusg | 2847 |
| AD-1331288.1 | asgsuuggaaGfAfCfuggaaagacaL96 | 2824 | usdGsucdTudTccagdTcUfuccaacuscsa | 2848 |
| AD-1331289.1 | usgsgaaagaCfAfAfcaaacauuauL96 | 2825 | asdTsaadTgdTuugudTgUfcuuuccasgsu | 2849 |
| AD-1331290.1 | ususuacuugGfGfAfaaucacgaaaL96 | 2826 | usdTsucdGudGauuudCcCfaaguaaasasa | 2850 |
| AD-1331291.1 | gsgsgaaaucAfCfGfaaaccaacuaL96 | 2827 | usdAsgudTgdGuuucdGuGfauuucccsasa | 2851 |
| AD-1331292.1 | gsasaaucacGfAfAfaccaacuauaL96 | 2828 | usdAsuadGudTgguudTcGfugauuucscsc | 2852 |
| AD-1331293.1 | csgsaaaccaAfCfUfauacgcuacaL96 | 2829 | usdGsuadGcdGuauadGuUfgguuucgsusg | 2853 |
| AD-1331294.1 | asuscaaccaAfAfAfuguugauccaL96 | 2830 | usdGsgadTcdAacaudTuUfgguugaususu | 2854 |
| AD-1331295.1 | ususaaaacuCfUfAfaacuugacuaL96 | 2831 | usdAsgudCadAguuudTgAfguuuuaascsa | 2855 |
| AD-1331296.1 | csasaaacuuGfAfAfagccuccuauL96 | 2832 | asdTsagdGadGgcuudTcAfaguuuugsasg | 2856 |
| AD-1331297.1 | uscsaacaucGfAfAfuagauggauuL96 | 2833 | asdAsucdCadTcuaudTcGfauguugasasu | 2857 |
| AD-1331298.1 | csasaaacuuCfAfAfugaaacguguL96 | 2834 | asdCsacdGudTucaudTgAfaguuuugsusg | 2858 |
| AD-1331299.1 | asasucacgaAfAfCfcaacuauacuL96 | 2835 | asdGsuadTadGuuggdTuUfcgugauususc | 2859 |
| AD-1331300.1 | gsgsgaaucaAfUfUfuuagaugguuL96 | 2836 | asdAsccdAudCuaaadAuUfgauucccsasc | 2860 |
| AD-1331301.1 | csasaaauguUfGfAfuccauccaauL96 | 2837 | asdTsugdGadTggaudCaAfcauuuugsgsu | 2861 |
| AD-1331302.1 | usgsgacauuAfAfUfucaacaucgaL96 | 2838 | usdCsgadTgdTugaadTuAfauguccasusg | 2862 |
| AD-1331328.1 | asasuguucaCfAfAfuuaagcuccuL96 | 2815 | asdGsgadGcdTuaaudTgdTgdAacauususu | 2863 |
| AD-1331329.1 | asusuugcuaUfGfUfuagacgauguL96 | 2816 | asdCsaudCgdTcuaadCadTadGcaaauscsu | 2864 |
| AD-1331330.1 | ususgcuaugUfUfAfgacgauguaaL96 | 2817 | usdTsacdAudCgucudAadCadTagcaasasu | 2865 |
| AD-1331306.1 | usgscuauguUfAfGfacgauguaaaL96 | 2818 | usdTsuadCadTcgucdTadAcdAuagcasasa | 2866 |
| AD-1331331.1 | asascugagaAfGfAfacuacauauaL96 | 2819 | usdAsuadTgdTaguudCudTcdTcaguuscsc | 2867 |
| AD-1331332.1 | asasccaacaGfCfAfuagucaaauaL96 | 2820 | usdAsuudTgdAcuaudGcdTgdTugguususa | 2868 |
| AD-1331333.1 | cscscacagaAfAfUfuucucuaucuL96 | 2821 | asdGsaudAgdAgaaadTudTcdTgugggsusu | 2869 |
| AD-1331334.1 | csasgguaguCfCfAfuggacauuaaL96 | 2822 | usdTsaadTgdTccaudGgdAcdTaccugsasu | 2870 |
| AD-1331311.1 | gsgsuaguccAfUfGfgacauuaauuL96 | 2823 | asdAsuudAadTguccdAudGgdAcuaccsusg | 2871 |
| AD-1331335.1 | asgsuuggaaGfAfCfuggaaagacaL96 | 2824 | usdGsucdTudTccagdTcdTudCcaacuscsa | 2872 |
| AD-1331336.1 | usgsgaaagaCfAfAfcaaacauuauL96 | 2825 | asdTsaadTgdTuugudTgdTcdTuuccasgsu | 2873 |
| AD-1331314.1 | ususuacuugGfGfAfaaucacgaaaL96 | 2826 | usdTsucdGudGauuudCcdCadAguaaasasa | 2874 |
| AD-1331337.1 | gsgsgaaaucAfCfGfaaaccaacuaL96 | 2827 | usdAsgudTgdGuuucdGudGadTuucccsasa | 2875 |
| AD-1331316.1 | gsasaaucacGfAfAfaccaacuauaL96 | 2828 | usdAsuadGudTgguudTcdGudGauuucscsc | 2876 |
| AD-1331338.1 | csgsaaaccaAfCfUfauacgcuacaL96 | 2829 | usdGsuadGcdGuauadGudTgdGuuucgsusg | 2877 |
| AD-1331339.1 | asuscaaccaAfAfAfuguugauccaL96 | 2830 | usdGsgadTcdAacaudTudTgdGuugaususu | 2878 |
| AD-1331340.1 | ususaaaacuCfUfAfaacuugacuaL96 | 2831 | usdAsgudCadAguuudTgdAgdTuuuaascsa | 2879 |
| AD-1331320.1 | csasaaacuuGfAfAfagccuccuauL96 | 2832 | asdTsagdGadGgcuudTcdAadGuuuugsasg | 2880 |
| AD-1331341.1 | uscsaacaucGfAfAfuagauggauuL96 | 2833 | asdAsucdCadTcuaudTcdGadTguugasasu | 2881 |
| AD-1331322.1 | csasaaacuuCfAfAfugaaacguguL96 | 2834 | asdCsacdGudTucaudTgdAadGuuuugsusg | 2882 |
| AD-1331342.1 | asasucacgaAfAfCfcaacuauacuL96 | 2835 | asdGsuadTadGuuggdTudTcdGugauususc | 2883 |
| AD-1331343.1 | gsgsgaaucaAfUfUfuuagaugguuL96 | 2836 | asdAsccdAudCuaaadAudTgdAuucccsasc | 2884 |
| AD-1331325.1 | csasaaauguUfGfAfuccauccaauL96 | 2837 | asdTsugdGadTggaudCadAcdAuuuugsgsu | 2885 |
| AD-1331344.1 | usgsgacauuAfAfUfucaacaucgaL96 | 2838 | usdCsgadTgdTugaadTudAadTguccasusg | 2886 |

TABLE 22
Exemplary dsRNA molecules

| | | SEQ | | ISEQ |
|---|---|---|---|---|
| | | ID | | ID |
| Duplex ID | Sense sequence (5′→3′) | NO: | Antisense sequence (5′→3′) | NO: |
| AD-1554875 | gscscugugaGfGfAfcuccaagaguL96 | 2887 | asdCsucdTudGgagudCcUfcacaggcscsu | 3013 |
| AD-1554909 | gsgsugcuacUfCfUfgguauuuccuL96 | 2888 | asdGsgadAadTaccadGaGfuagcaccscsc | 3014 |
| AD-1554910 | gsusgcuacuCfUfGfguauuuccuuL96 | 2889 | asdAsggdAadAuaccdAgAfguagcacscsc | 3015 |
| AD-1554911 | usgscuacucUfGfGfuauuuccuauL96 | 2890 | asdTsagdGadAauacdCaGfaguagcascsc | 3016 |
| AD-1554912 | gscsuacucuGfGfUfauuuccuaguL96 | 2891 | asdCsuadGgdAaauadCcAfgaguagcsasc | 3017 |
| AD-1554913 | csusacucugGfUfAfuuuccuagguL96 | 2892 | asdCscudAgdGaaaudAcCfagaguagscsa | 3018 |
| AD-1554914 | usascucuggUfAfUfuuccuaggguL96 | 2893 | asdCsccdTadGgaaadTaCfcagaguasgsc | 3019 |
| AD-1554915 | ascsucugguAfUfUfuccuaggguuL96 | 2894 | asdAsccdCudAggaadAuAfccagagusasg | 3020 |
| AD-1554916 | csuscugguaUfUfUfccuaggguauL96 | 2895 | asdTsacdCcdTaggadAaUfaccagagsusa | 3021 |
| AD-1554917 | uscsugguauUfUfCfcuaggguacuL96 | 2896 | asdGsuadCcdCuaggdAaAfuaccagasgsu | 3022 |
| AD-1554923 | asusuuccuaGfGfGfuacaaggcguL96 | 2897 | asdCsgcdCudTguacdCcUfaggaaausasc | 3023 |
| AD-1554951 | gsgsucagccAfGfGfuguacucaguL96 | 2898 | asdCsugdAgdTacacdCuGfgcugaccsasu | 3024 |
| AD-1554955 | asgsccagguGfUfAfcucaggcaguL96 | 2899 | asdCsugdCcdTgagudAcAfccuggcusgsa | 3025 |
| AD-1554992 | gscscacuucUfCfCfcaggaucuuuL96 | 2900 | asdAsagdAudCcuggdGaGfaaguggcsgsa | 3026 |
| AD-1554997 | ususcucccaGfGfAfucuuacccguL96 | 2901 | asdCsggdGudAagaudCcUfgggagaasgsu | 3027 |
| AD-1555000 | uscsccaggaUfCfUfuacccgccguL96 | 2902 | asdCsggdCgdGguaadGaUfccugggasgsa | 3028 |
| AD-1555030 | gscscuuccgCfAfGfugaaaccgcuL96 | 2903 | asdGscgdGudTucacdTgCfggaaggcsasc | 3029 |
| AD-1555106 | csasacuccaGfCfUfccgucuauuuL96 | 2904 | asdAsaudAgdAcggadGcUfggaguugsusa | 3030 |
| AD-1555112 | csasgcuccgUfCfUfauuccuuuguL96 | 2905 | asdCsaadAgdGaauadGaCfggagcugsgsa | 3031 |
| AD-1555114 | csuscaccugCfUfUfcuucugguuuL96 | 2906 | asdAsacdCadGaagadAgCfaggugagsgsg | 3032 |
| AD-1555115 | uscsaccugcUfUfCfuucugguucuL96 | 2907 | asdGsaadCcdAgaagdAaGfcaggugasgsg | 3033 |
| AD-1555117 | ascscugcuuCfUfUfcugguucauuL96 | 2908 | asdAsugdAadCcagadAgAfagcaggusgsa | 3034 |
| AD-1555118 | cscsugcuucUfUfCfugguucauuuL96 | 2909 | asdAsaudGadAccagdAaGfaagcaggsusg | 3035 |
| AD-1555120 | usgscuucuuCfUfGfguucauucuuL96 | 2910 | asdAsgadAudGaaccdAgAfagaagcasgsg | 3036 |
| AD-1555121 | gscsuucuucUfGfGfuucauucucuL96 | 2911 | asdGsagdAadTgaacdCaGfaagaagcsasg | 3037 |
| AD-1555122 | csusucuucuGfGfUfucauucuccuL96 | 2912 | asdGsgadGadAugaadCcAfgaagaagscsa | 3038 |
| AD-1555123 | ususcuucugGfUfUfcauucuccauL96 | 2913 | asdTsggdAgdAaugadAcCfagaagaasgsc | 3039 |
| AD-1555128 | csusgguucaUfUfCfuccaaauccuL96 | 2914 | asdGsgadTudTggagdAaUfgaaccagsasa | 3040 |
| AD-1555184 | ascsagggccGfAfGfuacgaaguguL96 | 2915 | asdCsacdTudCguacdTcGfgcccugusasg | 3041 |
| AD-1555185 | csasgggccgAfGfUfacgaagugguL96 | 2916 | asdCscadCudTcguadCuCfggcccugsusa | 3042 |
| AD-1555212 | cscsagugugAfAfAfgacauagcuuL96 | 2917 | asdAsgcdTadTgucudTuCfacacuggscsu | 3043 |
| AD-1555213 | csasgugugaAfAfGfacauagcuguL96 | 2918 | asdCsagdCudAugucdTuUfcacacugsgsc | 3044 |
| AD-1555234 | asusugaauuCfCfAfcgcuggguuuL96 | 2919 | asdAsacdCcdAgcgudGgAfauucaausgsc | 3045 |
| AD-1555235 | ususgaauucCfAfCfgcuggguuguL96 | 2920 | asdCsaadCcdCagcgdTgGfaauucaasusg | 3046 |
| AD-1555236 | usgsaauuccAfCfGfcuggguuguuL96 | 2921 | asdAscadAcdCcagcdGuGfgaauucasasu | 3047 |
| AD-1555238 | asasuuccacGfCfUfggguuguuauL96 | 2922 | asdTsaadCadAcccadGcGfuggaauuscsa | 3048 |
| AD-1555241 | uscscacgcuGfGfGfuuguuaccguL96 | 2923 | asdCsggdTadAcaacdCcAfgcguggasasu | 3049 |
| AD-1555242 | cscsacgcugGfGfUfuguuaccgcuL96 | 2924 | asdGscgdGudAacaadCcCfagcguggsasa | 3050 |
| AD-1555243 | csascgcuggGfUfUfguuaccgcuuL96 | 2925 | asdAsgcdGgdTaacadAcCfcagcgugsgsa | 3051 |
| AD-1555247 | csusggguugUfUfAfccgcuacaguL96 | 2926 | asdCsugdTadGcggudAaCfaacccagscsg | 3052 |
| AD-1555342 | gsgsgaccgaCfUfGfgccauguauuL96 | 2927 | asdAsuadCadTggccdAgUfcggucccsgsg | 3053 |
| AD-1555343 | gsgsaccgacUfGfGfccauguauguL96 | 2928 | asdCsaudAcdAuggcdCaGfucgguccscsg | 3054 |
| AD-1555345 | ascscgacugGfCfCfauguaugacuL96 | 2929 | asdGsucdAudAcaugdGcCfagucgguscsc | 3055 |
| AD-1555346 | cscsgacuggCfCfAfuguaugacguL96 | 2930 | asdCsgudCadTacaudGgCfcagucggsusc | 3056 |
| AD-1555348 | gsascuggccAfUfGfuaugacguguL96 | 2931 | asdCsacdGudCauacdAuGfgccagucsgsg | 3057 |
| AD-1555349 | ascsuggccaUfGfUfaugacgugguL96 | 2932 | asdCscadCgdTcauadCaUfggccaguscsg | 3058 |
| AD-1555350 | csusggccauGfUfAfugacguggcuL96 | 2933 | asdGsccdAcdGucaudAcAfuggccagsusc | 3059 |
| AD-1555366 | asgsgcucauCfAfCfcucgguguauL96 | 2934 | asdTsacdAcdCgaggdTgAfugagccuscsu | 3060 |
| AD-1555428 | gscscugcacAfGfCfuacuacgacuL96 | 2935 | asdGsucdGudAguagdCuGfugcaggcscsc | 3061 |
| AD-1555429 | cscsugcacaGfCfUfacuacgaccuL96 | 2936 | asdGsgudCgdTaguadGcUfgugcaggscsc | 3062 |
| AD-1555535 | cscsucucugGfAfCfuacggcuuguL96 | 2937 | asdCsaadGcdCguagdTcCfagagaggsgsc | 3063 |
| AD-1555537 | uscsucuggaCfUfAfcggcuuggcuL96 | 2938 | asdGsccdAadGccgudAgUfccagagasgsg | 3064 |
| AD-1555546 | usascggcuuGfGfCfccucugguuuL96 | 2939 | asdAsacdCadGagggdCcAfagccguasgsu | 3065 |
| AD-1555547 | ascsggcuugGfCfCfcucugguuuuL96 | 2940 | asdAsaadCcdAgaggdGcCfaagccgusasg | 3066 |
| AD-1555548 | csgsgcuuggCfCfCfucugguuuguL96 | 2941 | asdCsaadAcdCagagdGgCfcaagccgsusa | 3067 |
| AD-1555549 | gsgscuuggcCfCfUfcugguuugauL96 | 2942 | asdTscadAadCcagadGgGfccaagccsgsu | 3068 |
| AD-1555581 | gsasggaggcAfGfAfaguaugauuuL96 | 2943 | asdAsaudCadTacuudCuGfccuccucsasg | 3069 |
| AD-1555583 | gsgsaggcagAfAfGfuaugauuuguL96 | 2944 | asdCsaadAudCauacdTuCfugccuccsusc | 3070 |
| AD-1555584 | gsasggcagaAfGfUfaugauuugcuL96 | 2945 | asdGscadAadTcauadCuUfcugccucscsu | 3071 |
| AD-1555585 | asgsgcagaaGfUfAfugauuugccuL96 | 2946 | asdGsgcdAadAucaudAcUfucugccuscsc | 3072 |
| AD-1555586 | gsgscagaagUfAfUfgauuugccguL96 | 2947 | asdCsggdCadAaucadTaCfuucugccsusc | 3073 |
| AD-1555587 | gscsagaaguAfUfGfauuugccguuL96 | 2948 | asdAscgdGcdAaaucdAuAfcuucugcscsu | 3074 |
| AD-1555588 | csasgaaguaUfGfAfuuugccguguL96 | 2949 | asdCsacdGgdCaaaudCaUfacuucugscsc | 3075 |
| AD-1555589 | asgsaaguauGfAfUfuugccgugcuL96 | 2950 | asdGscadCgdGcaaadTcAfuacuucusgsc | 3076 |
| AD-1555590 | gsasaguaugAfUfUfugccgugcauL96 | 2951 | asdTsgcdAcdGgcaadAuCfauacuucsusg | 3077 |
| AD-1555615 | csasguggacGfAfUfccagaacaguL96 | 2952 | asdCsugdTudCuggadTcGfuccacugsgsc | 3078 |
| AD-1555616 | asgsuggacgAfUfCfcagaacagguL96 | 2953 | asdCscudGudTcuggdAuCfguccacusgsg | 3079 |
| AD-1555626 | cscsagaacaGfGfAfggcuguguguL96 | 2954 | asdCsacdAcdAgccudCcUfguucuggsasu | 3080 |
| AD-1555628 | asgsaacaggAfGfGfcuguguggcuL96 | 2955 | asdGsccdAcdAcagcdCuCfcuguucusgsg | 3081 |
| AD-1555706 | usgsugcgggUfGfCfacuauggcuuL96 | 2956 | asdAsgcdCadTagugdCaCfccgcacascsc | 3082 |
| AD-1555707 | gsusgcggguGfCfAfcuauggcuuuL96 | 2957 | asdAsagdCcdAuagudGcAfcccgcacsasc | 3083 |
| AD-1555709 | gscsgggugcAfCfUfauggcuuguuL96 | 2958 | asdAscadAgdCcauadGuGfcacccgcsasc | 3084 |
| AD-1555711 | gsgsgugcacUfAfUfggcuuguacuL96 | 2959 | asdGsuadCadAgccadTaGfugcacccsgsc | 3085 |
| AD-1555717 | ascsuauggcUfUfGfuacaaccaguL96 | 2960 | asdCsugdGudTguacdAaGfccauagusgsc | 3086 |
| AD-1555723 | gscsuuguacAfAfCfcagucggacuL96 | 2961 | asdGsucdCgdAcuggdTuGfuacaagcscsa | 3087 |
| AD-1555725 | csusgcccugGfAfGfaguuccucuuL96 | 2962 | asdAsgadGgdAacucdTcCfagggcagsgsg | 3088 |
| AD-1555768 | gscscuggauGfAfGfagaaacugcuL96 | 2963 | asdGscadGudTucucdTcAfuccaggcscsg | 3089 |
| AD-1555771 | usgsgaugagAfGfAfaacugcguuuL96 | 2964 | asdAsacdGcdAguuudCuCfucauccasgsg | 3090 |
| AD-1555772 | gsgsaugagaGfAfAfacugcguuuuL96 | 2965 | asdAsaadCgdCaguudTcUfcucauccsasg | 3091 |
| AD-1555776 | gsasgagaaaCfUfGfcguuugcaguL96 | 2966 | asdCsugdCadAacgcdAgUfuucucucsasu | 3092 |
| AD-1555789 | ususugcagaGfCfCfacauuccaguL96 | 2967 | asdCsugdGadAugugdGcUfcugcaaascsg | 3093 |
| AD-1555894 | gsusgggacaUfUfCfaccuuccaguL96 | 2968 | asdCsugdGadAggugdAaUfgucccacsasu | 3094 |
| AD-1555895 | usgsggacauUfCfAfccuuccaguuL96 | 2969 | asdAscudGgdAaggudGaAfugucccascsa | 3095 |
| AD-1555897 | gsgsacauucAfCfCfuuccaguguuL96 | 2970 | asdAscadCudGgaagdGuGfaauguccscsa | 3096 |
| AD-1555898 | gsascauucaCfCfUfuccaguguguL96 | 2971 | asdCsacdAcdTggaadGgUfgaaugucscsc | 3097 |
| AD-1555899 | ascsauucacCfUfUfccagugugauL96 | 2972 | asdTscadCadCuggadAgGfugaauguscsc | 3098 |
| AD-1555900 | csasuucaccUfUfCfcagugugaguL96 | 2973 | asdCsucdAcdAcuggdAaGfgugaaugsusc | 3099 |
| AD-1556052 | asuscgcugaCfCfGfcugggugauuL96 | 2974 | asdAsucdAcdCcagcdGgUfcagcgausgsa | 3100 |
| AD-1556057 | usgsaccgcuGfGfGfugauaacaguL96 | 2975 | asdCsugdTudAucacdCcAfgcggucasgsc | 3101 |
| AD-1556126 | csgsuguuccUfGfGfgcaagguguuL96 | 2976 | asdAscadCcdTugccdCaGfgaacacgsgsu | 3102 |
| AD-1556127 | gsusguuccuGfGfGfcaagguguguL96 | 2977 | asdCsacdAcdCuugcdCcAfggaacacsgsg | 3103 |
| AD-1556137 | gscsaaggugUfGfGfcagaacucguL96 | 2978 | asdCsgadGudTcugcdCaCfaccuugcscsc | 3104 |
| AD-1556139 | asasggugugGfCfAfgaacucgcguL96 | 2979 | asdCsgcdGadGuucudGcCfacaccuusgsc | 3105 |
| AD-1556163 | csusggagagGfUfGfuccuucaaguL96 | 2980 | asdCsuudGadAggacdAcCfucuccagsgsc | 3106 |
| AD-1556164 | usgsgagaggUfGfUfccuucaagguL96 | 2981 | asdCscudTgdAaggadCaCfcucuccasgsg | 3107 |
| AD-1556166 | gsasgaggugUfCfCfuucaagguguL96 | 2982 | asdCsacdCudTgaagdGaCfaccucucscsa | 3108 |
| AD-1556167 | asgsagguguCfCfUfucaaggugauL96 | 2983 | asdTscadCcdTugaadGgAfcaccucuscsc | 3109 |
| AD-1556319 | asuscccacaGfGfAfccugugcaguL96 | 2984 | asdCsugdCadCaggudCcUfgugggauscsa | 3110 |
| AD-1556359 | usgsacgccaCfGfCfaugcuguguuL96 | 2985 | asdAscadCadGcaugdCgUfggcgucascsc | 3111 |
| AD-1556360 | gsascgccacGfCfAfugcuguguguL96 | 2986 | asdCsacdAcdAgcaudGcGfuggcgucsasc | 3112 |
| AD-1556382 | gscsuaccgcAfAfGfggcaagaaguL96 | 2987 | asdCsuudCudTgcccdTuGfcgguagcscsg | 3113 |
| AD-1556383 | csusaccgcaAfGfGfgcaagaagguL96 | 2988 | asdCscudTcdTugccdCuUfgcgguagscsc | 3114 |
| AD-1556465 | gsgsccuaacUfAfCfuucggcgucuL96 | 2989 | asdGsacdGcdCgaagdTaGfuuaggccsgsg | 3115 |
| AD-1556466 | gscscuaacuAfCfUfucggcgucuuL96 | 2990 | asdAsgadCgdCcgaadGuAfguuaggcscsg | 3116 |
| AD-1556484 | csusacacccGfCfAfucacagguguL96 | 2991 | asdCsacdCudGugaudGcGfgguguagsasc | 3117 |
| AD-1556510 | gscsuggaucCfAfGfcaagugguguL96 | 2992 | asdCsacdCadCuugcdTgGfauccagcsusg | 3118 |
| AD-1556584 | usgsgcaggaGfGfUfggcaucuuguL96 | 2993 | asdCsaadGadTgccadCcUfccugccascsc | 3119 |
| AD-1556585 | gsgscaggagGfUfGfgcaucuuguuL96 | 2994 | asdAscadAgdAugccdAcCfuccugccsasc | 3120 |
| AD-1556586 | gscsaggaggUfGfGfcaucuugucuL96 | 2995 | asdGsacdAadGaugcdCaCfcuccugcscsa | 3121 |
| AD-1556587 | csasggagguGfGfCfaucuugucuuL96 | 2996 | asdAsgadCadAgaugdCcAfccuccugscsc | 3122 |
| AD-1556613 | usgsaugucuGfCfUfccagugauguL96 | 2997 | asdCsaudCadCuggadGcAfgacaucasgsg | 3123 |
| AD-1556677 | csasauucucUfCfUfccuccguccuL96 | 2998 | asdGsgadCgdGaggadGaGfagaauugsgsg | 3124 |
| AD-1556709 | gsgscucagcAfGfCfaagaaugcuuL96 | 2999 | asdAsgcdAudTcuugdCuGfcugagccsasc | 3125 |
| AD-1556710 | gscsucagcaGfCfAfagaaugcuguL96 | 3000 | asdCsagdCadTucuudGcUfgcugagcscsa | 3126 |
| AD-1556789 | csusggucuaAfCfUfugggaucuguL96 | 3001 | asdCsagdAudCccaadGuUfagaccagsgsg | 3127 |
| AD-1556790 | usgsgucuaaCfUfUfgggaucugguL96 | 3002 | asdCscadGadTcccadAgUfuagaccasgsg | 3128 |
| AD-1556791 | gsgsucuaacUfUfGfggaucuggguL96 | 3003 | asdCsccdAgdAucccdAaGfuuagaccsasg | 3129 |
| AD-1556795 | usasacuuggGfAfUfcugggaauguL96 | 3004 | asdCsaudTcdCcagadTcCfcaaguuasgsa | 3130 |
| AD-1556799 | ususgggaucUfGfGfgaauggaaguL96 | 3005 | asdCsuudCcdAuuccdCaGfaucccaasgsu | 3131 |
| AD-1556802 | gsgsaucuggGfAfAfuggaagguguL96 | 3006 | asdCsacdCudTccaudTcCfcagauccscsa | 3132 |
| AD-1556908 | usgsagcucaGfCfUfgcccuuugguL96 | 3007 | asdCscadAadGggcadGcUfgagcucascsc | 3133 |
| AD-1556909 | gsasgcucagCfUfGfcccuuuggauL96 | 3008 | asdTsccdAadAgggcdAgCfugagcucsasc | 3134 |
| AD-1556911 | gscsucagcuGfCfCfcuuuggaauuL96 | 3009 | asdAsuudCcdAaaggdGcAfgcugagcsusc | 3135 |
| AD-1556915 | asgscugcccUfUfUfggaauaaaguL96 | 3010 | asdCsuudTadTuccadAaGfggcagcusgsa | 3136 |
| AD-1556917 | csusgcccuuUfGfGfaauaaagcuuL96 | 3011 | asdAsgcdTudTauucdCaAfagggcagscsu | 3137 |
| AD-1556918 | usgscccuuuGfGfAfauaaagcuguL96 | 3012 | asdCsagdCudTuauudCcAfaagggcasgsc | 3138 |

TABLE 23
Exemplary dsRNA molecules

| | | SEQ | | SEQ |
|---|---|---|---|---|
| | | ID | | ID |
| Duplex ID | Sense sequence (5′→3′) | NO: | Antisense sequence(5′→3′) | NO: |
| AD- | ascsagacAfaGfAfCfcaucuacacuL96 | 3139 | asdGsugdTadGauggucUfudGucugusgsc | 3153 |
| 1181513.1 | | | | |
| AD- | cscsgagcCfgUfUfCfucuacaauuuL96 | 128 | asdAsaudTgdTagagaaCfgdGcucggsasu | 3154 |
| 1181527.1 | | | | |
| AD- | gsasugccAfaGfAfAfcacuaugauuL96 | 3140 | asdAsucdAudAguguucUfudGgcaucscsu | 3155 |
| 1181564.1 | | | | |
| AD- | ascsagagdAaaUfUfcuacuacauuL96 | 3141 | asdAsugdTadGuagaauUfuCfucugusgsg | 3156 |
| 1181612.1 | | | | |
| AD- | gsasaauuCfuaCfUfacaucuauauL96 | 3142 | asUfsaudAgdAuguadGudAgdAauuucsusc | 3157 |
| 1181618.1 | | | | |
| AD- | csgsagccGfuUfCfUfcuacaauuauL96 | 3143 | asUfsaadTudGuagagadAcdGgcucgsgsc | 3158 |
| 1181537.1 | | | | |
| AD- | csasagccUfuGfGfCfucaauaccauL96 | 3144 | asUfsggdTadTugagccdAadGgcuugsgsc | 3159 |
| 1181552.1 | | | | |
| AD- | asgsaaauUfcUfAfCfuacaucuauuL96 | 84 | asAfsuadGadTguagdTadGaAfuuucuscsu | 3160 |
| 1181601.1 | | | | |
| AD- | gsasgaaaUfuCfUfdAcuacaucuauL96 | 3145 | asUfsagdAudGuagudAgdAaUfuucucsusg | 3161 |
| 1181614.1 | | | | |
| AD- | asgsauccdGagCfCfuacuaugaauL96 | 3146 | asUfsucdAudAguaggcUfcdGgaucususc | 3162 |
| 1181524.1 | | | | |
| AD- | cscsgagcCfgUfUfCfucuacaauuuL96 | 128 | asAfsaudTgdTagagaaCfgdGcucggsasu | 3163 |
| 1181528.1 | | | | |
| AD- | cscsgagcCfgUfUfCfucuacaauuuL96 | 128 | asAfsaudTgdTagagaaCfgdGcucggsgsc | 3164 |
| 1181529.1 | | | | |
| AD- | asgsagaaAfuUfCfUfacuauaucuuL96 | 3147 | asAfsgadTadTaguagaAfuUfucucusgsu | 3165 |
| 1181598.1 | | | | |
| AD- | csasagguCfuUfCfUfcucuggcuguL96 | 3148 | asCfsagdCcdAgagagaAfgAfccuugsgsc | 3166 |
| 1181604.1 | | | | |
| AD- | cscsuacadGagAfAfauucuacuauL96 | 3149 | asUfsagdTadGaauuucUfcUfguaggscsu | 3167 |
| 1181609.1 | | | | |
| AD- | ascsagagAfaAfUfUfcuacuacauuL96 | 3150 | asAfsugdTadGuagaauUfuCfucugusgsg | 3168 |
| 1181611.1 | | | | |
| AD- | gsasaauuCfuAfCfUfacaucuauauL96 | 3151 | asUfsaudAgdAuguaguAfgAfauuucsusc | 3169 |
| 1181617.1 | | | | |
| AD- | ascsccuaCfuCfUfdGuuguucgaauL96 | 3152 | asUfsucdGadAcaacagAfgUfagggusgsg | 3170 |
| 1181619.1 | | | | |
| AD- | csasagguCfuUfCfUfcucuggcuguL96 | 3148 | asCfsagdCcdAgagagaAfgAfccuugsgsc | 3166 |
| 1181604.2 | | | | |

TABLE 24
Exemplary dsRNA molecules

| | | SEQ | | SEQ |
|---|---|---|---|---|
| | | ID | | ID |
| Duplex ID | Sense sequence (5′→3′) | NO: | Antisense sequence (5′→3′) | NO: |
| AD- | csusuaaaAfgGfGfAfcaguauucuaL96 | 3171 | usdAsgadAudAcuguccCfuUfuuaagscsc | 3179 |
| 1143243.1 | | | | |
| AD- | gscsuuaaAfaGfGfGfacaguauucuL96 | 3172 | asdGsaadTadCugucccUfuUfuaagcsasa | 3180 |
| 1143252.1 | | | | |
| AD- | gscsuuaaAfaGfGfGfacaguauucuL96 | 3172 | asdGsaadTadCugucccUfuUfuaagcsgsc | 3181 |
| 1143254.1 | | | | |
| AD- | csusuaaaAfgGfGfAfcaguauucuuL96 | 3173 | asdAsgadAudAcuguccCfuUfuuaagscsc | 3182 |
| 1143278.1 | | | | |
| AD- | usasaaAfgGfGfAfcaguauucuaL96 | 3174 | usdAsgadAudAcuguccCfuUfuuasasg | 3183 |
| 1143245.1 | | | | |
| AD- | ususaaAfaGfGfGfacaguauucuL96 | 3175 | asdGsaadTadCugucccUfuUfuaasgsc | 3184 |
| 1143256.1 | | | | |
| AD- | usasaaAfgGfGfAfcaguauucuuL96 | 3176 | asdAsgadAudAcuguccCfuUfuuasasg | 3185 |
| 1143280.1 | | | | |
| AD- | asasaaggGfaCfAfGfuauucucaguL96 | 3177 | asCfsugdAgdAauacugUfcCfcuuuusasa | 3186 |
| 1143286.1 | | | | |
| AD- | asasaaggGfaCfAfGfuauucucaguL96 | 3177 | asCfsugdAgdAauacugUfcCfcuuuusgsc | 3187 |
| 1143287.1 | | | | |
| AD- | asasaaggGfaCfAfGfuauucucaguL96 | 3177 | asCfsugdAgdAauacugUfcCfcuuuuscsc | 3188 |
| 1143288.1 | | | | |
| AD- | asasggGfaCfAfGfuauucucaguL96 | 3178 | asCfsugdAgdAauacugUfcCfcuususu | 3189 |
| 1143289.1 | | | | |

TABLE 25
Exemplary dsRNA molecules

| | | SEQ | | SEQ |
|---|---|---|---|---|
| | | ID | | ID |
| Duplex ID | Sense sequence (5′→3′) | NO: | Antisense sequence (5′→3′) | NO: |
| AD- | csusuc(Uhd)gGfuCfUfAfaacaaacusasa | 3190 | VPusUfsagdTudTguuuagAfcCfagaagsasu | 3212 |
| 1231468 | | | | |
| AD- | ususaau(Chd)UfcAfCfAfuaguaaucsusa | 3191 | VPusAfsgadTudAcuauguGfaGfauuaasasg | 3213 |
| 1231470 | | | | |
| AD- | usasacu(Uhd)UfaAfUfCfucauauagscsa | 3192 | VPusGfscudAudAugagauUfaAfaguuasasc | 3214 |
| 1231471 | | | | |
| AD- | cscsuga(Uhd)CfuUfCfUfgguuuaaascsa | 3193 | VPusGfsuudTadAaccagaAfgAfucaggsasa | 3215 |
| 1231472 | | | | |
| AD- | usgsua(Chd)aGfuAfAfGfugauaacasgsa | 3194 | VPusCfsugdTudAucacuuAfcUfguacasasg | 3216 |
| 1231473 | | | | |
| AD- | ususcgugGfuAfUfUfcuua(Chd)uagsusa | 3195 | VPusAfscudAgdTaagaauAfcCfacgaasasg | 3217 |
| 1231478 | | | | |
| AD- | uscsuuc(Uhd)GfgUfCfUfaaaugaacsusa | 3196 | VPusAfsgudTcdAuuuagaCfcAfgaagasusc | 3218 |
| 1231479 | | | | |
| AD- | uscsgug(Chd)UfaCfAfAfcuuucucasasa | 3197 | VPusUfsugdAgdAaaguugUfaGfcacgasusu | 3219 |
| 1231485 | | | | |
| AD | gsusugugUfaCfAfCfacau(Uhd)ggusasa | 3198 | VPusUfsacdCadAugugugUfaCfacaacsasu | 3220 |
| 1231492 | | | | |
| AD- | csgsugc(Uhd)AfcAfAfCfuucuucaasgsa | 3199 | VPusCfsuudGadAgaaguuGfuAfgcacgsasu | 3221 |
| 1231496 | | | | |
| AD- | usgsaaa(Uhd)UfgUfUfGfacauugugsasa | 3200 | VPusUfscadCadAugucaaCfaAfuuucasgsc | 3222 |
| 1231497 | | | | |
| AD- | ascsaaug(Uhd)uGfCfUfuuuuaaacsusa | 3201 | VPusAfsgudTudAaaaagcAfaCfauugususa | 3223 |
| 1231498 | | | | |
| AD- | asasauugUfuGfAfCfacua(Uhd)gagsusa | 3202 | VPusAfscudCadTagugucAfaCfaauuuscsa | 3224 |
| 1231499 | | | | |
| AD- | gscsugaaAfuUfGfUfugaua(Chd)ugsusa | 3203 | VPusAfscadGudAucaacaAfuUfucagcsasg | 3225 |
| 1231502 | | | | |
| AD- | usgsuugaCfaCfUfGfugaa(Uhd)gcususa | 3204 | VPusAfsagdCadTucacagUfgUfcaacasasu | 3226 |
| 1231505 | | | | |
| AD- | ascsauugCfcAfAfAfaggu(Uhd)ucusasa | 3205 | VPusUfsagdAadAccuuuuGfgCfaaugususg | 3227 |
| 1231507 | | | | |
| AD- | ususcug(Chd)UfaAfUfCfuuguugcusasa | 3206 | VPusUfsagdCadAcaagauUfaGfcagaasgsc | 3228 |
| 1231513 | | | | |
| AD- | asuscuugUfuUfUfCfucua(Uhd)ucasasa | 3207 | VPusUfsugdAadTagagaaAfaCfaagausgsa | 3229 |
| 1231519 | | | | |
| AD- | csasgucaUfaAfUfCfuaua(Uhd)uaasasa | 3208 | VPusUfsuudAadTauagauUfaUfgacugsusg | 3230 |
| 1231520 | | | | |
| AD- | usgsguaaCfaCfUfAfauaa(Uhd)aaasasa | 3209 | VPusUfsuudTadTuauuagUfgUfuaccascsa | 3231 |
| 1231521 | | | | |
| AD- | cscsucauGfgAfAfGfagaagcagauL96 | 3210 | asUfscudGcdTucucuuCfcAfugaggscsu | 3232 |
| 1523180.1 | | | | |
| AD- | cscsggu(Chd)AfgGfUfUfcugcuuuusasa | 3211 | VPusUfsaadAadGcagaacCfudGaccggscsc | 3233 |
| 1498528 | | | | |

All of the U.S. patents, U.S. patent application publications, foreign patents, foreign patent applications and non-patent publications referred to in this specification are incorporated herein by reference, in their entirety. Aspects of the embodiments can be modified, if necessary to employ concepts of the various patents, applications and publications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

1. A dsRNA agent comprising a sense strand and antisense, each strand independently having a length of 15-35 nucleotides wherein each nucleotide is independently modified or unmodified, wherein
the sense strand comprises a 2′-fluoro nucleotide at position 10, counting from 5′-end of the sense strand, and
the antisense strand comprises a 2′-deoxy nucleotide at positions 2, 5, 7 and 12, counting from 5′-end of the antisense strand, and
wherein:
(i) the antisense strand comprises a 2′-fluoro nucleotide at position 14 and a nucleotide other than a 2′-deoxy or 2′-fluoro nucleotide at position 16, counting from the 5′-end of the antisense strand; or
(ii) the antisense strand comprises a 2′-deoxy nucleotide at position 14 or 16, counting from the 5′-end of the antisense strand, and the sense strand comprises a nucleotide other than a 2′-fluoro nucleotide at position 7, counting from the 5′-end of the sense strand.

2. The dsRNA agent of claim 1, wherein the sense strand further comprises a 2′-fluoro nucleotide at position 9 or 11, counting from 5′-end of the sense strand.

3. (canceled)

4. The dsRNA of claim 1, wherein the sense strand further comprises a 2′-fluoro nucleotide at positions 9 and 11, counting from 5-end of the sense strand.

5. The dsRNA agent of claim 1, wherein the sense strand comprises a 2′-fluoro nucleotide at positions 8 and 9 or positions 11 and 12, counting from 5-end of the sense strand.

6. (canceled)

7. The dsRNA agent of claim 1, wherein the sense strand comprises a nucleotide other than a 2′-fluoro at position 7, counting from the 5′-end of the sense strand.

8. The dsRNA of claim 1, wherein the sense strand comprises a 2′-fluoro nucleotide at positions 9, 10 and 11, and a nucleotide other than a 2′-fluoro at position 7, counting from the 5′-end of the sense strand.

9. The dsRNA agent of claim 1, wherein the sense strand comprises at least one 2′-OMe nucleotide.

10. The dsRNA agent of claim 1, wherein the sense strand comprises a 2′-OMe nucleotide at position 7, counting from the 5′-end of the sense strand.

11. The dsRNA agent of claim 1, wherein the sense strand comprises a 2′-fluoro nucleotide at positions 9, 10 and 11, and a 2′-OMe nucleotide at position 7, counting from the 5′-end of the sense strand.

12. (canceled)

13. (canceled)

14. The dsRNA agent of claim 1, wherein the antisense strand comprises a 2′-OMe nucleotide at position 16, counting from the 5′-end of the antisense strand.

15. (canceled)

16. (canceled)

17. (canceled)

18. (canceled)

19. (canceled)

20. (canceled)

21. (canceled)

22. (canceled)

23. (canceled)

24. (canceled)

25. (canceled)

26. The dsRNA agent of claim 1, wherein the dsRNA agent comprises a ligand.

27. The dsRNA agent of claim 1, wherein the sense strand comprises a ligand.

28. The dsRNA agent of claim 27, wherein the ligand is at 3′-end of the sense strand.

29. The dsRNA agent of claim 27, wherein the ligand is at 5′-end of the sense strand.

30. The dsRNA agent of claim 27, wherein the ligand comprises an ASGPR ligand.

31. The dsRNA agent of claim 27, wherein the ligand is lipophilic group.

32. The dsRNA agent of claim 31, wherein the ligand is a C₁₀₋₃₀aliphatic group.

33. The dsRNA agent of claim 32, wherein the C₁₀₋₃₀aliphatic group is a C₁₀₋₃₀alkyl group.

34. The dsRNA agent of claim 33, wherein the C₁₀₋₃₀alkyl group is a straight-chain or branched tetradecyl, hexadecyl, octadecyl, icosyl, docosyl, or tetracosyl group.

35. The dsRNA agent of claim 23, wherein the ligand is conjugated to a non-terminal nucleotide of the sense strand.

36. The dsRNA agent of claim 35, wherein the ligand is conjugated to the 2′-position of a non-terminal nucleotide of the sense strand, optionally conjugated to one of positions 5, 6, 7, or 8 of the sense strand, counting from the 5′ end).

37. The dsRNA agent of claim 26, wherein the ligand comprises an abasic nucleotide, optionally the abasic nucleotide is an inverted nucleotide and linked via a 5′->5′ or a 3′->3′ linkage to a strand of the dsRNA agent.

38. (canceled)

39. (canceled)

40. (canceled)

41. (canceled)

42. (canceled)

43. (canceled)

44. The dsRNA agent of claim 1, wherein the sense strand comprises at least two phosphorothioate internucleotide linkages between the first five nucleotides counting from the 5′ end of the sense strand.

45. The dsRNA agent of claim 1, wherein the antisense strand comprises at least two phosphorothioate internucleotide linkages between the first five nucleotides counting from the 5′ end of the antisense strand and at least two phosphorothioate internucleotide linkages between the first five nucleotides counting from the 3′ end of the antisense strand.

46. The dsRNA agent of claim 1, wherein the dsRNA has a duplex region of from 18 to about 25 basepairs.

47. (canceled)

48. (canceled)

49. A dsRNA agent comprising a sense strand and an antisense strand,
wherein the sense strand is 18-23 nucleotides in length and comprises a 2′-fluoro nucleotide at position 10, counting from 5′-end of the sense strand and a 2′-fluoro nucleotide at position 9 or 11, counting from 5′-end of the sense strand, and the antisense strand is 18-25 nucleotide in length and comprises a 2′-deoxy nucleotide at positions 2, 5, 7, and 12, counting from 5′-end of the antisense strand,
wherein:
the antisense strand comprises a 2′-fluoro nucleotide at position 14 and a nucleotide other than a 2′-deoxy or 2′-fluoro nucleotide at position 16, counting from the 5′-end of the antisense strand.

50. The dsRNA agent of claim 49, wherein the sense strand comprises a 2′-fluoro nucleotide at positions 9, 10 and 11, counting from 5′-end of the sense strand.

51. The dsRNA agent of claim 1, comprising a phosphate mimic at the 5′-end of the antisense strand.

52. The dsRNA agent of claim 51, wherein the phosphate mimic is a 5′-E-vinyl phosphonate.

53. (canceled)

54. The dsRNA agent of claim 49, wherein remaining nucleotides in the sense strand are unmodified nucleotides or modified nucleotides, optionally selected from the groups consisting of 2′-OMe, 2′-F, 2′-H, and an 2′-O—C₁₀₋₃₀aliphatic group, provided no more than one modified nucleotide is an 2′-O—C₁₀₋₃₀aliphatic group; and the remaining nucleotides in the antisense strand are unmodified nucleotides or modified nucleotides, optionally selected from the group consisting of 2′-OMe, 2′-F, 2′-H, GNA and 3′-RNA, the 3′-RNA being optionally 3′-OH, provided no more than one modified nucleotide is GNA or 3′-RNA.

55. The dsRNA agent of claim 54, wherein remaining nucleotides (i.e., nucleotides at positions not otherwise defined) in the sense strand are modified nucleotides selected from the group consisting of 2′-OMe, 2′-F, 2′-H, and an 2′-O—C₁₀₋₃₀aliphatic group, provided no more than one modified nucleotide is an 2′-O—C₁₀₋₃₀aliphatic group; and the remaining nucleotides in the antisense strand are modified nucleotides selected from the group consisting of 2′-OMe, 2′-F, 2′-H, GNA, and 3′-RNA, the 3′-RNA being optionally 3′-OH, provided no more than one modified nucleotide is GNA or 3′-RNA.

56. (canceled)

57. (canceled)
